SEQUENCE LISTING (750 sequences)

SEQ ID NO: 1 - protein, 1483 aa, synthetic construct
  REGION 1..1483: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRADALDDFDLDMLGSDALDDFDLDMLGSDALDDFDLDMLGSDALDDFDLDMLINYPYDVPDYAS

SEQ ID NO: 2 - DNA, 350 bp, synthetic construct
  misc_feature 1..350: note = Description of Artificial Sequence: Synthetic polynucleotide
  source 1..350: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttatcgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttt

SEQ ID NO: 3 - DNA, 338 bp, Homo sapiens
  source 1..338: mol_type = genomic DNA
acccccggcagcagcccgccccgagcgcgccgcctgtttattcagccgggagtccggcacgcgccaggcgcacgcactgcaacaacaaacccagctgaatggagagtttgcaaggagcgggagaaaggaacgggagggggggagaggagaggaggagggggagtttagggagtgggtgggaggaagaggtaagaggaggggggggagtgggggctgcagccgctcgctgcagcagcggggagtggggggcgaggcggggccagggctgcgcgtggggctgggtgtcccattgaaaaggcggacgcactccggcagcccagcactctctcacttctggc

SEQ ID NO: 4 - DNA, 305 bp, Mus musculus
  source 1..305: mol_type = genomic DNA
ccgccccgagcgcgcggcctgtttattcagccgggagtccggcacgcgccgggcgcacgcactgcaacaacaaaccaggctgaatggagagtttgcaaggagcgggcgcgggcaactggagggggggggggcgagagggagggagctgaggaggtgggggaagaggaggggtagtgggggctgcagccgctcgctgcagcagcggggagtggggggcgaggcggggccagggctgcgcgtggggctgggtgtcccattgaaaaggcggccgcaccgcagccgcccagcagtctctcacttctggc

SEQ ID NO: 5 - DNA, 19 bp, synthetic construct
  misc_feature 1..19: note = Description of Artificial Sequence: Synthetic oligonucleotide
  source 1..19: mol_type = other DNA
gctgggtgtcccattgaaa

SEQ ID NO: 6 - DNA, 19 bp, synthetic construct
  misc_feature 1..19: note = Description of Artificial Sequence: Synthetic oligonucleotide
  source 1..19: mol_type = other DNA
cagccgctcgctgcagcag

SEQ ID NO: 7 - DNA, 19 bp, synthetic construct
  misc_feature 1..19: note = Description of Artificial Sequence: Synthetic oligonucleotide
  source 1..19: mol_type = other DNA
tggagagtttgcaaggagc

SEQ ID NO: 8 - DNA, 19 bp, synthetic construct
  misc_feature 1..19: note = Description of Artificial Sequence: Synthetic oligonucleotide
  source 1..19: mol_type = other DNA
gtttattcagccgggagtc

SEQ ID NO: 9 - DNA, 20 bp, synthetic construct
  misc_feature 1..20: note = Description of Artificial Sequence: Synthetic oligonucleotide
  source 1..20: mol_type = other DNA
cgccaggaggggtgggtcta

SEQ ID NO: 10 - DNA, 19 bp, synthetic construct
  misc_feature 1..19: note = Description of Artificial Sequence: Synthetic oligonucleotide
  source 1..19: mol_type = other DNA
ccttggtgagactggtaga

SEQ ID NO: 11 - DNA, 19 bp, synthetic construct
  misc_feature 1..19: note = Description of Artificial Sequence: Synthetic oligonucleotide
  source 1..19: mol_type = other DNA
gtcttcaggttctgttgct

SEQ ID NO: 12 - DNA, 19 bp, synthetic construct
  misc_feature 1..19: note = Description of Artificial Sequence: Synthetic oligonucleotide
  source 1..19: mol_type = other DNA
atattcctgatttaaaagt

SEQ ID NO: 13 - DNA, 19 bp, synthetic construct
  misc_feature 1..19: note = Description of Artificial Sequence: Synthetic oligonucleotide
  source 1..19: mol_type = other DNA
ttaaaagtcggctggtagc

SEQ ID NO: 14 - DNA, 19 bp, synthetic construct
  misc_feature 1..19: note = Description of Artificial Sequence: Synthetic oligonucleotide
  source 1..19: mol_type = other DNA
cgggccgggggcggggtcc

SEQ ID NO: 15 - DNA, 19 bp, synthetic construct
  misc_feature 1..19: note = Description of Artificial Sequence: Synthetic oligonucleotide
  source 1..19: mol_type = other DNA
gcccgagccgcgtgtggaa

SEQ ID NO: 16 - DNA, 19 bp, synthetic construct
  misc_feature 1..19: note = Description of Artificial Sequence: Synthetic oligonucleotide
  source 1..19: mol_type = other DNA
ccttcattgcggcgggctg

SEQ ID NO: 17 - DNA, 19 bp, synthetic construct
  misc_feature 1..19: note = Description of Artificial Sequence: Synthetic oligonucleotide
  source 1..19: mol_type = other DNA
ccgacccctcccgggtccc

SEQ ID NO: 18 - DNA, 19 bp, synthetic construct
  misc_feature 1..19: note = Description of Artificial Sequence: Synthetic oligonucleotide
  source 1..19: mol_type = other DNA
caggaccgcgcttcccacg

SEQ ID NO: 19 - DNA, 19 bp, synthetic construct
  misc_feature 1..19: note = Description of Artificial Sequence: Synthetic oligonucleotide
  source 1..19: mol_type = other DNA
tgcaccctgggagcgcgag

SEQ ID NO: 20 - DNA, 19 bp, synthetic construct
  misc_feature 1..19: note = Description of Artificial Sequence: Synthetic oligonucleotide
  source 1..19: mol_type = other DNA
ccgcacgcacctgttccca

SEQ ID NO: 21 - DNA, 19 bp, synthetic construct
  misc_feature 1..19: note = Description of Artificial Sequence: Synthetic oligonucleotide
  source 1..19: mol_type = other DNA
aaaacagcgagggagaaac

SEQ ID NO: 22 - DNA, 19 bp, synthetic construct
  misc_feature 1..19: note = Description of Artificial Sequence: Synthetic oligonucleotide
  source 1..19: mol_type = other DNA
ttaacttgattgtgaaatc

SEQ ID NO: 23 - DNA, 19 bp, synthetic construct
  misc_feature 1..19: note = Description of Artificial Sequence: Synthetic oligonucleotide
  source 1..19: mol_type = other DNA
aaaacaatgcatatttgca

SEQ ID NO: 24 - DNA, 19 bp, synthetic construct
  misc_feature 1..19: note = Description of Artificial Sequence: Synthetic oligonucleotide
  source 1..19: mol_type = other DNA
aaaatccagtattttaatg

SEQ ID NO: 25 - DNA, 19 bp, synthetic construct
  misc_feature 1..19: note = Description of Artificial Sequence: Synthetic oligonucleotide
  source 1..19: mol_type = other DNA
acccagcactgcagcctgg

SEQ ID NO: 26 - DNA, 19 bp, synthetic construct
  misc_feature 1..19: note = Description of Artificial Sequence: Synthetic oligonucleotide
  source 1..19: mol_type = other DNA
aacttatgcggcgtttcct

SEQ ID NO: 27 - DNA, 19 bp, synthetic construct
  misc_feature 1..19: note = Description of Artificial Sequence: Synthetic oligonucleotide
  source 1..19: mol_type = other DNA
tcactttaaaaccacctct

SEQ ID NO: 28 - DNA, 19 bp, synthetic construct
  misc_feature 1..19: note = Description of Artificial Sequence: Synthetic oligonucleotide
  source 1..19: mol_type = other DNA
gcatctttttctctttaat

SEQ ID NO: 29 - DNA, 19 bp, synthetic construct
  misc_feature 1..19: note = Description of Artificial Sequence: Synthetic oligonucleotide
  source 1..19: mol_type = other DNA
tgtactctctgaggtgctc

SEQ ID NO: 30 - DNA, 19 bp, synthetic construct
  misc_feature 1..19: note = Description of Artificial Sequence: Synthetic oligonucleotide
  source 1..19: mol_type = other DNA
acgcagataagaaccagtt

SEQ ID NO: 31 - DNA, 19 bp, synthetic construct
  misc_feature 1..19: note = Description of Artificial Sequence: Synthetic oligonucleotide
  source 1..19: mol_type = other DNA
catcaagtcagccatcagc

SEQ ID NO: 32 - DNA, 19 bp, synthetic construct
  misc_feature 1..19: note = Description of Artificial Sequence: Synthetic oligonucleotide
  source 1..19: mol_type = other DNA
gagtcaccctcctggaaac

SEQ ID NO: 33 - DNA, 19 bp, synthetic construct
  misc_feature 1..19: note = Description of Artificial Sequence: Synthetic oligonucleotide
  source 1..19: mol_type = other DNA
gctagggatgaagaataaa

SEQ ID NO: 34 - DNA, 19 bp, synthetic construct
  misc_feature 1..19: note = Description of Artificial Sequence: Synthetic oligonucleotide
  source 1..19: mol_type = other DNA
ttgaccaatagccttgaca

SEQ ID NO: 35 - DNA, 19 bp, synthetic construct
  misc_feature 1..19: note = Description of Artificial Sequence: Synthetic oligonucleotide
  source 1..19: mol_type = other DNA
tgcaaatatctgtctgaaa

SEQ ID NO: 36 - DNA, 19 bp, synthetic construct
  misc_feature 1..19: note = Description of Artificial Sequence: Synthetic oligonucleotide
  source 1..19: mol_type = other DNA
aaattagcagtatcctctt

SEQ ID NO: 37 - DNA, 19 bp, synthetic construct
  misc_feature 1..19: note = Description of Artificial Sequence: Synthetic oligonucleotide
  source 1..19: mol_type = other DNA
cctgggctccggggcgttt

SEQ ID NO: 38 - DNA, 19 bp, synthetic construct
  misc_feature 1..19: note = Description of Artificial Sequence: Synthetic oligonucleotide
  source 1..19: mol_type = other DNA
ggcccctgcggccaccccg

SEQ ID NO: 39 - DNA, 19 bp, synthetic construct
  misc_feature 1..19: note = Description of Artificial Sequence: Synthetic oligonucleotide
  source 1..19: mol_type = other DNA
ctccctccctgcccggtag

SEQ ID NO: 40 - DNA, 19 bp, synthetic construct
  misc_feature 1..19: note = Description of Artificial Sequence: Synthetic oligonucleotide
  source 1..19: mol_type = other DNA
aggtttggaaagggcgtgc

SEQ ID NO: 41 - DNA, 20 bp, synthetic construct
  misc_feature 1..20: note = Description of Artificial Sequence: Synthetic primer
  source 1..20: mol_type = other DNA
ggagcttctcgacttcacca

SEQ ID NO: 42 - DNA, 20 bp, synthetic construct
  misc_feature 1..20: note = Description of Artificial Sequence: Synthetic primer
  source 1..20: mol_type = other DNA
gatttgtgggcctgaagaaa

SEQ ID NO: 43 - DNA, 20 bp, synthetic construct
  misc_feature 1..20: note = Description of Artificial Sequence: Synthetic primer
  source 1..20: mol_type = other DNA
aaggaggagggcagaatcat

SEQ ID NO: 44 - DNA, 21 bp, synthetic construct
  misc_feature 1..21: note = Description of Artificial Sequence: Synthetic primer
  source 1..21: mol_type = other DNA
aaaccttcctcagctatgccc

SEQ ID NO: 45 - DNA, 20 bp, synthetic construct
  misc_feature 1..20: note = Description of Artificial Sequence: Synthetic primer
  source 1..20: mol_type = other DNA
agctgatggccctaaacaga

SEQ ID NO: 46 - DNA, 20 bp, synthetic construct
  misc_feature 1..20: note = Description of Artificial Sequence: Synthetic primer
  source 1..20: mol_type = other DNA
caggaggactctggcaccta

SEQ ID NO: 47 - DNA, 20 bp, synthetic construct
  misc_feature 1..20: note = Description of Artificial Sequence: Synthetic primer
  source 1..20: mol_type = other DNA
ggaatccatggagggaagat

SEQ ID NO: 48 - DNA, 21 bp, synthetic construct
  misc_feature 1..21: note = Description of Artificial Sequence: Synthetic primer
  source 1..21: mol_type = other DNA
gctgagtgaactgcactgtga

SEQ ID NO: 49 - DNA, 20 bp, synthetic construct
  misc_feature 1..20: note = Description of Artificial Sequence: Synthetic primer
  source 1..20: mol_type = other DNA
ctctctgctcctttgccaca

SEQ ID NO: 50 - DNA, 20 bp, synthetic construct
  misc_feature 1..20: note = Description of Artificial Sequence: Synthetic primer
  source 1..20: mol_type = other DNA
caatgaccccttcattgacc

SEQ ID NO: 51 - DNA, 20 bp, synthetic construct
  misc_feature 1..20: note = Description of Artificial Sequence: Synthetic primer
  source 1..20: mol_type = other DNA
ggaacaagagctgctggact

SEQ ID NO: 52 - DNA, 20 bp, synthetic construct
  misc_feature 1..20: note = Description of Artificial Sequence: Synthetic primer
  source 1..20: mol_type = other DNA
aactttggcattgtggaagg

SEQ ID NO: 53 - DNA, 20 bp, synthetic construct
  misc_feature 1..20: note = Description of Artificial Sequence: Synthetic primer
  source 1..20: mol_type = other DNA
aacgccactgacaagaaagc

SEQ ID NO: 54 - DNA, 20 bp, synthetic construct
  misc_feature 1..20: note = Description of Artificial Sequence: Synthetic primer
  source 1..20: mol_type = other DNA
cagatccatggaggaaggaa

SEQ ID NO: 55 - DNA, 21 bp, synthetic construct
  misc_feature 1..21: note = Description of Artificial Sequence: Synthetic primer
  source 1..21: mol_type = other DNA
gggtactcctggaagatgtcc

SEQ ID NO: 56 - DNA, 20 bp, synthetic construct
  misc_feature 1..20: note = Description of Artificial Sequence: Synthetic primer
  source 1..20: mol_type = other DNA
gtttgcgacgcatgttcctc

SEQ ID NO: 57 - DNA, 20 bp, synthetic construct
  misc_feature 1..20: note = Description of Artificial Sequence: Synthetic primer
  source 1..20: mol_type = other DNA
aagcccttgctgtagtggtg

SEQ ID NO: 58 - DNA, 20 bp, synthetic construct
  misc_feature 1..20: note = Description of Artificial Sequence: Synthetic primer
  source 1..20: mol_type = other DNA
cggcaggaaagcatctgtat

SEQ ID NO: 59 - DNA, 20 bp, synthetic construct
  misc_feature 1..20: note = Description of Artificial Sequence: Synthetic primer
  source 1..20: mol_type = other DNA
tgttctcgctcaggtcagtg

SEQ ID NO: 60 - DNA, 20 bp, synthetic construct
  misc_feature 1..20: note = Description of Artificial Sequence: Synthetic primer
  source 1..20: mol_type = other DNA
gaattctttgccgaaatgga

SEQ ID NO: 61 - DNA, 20 bp, synthetic construct
  misc_feature 1..20: note = Description of Artificial Sequence: Synthetic primer
  source 1..20: mol_type = other DNA
gtgctcttcgggtttcagga

SEQ ID NO: 62 - DNA, 20 bp, synthetic construct
  misc_feature 1..20: note = Description of Artificial Sequence: Synthetic primer
  source 1..20: mol_type = other DNA
ttgattttggagggatctcg

SEQ ID NO: 63 - DNA, 20 bp, synthetic construct
  misc_feature 1..20: note = Description of Artificial Sequence: Synthetic primer
  source 1..20: mol_type = other DNA
gtttttctgcctccccattt

SEQ ID NO: 64 - DNA, 20 bp, synthetic construct
  misc_feature 1..20: note = Description of Artificial Sequence: Synthetic primer
  source 1..20: mol_type = other DNA
ggatgcagggatgatgttct

SEQ ID NO: 65 - DNA, 20 bp, synthetic construct
  misc_feature 1..20: note = Description of Artificial Sequence: Synthetic oligonucleotide
  source 1..20: mol_type = other DNA
gattggctttgatttcccta

SEQ ID NO: 66 - DNA, 23 bp, synthetic construct
  misc_feature 1..23: note = Description of Artificial Sequence: Synthetic oligonucleotide
  source 1..23: mol_type = other DNA
gtgtagagtaagtcagcctatgg

SEQ ID NO: 67 - DNA, 20 bp, synthetic construct
  misc_feature 1..20: note = Description of Artificial Sequence: Synthetic oligonucleotide
  source 1..20: mol_type = other DNA
gcctactcagactgttactc

SEQ ID NO: 68 - DNA, 23 bp, synthetic construct
  misc_feature 1..23: note = Description of Artificial Sequence: Synthetic oligonucleotide
  source 1..23: mol_type = other DNA
gttggacagaacttaccgactgg

SEQ ID NO: 69 - DNA, 20 bp, synthetic construct
  misc_feature 1..20: note = Description of Artificial Sequence: Synthetic oligonucleotide
  source 1..20: mol_type = other DNA
gcagttgcctaagaactggt

SEQ ID NO: 70 - DNA, 20 bp, synthetic construct
  misc_feature 1..20: note = Description of Artificial Sequence: Synthetic oligonucleotide
  source 1..20: mol_type = other DNA
ggggctccaccctcacgagt

SEQ ID NO: 71 - DNA, 23 bp, synthetic construct
  misc_feature 1..23: note = Description of Artificial Sequence: Synthetic oligonucleotide
  source 1..23: mol_type = other DNA
gtttgcttcgctataaaacgagg

SEQ ID NO: 72 - DNA, 23 bp, synthetic construct
  misc_feature 1..23: note = Description of Artificial Sequence: Synthetic oligonucleotide
  source 1..23: mol_type = other DNA
gtctgaggatggggccgcaatgg

SEQ ID NO: 73 - DNA, 23 bp, synthetic construct
  misc_feature 1..23: note = Description of Artificial Sequence: Synthetic oligonucleotide
  source 1..23: mol_type = other DNA
ggatctgtcaaatcgcctgcagg

SEQ ID NO: 74 - DNA, 23 bp, synthetic construct
  misc_feature 1..23: note = Description of Artificial Sequence: Synthetic oligonucleotide
  source 1..23: mol_type = other DNA
gccaggatggcattgggcagcgg

SEQ ID NO: 75 - DNA, 23 bp, synthetic construct
  misc_feature 1..23: note = Description of Artificial Sequence: Synthetic oligonucleotide
  source 1..23: mol_type = other DNA
gctgaatctgcggtggcaggagg

SEQ ID NO: 76 - DNA, 23 bp, synthetic construct
  misc_feature 1..23: note = Description of Artificial Sequence: Synthetic oligonucleotide
  source 1..23: mol_type = other DNA
gttcttttgttcttctagcctgg

SEQ ID NO: 77 - DNA, 23 bp, synthetic construct
  misc_feature 1..23: note = Description of Artificial Sequence: Synthetic oligonucleotide
  source 1..23: mol_type = other DNA
ggaaaagcttgagcaagtcaagg

SEQ ID NO: 78 - DNA, 23 bp, synthetic construct
  misc_feature 1..23: note = Description of Artificial Sequence: Synthetic oligonucleotide
  source 1..23: mol_type = other DNA
ggaagagttgcccctgcgccagg

SEQ ID NO: 79 - DNA, 23 bp, synthetic construct
  misc_feature 1..23: note = Description of Artificial Sequence: Synthetic oligonucleotide
  source 1..23: mol_type = other DNA
gacaaatctccagtggataaagg

SEQ ID NO: 80 - DNA, 23 bp, synthetic construct
  misc_feature 1..23: note = Description of Artificial Sequence: Synthetic oligonucleotide
  source 1..23: mol_type = other DNA
gtgtttctcaggtaaagctctgg

SEQ ID NO: 81 - DNA, 23 bp, synthetic construct
  misc_feature 1..23: note = Description of Artificial Sequence: Synthetic oligonucleotide
  source 1..23: mol_type = other DNA
ggaaggaccatttgacgttaagg

SEQ ID NO: 82 - DNA, 23 bp, synthetic construct
  misc_feature 1..23: note = Description of Artificial Sequence: Synthetic oligonucleotide
  source 1..23: mol_type = other DNA
gaactgctatttcagtttcctgg

SEQ ID NO: 83 - DNA, 23 bp, synthetic construct
  misc_feature 1..23: note = Description of Artificial Sequence: Synthetic oligonucleotide
  source 1..23: mol_type = other DNA
gccagccactcagccagtgaagg

SEQ ID NO: 84 - DNA, 23 bp, synthetic construct
  misc_feature 1..23: note = Description of Artificial Sequence: Synthetic oligonucleotide
  source 1..23: mol_type = other DNA
ggtatgcttttctgttaaagagg

SEQ ID NO: 85 - DNA, 23 bp, synthetic construct
  misc_feature 1..23: note = Description of Artificial Sequence: Synthetic oligonucleotide
  source 1..23: mol_type = other DNA
gctcctggactgaccactattgg

SEQ ID NO: 86 - DNA, 23 bp, synthetic construct
  misc_feature 1..23: note = Description of Artificial Sequence: Synthetic oligonucleotide
  source 1..23: mol_type = other DNA
ggaacagaggcgtccccagttgg

SEQ ID NO: 87 - DNA, 23 bp, synthetic construct
  misc_feature 1..23: note = Description of Artificial Sequence: Synthetic oligonucleotide
  source 1..23: mol_type = other DNA
ggaggctagaacaatcattacgg

SEQ ID NO: 88 - DNA, 23 bp, synthetic construct
  misc_feature 1..23: note = Description of Artificial Sequence: Synthetic oligonucleotide
  source 1..23: mol_type = other DNA
gacaagaacaccttcagaaccgg

SEQ ID NO: 89 - DNA, 23 bp, synthetic construct
  misc_feature 1..23: note = Description of Artificial Sequence: Synthetic oligonucleotide
  source 1..23: mol_type = other DNA
gggtttctgtgattttcttttgg

SEQ ID NO: 90 - DNA, 23 bp, synthetic construct
  misc_feature 1..23: note = Description of Artificial Sequence: Synthetic oligonucleotide
  source 1..23: mol_type = other DNA
gggccaaagacctccgccagtgg

SEQ ID NO: 91 - DNA, 23 bp, synthetic construct
  misc_feature 1..23: note = Description of Artificial Sequence: Synthetic oligonucleotide
  source 1..23: mol_type = other DNA
gttggagaagcattcataaaagg

SEQ ID NO: 92 - DNA, 23 bp, synthetic construct
  misc_feature 1..23: note = Description of Artificial Sequence: Synthetic oligonucleotide
  source 1..23: mol_type = other DNA
gtcgctcactcaccctgcaaagg

SEQ ID NO: 93 - DNA, 23 bp, synthetic construct
  misc_feature 1..23: note = Description of Artificial Sequence: Synthetic oligonucleotide
  source 1..23: mol_type = other DNA
gaaaagagctgatgaaacaatgg

SEQ ID NO: 94 - DNA, 23 bp, synthetic construct
  misc_feature 1..23: note = Description of Artificial Sequence: Synthetic oligonucleotide
  source 1..23: mol_type = other DNA
gtacacttttcaaaatgctttgg

SEQ ID NO: 95 - DNA, 23 bp, synthetic construct
  misc_feature 1..23: note = Description of Artificial Sequence: Synthetic oligonucleotide
  source 1..23: mol_type = other DNA
ggagatgatcatcaagcagaagg

SEQ ID NO: 96 - DNA, 23 bp, synthetic construct
  misc_feature 1..23: note = Description of Artificial Sequence: Synthetic oligonucleotide
  source 1..23: mol_type = other DNA
gctttgaaagagcaataaaatgg

SEQ ID NO: 97 - DNA, 23 bp, synthetic construct
  misc_feature 1..23: note = Description of Artificial Sequence: Synthetic oligonucleotide
  source 1..23: mol_type = other DNA
gcacaaaagtcaaatcggaatgg

SEQ ID NO: 98 - DNA, 23 bp, synthetic construct
  misc_feature 1..23: note = Description of Artificial Sequence: Synthetic oligonucleotide
  source 1..23: mol_type = other DNA
gatttcaatataagattcggagg

SEQ ID NO: 99 - DNA, 23 bp, synthetic construct
  misc_feature 1..23: note = Description of Artificial Sequence: Synthetic oligonucleotide
  source 1..23: mol_type = other DNA
gcttaagcaatcccgaactctgg

SEQ ID NO: 100 - DNA, 20 bp, synthetic construct
  misc_feature 1..20: note = Description of Artificial Sequence: Synthetic oligonucleotide
  source 1..20: mol_type = other DNA
gccttctttatcccctatcg

SEQ ID NO: 101-103 - skipped (3 entries)

SEQ ID NO: 104 - DNA, 25 bp, synthetic construct
  misc_feature 1..25: note = Description of Artificial Sequence: Synthetic oligonucleotide
  variation 21..22: note = a, c, t, g, unknown or other
  source 1..25: mol_type = other DNA
gaggccaaacctcggcttacnngrr

SEQ ID NO: 105 - DNA, 25 bp, synthetic construct
  misc_feature 1..25: note = Description of Artificial Sequence: Synthetic oligonucleotide
  variation 21..22: note = a, c, t, g, unknown or other
  source 1..25: mol_type = other DNA
gttcgaaaatttcaggtaagnngrr

SEQ ID NO: 106 - DNA, 26 bp, synthetic construct
  misc_feature 1..26: note = Description of Artificial Sequence: Synthetic oligonucleotide
  variation 21..22: note = a, c, t, g, unknown or other
  source 1..26: mol_type = other DNA
ggcagaacaggagataacagnngrrt

SEQ ID NO: 107 - DNA, 26 bp, synthetic construct
  misc_feature 1..26: note = Description of Artificial Sequence: Synthetic oligonucleotide
  source 1..26: mol_type = other DNA
ggcggccctcgcccttctctggggat

SEQ ID NO: 108 - DNA, 23 bp, synthetic construct
  misc_feature 1..23: note = Description of Artificial Sequence: Synthetic oligonucleotide
  source 1..23: mol_type = other DNA
gtagtgatcgtggatacgagagg

SEQ ID NO: 109 - DNA, 23 bp, synthetic construct
  misc_feature 1..23: note = Description of Artificial Sequence: Synthetic oligonucleotide
  source 1..23: mol_type = other DNA
gtacagccctcggtgtatattgg

SEQ ID NO: 110 - DNA, 23 bp, synthetic construct
  misc_feature 1..23: note = Description of Artificial Sequence: Synthetic oligonucleotide
  source 1..23: mol_type = other DNA
gggaaggaattaagcccgaatgg

SEQ ID NO: 111 - DNA, 23 bp, synthetic construct
  misc_feature 1..23: note = Description of Artificial Sequence: Synthetic oligonucleotide
  source 1..23: mol_type = other DNA
gggaacagctttcgtagttgagg

SEQ ID NO: 112 - DNA, 23 bp, synthetic construct
  misc_feature 1..23: note = Description of Artificial Sequence: Synthetic oligonucleotide
  source 1..23: mol_type = other DNA
gataaagtccagtgtcgatcagg

SEQ ID NO: 113 - DNA, 23 bp, synthetic construct
  misc_feature 1..23: note = Description of Artificial Sequence: Synthetic oligonucleotide
  source 1..23: mol_type = other DNA
gaaaaccagagcttcggtcaagg

SEQ ID NO: 114 - DNA, 34 bp, synthetic construct
  misc_feature 1..34: note = Description of Artificial Sequence: Synthetic oligonucleotide
  source 1..34: mol_type = other DNA
ggagtcttctgggcaggcttaaaggctaacctgg

SEQ ID NO: 115 - DNA, 34 bp, synthetic construct
  misc_feature 1..34: note = Description of Artificial Sequence: Synthetic oligonucleotide
  source 1..34: mol_type = other DNA
gtcgggtgagcatgtctttaatctacctcgatgg

SEQ ID NO: 116 - DNA, 26 bp, synthetic construct
  misc_feature 1..26: note = Description of Artificial Sequence: Synthetic oligonucleotide
  source 1..26: mol_type = other DNA
ggtgtcaccagagtaacagtctgagt

SEQ ID NO: 117 - DNA, 25 bp, synthetic construct
  misc_feature 1..25: note = Description of Artificial Sequence: Synthetic oligonucleotide
  source 1..25: mol_type = other DNA
gtgatcatcaagcagaaggtatgag

SEQ ID NO: 118 - DNA, 29 bp, synthetic construct
  misc_feature 1..29: note = Description of Artificial Sequence: Synthetic oligonucleotide
  source 1..29: mol_type = other DNA
gaacttcgaaaatttcaggtaagccgagg

SEQ ID NO: 119 - DNA, 29 bp, synthetic construct
  misc_feature 1..29: note = Description of Artificial Sequence: Synthetic oligonucleotide
  source 1..29: mol_type = other DNA
ggaaactcatcaaatatgcgtgttagtgt

SEQ ID NO: 120 - DNA, 29 bp, synthetic construct
  misc_feature 1..29: note = Description of Artificial Sequence: Synthetic oligonucleotide
  source 1..29: mol_type = other DNA
gtcatttacactaacacgcatatttgatg

SEQ ID NO: 121 - DNA, 29 bp, synthetic construct
  misc_feature 1..29: note = Description of Artificial Sequence: Synthetic oligonucleotide
  source 1..29: mol_type = other DNA
ggaatgaaactcatcaaatatgcgtgtta

SEQ ID NO: 122 - DNA, 29 bp, synthetic construct
  misc_feature 1..29: note = Description of Artificial Sequence: Synthetic oligonucleotide
  source 1..29: mol_type = other DNA
gtcatcaatatctttgaaggactctgggt

SEQ ID NO: 123 - DNA, 29 bp, synthetic construct
  misc_feature 1..29: note = Description of Artificial Sequence: Synthetic oligonucleotide
  source 1..29: mol_type = other DNA
gtgttttcataggaaaaataggcaagttg

SEQ ID NO: 124 - DNA, 29 bp, synthetic construct
  misc_feature 1..29: note = Description of Artificial Sequence: Synthetic oligonucleotide
  source 1..29: mol_type = other DNA
gaattggaaaatgtgatgggaaacagata

SEQ ID NO: 125 - DNA, 29 bp, synthetic construct
  misc_feature 1..29: note = Description of Artificial Sequence: Synthetic oligonucleotide
  source 1..29: mol_type = other DNA
gatgatcatcaagcagaaggtatgagaaa

SEQ ID NO: 126 - DNA, 29 bp, synthetic construct
  misc_feature 1..29: note = Description of Artificial Sequence: Synthetic oligonucleotide
  source 1..29: mol_type = other DNA
gagatgatcatcaagcagaaggtatgaga

SEQ ID NO: 127 - DNA, 29 bp, synthetic construct
  misc_feature 1..29: note = Description of Artificial Sequence: Synthetic oligonucleotide
  source 1..29: mol_type = other DNA
gcattttttctcataccttctgcttgatg

SEQ ID NO: 128 - DNA, 29 bp, synthetic construct
  misc_feature 1..29: note = Description of Artificial Sequence: Synthetic oligonucleotide
  source 1..29: mol_type = other DNA
gtcctactcagactgttactctggtgaca

SEQ ID NO: 129 - DNA, 29 bp, synthetic construct
  misc_feature 1..29: note = Description of Artificial Sequence: Synthetic oligonucleotide
  source 1..29: mol_type = other DNA
gacaggttgtgtcaccagagtaacagtct

SEQ ID NO: 130 - DNA, 29 bp, synthetic construct
  misc_feature 1..29: note = Description of Artificial Sequence: Synthetic oligonucleotide
  source 1..29: mol_type = other DNA
gttatcattttttctcataccttctgctt

SEQ ID NO: 131 - DNA, 29 bp, synthetic construct
  misc_feature 1..29: note = Description of Artificial Sequence: Synthetic oligonucleotide
  source 1..29: mol_type = other DNA
gttgcctaagaactggtgggaaatggtct

SEQ ID NO: 132 - DNA, 29 bp, synthetic construct
  misc_feature 1..29: note = Description of Artificial Sequence: Synthetic oligonucleotide
  source 1..29: mol_type = other DNA
gaaacagttgcctaagaactggtgggaaa

SEQ ID NO: 133 - DNA, 29 bp, synthetic construct
  misc_feature 1..29: note = Description of Artificial Sequence: Synthetic oligonucleotide
  source 1..29: mol_type = other DNA
gtttcccaccagttcttaggcaactgttt

SEQ ID NO: 134 - DNA, 29 bp, synthetic construct
  misc_feature 1..29: note = Description of Artificial Sequence: Synthetic oligonucleotide
  source 1..29: mol_type = other DNA
gtggctttgatttccctagggtccagctt

SEQ ID NO: 135 - DNA, 29 bp, synthetic construct
  misc_feature 1..29: note = Description of Artificial Sequence: Synthetic oligonucleotide
  source 1..29: mol_type = other DNA
gtagggaaatcaaagccaatgaaacgttc

SEQ ID NO: 136 - DNA, 29 bp, synthetic construct
  misc_feature 1..29: note = Description of Artificial Sequence: Synthetic oligonucleotide
  source 1..29: mol_type = other DNA
ggaccctagggaaatcaaagccaatgaaa

SEQ ID NO: 137 - DNA, 29 bp, synthetic construct
  misc_feature 1..29: note = Description of Artificial Sequence: Synthetic oligonucleotide
  source 1..29: mol_type = other DNA
gtgagggctccaccctcacgagtgggttt

SEQ ID NO: 138 - DNA, 29 bp, synthetic construct
  misc_feature 1..29: note = Description of Artificial Sequence: Synthetic oligonucleotide
  source 1..29: mol_type = other DNA
gaaggattgagggctccaccctcacgagt

SEQ ID NO: 139 - DNA, 29 bp, synthetic construct
  misc_feature 1..29: note = Description of Artificial Sequence: Synthetic oligonucleotide
  source 1..29: mol_type = other DNA
ggctccaccctcacgagtgggtttggttc

SEQ ID NO: 140 - DNA, 29 bp, synthetic construct
  misc_feature 1..29: note = Description of Artificial Sequence: Synthetic oligonucleotide
  source 1..29: mol_type = other DNA
gtatcccctatcgaggaaaccacgagttt

SEQ ID NO: 141 - DNA, 29 bp, synthetic construct
  misc_feature 1..29: note = Description of Artificial Sequence: Synthetic oligonucleotide
  source 1..29: mol_type = other DNA
ggataaagaaggcctatttcatagagttg

SEQ ID NO: 142 - DNA, 29 bp, synthetic construct
  misc_feature 1..29: note = Description of Artificial Sequence: Synthetic oligonucleotide
  source 1..29: mol_type = other DNA
gaggccttctttatcccctatcgaggaaa

SEQ ID NO: 143 - DNA, 27 bp, synthetic construct
  misc_feature 1..27: note = Description of Artificial Sequence: Synthetic oligonucleotide
  source 1..27: mol_type = other DNA
gtgagggctccaccctcacgagtgggt

SEQ ID NO: 144 - DNA, 27 bp, synthetic construct
  misc_feature 1..27: note = Description of Artificial Sequence: Synthetic oligonucleotide
  source 1..27: mol_type = other DNA
ggataaagaaggcctatttcatagagt

SEQ ID NO: 145 - DNA, 4932 bp, synthetic construct
  misc_feature 1..4932: note = Description of Artificial Sequence: Synthetic polynucleotide
  source 1..4932: mol_type = other DNA
gccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgagggcagaggaagtcttctaacatgcggtgacgtggaggagaatcccggccctggtaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaccggttag

SEQ ID NO: 146-149 - skipped (4 entries)

SEQ ID NO: 150 - DNA, 23 bp, synthetic construct
  misc_feature 1..23: note = Description of Artificial Sequence: Synthetic oligonucleotide
  source 1..23: mol_type = other DNA
tcctactcagactgttactctgg

SEQ ID NO: 151 - DNA, 23 bp, synthetic construct
  misc_feature 1..23: note = Description of Artificial Sequence: Synthetic oligonucleotide
  source 1..23: mol_type = other DNA
tcctactcacactgttactcagg

SEQ ID NO: 152 - DNA, 23 bp, synthetic construct
  misc_feature 1..23: note = Description of Artificial Sequence: Synthetic oligonucleotide
  source 1..23: mol_type = other DNA
acctgctcacactgttactccag

SEQ ID NO: 153 - DNA, 23 bp, synthetic construct
  misc_feature 1..23: note = Description of Artificial Sequence: Synthetic oligonucleotide
  source 1..23: mol_type = other DNA
gcattctcaaactgttactcagg

SEQ ID NO: 154 - DNA, 23 bp, synthetic construct
  misc_feature 1..23: note = Description of Artificial Sequence: Synthetic oligonucleotide
  source 1..23: mol_type = other DNA
ggattctcacactgttactcggg

SEQ ID NO: 155 - DNA, 23 bp, synthetic construct
  misc_feature 1..23: note = Description of Artificial Sequence: Synthetic oligonucleotide
  source 1..23: mol_type = other DNA
acatacttatactgttactctag

SEQ ID NO: 156 - DNA, 23 bp, synthetic construct
  misc_feature 1..23: note = Description of Artificial Sequence: Synthetic oligonucleotide
  source 1..23: mol_type = other DNA
tattcctaagactgttactcaag

SEQ ID NO: 157 - DNA, 23 bp, synthetic construct
  misc_feature 1..23: note = Description of Artificial Sequence: Synthetic oligonucleotide
  source 1..23: mol_type = other DNA
aaggactaagactgttactcggg

SEQ ID NO: 158 - DNA, 23 bp, synthetic construct
  misc_feature 1..23: note = Description of Artificial Sequence: Synthetic oligonucleotide
  source 1..23: mol_type = other DNA
gagctctcatactgttactctag

SEQ ID NO: 159 - DNA, 23 bp, synthetic construct
  misc_feature 1..23: note = Description of Artificial Sequence: Synthetic oligonucleotide
  source 1..23: mol_type = other DNA
gcaaaatgagactgttactccag

SEQ ID NO: 160 - DNA, 23 bp, synthetic construct
  misc_feature 1..23: note = Description of Artificial Sequence: Synthetic oligonucleotide
  source 1..23: mol_type = other DNA
cctcattcagactgttactcaag

SEQ ID NO: 161 - DNA, 23 bp, synthetic construct
  misc_feature 1..23: note = Description of Artificial Sequence: Synthetic oligonucleotide
  source 1..23: mol_type = other DNA
cattggctttgatttccctaggg

SEQ ID NO: 162 - DNA, 23 bp, synthetic construct
  misc_feature 1..23: note = Description of Artificial Sequence: Synthetic oligonucleotide
  source 1..23: mol_type = other DNA
aattggcattgatttccctagag

SEQ ID NO: 163 - DNA, 23 bp, synthetic construct
  misc_feature 1..23: note = Description of Artificial Sequence: Synthetic oligonucleotide
  source 1..23: mol_type = other DNA
cattggctttaatttccctatag

SEQ ID NO: 164 - DNA, 23 bp, synthetic construct
  misc_feature 1..23: note = Description of Artificial Sequence: Synthetic oligonucleotide
  source 1..23: mol_type = other DNA
gataggctgtgatttccctagag

SEQ ID NO: 165 - DNA, 23 bp, synthetic construct
  misc_feature 1..23: note = Description of Artificial Sequence: Synthetic oligonucleotide
  source 1..23: mol_type = other DNA
gaatagccttgatttccctaaag

SEQ ID NO: 166 - DNA, 23 bp, synthetic construct
  misc_feature 1..23: note = Description of Artificial Sequence: Synthetic oligonucleotide
  source 1..23: mol_type = other DNA
aatttgctttgatttccctgagg

SEQ ID NO: 167 - DNA, 23 bp, synthetic construct
  misc_feature 1..23: note = Description of Artificial Sequence: Synthetic oligonucleotide
  source 1..23: mol_type = other DNA
gatgtgctttgatttcccttggg

SEQ ID NO: 168 - DNA, 23 bp, synthetic construct
  misc_feature 1..23: note = Description of Artificial Sequence: Synthetic oligonucleotide
  source 1..23: mol_type = other DNA
aattggttttaatttccctaaag

SEQ ID NO: 169 - DNA, 23 bp, synthetic construct
  misc_feature 1..23: note = Description of Artificial Sequence: Synthetic oligonucleotide
  source 1..23: mol_type = other DNA
aattgggtttgatttccctttgg

SEQ ID NO: 170 - DNA, 23 bp, synthetic construct
  misc_feature 1..23: note = Description of Artificial Sequence: Synthetic oligonucleotide
  source 1..23: mol_type = other DNA
gatgggtttttatttccctagag

SEQ ID NO: 171 - DNA, 23 bp, synthetic construct
  misc_feature 1..23: note = Description of Artificial Sequence: Synthetic oligonucleotide
  source 1..23: mol_type = other DNA
gaatggttttgatttccctggag

SEQ ID NO: 172 - DNA, 23 bp, synthetic construct
  misc_feature 1..23: note = Description of Artificial Sequence: Synthetic oligonucleotide
  source 1..23: mol_type = other DNA
acagttgcctaagaactggtggg

SEQ ID NO: 173 - DNA, 23 bp, synthetic construct
  misc_feature 1..23: note = Description of Artificial Sequence: Synthetic oligonucleotide
  source 1..23: mol_type = other DNA
ccagttgtctaagaactggggag

SEQ ID NO: 174 - DNA, 23 bp, synthetic construct
  misc_feature 1..23: note = Description of Artificial Sequence: Synthetic oligonucleotide
  source 1..23: mol_type = other DNA
gcagttgcctgtgaactggtagg

SEQ ID NO: 175 - DNA, 23 bp, synthetic construct
  misc_feature 1..23: note = Description of Artificial Sequence: Synthetic oligonucleotide
  source 1..23: mol_type = other DNA
gcagatgcagaagaactggtgag

SEQ ID NO: 176 - DNA, 23 bp, synthetic construct
  misc_feature 1..23: note = Description of Artificial Sequence: Synthetic oligonucleotide
  source 1..23: mol_type = other DNA
gcagttccagaagaactggtgag

SEQ ID NO: 177 - DNA, 23 bp, synthetic construct
  misc_feature 1..23: note = Description of Artificial Sequence: Synthetic oligonucleotide
  source 1..23: mol_type = other DNA
caacttgcctatgaactggtagg

SEQ ID NO: 178 - DNA, 23 bp, synthetic construct
  misc_feature 1..23: note = Description of Artificial Sequence: Synthetic oligonucleotide
  source 1..23: mol_type = other DNA
acacctgcctaagaactggaggg

SEQ ID NO: 179 - DNA, 23 bp, synthetic construct
  misc_feature 1..23: note = Description of Artificial Sequence: Synthetic oligonucleotide
  source 1..23: mol_type = other DNA
tcaggtggctaagaactgggtgg

SEQ ID NO: 180 - DNA, 23 bp, synthetic construct
  misc_feature 1..23: note = Description of Artificial Sequence: Synthetic oligonucleotide
  source 1..23: mol_type = other DNA
gaagttggccaagaactggagag

SEQ ID NO: 181 - DNA, 23 bp, synthetic construct
  misc_feature 1..23: note = Description of Artificial Sequence: Synthetic oligonucleotide
  source 1..23: mol_type = other DNA
gctgctgcccaagaactggcagg

SEQ ID NO: 182 - DNA, 23 bp, synthetic construct
  misc_feature 1..23: note = Description of Artificial Sequence: Synthetic oligonucleotide
  source 1..23: mol_type = other DNA
tcagctggctaagaacgggtaag

SEQ ID NO: 183 - DNA, 23 bp, synthetic construct
  misc_feature 1..23: note = Description of Artificial Sequence: Synthetic oligonucleotide
  source 1..23: mol_type = other DNA
agggctccaccctcacgagtggg

SEQ ID NO: 184 - DNA, 23 bp, synthetic construct
  misc_feature 1..23: note = Description of Artificial Sequence: Synthetic oligonucleotide
  source 1..23: mol_type = other DNA
gcagctcagccctcacgagtcag

SEQ ID NO: 185 - DNA, 23 bp, synthetic construct
  misc_feature 1..23: note = Description of Artificial Sequence: Synthetic oligonucleotide
  source 1..23: mol_type = other DNA
ggggcttcagcatcacgagtgag

SEQ ID NO: 186 - DNA, 23 bp, synthetic construct
  misc_feature 1..23: note = Description of Artificial Sequence: Synthetic oligonucleotide
  source 1..23: mol_type = other DNA
ggggctctcccctcactagtgag

SEQ ID NO: 187 - DNA, 23 bp, synthetic construct
  misc_feature 1..23: note = Description of Artificial Sequence: Synthetic oligonucleotide
  source 1..23: mol_type = other DNA
ggggatccaccttcaccagtcag

SEQ ID NO: 188 - DNA, 23 bp, synthetic construct
  misc_feature 1..23: note = Description of Artificial Sequence: Synthetic oligonucleotide
  source 1..23: mol_type = other DNA
agggctggaccctcacaagtaag

SEQ ID NO: 189 - DNA, 23 bp, synthetic construct
  misc_feature 1..23: note = Description of Artificial Sequence: Synthetic oligonucleotide
  source 1..23: mol_type = other DNA
tggtctcctcccccacgagtggg

SEQ ID NO: 190 - DNA, 23 bp, synthetic construct
  misc_feature 1..23: note = Description of Artificial Sequence: Synthetic oligonucleotide
  source 1..23: mol_type = other DNA
agggctcccaccccacgagtgag

SEQ ID NO: 191 - DNA, 23 bp, synthetic construct
  misc_feature 1..23: note = Description of Artificial Sequence: Synthetic oligonucleotide
  source 1..23: mol_type = other DNA
gaggctccatactcaccagtgag

SEQ ID NO: 192 - DNA, 23 bp, synthetic construct
  misc_feature 1..23: note = Description of Artificial Sequence: Synthetic oligonucleotide
  source 1..23: mol_type = other DNA
ggagctgccccttcacgagtggg

SEQ ID NO: 193 - DNA, 23 bp, synthetic construct
  misc_feature 1..23: note = Description of Artificial Sequence: Synthetic oligonucleotide
  source 1..23: mol_type = other DNA
atgactccaccctcaagagtaag

SEQ ID NO: 194 - DNA, 23 bp, synthetic construct
  misc_feature 1..23: note = Description of Artificial Sequence: Synthetic oligonucleotide
  source 1..23: mol_type = other DNA
gccttctttatcccctatcgagg

SEQ ID NO: 195 - DNA, 23 bp, synthetic construct
  misc_feature 1..23: note = Description of Artificial Sequence: Synthetic oligonucleotide
  source 1..23: mol_type = other DNA
gtctgctgtgtcccctatcgggg

SEQ ID NO: 196 - DNA, 23 bp, synthetic construct
  misc_feature 1..23: note = Description of Artificial Sequence: Synthetic oligonucleotide
  source 1..23: mol_type = other DNA
cccttctctatcccctgtcgtgg

SEQ ID NO: 197 - DNA, 23 bp, synthetic construct
  misc_feature 1..23: note = Description of Artificial Sequence: Synthetic oligonucleotide
  source 1..23: mol_type = other DNA
gccttctttatcccctctcttgg

SEQ ID NO: 198 - DNA, 23 bp, synthetic construct
  misc_feature 1..23: note = Description of Artificial Sequence: Synthetic oligonucleotide
  source 1..23: mol_type = other DNA
gcgctctttttcccctatcttag

SEQ ID NO: 199 - DNA, 23 bp, synthetic construct
  misc_feature 1..23: note = Description of Artificial Sequence: Synthetic oligonucleotide
  source 1..23: mol_type = other DNA
gccctctgtctcccctgtcgcag

SEQ ID NO: 200 - DNA, 23 bp, synthetic construct
  misc_feature 1..23: note = Description of Artificial Sequence: Synthetic oligonucleotide
  source 1..23: mol_type = other DNA
tccatctttgtcccctattgagg

SEQ ID NO: 201 - DNA, 23 bp, synthetic construct
  misc_feature 1..23: note = Description of Artificial Sequence: Synthetic oligonucleotide
  source 1..23: mol_type = other DNA
accttctctctcccctatagagg

SEQ ID NO: 202 - DNA, 23 bp, synthetic construct
  misc_feature 1..23: note = Description of Artificial Sequence: Synthetic oligonucleotide
  source 1..23: mol_type = other DNA
gttttctttttcccctatgggag

SEQ ID NO: 203 - DNA, 23 bp, synthetic construct
  misc_feature 1..23: note = Description of Artificial Sequence: Synthetic oligonucleotide
  source 1..23: mol_type = other DNA
tgcttcttaatcccctatcaaag

SEQ ID NO: 204 - DNA, 23 bp, synthetic construct
  misc_feature 1..23: note = Description of Artificial Sequence: Synthetic oligonucleotide
  source 1..23: mol_type = other DNA
accttcttactcccctatccggg

SEQ ID NO: 205 - DNA, 22 bp, synthetic construct
  misc_feature 1..22: note = Description of Artificial Sequence: Synthetic primer
  source 1..22: mol_type = other DNA
gagaggttatgtggctttacca

SEQ ID NO: 206 - DNA, 20 bp, synthetic construct
  misc_feature 1..20: note = Description of Artificial Sequence: Synthetic primer
  source 1..20: mol_type = other DNA
aaaaatgcttcccactttgc

SEQ ID NO: 207 - DNA, 20 bp, synthetic construct
  misc_feature 1..20: note = Description of Artificial Sequence: Synthetic primer
  source 1..20: mol_type = other DNA
ctcattctcatgcctggaca

SEQ ID NO: 208 - DNA, 25 bp, synthetic construct
  misc_feature 1..25: note = Description of Artificial Sequence: Synthetic primer
  source 1..25: mol_type = other DNA
gagtttggctcaaattgttactctt

SEQ ID NO: 209 - DNA, 25 bp, synthetic construct
  misc_feature 1..25: note = Description of Artificial Sequence: Synthetic primer
  source 1..25: mol_type = other DNA
gggaaatggtctaggagagtaaagt

SEQ ID NO: 210 - DNA, 25 bp, synthetic construct
  misc_feature 1..25: note = Description of Artificial Sequence: Synthetic primer
  source 1..25: mol_type = other DNA
gtttggctcaaattgttactcttca

SEQ ID NO: 211 - DNA, 25 bp, synthetic construct
  misc_feature 1..25: note = Description of Artificial Sequence: Synthetic primer
  source 1..25: mol_type = other DNA
gtgagagtaatgtgtttgctgagag

SEQ ID NO: 212 - DNA, 20 bp, synthetic construct
  misc_feature 1..20: note = Description of Artificial Sequence: Synthetic primer
  source 1..20: mol_type = other DNA
cgggcttggacagaacttac

SEQ ID NO: 213 - DNA, 20 bp, synthetic construct
  misc_feature 1..20: note = Description of Artificial Sequence: Synthetic primer
  source 1..20: mol_type = other DNA
ctgcgtagtgccaaaacaaa

SEQ ID NO: 214 - DNA, 24 bp, synthetic construct
  misc_feature 1..24: note = Description of Artificial Sequence: Synthetic primer
  source 1..24: mol_type = other DNA
taatttcattgaagagtggctgaa

SEQ ID NO: 215 - DNA, 24 bp, synthetic construct
  misc_feature 1..24: note = Description of Artificial Sequence: Synthetic primer
  source 1..24: mol_type = other DNA
aagccctgtgtggtagtagtcagt

SEQ ID NO: 216 - DNA, 24 bp, synthetic construct
  misc_feature 1..24: note = Description of Artificial Sequence: Synthetic primer
  source 1..24: mol_type = other DNA
tgagtcatgttggataaccagtct

SEQ ID NO: 217 - DNA, 24 bp, synthetic construct
  misc_feature 1..24: note = Description of Artificial Sequence: Synthetic primer
  source 1..24: mol_type = other DNA
gaaggtcaggaacatacaattcaa

SEQ ID NO: 218 - DNA, 24 bp, synthetic construct
  misc_feature 1..24: note = Description of Artificial Sequence: Synthetic primer
  source 1..24: mol_type = other DNA
gatatgggcatgtcagtttcatag

SEQ ID NO: 219 - DNA, 24 bp, synthetic construct
  misc_feature 1..24: note = Description of Artificial Sequence: Synthetic primer
  source 1..24: mol_type = other DNA
tgctgttgattaatggttgatagg

SEQ ID NO: 220 - DNA, 23 bp, synthetic construct
  misc_feature 1..23: note = Description of Artificial Sequence: Synthetic primer
  source 1..23: mol_type = other DNA
ttttaaattgccatgtttgtgtc

SEQ ID NO: 221 - DNA, 24 bp, synthetic construct
  misc_feature 1..24: note = Description of Artificial Sequence: Synthetic primer
  source 1..24: mol_type = other DNA
atgaataacctaatgggcagaaaa

SEQ ID NO: 222 - DNA, 24 bp, synthetic construct
  misc_feature 1..24: note = Description of Artificial Sequence: Synthetic primer
  source 1..24: mol_type = other DNA
tcaagtcgcttcattttgatagac

SEQ ID NO: 223 - DNA, 24 bp, synthetic construct
  misc_feature 1..24: note = Description of Artificial Sequence: Synthetic primer
  source 1..24: mol_type = other DNA
cacaacaaaacatatagccaaagc

SEQ ID NO: 224 - DNA, 25 bp, synthetic construct
  misc_feature 1..25: note = Description of Artificial Sequence: Synthetic primer
  source 1..25: mol_type = other DNA
tgctgctaaaataacacaaatcagt

SEQ ID NO: 225 - DNA, 23 bp, synthetic construct
  misc_feature 1..23: note = Description of Artificial Sequence: Synthetic primer
  source 1..23: mol_type = other DNA
ctgtgcctattgtggttatcctg

SEQ ID NO: 226 - DNA, 24 bp, synthetic construct
  misc_feature 1..24: note = Description of Artificial Sequence: Synthetic primer
  source 1..24: mol_type = other DNA
attgatctgcaatacatgtggagt

SEQ ID NO: 227 - DNA, 24 bp, synthetic construct
  misc_feature 1..24: note = Description of Artificial Sequence: Synthetic primer
  source 1..24: mol_type = other DNA
tttgcctctgctattacagtatgg

SEQ ID NO: 228 - DNA, 24 bp, synthetic construct
  misc_feature 1..24: note = Description of Artificial Sequence: Synthetic primer
  source 1..24: mol_type = other DNA
tgtagggtggttggctaaaataat

SEQ ID NO: 229 - DNA, 24 bp, synthetic construct
  misc_feature 1..24: note = Description of Artificial Sequence: Synthetic primer
  source 1..24: mol_type = other DNA
tttttgcacagtcaataacacaaa

SEQ ID NO: 230 - DNA, 24 bp, synthetic construct
  misc_feature 1..24: note = Description of Artificial Sequence: Synthetic primer
  source 1..24: mol_type = other DNA
ggctggtctcacaattgtacttta

SEQ ID NO: 231 - DNA, 24 bp, synthetic construct
  misc_feature 1..24: note = Description of Artificial Sequence: Synthetic primer
  source 1..24: mol_type = other DNA
cattatggactgaaaatctcagca

SEQ ID NO: 232 - DNA, 24 bp, synthetic construct
  misc_feature 1..24: note = Description of Artificial Sequence: Synthetic primer
  source 1..24: mol_type = other DNA
atcatcctagccataacacaatga

SEQ ID NO: 233 - DNA, 24 bp, synthetic construct
  misc_feature 1..24: note = Description of Artificial Sequence: Synthetic primer
  source 1..24: mol_type = other DNA
ttcagctttaacgtgattttctgt

SEQ ID NO: 234 - DNA, 24 bp, synthetic construct
  misc_feature 1..24: note = Description of Artificial Sequence: Synthetic primer
  source 1..24: mol_type = other DNA
ggattcagaagctgtttacgaagt

SEQ ID NO: 235 - DNA, 24 bp, synthetic construct
  misc_feature 1..24: note = Description of Artificial Sequence: Synthetic primer
  source 1..24: mol_type = other DNA
tttagctggattggaaaaacaaat

SEQ ID NO: 236 - DNA, 24 bp, synthetic construct
  misc_feature 1..24: note = Description of Artificial Sequence: Synthetic primer
  source 1..24: mol_type = other DNA
aactcaccccattgttggtatatt

SEQ ID NO: 237 - DNA, 24 bp, synthetic construct
  misc_feature 1..24: note = Description of Artificial Sequence: Synthetic primer
  source 1..24: mol_type = other DNA
ccttgtccaaataccgaaatacat

SEQ ID NO: 238 - DNA, 24 bp, synthetic construct
  misc_feature 1..24: note = Description of Artificial Sequence: Synthetic primer
  source 1..24: mol_type = other DNA
cacataattcatgaacttggcttc

SEQ ID NO: 239 - DNA, 24 bp, synthetic construct
  misc_feature 1..24: note = Description of Artificial Sequence: Synthetic primer
  source 1..24: mol_type = other DNA
tagtagctggggaggaagatacag

SEQ ID NO: 240 - DNA, 24 bp, synthetic construct
  misc_feature 1..24: note = Description of Artificial Sequence: Synthetic primer
  source 1..24: mol_type = other DNA
tttttgttttaattgcgactgtgt

SEQ ID NO: 241 - DNA, 24 bp, synthetic construct
  misc_feature 1..24: note = Description of Artificial Sequence: Synthetic primer
  source 1..24: mol_type = other DNA
agaaaaggggttttcttttgactt

SEQ ID NO: 242 - DNA, 24 bp, synthetic construct
  misc_feature 1..24: note = Description of Artificial Sequence: Synthetic primer
  source 1..24: mol_type = other DNA
cattgtgactggatgagaagaaac

SEQ ID NO: 243 - DNA, 24 bp, synthetic construct
  misc_feature 1..24: note = Description of Artificial Sequence: Synthetic primer
  source 1..24: mol_type = other DNA
aacggctgttattaaagtcctcag

SEQ ID NO: 244 - DNA, 24 bp, synthetic construct
  misc_feature 1..24: note = Description of Artificial Sequence: Synthetic primer
  source 1..24: mol_type = other DNA
caagtcagaagtcacttgctttgt

SEQ ID NO: 245 - DNA, 24 bp, synthetic construct
  misc_feature 1..24: note = Description of Artificial Sequence: Synthetic primer
  source 1..24: mol_type = other DNA
ttttatgtgcaggaatcagtctgt

SEQ ID NO: 246 - DNA, 18 bp, synthetic construct
  misc_feature 1..18: note = Description of Artificial Sequence: Synthetic primer
  source 1..18: mol_type = other DNA
tggcggcgttttcattat

SEQ ID NO: 247 - DNA, 26 bp, synthetic construct
  misc_feature 1..26: note = Description of Artificial Sequence: Synthetic primer
  source 1..26: mol_type = other DNA
ttcgatccgtaatgattgttctagcc

SEQ ID NO: 248 - DNA, 20 bp, synthetic construct
  misc_feature 1..20: note = Description of Artificial Sequence: Synthetic primer
  source 1..20: mol_type = other DNA
ggtcttccagagtgctgagg

SEQ ID NO: 249 - DNA, 24 bp, synthetic construct
  misc_feature 1..24: note = Description of Artificial Sequence: Synthetic primer
  source 1..24: mol_type = other DNA
tgtgtgcttctgtacacatcatct

SEQ ID NO: 250 - DNA, 24 bp, synthetic construct
  misc_feature 1..24: note = Description of Artificial Sequence: Synthetic primer
  source 1..24: mol_type = other DNA
agatttcaaccctcaaaaactgag

SEQ ID NO: 251 - DNA, 24 bp, synthetic construct
  misc_feature 1..24: note = Description of Artificial Sequence: Synthetic primer
  source 1..24: mol_type = other DNA
taaactctttcttttccgcaattc

SEQ ID NO: 252 - DNA, 24 bp, synthetic construct
  misc_feature 1..24: note = Description of Artificial Sequence: Synthetic primer
  source 1..24: mol_type = other DNA
caaggtgacctgctacctaaaaat

SEQ ID NO: 253 - DNA, 24 bp, synthetic construct
  misc_feature 1..24: note = Description of Artificial Sequence: Synthetic primer
  source 1..24: mol_type = other DNA
tatgaccaaggctatgtgttcact

SEQ ID NO: 254 - DNA, 24 bp, synthetic construct
  misc_feature 1..24: note = Description of Artificial Sequence: Synthetic primer
  source 1..24: mol_type = other DNA
acagcctctctccagtaacattct

SEQ ID NO: 255 - DNA, 24 bp, synthetic construct
  misc_feature 1..24: note = Description of Artificial Sequence: Synthetic primer
  source 1..24: mol_type = other DNA
tattcttgcagtggtttcacattt

SEQ ID NO: 256 - DNA, 24 bp, synthetic construct
  misc_feature 1..24: note = Description of Artificial Sequence: Synthetic primer
  source 1..24: mol_type = other DNA
atattttaagccaagacccaacaa

SEQ ID NO: 257 - DNA, 24 bp, synthetic construct
  misc_feature 1..24: note = Description of Artificial Sequence: Synthetic primer
  source 1..24: mol_type = other DNA
ctttcaactgtctgtctgattgct

SEQ ID NO: 258 - DNA, 24 bp, synthetic construct
  misc_feature 1..24: note = Description of Artificial Sequence: Synthetic primer
  source 1..24: mol_type = other DNA
aacagcctctcttcattgttctct

SEQ ID NO: 259 - DNA, 24 bp, synthetic construct
  misc_feature 1..24: note = Description of Artificial Sequence: Synthetic primer
  source 1..24: mol_type = other DNA
ctctggaacttgtctctgtcttga

SEQ ID NO: 260 - DNA, 24 bp, synthetic construct
  misc_feature 1..24: note = Description of Artificial Sequence: Synthetic primer
  source 1..24: mol_type = other DNA
ctttcctgcgttctcatgttacta

SEQ ID NO: 261 - DNA, 24 bp, synthetic construct
  misc_feature 1..24: note = Description of Artificial Sequence: Synthetic primer
  source 1..24: mol_type = other DNA
ccttatatccgtatcgctcactct

SEQ ID NO: 262 - DNA, 24 bp, synthetic construct
  misc_feature 1..24: note = Description of Artificial Sequence: Synthetic primer
  source 1..24: mol_type = other DNA
catatctgtctaacttccgcacac

SEQ ID NO: 263 - DNA, 24 bp, synthetic construct
  misc_feature 1..24: note = Description of Artificial Sequence: Synthetic primer
  source 1..24: mol_type = other DNA
acaggtgttatgttgtctgcatct

SEQ ID NO: 264 - DNA, 24 bp, synthetic construct
  misc_feature 1..24: note = Description of Artificial Sequence: Synthetic primer
  source 1..24: mol_type = other DNA
actccattcccagattagttatgc

SEQ ID NO: 265 - DNA, 24 bp, synthetic construct
  misc_feature 1..24: note = Description of Artificial Sequence: Synthetic primer
  source 1..24: mol_type = other DNA
ctgttttctttgtgagagtggaga

SEQ ID NO: 266 - DNA, 24 bp, synthetic construct
  misc_feature 1..24: note = Description of Artificial Sequence: Synthetic primer
  source 1..24: mol_type = other DNA
tgtaaggtggtcaaacttgctcta

SEQ ID NO: 267 - DNA, 25 bp, synthetic construct
  misc_feature 1..25: note = Description of Artificial Sequence: Synthetic primer
  source 1..25: mol_type = other DNA
tttttcctagtacccacagattttt

SEQ ID NO: 268 - DNA, 24 bp, synthetic construct
  misc_feature 1..24: note = Description of Artificial Sequence: Synthetic primer
  source 1..24: mol_type = other DNA
tccctgattctctcatttgtgtta

SEQ ID NO: 269 - DNA, 24 bp, synthetic construct
  misc_feature 1..24: note = Description of Artificial Sequence: Synthetic primer
  source 1..24: mol_type = other DNA
ttgggaacatcagagaaagtatga

SEQ ID NO: 270 - DNA, 24 bp, synthetic construct
  misc_feature 1..24: note = Description of Artificial Sequence: Synthetic primer
  source 1..24: mol_type = other DNA
acaaattacagtctcctgggaaag

SEQ ID NO: 271 - DNA, 24 bp, synthetic construct
  misc_feature 1..24: note = Description of Artificial Sequence: Synthetic primer
  source 1..24: mol_type = other DNA
agtagcttaccttggcagagaaaa

SEQ ID NO: 272 - DNA, 24 bp, synthetic construct
  misc_feature 1..24: note = Description of Artificial Sequence: Synthetic primer
  source 1..24: mol_type = other DNA
tgacatactgttaccctttgcagt

SEQ ID NO: 273 - DNA, 23 bp, synthetic construct
  misc_feature 1..23: note = Description of Artificial Sequence: Synthetic primer
  source 1..23: mol_type = other DNA
gaaaggctcagtgaatgtttgtt

SEQ ID NO: 274 - DNA, 24 bp, synthetic construct
  misc_feature 1..24: note = Description of Artificial Sequence: Synthetic primer
  source 1..24: mol_type = other DNA
cactgcatcatctcattaaatcaa

SEQ ID NO: 275 - DNA, 24 bp, synthetic construct
  misc_feature 1..24: note = Description of Artificial Sequence: Synthetic primer
  source 1..24: mol_type = other DNA
cccatatattcatgattacccaca

SEQ ID NO: 276 - DNA, 24 bp, synthetic construct
  misc_feature 1..24: note = Description of Artificial Sequence: Synthetic primer
  source 1..24: mol_type = other DNA
tatcagaacgagcactaaaagcac

SEQ ID NO: 277 - DNA, 20 bp, synthetic construct
  misc_feature 1..20: note = Description of Artificial Sequence: Synthetic primer
  source 1..20: mol_type = other DNA
ttgggaggctgaggtacaag

SEQ ID NO: 278 - DNA, 24 bp, synthetic construct
  misc_feature 1..24: note = Description of Artificial Sequence: Synthetic primer
  source 1..24: mol_type = other DNA
gaatgaaaaacaaacagaaggtga

SEQ ID NO: 279 - DNA, 24 bp, synthetic construct
  misc_feature 1..24: note = Description of Artificial Sequence: Synthetic primer
  source 1..24: mol_type = other DNA
ctcctcatctgtacccttcaatct

SEQ ID NO: 280 - DNA, 24 bp, synthetic construct
  misc_feature 1..24: note = Description of Artificial Sequence: Synthetic primer
  source 1..24: mol_type = other DNA
agagtggcatctagtgtcagtgag

SEQ ID NO: 281 - DNA, 24 bp, synthetic construct
  misc_feature 1..24: note = Description of Artificial Sequence: Synthetic primer
  source 1..24: mol_type = other DNA
taccaaaagcttctcctgtttacc

SEQ ID NO: 282 - DNA, 24 bp, synthetic construct
  misc_feature 1..24: note = Description of Artificial Sequence: Synthetic primer
  source 1..24: mol_type = other DNA
gtaagttggatggcctattctttg

SEQ ID NO: 283 - DNA, 24 bp, synthetic construct
  misc_feature 1..24: note = Description of Artificial Sequence: Synthetic primer
  source 1..24: mol_type = other DNA
gaaggaaatgcaaggatacaagat

SEQ ID NO: 284 - DNA, 24 bp, synthetic construct
  misc_feature 1..24: note = Description of Artificial Sequence: Synthetic primer
  source 1..24: mol_type = other DNA
tgattgaaagaatcattccagaaa

SEQ ID NO: 285 - DNA, 24 bp, synthetic construct
  misc_feature 1..24: note = Description of Artificial Sequence: Synthetic primer
  source 1..24: mol_type = other DNA
tcagaaggaaaattgaaattggtt

SEQ ID NO: 286 - DNA, 24 bp, synthetic construct
  misc_feature 1..24: note = Description of Artificial Sequence: Synthetic primer
  source 1..24: mol_type = other DNA
cagatgtgttcttcatcattcctc

SEQ ID NO: 287 - DNA, 24 bp, synthetic construct
  misc_feature 1..24: note = Description of Artificial Sequence: Synthetic primer
  source 1..24: mol_type = other DNA
ttctctttagggaaagctctcaaa

SEQ ID NO: 288 - DNA, 24 bp, synthetic construct
  misc_feature 1..24: note = Description of Artificial Sequence: Synthetic primer
  source 1..24: mol_type = other DNA
gggtatagatcatatggagggaag

SEQ ID NO: 289 - DNA, 20 bp, synthetic construct
  misc_feature 1..20: note = Description of Artificial Sequence: Synthetic primer
  source 1..20: mol_type = other DNA
agatgatctgcccacctcag

SEQ ID NO: 290 - DNA, 24 bp, synthetic construct
  misc_feature 1..24: note = Description of Artificial Sequence: Synthetic primer
  source 1..24: mol_type = other DNA
ctttcttcctcatttagtggcaat

SEQ ID NO: 291 - DNA, 24 bp, synthetic construct
  misc_feature 1..24: note = Description of Artificial Sequence: Synthetic primer
  source 1..24: mol_type = other DNA
atgaattgcagattgatggtactg

SEQ ID NO: 292 - DNA, 24 bp, synthetic construct
  misc_feature 1..24: note = Description of Artificial Sequence: Synthetic primer
  source 1..24: mol_type = other DNA
tctcaccaagaaccaaattgtcta

SEQ ID NO: 293 - DNA, 24 bp, synthetic construct
  misc_feature 1..24: note = Description of Artificial Sequence: Synthetic primer
  source 1..24: mol_type = other DNA
gtaggataccttggcaacagtctt

SEQ ID NO: 294 - DNA, 24 bp, synthetic construct
  misc_feature 1..24: note = Description of Artificial Sequence: Synthetic primer
  source 1..24: mol_type = other DNA
ttaacgaattgtgagatttgctgt

SEQ ID NO: 295 - DNA, 24 bp, synthetic construct
  misc_feature 1..24: note = Description of Artificial Sequence: Synthetic primer
  source 1..24: mol_type = other DNA
tcagaaagtcaagtagcacacaca

SEQ ID NO: 296 - DNA, 23 bp, synthetic construct
  misc_feature 1..23: note = Description of Artificial Sequence: Synthetic primer
  source 1..23: mol_type = other DNA
agaagcacacactcaggtaaagc

SEQ ID NO: 297 - DNA, 24 bp, synthetic construct
  misc_feature 1..24: note = Description of Artificial Sequence: Synthetic primer
  source 1..24: mol_type = other DNA
tctttgggggaataatgactaaaa

SEQ ID NO: 298 - DNA, 24 bp, synthetic construct
  misc_feature 1..24: note = Description of Artificial Sequence: Synthetic primer
  source 1..24: mol_type = other DNA
tttggcatttatgggaataaaact

SEQ ID NO: 299 - DNA, 23 bp, synthetic construct
  misc_feature 1..23: note = Description of Artificial Sequence: Synthetic primer
  source 1..23: mol_type = other DNA
actaattctggtcaagcccatca

SEQ ID NO: 300 - DNA, 24 bp, synthetic construct
  misc_feature 1..24: note = Description of Artificial Sequence: Synthetic primer
  source 1..24: mol_type = other DNA
ttaagacatcggatgaacagaaag

SEQ ID NO: 301 - DNA, 23 bp, synthetic construct
  misc_feature 1..23: note = Description of Artificial Sequence: Synthetic primer
  source 1..23: mol_type = other DNA
agaagctttctgacatgatctgc

SEQ ID NO: 302 - DNA, 24 bp, synthetic construct
  misc_feature 1..24: note = Description of Artificial Sequence: Synthetic primer
  source 1..24: mol_type = other DNA
tcaattgcattaggacttagacca

SEQ ID NO: 303 - DNA, 24 bp, synthetic construct
  misc_feature 1..24: note = Description of Artificial Sequence: Synthetic primer
  source 1..24: mol_type = other DNA
gttaaattacctgtgaagcccttg

SEQ ID NO: 304 - DNA, 24 bp, synthetic construct
  misc_feature 1..24: note = Description of Artificial Sequence: Synthetic primer
  source 1..24: mol_type = other DNA
cggaaaacagatccactttatgat

SEQ ID NO: 305 - DNA, 24 bp, synthetic construct
  misc_feature 1..24: note = Description of Artificial Sequence: Synthetic primer
  source 1..24: mol_type = other DNA
aaatccactggaaacatcttgagt

SEQ ID NO: 306 - DNA, 24 bp, synthetic construct
  misc_feature 1..24: note = Description of Artificial Sequence: Synthetic primer
  source 1..24: mol_type = other DNA
agtctcttcagaatcatgccctat

SEQ ID NO: 307 - DNA, 22 bp, synthetic construct
  misc_feature 1..22: note = Description of Artificial Sequence: Synthetic primer
  source 1..22: mol_type = other DNA
gcttggtggcacatacctgtag

SEQ ID NO: 308 - DNA, 24 bp, synthetic construct
  misc_feature 1..24: note = Description of Artificial Sequence: Synthetic primer
  source 1..24: mol_type = other DNA
ggtaggtagatttgcttgcttgtt

SEQ ID NO: 309 - DNA, 24 bp, synthetic construct
  misc_feature 1..24: note = Description of Artificial Sequence: Synthetic primer
  source 1..24: mol_type = other DNA
agctctcagcagagtagggattta

SEQ ID NO: 310 - DNA, 21 bp, synthetic construct
  misc_feature 1..21: note = Description of Artificial Sequence: Synthetic primer
  source 1..21: mol_type = other DNA
gtgagtctactgcaccccatc

SEQ ID NO: 311 - DNA, 24 bp, synthetic construct
  misc_feature 1..24: note = Description of Artificial Sequence: Synthetic primer
  source 1..24: mol_type = other DNA
tgacactgtgaagtcaattctgtc

SEQ ID NO: 312 - DNA, 24 bp, synthetic construct
  misc_feature 1..24: note = Description of Artificial Sequence: Synthetic primer
  source 1..24: mol_type = other DNA
tcaagaacttgacaatgagcaaat

SEQ ID NO: 313 - DNA, 22 bp, synthetic construct
  misc_feature 1..22: note = Description of Artificial Sequence: Synthetic primer
  source 1..22: mol_type = other DNA
tatccgatccactgttgtgtgt

SEQ ID NO: 314 - DNA, 23 bp, synthetic construct
  misc_feature 1..23: note = Description of Artificial Sequence: Synthetic primer
  source 1..23: mol_type = other DNA
caggagacccaaaaccactctac

SEQ ID NO: 315 - DNA, 24 bp, synthetic construct
  misc_feature 1..24: note = Description of Artificial Sequence: Synthetic primer
  source 1..24: mol_type = other DNA
ttgttctacaaatagggcttcctt

SEQ ID NO: 316 - DNA, 24 bp, synthetic construct
  misc_feature 1..24: note = Description of Artificial Sequence: Synthetic primer
  source 1..24: mol_type = other DNA
tgttaagtttgggcttatgttcct

SEQ ID NO: 317 - DNA, 23 bp, synthetic construct
  misc_feature 1..23: note = Description of Artificial Sequence: Synthetic primer
  source 1..23: mol_type = other DNA
cacaagtctcactgcacaaacat

SEQ ID NO: 318 - DNA, 24 bp, synthetic construct
  misc_feature 1..24: note = Description of Artificial Sequence: Synthetic primer
  source 1..24: mol_type = other DNA
tgacccatgattatctctctttga

SEQ ID NO: 319 - DNA, 24 bp, synthetic construct
  misc_feature 1..24: note = Description of Artificial Sequence: Synthetic primer
  source 1..24: mol_type = other DNA
ttcagcttctgattggttttaatg

SEQ ID NO: 320 - DNA, 24 bp, synthetic construct
  misc_feature 1..24: note = Description of Artificial Sequence: Synthetic primer
  source 1..24: mol_type = other DNA
ccaattccttaattttccctacag

SEQ ID NO: 321 - DNA, 21 bp, synthetic construct
  misc_feature 1..21: note = Description of Artificial Sequence: Synthetic primer
  source 1..21: mol_type = other DNA
atctcagaccaggagggagac

SEQ ID NO: 322 - DNA, 24 bp, synthetic construct
  misc_feature 1..24: note = Description of Artificial Sequence: Synthetic primer
  source 1..24: mol_type = other DNA
cctcagggtcagtacatttttcag

SEQ ID NO: 323 - DNA, 24 bp, synthetic construct
  misc_feature 1..24: note = Description of Artificial Sequence: Synthetic primer
  source 1..24: mol_type = other DNA
ttcttaggacattgctccacatac

SEQ ID NO: 324 - DNA, 24 bp, synthetic construct
  misc_feature 1..24: note = Description of Artificial Sequence: Synthetic primer
  source 1..24: mol_type = other DNA
gcaaacataatgcaactcgtaatc

SEQ ID NO: 325 - DNA, 21 bp, synthetic construct
  misc_feature 1..21: note = Description of Artificial Sequence: Synthetic primer
  source 1..21: mol_type = other DNA
gcaagggagtctgtgtctttg

SEQ ID NO: 326 - DNA, 24 bp, synthetic construct
  misc_feature 1..24: note = Description of Artificial Sequence: Synthetic primer
  source 1..24: mol_type = other DNA
tcatttaagtggctgttctgtgtt

SEQ ID NO: 327 - DNA, 24 bp, synthetic construct
  misc_feature 1..24: note = Description of Artificial Sequence: Synthetic primer
  source 1..24: mol_type = other DNA
acaaaacagagagaaaaggcagag

SEQ ID NO: 328 - DNA, 23 bp, synthetic construct
  misc_feature 1..23: note = Description of Artificial Sequence: Synthetic primer
  source 1..23: mol_type = other DNA
gttttgatttctggtgcctacag

SEQ ID NO: 329 - DNA, 20 bp, synthetic construct
  misc_feature 1..20: note = Description of Artificial Sequence: Synthetic primer
  source 1..20: mol_type = other DNA
actgaagctgaagcccagtc

SEQ ID NO: 330 - DNA, 23 bp, synthetic construct
  misc_feature 1..23: note = Description of Artificial Sequence: Synthetic primer
  source 1..23: mol_type = other DNA
acatgagctctcaggtttctgac

SEQ ID NO: 331 - DNA, 25 bp, synthetic construct
  misc_feature 1..25: note = Description of Artificial Sequence: Synthetic primer
  source 1..25: mol_type = other DNA
tcaaacttagatggttccctatgtt

SEQ ID NO: 332 - DNA, 24 bp, synthetic construct
  misc_feature 1..24: note = Description of Artificial Sequence: Synthetic primer
  source 1..24: mol_type = other DNA
gtaccctgaaaatgtagggtgact

SEQ ID NO: 333 - DNA, 20 bp, synthetic construct
  misc_feature 1..20: note = Description of Artificial Sequence: Synthetic primer
  source 1..20: mol_type = other DNA
cacttcccaagtgaggcaat

SEQ ID NO: 334 - DNA, 24 bp, synthetic construct
  misc_feature 1..24: note = Description of Artificial Sequence: Synthetic primer
  source 1..24: mol_type = other DNA
ctatacttggggctgacttgctac

SEQ ID NO: 335 - DNA, 24 bp, synthetic construct
  misc_feature 1..24: note = Description of Artificial Sequence: Synthetic primer
  source 1..24: mol_type = other DNA
tcgtataggttactttggctcaca

SEQ ID NO: 336 - DNA, 24 bp, synthetic construct
  misc_feature 1..24: note = Description of Artificial Sequence: Synthetic primer
  source 1..24: mol_type = other DNA
agggatctttactcctcagtgtgt

SEQ ID NO: 337 - DNA, 24 bp, synthetic construct
  misc_feature 1..24: note = Description of Artificial Sequence: Synthetic primer
  source 1..24: mol_type = other DNA
tgtagaagttggaatatcctgctg

SEQ ID NO: 338 - DNA, 24 bp, synthetic construct
  misc_feature 1..24: note = Description of Artificial Sequence: Synthetic primer
  source 1..24: mol_type = other DNA
gtcaacaatttgatctcaggcttc

SEQ ID NO: 339 - DNA, 24 bp, synthetic construct
  misc_feature 1..24: note = Description of Artificial Sequence: Synthetic primer
  source 1..24: mol_type = other DNA
ctcagtactaaagatggacgcttg

SEQ ID NO: 340 - DNA, 24 bp, synthetic construct
  misc_feature 1..24: note = Description of Artificial Sequence: Synthetic primer
  source 1..24: mol_type = other DNA
aatcatttcagtcttcccaacaat

SEQ ID NO: 341 - DNA, 24 bp, synthetic construct
  misc_feature 1..24: note = Description of Artificial Sequence: Synthetic primer
  source 1..24: mol_type = other DNA
gggaatcacagtagatgtttgtca

SEQ ID NO: 342 - DNA, 24 bp, synthetic construct
  misc_feature 1..24: note = Description of Artificial Sequence: Synthetic primer
  source 1..24: mol_type = other DNA
agaccaggaggtaagaacattttg

SEQ ID NO: 343 - DNA, 24 bp, synthetic construct
  misc_feature 1..24: note = Description of Artificial Sequence: Synthetic primer
  source 1..24: mol_type = other DNA
ccacatagaaagagacttgcagaa

SEQ ID NO: 344 - DNA, 24 bp, synthetic construct
  misc_feature 1..24: note = Description of Artificial Sequence: Synthetic primer
  source 1..24: mol_type = other DNA
agagatgccaaaagaacagtcaat

SEQ ID NO: 345 - DNA, 24 bp, synthetic construct
  misc_feature 1..24: note = Description of Artificial Sequence: Synthetic primer
  source 1..24: mol_type = other DNA
tgtgccttaggctatgtaaactgt

SEQ ID NO: 346 - DNA, 24 bp, synthetic construct
  misc_feature 1..24: note = Description of Artificial Sequence: Synthetic primer
  source 1..24: mol_type = other DNA
aaacccttgtaaccaaaattacca

SEQ ID NO: 347 - DNA, 24 bp, synthetic construct
  misc_feature 1..24: note = Description of Artificial Sequence: Synthetic primer
  source 1..24: mol_type = other DNA
taactgcatcagaagtccttgcta

SEQ ID NO: 348 - DNA, 22 bp, synthetic construct
  misc_feature 1..22: note = Description of Artificial Sequence: Synthetic primer
  source 1..22: mol_type = other DNA
ggagaccaagctgctaaagtca

SEQ ID NO: 349 - DNA, 37 bp, synthetic construct
  misc_feature 1..37: note = Description of Artificial Sequence: Synthetic primer
  source 1..37: mol_type = other DNA
gtggtgccgcgggagtttggctcaaattgttactctt

SEQ ID NO: 350 - DNA, 37 bp, synthetic construct
  misc_feature 1..37: note = Description of Artificial Sequence: Synthetic primer
  source 1..37: mol_type = other DNA
gtggtgccgcgggggaaatggtctaggagagtaaagt

SEQ ID NO: 351 - DNA, 34 bp, synthetic construct
  misc_feature 1..34: note = Description of Artificial Sequence: Synthetic primer
  source 1..34: mol_type = other DNA
gtggtgccgcgggagaggttatgtggctttacca

SEQ ID NO: 352 - DNA, 32 bp, synthetic construct
  misc_feature 1..32: note = Description of Artificial Sequence: Synthetic primer
  source 1..32: mol_type = other DNA
gtggtgccgcggctcattctcatgcctggaca

SEQ ID NO: 353 - DNA, 32 bp, synthetic construct
  misc_feature 1..32: note = Description of Artificial Sequence: Synthetic primer
  source 1..32: mol_type = other DNA
gtggtgccgcggcgggcttggacagaacttac

SEQ ID NO: 354 - DNA, 32 bp, synthetic construct
  misc_feature 1..32: note = Description of Artificial Sequence: Synthetic primer
  source 1..32: mol_type = other DNA
gtggtgccgcggctgcgtagtgccaaaacaaa

SEQ ID NO: 355 - DNA, 36 bp, synthetic construct
  misc_feature 1..36: note = Description of Artificial Sequence: Synthetic primer
  source 1..36: mol_type = other DNA
gtggtgccgcggtaatttcattgaagagtggctgaa

SEQ ID NO: 356 - DNA, 36 bp, synthetic construct
  misc_feature 1..36: note = Description of Artificial Sequence: Synthetic primer
  source 1..36: mol_type = other DNA
gtggtgccgcggaagccctgtgtggtagtagtcagt

SEQ ID NO: 357 - DNA, 36 bp, synthetic construct
  misc_feature 1..36: note = Description of Artificial Sequence: Synthetic primer
  source 1..36: mol_type = other DNA
gtggtgccgcggcaagtcagaagtcacttgctttgt

SEQ ID NO: 358 - DNA, 36 bp, synthetic construct
  misc_feature 1..36: note = Description of Artificial Sequence: Synthetic primer
  source 1..36: mol_type = other DNA
gtggtgccgcggttttatgtgcaggaatcagtctgt

SEQ ID NO: 359 - DNA, 36 bp, synthetic construct
  misc_feature 1..36: note = Description of Artificial Sequence: Synthetic primer
  source 1..36: mol_type = other DNA
gtggtgccgcggtgtgtgcttctgtacacatcatct

SEQ ID NO: 360 - DNA, 36 bp, synthetic construct
  misc_feature 1..36: note = Description of Artificial Sequence: Synthetic primer
  source 1..36: mol_type = other DNA
gtggtgccgcggagatttcaaccctcaaaaactgag

SEQ ID NO: 361 - DNA, 36 bp, synthetic construct
  misc_feature 1..36: note = Description of Artificial Sequence: Synthetic primer
  source 1..36: mol_type = other DNA
gtggtgccgcggttgggaacatcagagaaagtatga

SEQ ID NO: 362 - DNA, 36 bp, synthetic construct
  misc_feature 1..36: note = Description of Artificial Sequence: Synthetic primer
  source 1..36: mol_type = other DNA
gtggtgccgcggacaaattacagtctcctgggaaag

SEQ ID NO: 363 - DNA, 32 bp, synthetic construct
  misc_feature 1..32: note = Description of Artificial Sequence: Synthetic primer
  source 1..32: mol_type = other DNA
gtggtgccgcggcacttcccaagtgaggcaat

SEQ ID NO: 364 - DNA, 36 bp, synthetic construct
  misc_feature 1..36: note = Description of Artificial Sequence: Synthetic primer
  source 1..36: mol_type = other DNA
gtggtgccgcggctatacttggggctgacttgctac

SEQ ID NO: 365 - DNA, 34 bp, synthetic construct
  misc_feature 1..34: note = Description of Artificial Sequence: Synthetic primer
  source 1..34: mol_type = other DNA
gtggtgccgcggttggctctttagcttgtgtttc

SEQ ID NO: 366 - DNA, 32 bp, synthetic construct
  misc_feature 1..32: note = Description of Artificial Sequence: Synthetic primer
  source 1..32: mol_type = other DNA
gtggtgccgcggtgagactcccaaaggcaatc

SEQ ID NO: 367 - DNA, 34 bp, synthetic construct; identical to SEQ ID NO: 365
  misc_feature 1..34: note = Description of Artificial Sequence: Synthetic primer
  source 1..34: mol_type = other DNA

SEQ ID NO: 368 - DNA, 32 bp, synthetic construct
  misc_feature 1..32: note = Description of Artificial Sequence: Synthetic primer
  source 1..32: mol_type = other DNA
gtggtgccgcggactgaggggtgatcttggtg

SEQ ID NO: 369 - DNA, 32 bp, synthetic construct
  misc_feature 1..32: note = Description of Artificial Sequence: Synthetic primer
  source 1..32: mol_type = other DNA
gtggtgccgcgggcagagaaagccagtcggta

SEQ ID NO: 370 - DNA, 32 bp, synthetic construct; identical to SEQ ID NO: 366
  misc_feature 1..32: note = Description of Artificial Sequence: Synthetic primer
  source 1..32: mol_type = other DNA

SEQ ID NO: 371 - DNA, 32 bp, synthetic construct; identical to SEQ ID NO: 369
  misc_feature 1..32: note = Description of Artificial Sequence: Synthetic primer
  source 1..32: mol_type = other DNA

SEQ ID NO: 372 - DNA, 32 bp, synthetic construct; identical to SEQ ID NO: 368
  misc_feature 1..32: note = Description of Artificial Sequence: Synthetic primer
  source 1..32: mol_type = other DNA

SEQ ID NO: 373 - DNA, 32 bp, synthetic construct
  misc_feature 1..32: note = Description of Artificial Sequence: Synthetic primer
  source 1..32: mol_type = other DNA
gtggtgccgcggccagagttcctagggcagag

SEQ ID NO: 374 - DNA, 32 bp, synthetic construct
  misc_feature 1..32: note = Description of Artificial Sequence: Synthetic primer
  source 1..32: mol_type = other DNA
gtggtgccgcggagctagtccccacattccac

SEQ ID NO: 375 - DNA, 32 bp, synthetic construct; identical to SEQ ID NO: 373
  misc_feature 1..32: note = Description of Artificial Sequence: Synthetic primer
  source 1..32: mol_type = other DNA

SEQ ID NO: 376 - DNA, 32 bp, synthetic construct
  misc_feature 1..32: note = Description of Artificial Sequence: Synthetic primer
  source 1..32: mol_type = other DNA
gtggtgccgcggggtggagggaaactttaggc

SEQ ID NO: 377 - DNA, 32 bp, synthetic construct; identical to SEQ ID NO: 352
  misc_feature 1..32: note = Description of Artificial Sequence: Synthetic primer
  source 1..32: mol_type = other DNA

SEQ ID NO: 378 - DNA, 32 bp, synthetic construct; identical to SEQ ID NO: 374
  misc_feature 1..32: note = Description of Artificial Sequence: Synthetic primer
  source 1..32: mol_type = other DNA

SEQ ID NO: 379 - DNA, 34 bp, synthetic construct
  misc_feature 1..34: note = Description of Artificial Sequence: Synthetic primer
  source 1..34: mol_type = other DNA
gtggtgccgcggtctcatgcctggacaagtaact

SEQ ID NO: 380 - DNA, 32 bp, synthetic construct; identical to SEQ ID NO: 376
  misc_feature 1..32: note = Description of Artificial Sequence: Synthetic primer
  source 1..32: mol_type = other DNA

SEQ ID NO: 381 - DNA, 32 bp, synthetic construct
  misc_feature 1..32: note = Description of Artificial Sequence: Synthetic primer
  source 1..32: mol_type = other DNA
gtggtgccgcggggcttggacagaacttaccg

SEQ ID NO: 382 - DNA, 32 bp, synthetic construct
  misc_feature 1..32: note = Description of Artificial Sequence: Synthetic primer
  source 1..32: mol_type = other DNA
gtggtgccgcggcaccactgtctgcctaagga

SEQ ID NO: 383 - DNA, 32 bp, synthetic construct; identical to SEQ ID NO: 381
  misc_feature 1..32: note = Description of Artificial Sequence: Synthetic primer
  source 1..32: mol_type = other DNA

SEQ ID NO: 384 - DNA, 32 bp, synthetic construct; identical to SEQ ID NO: 376
  misc_feature 1..32: note = Description of Artificial Sequence: Synthetic primer
  source 1..32: mol_type = other DNA

SEQ ID NO: 385 - DNA, 33 bp, synthetic construct
  misc_feature 1..33: note = Description of Artificial Sequence: Synthetic primer
  source 1..33: mol_type = other DNA
gtggtgccgcggcgtagtgccaaaacaaacagt

SEQ ID NO: 386 - DNA, 32 bp, synthetic construct; identical to SEQ ID NO: 382
  misc_feature 1..32: note = Description of Artificial Sequence: Synthetic primer
  source 1..32: mol_type = other DNA

SEQ ID NO: 387 - DNA, 33 bp, synthetic construct; identical to SEQ ID NO: 385
  misc_feature 1..33: note = Description of Artificial Sequence: Synthetic primer
  source 1..33: mol_type = other DNA

SEQ ID NO: 388 - DNA, 32 bp, synthetic construct; identical to SEQ ID NO: 376
  misc_feature 1..32: note = Description of Artificial Sequence: Synthetic primer
  source 1..32: mol_type = other DNA

SEQ ID NO: 389 - DNA, 32 bp, synthetic construct
  misc_feature 1..32: note = Description of Artificial Sequence: Synthetic primer
  source 1..32: mol_type = other DNA
gtggtgccgcgggcgagggcctacttgatatg

SEQ ID NO: 390 - DNA, 32 bp, synthetic construct
  misc_feature 1..32: note = Description of Artificial Sequence: Synthetic primer
  source 1..32: mol_type = other DNA
gtggtgccgcggcttcccaagtgaggcaatgc

SEQ ID NO: 391 - DNA, 33 bp, synthetic construct
  misc_feature 1..33: note = Description of Artificial Sequence: Synthetic primer
  source 1..33: mol_type = other DNA
gtggtgccgcggacgttttgtgctgctgtaaca

SEQ ID NO: 392 - DNA, 32 bp, synthetic construct
  misc_feature 1..32: note = Description of Artificial Sequence: Synthetic primer
  source 1..32: mol_type = other DNA
gtggtgccgcggctgcaggcacattctcttcc

SEQ ID NO: 393 - DNA, 32 bp, synthetic construct
  misc_feature 1..32: note = Description of Artificial Sequence: Synthetic primer
  source 1..32: mol_type = other DNA
gtggtgccgcgggccctgtgtggtagtagtca

SEQ ID NO: 394 - DNA, 32 bp, synthetic construct; identical to SEQ ID NO: 390
  misc_feature 1..32: note = Description of Artificial Sequence: Synthetic primer
  source 1..32: mol_type = other DNA

SEQ ID NO: 395 - DNA, 33 bp, synthetic construct
  misc_feature 1..33: note = Description of Artificial Sequence: Synthetic primer
  source 1..33: mol_type = other DNA
gtggtgccgcggcagtattaaggggtgggagct

SEQ ID NO: 396 - DNA, 32 bp, synthetic construct
  misc_feature 1..32: note = Description of Artificial Sequence: Synthetic primer
  source 1..32: mol_type = other DNA
gtggtgccgcggtctcttcctcacacagctga

SEQ ID NO: 397 - DNA, 32 bp, synthetic construct
  misc_feature 1..32: note = Description of Artificial Sequence: Synthetic primer
  source 1..32: mol_type = other DNA
gtggtgccgcggggagcttggagggaagagaa

SEQ ID NO: 398 - DNA, 32 bp, synthetic construct; identical to SEQ ID NO: 390
  misc_feature 1..32: note = Description of Artificial Sequence: Synthetic primer
  source 1..32: mol_type = other DNA

SEQ ID NO: 399 - DNA, 32 bp, synthetic construct
  misc_feature 1..32: note = Description of Artificial Sequence: Synthetic primer
  source 1..32: mol_type = other DNA
gtggtgccgcggatggatggggaagacactgg

SEQ ID NO: 400 - DNA, 32 bp, synthetic construct; identical to SEQ ID NO: 392
  misc_feature 1..32: note = Description of Artificial Sequence: Synthetic primer
  source 1..32: mol_type = other DNA

SEQ ID NO: 401 - DNA, 32 bp, synthetic construct
  misc_feature 1..32: note = Description of Artificial Sequence: Synthetic primer
  source 1..32: mol_type = other DNA
gtggtgccgcggggatgaaacagggcaggaac

SEQ ID NO: 402 - DNA, 31 bp, synthetic construct
  misc_feature 1..31: note = Description of Artificial Sequence: Synthetic primer
  source 1..31: mol_type = other DNA
gtggtgccgcggttcccaagtgaggcaatgc

SEQ ID NO: 403 - DNA, 32 bp, synthetic construct
  misc_feature 1..32: note = Description of Artificial Sequence: Synthetic primer
  source 1..32: mol_type = other DNA
gtggtgccgcggtttgcagagccatgatgagg

SEQ ID NO: 404 - DNA, 30 bp, synthetic construct
  misc_feature 1..30: note = Description of Artificial Sequence: Synthetic primer
  source 1..30: mol_type = other DNA
gtggtgccgcggcgacagccaaaacagccg

SEQ ID NO: 405 - DNA, 44 bp, synthetic construct
  misc_feature 1..44: note = Description of Artificial Sequence: Synthetic oligonucleotide
  source 1..44: mol_type = other DNA
aaaatattttagctcctactcagactgttactctggtgacacaa

SEQ ID NO: 406 - DNA, 44 bp, synthetic construct
  misc_feature 1..44: note = Description of Artificial Sequence: Synthetic oligonucleotide
  source 1..44: mol_type = other DNA
ttgtgtcaccagagtaacagtctgagtaggagctaaaatatttt

SEQ ID NO: 407 - DNA, 36 bp, synthetic construct
  misc_feature 1..36: note = Description of Artificial Sequence: Synthetic oligonucleotide
  source 1..36: mol_type = other DNA
tagctcctactcagactgttactctggtgacacaac

SEQ ID NO: 408 - DNA, 28 bp, synthetic construct
  misc_feature 1..28: note = Description of Artificial Sequence: Synthetic oligonucleotide
  source 1..28: mol_type = other DNA
tagctcctactcagactggtgacccaac

SEQ ID NO: 409 - DNA, 24 bp, synthetic construct
  misc_feature 1..24: note = Description of Artificial Sequence: Synthetic oligonucleotide
  source 1..24: mol_type = other DNA
tagctcctactctggtgacacaac

SEQ ID NO: 410 - DNA, 28 bp, synthetic construct
  misc_feature 1..28: note = Description of Artificial Sequence: Synthetic oligonucleotide
  source 1..28: mol_type = other DNA
tagctcctactcagactggtgacacaac

SEQ ID NO: 411 - DNA, 16 bp, synthetic construct
  misc_feature 1..16: note = Description of Artificial Sequence: Synthetic oligonucleotide
  source 1..16: mol_type = other DNA
tagctcctactcagac

SEQ ID NO: 412 - DNA, 27 bp, synthetic construct
  misc_feature 1..27: note = Description of Artificial Sequence: Synthetic oligonucleotide
  source 1..27: mol_type = other DNA
tagctcctactcagactgttacacaac

SEQ ID NO: 413 - DNA, 30 bp, synthetic construct
  misc_feature 1..30: note = Description of Artificial Sequence: Synthetic oligonucleotide
  source 1..30: mol_type = other DNA
tagctcctactcagactgtggtgaggtgac

SEQ ID NO: 414 - DNA, 32 bp, synthetic construct
  misc_feature 1..32: note = Description of Artificial Sequence: Synthetic oligonucleotide
  source 1..32: mol_type = other DNA
tagctcctactcagactctctggtgacacaac

SEQ ID NO: 415 - DNA, 31 bp, synthetic construct
  misc_feature 1..31: note = Description of Artificial Sequence: Synthetic oligonucleotide
  source 1..31: mol_type = other DNA
tagctcctactcagacctctggtgacacaac

SEQ ID NO: 416 - DNA, 32 bp, synthetic construct
  misc_feature 1..32: note = Description of Artificial Sequence: Synthetic oligonucleotide
  source 1..32: mol_type = other DNA
tagctcctactcaggctgtctggtgacacaac

SEQ ID NO: 417 - DNA, 33 bp, synthetic construct
  misc_feature 1..33: note = Description of Artificial Sequence: Synthetic oligonucleotide
  source 1..33: mol_type = other DNA
tagctcctactcagactactctggtgacacaac

SEQ ID NO: 418 - DNA, 28 bp, synthetic construct
  misc_feature 1..28: note = Description of Artificial Sequence: Synthetic oligonucleotide
  source 1..28: mol_type = other DNA
tagctcctactcagactgttgacacaac

SEQ ID NO: 419 - DNA, 13 bp, synthetic construct
  misc_feature 1..13: note = Description of Artificial Sequence: Synthetic oligonucleotide
  source 1..13: mol_type = other DNA
ctggtgacacaac

SEQ ID NO: 420 - DNA, 29 bp, synthetic construct
  misc_feature 1..29: note = Description of Artificial Sequence: Synthetic oligonucleotide
  source 1..29: mol_type = other DNA
tagctcctactcagactgttagacacaac

SEQ ID NO: 421 - DNA, 33 bp, synthetic construct
  misc_feature 1..33: note = Description of Artificial Sequence: Synthetic oligonucleotide
  source 1..33: mol_type = other DNA
tagctcctactcagactgctctggtgacacaac

SEQ ID NO: 422 - DNA, 20 bp, synthetic construct
  misc_feature 1..20: note = Description of Artificial Sequence: Synthetic oligonucleotide
  source 1..20: mol_type = other DNA
cagactgttactctggtgac

SEQ ID NO: 423 - DNA, 29 bp, synthetic construct
  misc_feature 1..29: note = Description of Artificial Sequence: Synthetic oligonucleotide
  source 1..29: mol_type = other DNA
cagaccacctgtggtctcctactggtgac

SEQ ID NO: 424 - DNA, 18 bp, synthetic construct
  misc_feature 1..18: note = Description of Artificial Sequence: Synthetic oligonucleotide
  source 1..18: mol_type = other DNA
gctttgatttccctaggg

SEQ ID NO: 425 - DNA, 25 bp, synthetic construct
  misc_feature 1..25: note = Description of Artificial Sequence: Synthetic oligonucleotide
  source 1..25: mol_type = other DNA
gctttgatttccagttcttaggcaa

SEQ ID NO: 426 - DNA, 18 bp, synthetic construct
  misc_feature 1..18: note = Description of Artificial Sequence: Synthetic oligonucleotide
  source 1..18: mol_type = other DNA
gctttgattcttaggcaa

SEQ ID NO: 427 - DNA, 62 bp, synthetic construct
  misc_feature 1..62: note = Description of Artificial Sequence: Synthetic oligonucleotide
  variation 13..53: note = a, c, t, g, unknown or other
  source 1..62: mol_type = other DNA
gctttgatttccnnnnnnnnnnnnnnnnnnnnnnnnnnnnnnnnnnnnnnnnncttaggcaa

SEQ ID NO: 428 - DNA, 18 bp, synthetic construct
  misc_feature 1..18: note = Description of Artificial Sequence: Synthetic oligonucleotide
  source 1..18: mol_type = other DNA
tcttaaccattaccatag

SEQ ID NO: 429 - DNA, 32 bp, synthetic construct
  misc_feature 1..32: note = Description of Artificial Sequence: Synthetic oligonucleotide
  source 1..32: mol_type = other DNA
tcttaaccattaccatagagttcttaggcaac

SEQ ID NO: 430 - DNA, 30 bp, synthetic construct
  misc_feature 1..30: note = Description of Artificial Sequence: Synthetic oligonucleotide
  source 1..30: mol_type = other DNA
tcttaaccattaccaaagttcttaggcaac

SEQ ID NO: 431 - DNA, 31 bp, synthetic construct
  misc_feature 1..31: note = Description of Artificial Sequence: Synthetic oligonucleotide
  source 1..31: mol_type = other DNA
tcttaaccattaccataggttcttaggcaac

SEQ ID NO: 432 - DNA, 14 bp, synthetic construct
  misc_feature 1..14: note = Description of Artificial Sequence: Synthetic oligonucleotide
  source 1..14: mol_type = other DNA
gaaccaaacccact

SEQ ID NO: 433 - DNA, 24 bp, synthetic construct
  misc_feature 1..24: note = Description of Artificial Sequence: Synthetic oligonucleotide
  source 1..24: mol_type = other DNA
gaaccaaacccacttaggggataa

SEQ ID NO: 434 - DNA, 2328 bp, synthetic construct
  misc_feature 1..2328: note = Description of Artificial Sequence: Synthetic polynucleotide
  source 1..2328: mol_type = other DNA
atgaggtctgactacaaagaccatgacggtgattataaagatcatgacatcgattacaaggatgacgatgacaagatggcccccaagaagaagaggaaggtgggcctcgagcccggagaaaaaccgtacaagtgccctgagtgcgggaaatcattctccgaccctggggcgctcgtccggcaccaaaggacgcatacaggggaaaagccgtataagtgccccgagtgtggaaagagcttctcgcagagagcccaccttgaacgacaccaaagaacacacactggtgagaaaccctataagtgtccagagtgcggcaaatcgtttagcagatccgatgacttggtgcgccaccagcggacacacacgggtgaaaagccctacaaatgcccggagtgtgggaagtcgttttcaaggtcggatcatctgactacccatcagcgcacccatacgggagcggccgcccgcgccctggtgaagagcgagctggaggagaagaagtccgagctgcggcacaagctgaagtacgtgccccacgagtacatcgagctgatcgagatcgccaggaaccccacccaggaccgcatcctggagatgaaggtgatggagttcttcatgaaggtgtacggctacaggggagagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcgagggcagaggaagtcttctaacatgcggtgacgtggaggagaatcccggccctagatctgactacaaagaccatgacggtgattataaagatcatgacatcgattacaaggatgacgatgacaagatggcccccaagaagaagaggaaggtgggcctcgagccgggagagaagccgtacaagtgtcccgaatgtggaaagagcttctcacagtcgggggaccttcggcgccaccagcgcacacatactggtgaaaagccgtataagtgtccagaatgtggcaaatcattctccacatcagggagcctggtcaggcaccagcgaacccacacgggtgagaagccctataagtgccccgaatgcgggaagtccttttcgcagagagcccacttggagaggcaccagaggacccatacgggggagaaaccttacaagtgccctgaatgcggaaagtcgttctcgacccatctggatctcatcagacatcagagaacgcacactggagagaaaccctacaaatgtcccgagtgtgggaagtcgtttagccgaaaggacaatctcaaaaaccatcaacggacacacacgggtgaaaaaccatacaaatgcccggagtgcggcaaatcgttttcccaacttgcgcacttgcgggcacaccaacgcacgcatactggagcggccgcccgcgccctggtgaagagcgagctggaggagaagaagtccgagctgcggcacaagctgaagtacgtgccccacgagtacatcgagctgatcgagatcgccaggaaccccacccaggaccgcatcctggagatgaaggtgatggagttcttcatgaaggtgtacggctacaggggagagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacacgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttctga

SEQ ID NO: 435 - protein, 775 aa, synthetic construct
  REGION 1..775: note = Description of Artificial Sequence: Synthetic polypeptide
MRSDYKDHDGDYKDHDIDYKDDDDKMAPKKKRKVGLEPGEKPYKCPECGKSFSDPGALVRHQRTHTGEKPYKCPECGKSFSQRAHLERHQRTHTGEKPYKCPECGKSFSRSDDLVRHQRTHTGEKPYKCPECGKSFSRSDHLTTHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFEGRGSLLTCGDVEENPGPRSDYKDHDGDYKDHDIDYKDDDDKMAPKKKRKVGLEPGEKPYKCPECGKSFSQSGDLRRHQRTHTGEKPYKCPECGKSFSTSGSLVRHQRTHTGEKPYKCPECGKSFSQRAHLERHQRTHTGEKPYKCPECGKSFSTHLDLIRHQRTHTGEKPYKCPECGKSFSRKDNLKNHQRTHTGEKPYKCPECGKSFSQLAHLRAHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 436 - DNA, 4725 bp, synthetic construct
  misc_feature 1..4725: note = Description of Artificial Sequence: Synthetic polynucleotide
  source 1..4725: mol_type = other DNA
tggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggcagtgacgtaacgcgaagcgcgcgaagcgagaccacgcctaccagctgcgtcagcagtcaggtgacccttttgcgacagtttgcgacaccacgtggccgctgagggtatatattctcgagtgagcgaaccaggagctccattttgaccgcgaaatttgaacgagcagcagccatgccggggttctacgagattgtcctgaaggtcccgagtgacctggacgagcacctgccgggcatttctaactcgtttgttaactgggtggccgagaaggaatgggagctgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttaaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacggcagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataaatgacttaaaccaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagcgcttcaacgggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaatcctggttaatcaataaaccgtttaattcgtttcagttgaactttggctcttgtgcacttcttatcttatcttgtttccatggctactgcgtagataagcagcggcctgcggcgcttgcgcttcgcggtttacaactgctggttaatatttaactctcgccatacctctagtgatggagttggccactccctctatgcgcactcgctcgctcggtggggccggacgtgcaaagcacgtccgtctggcgacctttggtcgccaggccccaccgagcgagcgagtgcgcatagagggagtggccaa

SEQ ID NO: 437 - protein, 737 aa, synthetic construct
  REGION 1..737: note = Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSASTGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 438 - protein, 406 aa, synthetic construct
  REGION 1..406: note = Description of Artificial Sequence: Synthetic polypeptide
MDYKDHDGDYKDHDIDYKDDDDKMAPKKKRKVGRLEPGEKPYKCPECGKSFSRKDALRGHQRTHTGEKPYKCPECGKSFSHRTTLTNHQRTHTGEKPYKCPECGKSFSQRNALAGHQRTHTGEKPYKCPECGKSFSHKNALQNHQRTHTGEKPYKCPECGKSFSDPGHLVRHQRTHTGEKPYKCPECGKSFSTSGNLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 439 - protein, 406 aa, synthetic construct
  REGION 1..406: note = Description of Artificial Sequence: Synthetic polypeptide
MDYKDHDGDYKDHDIDYKDDDDKMAPKKKRKVGRLEPGEKPYKCPECGKSFSQQRSLVGHQRTHTGEKPYKCPECGKSFSDKKDLTRHQRTHTGEKPYKCPECGKSFSTSGHLVRHQRTHTGEKPYKCPECGKSFSQRAHLERHQRTHTGEKPYKCPECGKSFSTSGSLVRHQRTHTGEKPYKCPECGKSFSTSGNLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 440 - protein, 320 aa, synthetic construct
  REGION 1..320: note = Description of Artificial Sequence: Synthetic polypeptide
MDYKDHDGDYKDHDIDYKDDDDKMAPKKKRKVGRLEPGERPFQCRICMRNFSSKQALAVHTRTHTGEKPFQCRICMRNFSQSTTLKRHLRTHTGEKPFQCRICMRNFSRSDHLSLHLKTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 441 - protein, 320 aa, synthetic construct
  REGION 1..320: note = Description of Artificial Sequence: Synthetic polypeptide
MDYKDHDGDYKDHDIDYKDDDDKMAPKKKRKVGRLEPGERPFQCRICMRNFSRRAHLQNHTRTHTGEKPFQCRICMRNFSQSTTLKRHLRTHTGEKPFQCRICMRNFSDGGHLTRHLKTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 442 - DNA, 42 bp, synthetic construct
  misc_feature 1..42: note = Description of Artificial Sequence: Synthetic oligonucleotide
  source 1..42: mol_type = other DNA
caaactagaaatgccatcttccttgatgttggaggtacctgc

SEQ ID NO: 443 - protein, 406 aa, synthetic construct; identical to SEQ ID NO: 438
  REGION 1..406: note = Description of Artificial Sequence: Synthetic polypeptide

SEQ ID NO: 444 - protein, 406 aa, synthetic construct; identical to SEQ ID NO: 439
  REGION 1..406: note = Description of Artificial Sequence: Synthetic polypeptide

SEQ ID NO: 445 - DNA, 25 bp, synthetic construct
  misc_feature 1..25: note = Description of Artificial Sequence: Synthetic oligonucleotide
  misc_feature 1: note = May or may not be present
  misc_feature 25: note = May or may not be present
  source 1..25: mol_type = other DNA
tatctgcccatgactggcgcaggga

SEQ ID NO: 446 - protein, 320 aa, synthetic construct; identical to SEQ ID NO: 440
  REGION 1..320: note = Description of Artificial Sequence: Synthetic polypeptide

SEQ ID NO: 447 - protein, 320 aa, synthetic construct; identical to SEQ ID NO: 441
  REGION 1..320: note = Description of Artificial Sequence: Synthetic polypeptide

SEQ ID NO: 448 - DNA, 42 bp, synthetic construct
  misc_feature 1..42: note = Description of Artificial Sequence: Synthetic oligonucleotide
  source 1..42: mol_type = other DNA
actagaaatgccatcttccttgatgttggaggtacctgctct

SEQ ID NO: 449 - protein, 406 aa, synthetic construct
  REGION 1..406: note = Description of Artificial Sequence: Synthetic polypeptide
MDYKDHDGDYKDHDIDYKDDDDKMAPKKKRKVGRLEPGEKPYKCPECGKSFSHRTTLTNHQRTHTGEKPYKCPECGKSFSQRNALAGHQRTHTGEKPYKCPECGKSFSHKNALQNHQRTHTGEKPYKCPECGKSFSDPGHLVRHQRTHTGEKPYKCPECGKSFSTSGNLVRHQRTHTGEKPYKCPECGKSFSQSSNLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 450 - protein, 406 aa, synthetic construct
  REGION 1..406: note = Description of Artificial Sequence: Synthetic polypeptide
MDYKDHDGDYKDHDIDYKDDDDKMAPKKKRKVGRLEPGEKPYKCPECGKSFSQRNALAGHQRTHTGEKPYKCPECGKSFSQQRSLVGHQRTHTGEKPYKCPECGKSFSDKKDLTRHQRTHTGEKPYKCPECGKSFSTSGHLVRHQRTHTGEKPYKCPECGKSFSQRAHLERHQRTHTGEKPYKCPECGKSFSTSGSLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 451 - protein, 378 aa, synthetic construct
  REGION 1..378: note = Description of Artificial Sequence: Synthetic polypeptide
MDYKDHDGDYKDHDIDYKDDDDKMAPKKKRKVGRLEPGEKPYKCPECGKSFSQRNALAGHQRTHTGEKPYKCPECGKSFSHKNALQNHQRTHTGEKPYKCPECGKSFSDPGHLVRHQRTHTGEKPYKCPECGKSFSTSGNLVRHQRTHTGEKPYKCPECGKSFSQSSNLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 452 - protein, 378 aa, synthetic construct
  REGION 1..378: note = Description of Artificial Sequence: Synthetic polypeptide
MDYKDHDGDYKDHDIDYKDDDDKMAPKKKRKVGRLEPGEKPYKCPECGKSFSQQRSLVGHQRTHTGEKPYKCPECGKSFSDKKDLTRHQRTHTGEKPYKCPECGKSFSTSGHLVRHQRTHTGEKPYKCPECGKSFSQRAHLERHQRTHTGEKPYKCPECGKSFSTSGSLVRHQRTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 453 - DNA, 26 bp, synthetic construct
  misc_feature 1..26: note = Description of Artificial Sequence: Synthetic oligonucleotide
  misc_feature 1: note = May or may not be present
  misc_feature 26: note = May or may not be present
  source 1..26: mol_type = other DNA
tgccatcttccttgatgttggaggta

SEQ ID NO: 454 - protein, 322 aa, synthetic construct
  REGION 1..322: note = Description of Artificial Sequence: Synthetic polypeptide
MDYKDHDGDYKDHDIDYKDDDDKMAPKKKRKVGRLEPGERPFQCRICMRNFSSPSKLARHTRTHTGEKPFQCRICMRNFSVRHNLTRHLRTHTGEKPFQCRICMRNFSQRNNLGRHLKTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 455 - protein, 322 aa, synthetic construct
  REGION 1..322: note = Description of Artificial Sequence: Synthetic polypeptide
MDYKDHDGDYKDHDIDYKDDDDKMAPKKKRKVGRLEPGERPFQCRICMRNFSIPNHLARHTRTHTGEKPFQCRICMRNFSQSAHLKRHLRTHTGEKPFQCRICMRNFSHHNSLTRHLKTHTGAAARALVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 456 - DNA, 6626 bp, synthetic construct
  misc_feature 1..6626: note = Description of Artificial Sequence: Synthetic polynucleotide
  source 1..6626: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtggctagcgtctataggcccacccccttggtggaattcgccatgaggtctgactacaaagaccatgacggtgattataaagatcatgacatcgattacaaggatgacgatgacaagatggcccccaagaagaagaggaaggtgggcctcgagcccggagaaaaaccgtacaagtgccctgagtgcgggaaatcattctccgaccctggggcgctcgtccggcaccaaaggacgcatacaggggaaaagccgtataagtgccccgagtgtggaaagagcttctcgcagagagcccaccttgaacgacaccaaagaacacacactggtgagaaaccctataagtgtccagagtgcggcaaatcgtttagcagatccgatgacttggtgcgccaccagcggacacacacgggtgaaaagccctacaaatgcccggagtgtgggaagtcgttttcaaggtcggatcatctgactacccatcagcgcacccatacgggagcggccgcccgcgccctggtgaagagcgagctggaggagaagaagtccgagctgcggcacaagctgaagtacgtgccccacgagtacatcgagctgatcgagatcgccaggaaccccacccaggaccgcatcctggagatgaaggtgatggagttcttcatgaaggtgtacggctacaggggagagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcgagggcagaggaagtcttctaacatgcggtgacgtggaggagaatcccggccctagatctgactacaaagaccatgacggtgattataaagatcatgacatcgattacaaggatgacgatgacaagatggcccccaagaagaagaggaaggtgggcctcgagccgggagagaagccgtacaagtgtcccgaatgtggaaagagcttctcacagtcgggggaccttcggcgccaccagcgcacacatactggtgaaaagccgtataagtgtccagaatgtggcaaatcattctccacatcagggagcctggtcaggcaccagcgaacccacacgggtgagaagccctataagtgccccgaatgcgggaagtccttttcgcagagagcccacttggagaggcaccagaggacccatacgggggagaaaccttacaagtgccctgaatgcggaaagtcgttctcgacccatctggatctcatcagacatcagagaacgcacactggagagaaaccctacaaatgtcccgagtgtgggaagtcgtttagccgaaaggacaatctcaaaaaccatcaacggacacacacgggtgaaaaaccatacaaatgcccggagtgcggcaaatcgttttcccaacttgcgcacttgcgggcacaccaacgcacgcatactggagcggccgcccgcgccctggtgaagagcgagctggaggagaagaagtccgagctgcggcacaagctgaagtacgtgccccacgagtacatcgagctgatcgagatcgccaggaaccccacccaggaccgcatcctggagatgaaggtgatggagttcttcatgaaggtgtacggctacaggggagagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacacgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttctgattaattaactaatctagagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 457 - DNA, 22 bp, synthetic construct
  misc_feature 1..22: note = Description of Artificial Sequence: Synthetic primer
  source 1..22: mol_type = other DNA
gagaggttatgtggctttacca

SEQ ID NO: 458 - DNA, 20 bp, synthetic construct
  misc_feature 1..20: note = Description of Artificial Sequence: Synthetic primer
  source 1..20: mol_type = other DNA
ctgcgtagtgccaaaacaaa

SEQ ID NO: 459 - DNA, 3252 bp, synthetic construct
  misc_feature 1..3252: note = Description of Artificial Sequence: Synthetic polynucleotide
  source 1..3252: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaa

SEQ ID NO: 460 - DNA, 83 bp, synthetic construct
  misc_feature 1..83: note = Description of Artificial Sequence: Synthetic oligonucleotide
  source 1..83: mol_type = other DNA
gttttagtactctggaaacagaatctactaaaacaaggcaaaatgccgtgtttatctcgtcaacttgttggcgagattttttt

SEQ ID NO: 461 - DNA, 3333 bp, synthetic construct
  misc_feature 1..3333: note = Description of Artificial Sequence: Synthetic polynucleotide
  source 1..3333: mol_type = other DNA
atggtgcctaagaagaagagaaaggtggctgccttcaaacctaattcaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgttcgccgcctgacccgccgtcgcgcccaccgcctgcttcggacccgccgcctattgaaacgcgaaggcgtattacaagccgccaattttgacgaaaacggcttgattaaatccttaccgaatacaccatggcaacttcgcgcagccgcattagaccgcaaactgacgcctttagagtggtcggcagtcttgttgcatttaatcaaacatcgcggctatttatcgcaacggaaaaacgagggcgaaactgccgataaggagcttggcgctttgcttaaaggcgtagccggcaatgcccatgccttacagacaggcgatttccgcacaccggccgaattggctttaaataaatttgagaaagaaagcggccatatccgcaatcagcgcagcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagacaaaaaatccccattaaacctttctcccgaattacaagacgaaatcggcacggcattctccctgttcaaaaccgatgaagacattacaggccgtctgaaagaccgtatacagcccgaaatcttagaagcgctgttgaaacacatcagcttcgataagttcgtccaaatttccttgaaagcattgcgccgaattgtgcctctaatggaacaaggcaaacgttacgatgaagcctgcgccgaaatctacggagaccattacggcaagaagaatacggaagaaaagatttatctgccgccgattcccgccgacgaaatccgcaaccccgtcgtcttgcgcgccttatctcaagcacgtaaggtcattaacggcgtggtacgccgttacggctccccagctcgtatccatattgaaactgcaagggaagtaggtaaatcgtttaaagaccgcaaagaaattgagaaacgccaagaagaaaaccgcaaagaccgggaaaaagccgccgccaaattccgagagtatttccccaattttgtcggagaacccaaatccaaagatattctgaaactgcgcctgtacgagcaacaacacggcaaatgcctgtattcgggcaaagaaatcaacttaggccgtctgaacgaaaaaggctatgtcgaaatcgaccatgccctgccgttctcgcgcacatgggacgacagtttcaacaataaagtactggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaacgcaatctgaacgacacgcgctacgtcaaccgtttcctgtgtcaatttgttgccgaccgtatgcggctgacaggtaaaggcaagaaacgtgtctttgcatccaacggacaaattaccaatctgttgcgcggcttttggggattgcgcaaagtgcgtgcggaaaacgaccgccatcacgccttggacgccgtcgtcgttgcctgctcgaccgttgccatgcagcagaaaattacccgttttgtacgctataaagagatgaacgcgtttgacggtaaaaccatagacaaagaaacaggagaagtgctgcatcaaaaaacacacttcccacaaccttgggaatttttcgcacaagaagtcatgattcgcgtcttcggcaaaccggacggcaaacccgaattcgaagaagccgataccctagaaaaactgcgcacgttgcttgccgaaaaattatcatctcgccccgaagccgtacacgaatacgttacgccactgtttgtttcacgcgcgcccaatcggaagatgagcgggcaagggcatatggagaccgtcaaatccgccaaacgactggacgaaggcgtcagcgtgttgcgcgtaccgctgacacagttaaaactgaaagacttggaaaaaatggtcaatcgggagcgcgaacctaagctatacgaagcactgaaagcacggctggaagcacataaagacgatcctgccaaagcctttgccgagccgttttacaaatacgataaagcaggcaaccgcacccaacaggtaaaagccgtacgcgtagagcaagtacagaaaaccggcgtatgggtgcgcaaccataacggtattgccgacaacgcaaccatggtgcgcgtagatgtgtttgagaaaggcgacaagtattatctggtaccgatttacagttggcaggtagcgaaagggattttgccggatagggctgttgtacaaggaaaagatgaagaagattggcaacttattgatgatagtttcaactttaaattctcattacaccctaatgatttagtcgaggttataacaaaaaaagctagaatgtttggttactttgccagctgccatcgaggcacaggtaatatcaatatacgcattcatgatcttgatcataaaattggcaaaaatggaatactggaaggtatcggcgtcaaaaccgccctttcattccaaaaataccaaattgacgaactgggcaaagaaatcagaccatgccgtctgaaaaaacgcccgcctgtccgttacccatacgatgttccagattacgctgcagctccagcagcgaagaaaaagaagctggattaa

SEQ ID NO: 462 - DNA, 120 bp, synthetic construct
  misc_feature 1..120: note = Description of Artificial Sequence: Synthetic polynucleotide
  source 1..120: mol_type = other DNA
gttgtagctccctttctcatttcggaaacgaaatgagaaccgttgctacaataaggccgtctgaaaagatgtgccgcaacgctctgccccttaaagcttctgctttaaggggcttttttt

SEQ ID NO: 463 - DNA, 144 bp, synthetic construct
  misc_feature 1..144: note = Description of Artificial Sequence: Synthetic polynucleotide
  source 1..144: mol_type = other DNA
gttgtagctccctttctcatttcgcagtgctacaatgaaaattgtcgcactgcgaaatgagaaccgttgctacaataaggccgtctgaaaagatgtgccgcaacgctctgccccttaaagcttctgctttaaggggcttttttt

SEQ ID NO: 464 - DNA, 24 bp, synthetic construct
  misc_feature 1..24: note = Description of Artificial Sequence: Synthetic primer
  source 1..24: mol_type = other DNA
tcgggtttattacagggacagcag

SEQ ID NO: 465 - DNA, 24 bp, synthetic construct
  misc_feature 1..24: note = Description of Artificial Sequence: Synthetic primer
  source 1..24: mol_type = other DNA
tctaaggccgagtcttatgagcag

SEQ ID NO: 466 - DNA, 4564 bp, synthetic construct
  misc_feature 1..4564: note = Description of Artificial Sequence: Synthetic polynucleotide
  source 1..4564: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagttgctagcg

SEQ ID NO: 467 - DNA, 388 bp, synthetic construct
  misc_feature 1..388: note = Description of Artificial Sequence: Synthetic polynucleotide
  source 1..388: mol_type = other DNA
taagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatatt

SEQ ID NO: 468 - DNA, 19 bp, synthetic construct
  misc_feature 1..19: note = Description of Artificial Sequence: Synthetic oligonucleotide
  source 1..19: mol_type = other DNA
cagccgctcgctgcagcag

SEQ ID NO: 469 - DNA, 19 bp, synthetic construct
  misc_feature 1..19: note = Description of Artificial Sequence: Synthetic oligonucleotide
  source 1..19: mol_type = other DNA
ctgctgcagcgagcggctg

SEQ ID NO: 470 - DNA, 19 bp, synthetic construct
  misc_feature 1..19: note = Description of Artificial Sequence: Synthetic oligonucleotide
  source 1..19: mol_type = other DNA
gctgggtgtcccattgaaa

SEQ ID NO: 471 - DNA, 19 bp, synthetic construct
  misc_feature 1..19: note = Description of Artificial Sequence: Synthetic oligonucleotide
  source 1..19: mol_type = other DNA
tttcaatgggacacccagc

SEQ ID NO: 472 - DNA, 19 bp, synthetic construct
  misc_feature 1..19: note = Description of Artificial Sequence: Synthetic oligonucleotide
  source 1..19: mol_type = other DNA
gtttattcagccgggagtc

SEQ ID NO: 473 - DNA, 19 bp, synthetic construct
  misc_feature 1..19: note = Description of Artificial Sequence: Synthetic oligonucleotide
  source 1..19: mol_type = other DNA
gactcccggctgaataaac

SEQ ID NO: 474 - DNA, 19 bp, synthetic construct
  misc_feature 1..19: note = Description of Artificial Sequence: Synthetic oligonucleotide
  source 1..19: mol_type = other DNA
tggagagtttgcaaggagc

SEQ ID NO: 475 - DNA, 19 bp, synthetic construct
  misc_feature 1..19: note = Description of Artificial Sequence: Synthetic oligonucleotide
  source 1..19: mol_type = other DNA
gctccttgcaaactctcca

SEQ ID NO: 476 - DNA, 19 bp, synthetic construct
  misc_feature 1..19: note = Description of Artificial Sequence: Synthetic oligonucleotide
  source 1..19: mol_type = other DNA
ccctccagactttccacct

SEQ ID NO: 477 - DNA, 19 bp, synthetic construct
  misc_feature 1..19: note = Description of Artificial Sequence: Synthetic oligonucleotide
  source 1..19: mol_type = other DNA
aggtggaaagtctggaggg

SEQ ID NO: 478 - DNA, 19 bp, synthetic construct
  misc_feature 1..19: note = Description of Artificial Sequence: Synthetic oligonucleotide
  source 1..19: mol_type = other DNA
aattttcttccaagttctc

SEQ ID NO: 479 - DNA, 19 bp, synthetic construct
  misc_feature 1..19: note = Description of Artificial Sequence: Synthetic oligonucleotide
  source 1..19: mol_type = other DNA
gagaacttggaagaaaatt

SEQ ID NO: 480 - DNA, 19 bp, synthetic construct
  misc_feature 1..19: note = Description of Artificial Sequence: Synthetic oligonucleotide
  source 1..19: mol_type = other DNA
ctgcggagagaagaaaggg

SEQ ID NO: 481 - DNA, 19 bp, synthetic construct
  misc_feature 1..19: note = Description of Artificial Sequence: Synthetic oligonucleotide
  source 1..19: mol_type = other DNA
ccctttcttctctccgcag

SEQ ID NO: 482 - DNA, 19 bp, synthetic construct
  misc_feature 1..19: note = Description of Artificial Sequence: Synthetic oligonucleotide
  source 1..19: mol_type = other DNA
agagccaccccctggctcc

SEQ ID NO: 483 - DNA, 19 bp, synthetic construct
  misc_feature 1..19: note = Description of Artificial Sequence: Synthetic oligonucleotide
  source 1..19: mol_type = other DNA
ggagccagggggtggctct

SEQ ID NO: 484 - DNA, 19 bp, synthetic construct
  misc_feature 1..19: note = Description of Artificial Sequence: Synthetic oligonucleotide
  source 1..19: mol_type = other DNA
cgaagccaaccgcggcggg

SEQ ID NO: 485 - DNA, 19 bp, synthetic construct
  misc_feature 1..19: note = Description of Artificial Sequence: Synthetic oligonucleotide
  source 1..19: mol_type = other DNA
cccgccgcggttggcttcg

SEQ ID NO: 486 - DNA, 19 bp, synthetic construct
  misc_feature 1..19: note = Description of Artificial Sequence: Synthetic oligonucleotide
  source 1..19: mol_type = other DNA
agagggaagacgatcgccc

SEQ ID NO: 487 - DNA, 19 bp, synthetic construct
  misc_feature 1..19: note = Description of Artificial Sequence: Synthetic oligonucleotide
  source 1..19: mol_type = other DNA
gggcgatcgtcttccctct

SEQ ID NO: 488 - DNA, 19 bp, synthetic construct
  misc_feature 1..19: note = Description of Artificial Sequence: Synthetic oligonucleotide
  source 1..19: mol_type = other DNA
cccctttaactttcctccg

SEQ ID NO: 489 - DNA, 19 bp, synthetic construct
  misc_feature 1..19: note = Description of Artificial Sequence: Synthetic oligonucleotide
  source 1..19: mol_type = other DNA
cggaggaaagttaaagggg

SEQ ID NO: 490 - DNA, 19 bp, synthetic construct
  misc_feature 1..19: note = Description of Artificial Sequence: Synthetic oligonucleotide
  source 1..19: mol_type = other DNA
gcagccccgcttccttcaa

SEQ ID NO: 491 - DNA, 19 bp, synthetic construct
  misc_feature 1..19: note = Description of Artificial Sequence: Synthetic oligonucleotide
  source 1..19: mol_type = other DNA
ttgaaggaagcggggctgc

SEQ ID NO: 492 - DNA, 24 bp, synthetic construct
  misc_feature 1..24: note = Description of Artificial Sequence: Synthetic oligonucleotide
  source 1..24: mol_type = other DNA
caccgcagccgctcgctgcagcag

SEQ ID NO: 493 - DNA, 24 bp, synthetic construct
  misc_feature 1..24: note = Description of Artificial Sequence: Synthetic oligonucleotide
  source 1..24: mol_type = other DNA
aaacctgctgcagcgagcggctgc

SEQ ID NO: 494 - DNA, 24 bp, synthetic construct
  misc_feature 1..24: note = Description of Artificial Sequence: Synthetic oligonucleotide
  source 1..24: mol_type = other DNA
caccggctgggtgtcccattgaaa

SEQ ID NO: 495 - DNA, 24 bp, synthetic construct
  misc_feature 1..24: note = Description of Artificial Sequence: Synthetic oligonucleotide
  source 1..24: mol_type = other DNA
aaactttcaatgggacacccagcc

SEQ ID NO: 496 - DNA, 24 bp, synthetic construct
  misc_feature 1..24: note = Description of Artificial Sequence: Synthetic oligonucleotide
  source 1..24: mol_type = other DNA
caccggtttattcagccgggagtc

SEQ ID NO: 497 - DNA, 24 bp, synthetic construct
  misc_feature 1..24: note = Description of Artificial Sequence: Synthetic oligonucleotide
  source 1..24: mol_type = other DNA
aaacgactcccggctgaataaacc

SEQ ID NO: 498 - DNA, 24 bp, synthetic construct
  misc_feature 1..24: note = Description of Artificial Sequence: Synthetic oligonucleotide
  source 1..24: mol_type = other DNA
caccgtggagagtttgcaaggagc

SEQ ID NO: 499 - DNA, 24 bp, synthetic construct
  misc_feature 1..24: note = Description of Artificial Sequence: Synthetic oligonucleotide
  source 1..24: mol_type = other DNA
aaacgctccttgcaaactctccac

SEQ ID NO: 500 - DNA, 24 bp, synthetic construct
  misc_feature 1..24: note = Description of Artificial Sequence: Synthetic oligonucleotide
  source 1..24: mol_type = other DNA
caccgccctccagactttccacct

SEQ ID NO: 501 - DNA, 24 bp, synthetic construct
  misc_feature 1..24: note = Description of Artificial Sequence: Synthetic oligonucleotide
  source 1..24: mol_type = other DNA
aaacaggtggaaagtctggagggc

SEQ ID NO: 502 - DNA, 24 bp, synthetic construct
  misc_feature 1..24: note = Description of Artificial Sequence: Synthetic oligonucleotide
  source 1..24: mol_type = other DNA
caccgaattttcttccaagttctc

SEQ ID NO: 503 - DNA, 24 bp, synthetic construct
  misc_feature 1..24: note = Description of Artificial Sequence: Synthetic oligonucleotide
  source 1..24: mol_type = other DNA
aaacgagaacttggaagaaaattc

SEQ ID NO: 504 - DNA, 24 bp, synthetic construct
  misc_feature 1..24: note = Description of Artificial Sequence: Synthetic oligonucleotide
  source 1..24: mol_type = other DNA
caccgctgcggagagaagaaaggg

SEQ ID NO: 505 - DNA, 24 bp, synthetic construct
  misc_feature 1..24: note = Description of Artificial Sequence: Synthetic oligonucleotide
  source 1..24: mol_type = other DNA
aaacccctttcttctctccgcagc

SEQ ID NO: 506 - DNA, 24 bp, synthetic construct
  misc_feature 1..24: note = Description of Artificial Sequence: Synthetic oligonucleotide
  source 1..24: mol_type = other DNA
caccgagagccaccccctggctcc

SEQ ID NO: 507 - DNA, 24 bp, synthetic construct
  misc_feature 1..24: note = Description of Artificial Sequence: Synthetic oligonucleotide
  source 1..24: mol_type = other DNA
aaacggagccagggggtggctctc

SEQ ID NO: 508 - DNA, 24 bp, synthetic construct
  misc_feature 1..24: note = Description of Artificial Sequence: Synthetic oligonucleotide
  source 1..24: mol_type = other DNA
caccgcgaagccaaccgcggcggg

SEQ ID NO: 509 - DNA, 24 bp, synthetic construct
  misc_feature 1..24: note = Description of Artificial Sequence: Synthetic oligonucleotide
  source 1..24: mol_type = other DNA
aaaccccgccgcggttggcttcgc

SEQ ID NO: 510 - DNA, 24 bp, synthetic construct
  misc_feature 1..24: note = Description of Artificial Sequence: Synthetic oligonucleotide
  source 1..24: mol_type = other DNA
caccgagagggaagacgatcgccc

SEQ ID NO: 511 - DNA, 24 bp, synthetic construct
  misc_feature 1..24: note = Description of Artificial Sequence: Synthetic oligonucleotide
  source 1..24: mol_type = other DNA
aaacgggcgatcgtcttccctctc

SEQ ID NO: 512 - DNA, 24 bp, synthetic construct
  misc_feature 1..24: note = Description of Artificial Sequence: Synthetic oligonucleotide
  source 1..24: mol_type = other DNA
caccgcccctttaactttcctccg

SEQ ID NO: 513 - DNA, 24 bp, synthetic construct
  misc_feature 1..24: note = Description of Artificial Sequence: Synthetic oligonucleotide
  source 1..24: mol_type = other DNA
aaaccggaggaaagttaaaggggc

SEQ ID NO: 514 - DNA, 24 bp, synthetic construct
  misc_feature 1..24: note = Description of Artificial Sequence: Synthetic oligonucleotide
  source 1..24: mol_type = other DNA
caccggcagccccgcttccttcaa

SEQ ID NO: 515 - DNA, 24 bp, synthetic construct
  misc_feature 1..24: note = Description of Artificial Sequence: Synthetic oligonucleotide
  source 1..24: mol_type = other DNA
aaacttgaaggaagcggggctgcc

SEQ ID NO: 516 - DNA, 19 bp, synthetic construct
  misc_feature 1..19: note = Description of Artificial Sequence: Synthetic oligonucleotide
  source 1..19: mol_type = other DNA
cgagagcgagaggagggag

SEQ ID NO: 517 - DNA, 19 bp, synthetic construct
  misc_feature 1..19: note = Description of Artificial Sequence: Synthetic oligonucleotide
  source 1..19: mol_type = other DNA
ctccctcctctcgctctcg

SEQ ID NO: 518 - DNA, 19 bp, synthetic construct
  misc_feature 1..19: note = Description of Artificial Sequence: Synthetic oligonucleotide
  source 1..19: mol_type = other DNA
gagagagcttgagagcgcg

SEQ ID NO: 519 - DNA, 19 bp, synthetic construct
  misc_feature 1..19: note = Description of Artificial Sequence: Synthetic oligonucleotide
  source 1..19: mol_type = other DNA
cgcgctctcaagctctctc

SEQ ID NO: 520 - DNA, 19 bp, synthetic construct
  misc_feature 1..19: note = Description of Artificial Sequence: Synthetic oligonucleotide
  source 1..19: mol_type = other DNA
ggtggagggggcggggccc

SEQ ID NO: 521 - DNA, 19 bp, synthetic construct
  misc_feature 1..19: note = Description of Artificial Sequence: Synthetic oligonucleotide
  source 1..19: mol_type = other DNA
gggccccgccccctccacc

SEQ ID NO: 522 - DNA, 19 bp, synthetic construct
  misc_feature 1..19: note = Description of Artificial Sequence: Synthetic oligonucleotide
  source 1..19: mol_type = other DNA
ggtatccacgtaaatcaaa

SEQ ID NO: 523 - DNA, 19 bp, synthetic construct
  misc_feature 1..19: note = Description of Artificial Sequence: Synthetic oligonucleotide
  source 1..19: mol_type = other DNA
tttgatttacgtggatacc

SEQ ID NO: 524 - DNA, 19 bp, synthetic construct
  misc_feature 1..19: note = Description of Artificial Sequence: Synthetic oligonucleotide
  source 1..19: mol_type = other DNA
ccaatcactggctccggtc

SEQ ID NO: 525 - DNA, 19 bp, synthetic construct
  misc_feature 1..19: note = Description of Artificial Sequence: Synthetic oligonucleotide
  source 1..19: mol_type = other DNA
gaccggagccagtgattgg

SEQ ID NO: 526 - DNA, 19 bp, synthetic construct
  misc_feature 1..19: note = Description of Artificial Sequence: Synthetic oligonucleotide
  source 1..19: mol_type = other DNA
ggcgcccgagggaagaaga

SEQ ID NO: 527 - DNA, 19 bp, synthetic construct
  misc_feature 1..19: note = Description of Artificial Sequence: Synthetic oligonucleotide
  source 1..19: mol_type = other DNA
tcttcttccctcgggcgcc

SEQ ID NO: 528 - DNA, 19 bp, synthetic construct
  misc_feature 1..19: note = Description of Artificial Sequence: Synthetic oligonucleotide
  source 1..19: mol_type = other DNA
gggtgggggtaccagagga

SEQ ID NO: 529 - DNA, 19 bp, synthetic construct
  misc_feature 1..19: note = Description of Artificial Sequence: Synthetic oligonucleotide
  source 1..19: mol_type = other DNA
tcctctggtacccccaccc

SEQ ID NO: 530 - DNA, 19 bp, synthetic construct
  misc_feature 1..19: note = Description of Artificial Sequence: Synthetic oligonucleotide
  source 1..19: mol_type = other DNA
ccggggacagaagagaggg

SEQ ID NO: 531 - DNA, 19 bp, synthetic construct
  misc_feature 1..19: note = Description of Artificial Sequence: Synthetic oligonucleotide
  source 1..19: mol_type = other DNA
ccctctcttctgtccccgg

SEQ ID NO: 532 - DNA, 19 bp, synthetic construct
  misc_feature 1..19: note = Description of Artificial Sequence: Synthetic oligonucleotide
  source 1..19: mol_type = other DNA
gagagagagtgggagaagc

SEQ ID NO: 533 - DNA, 19 bp, synthetic construct
  misc_feature 1..19: note = Description of Artificial Sequence: Synthetic oligonucleotide
  source 1..19: mol_type = other DNA
gcttctcccactctctctc

SEQ ID NO: 534 - DNA, 19 bp, synthetic construct
  misc_feature 1..19: note = Description of Artificial Sequence: Synthetic oligonucleotide
  source 1..19: mol_type = other DNA
aaagtaactgtcaaatgcg

SEQ ID NO: 535 - DNA, 19 bp, synthetic construct
  misc_feature 1..19: note = Description of Artificial Sequence: Synthetic oligonucleotide
  source 1..19: mol_type = other DNA
cgcatttgacagttacttt

SEQ ID NO: 536 - DNA, 19 bp, synthetic construct
  misc_feature 1..19: note = Description of Artificial Sequence: Synthetic oligonucleotide
  source 1..19: mol_type = other DNA
ttaaccagagcgcccagtc

SEQ ID NO: 537 - DNA, 19 bp, synthetic construct
  misc_feature 1..19: note = Description of Artificial Sequence: Synthetic oligonucleotide
  source 1..19: mol_type = other DNA
gactgggcgctctggttaa

SEQ ID NO: 538 - DNA, 19 bp, synthetic construct
  misc_feature 1..19: note = Description of Artificial Sequence: Synthetic oligonucleotide
  source 1..19: mol_type = other DNA
cgtcggagctgcccgctag

SEQ ID NO: 539 - DNA, 19 bp, synthetic construct
  misc_feature 1..19: note = Description of Artificial Sequence: Synthetic oligonucleotide
  source 1..19: mol_type = other DNA
ctagcgggcagctccgacg

SEQ ID NO: 540 - DNA, 24 bp, synthetic construct
  misc_feature 1..24: note = Description of Artificial Sequence: Synthetic oligonucleotide
  source 1..24: mol_type = other DNA
caccgcgagagcgagaggagggag

SEQ ID NO: 541 - DNA, 24 bp, synthetic construct
  misc_feature 1..24: note = Description of Artificial Sequence: Synthetic oligonucleotide
  source 1..24: mol_type = other DNA
aaacctccctcctctcgctctcgc

SEQ ID NO: 542 - DNA, 24 bp, synthetic construct
  misc_feature 1..24: note = Description of Artificial Sequence: Synthetic oligonucleotide
  source 1..24: mol_type = other DNA
caccggagagagcttgagagcgcg

SEQ ID NO: 543 - DNA, 24 bp, synthetic construct
  misc_feature 1..24: note = Description of Artificial Sequence: Synthetic oligonucleotide
  source 1..24: mol_type = other DNA
aaaccgcgctctcaagctctctcc

SEQ ID NO: 544 - DNA, 24 bp, synthetic construct
  misc_feature 1..24: note = Description of Artificial Sequence: Synthetic oligonucleotide
  source 1..24: mol_type = other DNA
caccgggtggagggggcggggccc

SEQ ID NO: 545 - DNA, 24 bp, synthetic construct
  misc_feature 1..24: note = Description of Artificial Sequence: Synthetic oligonucleotide
  source 1..24: mol_type = other DNA
aaacgggccccgccccctccaccc

SEQ ID NO: 546 - DNA, 24 bp, synthetic construct
  misc_feature 1..24: note = Description of Artificial Sequence: Synthetic oligonucleotide
  source 1..24: mol_type = other DNA
caccgggtatccacgtaaatcaaa

SEQ ID NO: 547 - DNA, 24 bp, synthetic construct
  misc_feature 1..24: note = Description of Artificial Sequence: Synthetic oligonucleotide
  source 1..24: mol_type = other DNA
aaactttgatttacgtggataccc

SEQ ID NO: 548 - DNA, 24 bp, synthetic construct
  misc_feature 1..24: note = Description of Artificial Sequence: Synthetic oligonucleotide
  source 1..24: mol_type = other DNA
caccgccaatcactggctccggtc

SEQ ID NO: 549 - DNA, 24 bp, synthetic construct
  misc_feature 1..24: note = Description of Artificial Sequence: Synthetic oligonucleotide
  source 1..24: mol_type = other DNA
aaacgaccggagccagtgattggc

SEQ ID NO: 550 - DNA, 24 bp, synthetic construct
  misc_feature 1..24: note = Description of Artificial Sequence: Synthetic oligonucleotide
  source 1..24: mol_type = other DNA
caccgggcgcccgagggaagaaga

SEQ ID NO: 551 - DNA, 24 bp, synthetic construct
  misc_feature 1..24: note = Description of Artificial Sequence: Synthetic oligonucleotide
  source 1..24: mol_type = other DNA
aaactcttcttccctcgggcgccc

SEQ ID NO: 552 - DNA, 24 bp, synthetic construct
  misc_feature 1..24: note = Description of Artificial Sequence: Synthetic oligonucleotide
  source 1..24: mol_type = other DNA
caccggggtgggggtaccagagga

SEQ ID NO: 553 - DNA, 24 bp, synthetic construct
  misc_feature 1..24: note = Description of Artificial Sequence: Synthetic oligonucleotide
  source 1..24: mol_type = other DNA
aaactcctctggtacccccacccc

SEQ ID NO: 554 - DNA, 24 bp, synthetic construct
  misc_feature 1..24: note = Description of Artificial Sequence: Synthetic oligonucleotide
  source 1..24: mol_type = other DNA
caccgccggggacagaagagaggg

SEQ ID NO: 555 - DNA, 24 bp, synthetic construct
  misc_feature 1..24: note = Description of Artificial Sequence: Synthetic oligonucleotide
  source 1..24: mol_type = other DNA
aaacccctctcttctgtccccggc

SEQ ID NO: 556 - DNA, 24 bp, synthetic construct
  misc_feature 1..24: note = Description of Artificial Sequence: Synthetic oligonucleotide
  source 1..24: mol_type = other DNA
caccggagagagagtgggagaagc

SEQ ID NO: 557 - DNA, 24 bp, synthetic construct
  misc_feature 1..24: note = Description of Artificial Sequence: Synthetic oligonucleotide
  source 1..24: mol_type = other DNA
aaacgcttctcccactctctctcc

SEQ ID NO: 558 - DNA, 24 bp, synthetic construct
  misc_feature 1..24: note = Description of Artificial Sequence: Synthetic oligonucleotide
  source 1..24: mol_type = other DNA
caccgaaagtaactgtcaaatgcg

SEQ ID NO: 559 - DNA, 24 bp, synthetic construct
  misc_feature 1..24: note = Description of Artificial Sequence: Synthetic oligonucleotide
  source 1..24: mol_type = other DNA
aaaccgcatttgacagttactttc

SEQ ID NO: 560 - DNA, 24 bp, synthetic construct
  misc_feature 1..24: note = Description of Artificial Sequence: Synthetic oligonucleotide
  source 1..24: mol_type = other DNA
caccgttaaccagagcgcccagtc

SEQ ID NO: 561 - DNA, 24 bp, synthetic construct
  misc_feature 1..24: note = Description of Artificial Sequence: Synthetic oligonucleotide
  source 1..24: mol_type = other DNA
aaacgactgggcgctctggttaac

SEQ ID NO: 562 - DNA, 24 bp, synthetic construct
  misc_feature 1..24: note = Description of Artificial Sequence: Synthetic oligonucleotide
  source 1..24: mol_type = other DNA
caccgcgtcggagctgcccgctag

SEQ ID NO: 563 - DNA, 24 bp, synthetic construct
  misc_feature 1..24: note = Description of Artificial Sequence: Synthetic oligonucleotide
  source 1..24: mol_type = other DNA
aaacctagcgggcagctccgacgc

SEQ ID NO: 564 - DNA, 20 bp, synthetic construct
  misc_feature 1..20: note = Description of Artificial Sequence: Synthetic oligonucleotide
  source 1..20: mol_type = other DNA
gagacacacagaaatgtaac

SEQ ID NO: 565 - DNA, 20 bp, synthetic construct
  misc_feature 1..20: note = Description of Artificial Sequence: Synthetic oligonucleotide
  source 1..20: mol_type = other DNA
ggtggggcactgaccccgac

SEQ ID NO: 566 - DNA, 20 bp, synthetic construct
  misc_feature 1..20: note = Description of Artificial Sequence: Synthetic oligonucleotide
  source 1..20: mol_type = other DNA
ctagagtgatgactcctatc

SEQ ID NO: 567 - DNA, 20 bp, synthetic construct
  misc_feature 1..20: note = Description of Artificial Sequence: Synthetic oligonucleotide
  source 1..20: mol_type = other DNA
gactaaaactccacctcaaa

SEQ ID NO: 568 - DNA, 20 bp, synthetic construct
  misc_feature 1..20: note = Description of Artificial Sequence: Synthetic oligonucleotide
  source 1..20: mol_type = other DNA
aatatgtcacattctgtctc

SEQ ID NO: 569 - DNA, 20 bp, synthetic construct
  misc_feature 1..20: note = Description of Artificial Sequence: Synthetic oligonucleotide
  source 1..20: mol_type = other DNA
ggactatgggaggtcactaa

SEQ ID NO: 570 - DNA, 20 bp, synthetic construct
  misc_feature 1..20: note = Description of Artificial Sequence: Synthetic oligonucleotide
  source 1..20: mol_type = other DNA
gctcatgcttggactatggg

SEQ ID NO: 571 - DNA, 20 bp, synthetic construct
  misc_feature 1..20: note = Description of Artificial Sequence: Synthetic oligonucleotide
  source 1..20: mol_type = other DNA
gttctggccaggcccctgtc

SEQ ID NO: 572 - DNA, 20 bp, synthetic construct
  misc_feature 1..20: note = Description of Artificial Sequence: Synthetic oligonucleotide
  source 1..20: mol_type = other DNA
agtgccccacccccgccttc

SEQ ID NO: 573 - DNA, 20 bp, synthetic construct
  misc_feature 1..20: note = Description of Artificial Sequence: Synthetic oligonucleotide
  source 1..20: mol_type = other DNA
gtggggcactgaccccgaca

SEQ ID NO: 574 - DNA, 20 bp, synthetic construct
  misc_feature 1..20: note = Description of Artificial Sequence: Synthetic oligonucleotide
  source 1..20: mol_type = other DNA
aaccttctaagcaaaccttc

SEQ ID NO: 575 - DNA, 20 bp, synthetic construct
  misc_feature 1..20: note = Description of Artificial Sequence: Synthetic oligonucleotide
  source 1..20: mol_type = other DNA
gttacacagaaccagaaggc

SEQ ID NO: 576 - DNA, 20 bp, synthetic construct
  misc_feature 1..20: note = Description of Artificial Sequence: Synthetic oligonucleotide
  source 1..20: mol_type = other DNA
gaaggttacacagaaccaga

SEQ ID NO: 577 - DNA, 20 bp, synthetic construct
  misc_feature 1..20: note = Description of Artificial Sequence: Synthetic oligonucleotide
  source 1..20: mol_type = other DNA
agtcatgatgagtcatgctg

SEQ ID NO: 578 - DNA, 20 bp, synthetic construct
  misc_feature 1..20: note = Description of Artificial Sequence: Synthetic oligonucleotide
  source 1..20: mol_type = other DNA
gatgagtcatgctgaggctt

SEQ ID NO: 579 - DNA, 20 bp, synthetic construct
  misc_feature 1..20: note = Description of Artificial Sequence: Synthetic oligonucleotide
  source 1..20: mol_type = other DNA
actctaggctgagaacatct

SEQ ID NO: 580 - DNA, 20 bp, synthetic construct
  misc_feature 1..20: note = Description of Artificial Sequence: Synthetic oligonucleotide
  source 1..20: mol_type = other DNA
gtccccagcaggatgcttac

SEQ ID NO: 581 - DNA, 20 bp, synthetic construct
  misc_feature 1..20: note = Description of Artificial Sequence: Synthetic oligonucleotide
  source 1..20: mol_type = other DNA
gccctgtaagcatcctgctg

SEQ ID NO: 582 - DNA, 20 bp, synthetic construct
  misc_feature 1..20: note = Description of Artificial Sequence: Synthetic oligonucleotide
  source 1..20: mol_type = other DNA
cagggcagatggcaaaaaaa

SEQ ID NO: 583 - DNA, 20 bp, synthetic construct
  misc_feature 1..20: note = Description of Artificial Sequence: Synthetic oligonucleotide
  source 1..20: mol_type = other DNA
gaggtggagttttagtcagg

SEQ ID NO: 584 - DNA, 20 bp, synthetic construct
  misc_feature 1..20: note = Description of Artificial Sequence: Synthetic oligonucleotide
  source 1..20: mol_type = other DNA
aaacggcatcataaagaaaa

SEQ ID NO: 585 - DNA, 105 bp, synthetic construct
  misc_feature 1..105: note = Description of Artificial Sequence: Synthetic polynucleotide
  source 1..105: mol_type = other DNA
gagacacacagaaatgtaacgttttagagctagaaatagcaagttaaaataaggctagtccgttatcaacttgaaaaagtggcaccgagtcggtgctttttttcc

SEQ ID NO: 586 - DNA, 105 bp, synthetic construct
  misc_feature 1..105: note = Description of Artificial Sequence: Synthetic polynucleotide
  source 1..105: mol_type = other DNA
ggtggggcactgaccccgacgttttagagctagaaatagcaagttaaaataaggctagtccgttatcaacttgaaaaagtggcaccgagtcggtgctttttttcc

SEQ ID NO: 587 - DNA, 105 bp, synthetic construct
  misc_feature 1..105: note = Description of Artificial Sequence: Synthetic polynucleotide
  source 1..105: mol_type = other DNA
ctagagtgatgactcctatcgttttagagctagaaatagcaagttaaaataaggctagtccgttatcaacttgaaaaagtggcaccgagtcggtgctttttttcc

SEQ ID NO: 588 - DNA, 105 bp, synthetic construct
  misc_feature 1..105: note = Description of Artificial Sequence: Synthetic polynucleotide
  source 1..105: mol_type = other DNA
gactaaaactccacctcaaagttttagagctagaaatagcaagttaaaataaggctagtccgttatcaacttgaaaaagtggcaccgagtcggtgctttttttcc

SEQ ID NO: 589 - DNA, 105 bp, synthetic construct
  misc_feature 1..105: note = Description of Artificial Sequence: Synthetic polynucleotide
  source 1..105: mol_type = other DNA
aatatgtcacattctgtctcgttttagagctagaaatagcaagttaaaataaggctagtccgttatcaacttgaaaaagtggcaccgagtcggtgctttttttcc

SEQ ID NO: 590 - DNA, 105 bp, synthetic construct
  misc_feature 1..105: note = Description of Artificial Sequence: Synthetic polynucleotide
  source 1..105: mol_type = other DNA
ggactatgggaggtcactaagttttagagctagaaatagcaagttaaaataaggctagtccgttatcaacttgaaaaagtggcaccgagtcggtgctttttttcc

SEQ ID NO: 591 - DNA, 105 bp, synthetic construct
  misc_feature 1..105: note = Description of Artificial Sequence: Synthetic polynucleotide
  source 1..105: mol_type = other DNA
gctcatgcttggactatggggttttagagctagaaatagcaagttaaaataaggctagtccgttatcaacttgaaaaagtggcaccgagtcggtgctttttttcc

SEQ ID NO: 592 - DNA, 105 bp, synthetic construct
  misc_feature 1..105: note = Description of Artificial Sequence: Synthetic polynucleotide
  source 1..105: mol_type = other DNA
gttctggccaggcccctgtcgttttagagctagaaatagcaagttaaaataaggctagtccgttatcaacttgaaaaagtggcaccgagtcggtgctttttttcc

SEQ ID NO: 593 - DNA, 105 bp, synthetic construct
  misc_feature 1..105: note = Description of Artificial Sequence: Synthetic polynucleotide
  source 1..105: mol_type = other DNA
agtgccccacccccgccttcgttttagagctagaaatagcaagttaaaataaggctagtccgttatcaacttgaaaaagtggcaccgagtcggtgctttttttcc

SEQ ID NO: 594 - DNA, 105 bp, synthetic construct
  misc_feature 1..105: note = Description of Artificial Sequence: Synthetic polynucleotide
  source 1..105: mol_type = other DNA
gtggggcactgaccccgacagttttagagctagaaatagcaagttaaaataaggctagtccgttatcaacttgaaaaagtggcaccgagtcggtgctttttttcc

SEQ ID NO: 595 - DNA, 105 bp, synthetic construct
  misc_feature 1..105: note = Description of Artificial Sequence: Synthetic polynucleotide
  source 1..105: mol_type = other DNA
aaccttctaagcaaaccttcgttttagagctagaaatagcaagttaaaataaggctagtccgttatcaacttgaaaaagtggcaccgagtcggtgctttttttcc

SEQ ID NO: 596 - DNA, 105 bp, synthetic construct
  misc_feature 1..105: note = Description of Artificial Sequence: Synthetic polynucleotide
  source 1..105: mol_type = other DNA
gttacacagaaccagaaggcgttttagagctagaaatagcaagttaaaataaggctagtccgttatcaacttgaaaaagtggcaccgagtcggtgctttttttcc

SEQ ID NO: 597 - DNA, 105 bp, synthetic construct
  misc_feature 1..105: note = Description of Artificial Sequence: Synthetic polynucleotide
  source 1..105: mol_type = other DNA
gaaggttacacagaaccagagttttagagctagaaatagcaagttaaaataaggctagtccgttatcaacttgaaaaagtggcaccgagtcggtgctttttttcc

SEQ ID NO: 598 - DNA, 105 bp, synthetic construct
  misc_feature 1..105: note = Description of Artificial Sequence: Synthetic polynucleotide
  source 1..105: mol_type = other DNA
agtcatgatgagtcatgctggttttagagctagaaatagcaagttaaaataaggctagtccgttatcaacttgaaaaagtggcaccgagtcggtgctttttttcc

SEQ ID NO: 599 - DNA, 105 bp, synthetic construct
  misc_feature 1..105: note = Description of Artificial Sequence: Synthetic polynucleotide
  source 1..105: mol_type = other DNA
gatgagtcatgctgaggcttgttttagagctagaaatagcaagttaaaataaggctagtccgttatcaacttgaaaaagtggcaccgagtcggtgctttttttcc

SEQ ID NO: 600 - DNA, 105 bp, synthetic construct
  misc_feature 1..105: note = Description of Artificial Sequence: Synthetic polynucleotide
  source 1..105: mol_type = other DNA
actctaggctgagaacatctgttttagagctagaaatagcaagttaaaataaggctagtccgttatcaacttgaaaaagtggcaccgagtcggtgctttttttcc

SEQ ID NO: 601 - DNA, 105 bp, synthetic construct
  misc_feature 1..105: note = Description of Artificial Sequence: Synthetic polynucleotide
  source 1..105: mol_type = other DNA
gtccccagcaggatgcttacgttttagagctagaaatagcaagttaaaataaggctagtccgttatcaacttgaaaaagtggcaccgagtcggtgctttttttcc

SEQ ID NO: 602 - DNA, 105 bp, synthetic construct
  misc_feature 1..105: note = Description of Artificial Sequence: Synthetic polynucleotide
  source 1..105: mol_type = other DNA
gccctgtaagcatcctgctggttttagagctagaaatagcaagttaaaataaggctagtccgttatcaacttgaaaaagtggcaccgagtcggtgctttttttcc

SEQ ID NO: 603 - DNA, 105 bp, synthetic construct
  misc_feature 1..105: note = Description of Artificial Sequence: Synthetic polynucleotide
  source 1..105: mol_type = other DNA
cagggcagatggcaaaaaaagttttagagctagaaatagcaagttaaaataaggctagtccgttatcaacttgaaaaagtggcaccgagtcggtgctttttttcc

SEQ ID NO: 604 - DNA, 105 bp, synthetic construct
  misc_feature 1..105: note = Description of Artificial Sequence: Synthetic polynucleotide
  source 1..105: mol_type = other DNA
gaggtggagttttagtcagggttttagagctagaaatagcaagttaaaataaggctagtccgttatcaacttgaaaaagtggcaccgagtcggtgctttttttcc

SEQ ID NO: 605 - DNA, 105 bp, synthetic construct
  misc_feature 1..105: note = Description of Artificial Sequence: Synthetic polynucleotide
  source 1..105: mol_type = other DNA
aaacggcatcataaagaaaagttttagagctagaaatagcaagttaaaataaggctagtccgttatcaacttgaaaaagtggcaccgagtcggtgctttttttcc

SEQ ID NO: 606 - DNA, 19 bp, synthetic construct
  misc_feature 1..19: note = Description of Artificial Sequence: Synthetic oligonucleotide
  source 1..19: mol_type = other DNA
tgtactctctgaggtgctc

SEQ ID NO: 607 - DNA, 19 bp, synthetic construct
  misc_feature 1..19: note = Description of Artificial Sequence: Synthetic oligonucleotide
  source 1..19: mol_type = other DNA
acgcagataagaaccagtt

SEQ ID NO: 608 - DNA, 19 bp, synthetic construct
  misc_feature 1..19: note = Description of Artificial Sequence: Synthetic oligonucleotide
  source 1..19: mol_type = other DNA
catcaagtcagccatcagc

SEQ ID NO: 609 - DNA, 19 bp, synthetic construct
  misc_feature 1..19: note = Description of Artificial Sequence: Synthetic oligonucleotide
  source 1..19: mol_type = other DNA
gagtcaccctcctggaaac

SEQ ID NO: 610 - DNA, 19 bp, synthetic construct
  misc_feature 1..19: note = Description of Artificial Sequence: Synthetic oligonucleotide
  source 1..19: mol_type = other DNA
cctgggctccggggcgttt

SEQ ID NO: 611 - DNA, 19 bp, synthetic construct
  misc_feature 1..19: note = Description of Artificial Sequence: Synthetic oligonucleotide
  source 1..19: mol_type = other DNA
ggcccctgcggccaccccg

SEQ ID NO: 612 - DNA, 19 bp, synthetic construct
  misc_feature 1..19: note = Description of Artificial Sequence: Synthetic oligonucleotide
  source 1..19: mol_type = other DNA
ctccctccctgcccggtag

SEQ ID NO: 613 - DNA, 19 bp, synthetic construct
  misc_feature 1..19: note = Description of Artificial Sequence: Synthetic oligonucleotide
  source 1..19: mol_type = other DNA
aggtttggaaagggcgtgc

SEQ ID NO: 614 - DNA, 20 bp, synthetic construct
  misc_feature 1..20: note = Description of Artificial Sequence: Synthetic oligonucleotide
  source 1..20: mol_type = other DNA
actccactgcactccagtct

SEQ ID NO: 615 - DNA, 20 bp, synthetic construct
  misc_feature 1..20: note = Description of Artificial Sequence: Synthetic oligonucleotide
  source 1..20: mol_type = other DNA
tctgtgggggacctgcactg

SEQ ID NO: 616 - DNA, 20 bp, synthetic construct
  misc_feature 1..20: note = Description of Artificial Sequence: Synthetic oligonucleotide
  source 1..20: mol_type = other DNA
ggggcgccagttgtgtctcc

SEQ ID NO: 617 - DNA, 20 bp, synthetic construct
  misc_feature 1..20: note = Description of Artificial Sequence: Synthetic oligonucleotide
  source 1..20: mol_type = other DNA
acaccattgccaccaccatt

SEQ ID NO: 618 - DNA, 20 bp, synthetic construct
  misc_feature 1..20: note = Description of Artificial Sequence: Synthetic primer
  source 1..20: mol_type = other DNA
caatgaccccttcattgacc

SEQ ID NO: 619 - DNA, 20 bp, synthetic construct
  misc_feature 1..20: note = Description of Artificial Sequence: Synthetic primer
  source 1..20: mol_type = other DNA
ttgattttggagggatctcg

SEQ ID NO: 620 - DNA, 20 bp, synthetic construct
  misc_feature 1..20: note = Description of Artificial Sequence: Synthetic primer
  source 1..20: mol_type = other DNA
ggaatccatggagggaagat

SEQ ID NO: 621 - DNA, 20 bp, synthetic construct
  misc_feature 1..20: note = Description of Artificial Sequence: Synthetic primer
  source 1..20: mol_type = other DNA
tgttctcgctcaggtcagtg

SEQ ID NO: 622 - DNA, 20 bp, synthetic construct
  misc_feature 1..20: note = Description of Artificial Sequence: Synthetic primer
  source 1..20: mol_type = other DNA
ctctctgctcctttgccaca

SEQ ID NO: 623 - DNA, 20 bp, synthetic construct
  misc_feature 1..20: note = Description of Artificial Sequence: Synthetic primer
  source 1..20: mol_type = other DNA
gtgctcttcgggtttcagga

SEQ ID NO: 624 - DNA, 30 bp, synthetic construct
  misc_feature 1..30: note = Description of Artificial Sequence: Synthetic primer
  source 1..30: mol_type = other DNA
cgaaagagaaagcgaaccagtatcgagaac

SEQ ID NO: 625 - DNA, 27 bp, synthetic construct
  misc_feature 1..27: note = Description of Artificial Sequence: Synthetic primer
  source 1..27: mol_type = other DNA
cgttgtgcatagtcgctgcttgatcgc

SEQ ID NO: 626 - DNA, 25 bp, synthetic construct
  misc_feature 1..25: note = Description of Artificial Sequence: Synthetic primer
  source 1..25: mol_type = other DNA
gagtttggctcaaattgttactctt

SEQ ID NO: 627 - DNA, 25 bp, synthetic construct
  misc_feature 1..25: note = Description of Artificial Sequence: Synthetic primer
  source 1..25: mol_type = other DNA
gggaaatggtctaggagagtaaagt

SEQ ID NO: 628 - DNA, 23 bp, synthetic construct
  misc_feature 1..23: note = Description of Artificial Sequence: Synthetic oligonucleotide
  source 1..23: mol_type = other DNA
gattggctttgatttccctaggg

SEQ ID NO: 629 - DNA, 23 bp, synthetic construct
  misc_feature 1..23: note = Description of Artificial Sequence: Synthetic oligonucleotide
  source 1..23: mol_type = other DNA
gcctactcagactgttactctgg

SEQ ID NO: 630 - DNA, 23 bp, synthetic construct
  misc_feature 1..23: note = Description of Artificial Sequence: Synthetic oligonucleotide
  source 1..23: mol_type = other DNA
gcagttgcctaagaactggtggg

SEQ ID NO: 631 - DNA, 23 bp, synthetic construct
  misc_feature 1..23: note = Description of Artificial Sequence: Synthetic oligonucleotide
  source 1..23: mol_type = other DNA
ggggctccaccctcacgagtggg

SEQ ID NO: 632 - DNA, 23 bp, synthetic construct
  misc_feature 1..23: note = Description of Artificial Sequence: Synthetic oligonucleotide
  source 1..23: mol_type = other DNA
gccttctttatcccctatcgagg

SEQ ID NO: 633 - DNA, 48 bp, synthetic construct
  misc_feature 1..48: note = Description of Artificial Sequence: Synthetic oligonucleotide
  source 1..48: mol_type = other DNA
gaaggaccatttgacgttcagctcctactcagactgttactctggtga

SEQ ID NO: 634 - protein, 15 aa, synthetic construct
  REGION 1..15: note = Description of Artificial Sequence: Synthetic peptide
EGPFDVQLLLRLLLW

SEQ ID NO: 635 - DNA, 46 bp, synthetic construct
  misc_feature 1..46: note = Description of Artificial Sequence: Synthetic oligonucleotide
  source 1..46: mol_type = other DNA
gaaggaccatttgacgttcagctcctactcagactgtctctggtga

SEQ ID NO: 636 - protein, 15 aa, synthetic construct
  REGION 1..15: note = Description of Artificial Sequence: Synthetic peptide
EGPFDVQLLLRLSLV

SEQ ID NO: 637 - RNA, 20 nt, synthetic construct
  misc_feature 1..20: note = Description of Artificial Sequence: Synthetic oligonucleotide
  source 1..20: mol_type = other RNA
gcctactcagactgttactc

SEQ ID NO: 638 - DNA, 19 bp, synthetic construct
  misc_feature 1..19: note = Description of Artificial Sequence: Synthetic oligonucleotide
  source 1..19: mol_type = other DNA
cccaccagttcttaggcaa

SEQ ID NO: 639 - DNA, 20 bp, synthetic construct
  misc_feature 1..20: note = Description of Artificial Sequence: Synthetic oligonucleotide
  source 1..20: mol_type = other DNA
cccaccagttcttaggcaac

SEQ ID NO: 640 - DNA, 20 bp, synthetic construct
  misc_feature 1..20: note = Description of Artificial Sequence: Synthetic oligonucleotide
  source 1..20: mol_type = other DNA
tttgatttccagttcttagg

SEQ ID NO: 641 - DNA, 19 bp, synthetic construct
  misc_feature 1..19: note = Description of Artificial Sequence: Synthetic oligonucleotide
  source 1..19: mol_type = other DNA
attaccatagagttcttag

SEQ ID NO: 642 - DNA, 19 bp, synthetic construct
  misc_feature 1..19: note = Description of Artificial Sequence: Synthetic oligonucleotide
  source 1..19: mol_type = other DNA
gtggataaaggcaacaatg

SEQ ID NO: 643 - DNA, 16 bp, synthetic construct
  misc_feature 1..16: note = Description of Artificial Sequence: Synthetic oligonucleotide
  source 1..16: mol_type = other DNA
cctcgataggggataa

SEQ ID NO: 644 - DNA, 23 bp, synthetic construct
  misc_feature 1..23: note = Description of Artificial Sequence: Synthetic oligonucleotide
  source 1..23: mol_type = other DNA
accaaacccacttaggggataaa

SEQ ID NO: 645 - DNA, 21 bp, synthetic construct
  misc_feature 1..21: note = Description of Artificial Sequence: Synthetic oligonucleotide
  source 1..21: mol_type = other DNA
ggtatcttaaggacctccaag

SEQ ID NO: 646 - DNA, 47 bp, synthetic construct
  misc_feature 1..47: note = Description of Artificial Sequence: Synthetic oligonucleotide
  source 1..47: mol_type = other DNA
taggaagtaaattaatttgaagcttgccaattaatccaaatcttacc

SEQ ID NO: 647 - DNA, 83 bp, synthetic construct
  misc_feature 1..83: note = Description of Artificial Sequence: Synthetic primer
  source 1..83: mol_type = other DNA
tttcctttttgcaaaacccaaaatattttagctcctactcagactgttagagtaggaggagaaggagggctgtggctcttgtg

SEQ ID NO: 648 - DNA, 83 bp, synthetic construct
  misc_feature 1..83: note = Description of Artificial Sequence: Synthetic primer
  variation 20: note = a, c, t, g, unknown or other
  variation 31: note = a, c, t, g, unknown or other
  variation 69: note = a, c, t, g, unknown or other
  source 1..83: mol_type = other DNA
aaaacccccaaaccctcccnctttttcctcntactctaacagtgtgagtaggaggccacacttagctgnggctctagtaagtc

SEQ ID NO: 649 - DNA, 94 bp, synthetic construct
  misc_feature 1..94: note = Description of Artificial Sequence: Synthetic primer
  variation 50: note = a, c, t, g, unknown or other
  source 1..94: mol_type = other DNA
cttcttttttcctttttgcaaaaacccaaaatattttagctcctactcanaccgttttcggggggtattttgtatttggatgaaacacttgatg

SEQ ID NO: 650 - DNA, 93 bp, synthetic construct
  misc_feature 1..93: note = Description of Artificial Sequence: Synthetic primer
  source 1..93: mol_type = other DNA
tcttcttttttccttcccgcaaaaacccaaaatattttcgctcctactcaggacctggtcgtggggtattttgtatttggatgaaacacttga

SEQ ID NO: 651 - DNA, 104 bp, synthetic construct
  misc_feature 1..104: note = Description of Artificial Sequence: Synthetic primer
  variation 21..22: note = a, c, t, g, unknown or other
  source 1..104: mol_type = other DNA
tcaaggaagatggcatttctnntttggagatggcagtttccttagtaaccacaggttgtgtcaccatggccagtaagaaggatgccacacttgtgtgtgtcgct

SEQ ID NO: 652 - DNA, 86 bp, synthetic construct
  misc_feature 1..86: note = Description of Artificial Sequence: Synthetic primer
  variation 35..36: note = a, c, t, g, unknown or other
  variation 42..44: note = a, c, t, g, unknown or other
  source 1..86: mol_type = other DNA
ttggagatagcagtttccttggtaagtacaggttnntcaccnnnacagtgtgagtaggaggccacacttagctgtggctctagtaa

SEQ ID NO: 653 - DNA, 71 bp, synthetic construct
  misc_feature 1..71: note = Description of Artificial Sequence: Synthetic primer
  variation 54: note = a, c, t, g, unknown or other
  variation 61..63: note = a, c, t, g, unknown or other
  variation 69: note = a, c, t, g, unknown or other
  source 1..71: mol_type = other DNA
tttctagtttggagatggcagtttccttagtaaccacaggttgtgtcaccaggnctcgagnnntggttntg

SEQ ID NO: 654 - DNA, 87 bp, synthetic construct
  misc_feature 1..87: note = Description of Artificial Sequence: Synthetic primer
  source 1..87: mol_type = other DNA
cagttgccatagtcttcactaaccgcgtcaccagagctcaggacctggtcgtggggtattttgtatttggatgaaacacttgatggt

SEQ ID NO: 655 - DNA, 92 bp, synthetic construct
  misc_feature 1..92: note = Description of Artificial Sequence: Synthetic primer
  variation 52: note = a, c, t, g, unknown or other
  variation 59: note = a, c, t, g, unknown or other
  variation 78: note = a, c, t, g, unknown or other
  source 1..92: mol_type = other DNA
tattgtatcttggtttttcttcactgctggccagtttactaacaatctgaantaagccnattaatccaaatcttaccngacgaaaaatcaaa

SEQ ID NO: 656 - DNA, 89 bp, synthetic construct
  misc_feature 1..89: note = Description of Artificial Sequence: Synthetic primer
  variation 6: note = a, c, t, g, unknown or other
  variation 10: note = a, c, t, g, unknown or other
  variation 19: note = a, c, t, g, unknown or other
  variation 30: note = a, c, t, g, unknown or other
  variation 57: note = a, c, t, g, unknown or other
  source 1..89: mol_type = other DNA
atggtntttngtcattgcnggccagttttntaacaatgtgatcaatgccaattaatncaaatcttaccagaggaaaaatcaagccacag

SEQ ID NO: 657 - DNA, 94 bp, synthetic construct
  misc_feature 1..94: note = Description of Artificial Sequence: Synthetic primer
  variation 93..94: note = a, c, t, g, unknown or other
  source 1..94: mol_type = other DNA
aataaaaagaaaaaaatatgataaactgctcccagtataaaatacagagctaagacaagaacgtttcattggctgttaaaagtcggaaatcgnn

SEQ ID NO: 658 - DNA, 94 bp, synthetic construct
  misc_feature 1..94: note = Description of Artificial Sequence: Synthetic primer
  source 1..94: mol_type = other DNA
ttctgatttaaaattcccttgaataggaagtaaattaatttgaagcttgccaattaatccaaatcttaccagaggaaaaatcaagccacagata

SEQ ID NO: 659 - DNA, 90 bp, synthetic construct
  misc_feature 1..90: note = Description of Artificial Sequence: Synthetic primer
  source 1..90: mol_type = other DNA
caaaacaagatgggataaaatcttctttctgatttaaaattcccttgaataggaagtaaattactagagctgttcaaggcctgaactcta

SEQ ID NO: 660 - DNA, 96 bp, synthetic construct
  misc_feature 1..96: note = Description of Artificial Sequence: Synthetic primer
  variation 49: note = a, c, t, g, unknown or other
  variation 51..52: note = a, c, t, g, unknown or other
  variation 54: note = a, c, t, g, unknown or other
  source 1..96: mol_type = other DNA
ctgatttaaaattcccttgaataggaagtaaattactagagctgttcangnncngaactctaggctagcatttccagcccaaaacaaacacagttc

SEQ ID NO: 661-668 - skipped (8 entries)

SEQ ID NO: 669 - DNA, 26 bp, synthetic construct
  misc_feature 1..26: note = Description of Artificial Sequence: Synthetic oligonucleotide
  source 1..26: mol_type = other DNA
ttgtttgtcttcgagaagacctgttt

SEQ ID NO: 670 - DNA, 26 bp, synthetic construct
  misc_feature 1..26: note = Description of Artificial Sequence: Synthetic oligonucleotide
  source 1..26: mol_type = other DNA
caccgggtcttcgagaagacctgttt

SEQ ID NO: 671 - DNA, 26 bp, synthetic construct
  misc_feature 1..26: note = Description of Artificial Sequence: Synthetic oligonucleotide
  source 1..26: mol_type = other DNA
tcccaggtcttcgagaagacctgttt

SEQ ID NO: 672 - DNA, 26 bp, synthetic construct
  misc_feature 1..26: note = Description of Artificial Sequence: Synthetic oligonucleotide
  source 1..26: mol_type = other DNA
cctcgggtcttcgagaagacctgttt

SEQ ID NO: 673 - DNA, 25 bp, synthetic construct
  misc_feature 1..25: note = Description of Artificial Sequence: Synthetic oligonucleotide
  source 1..25: mol_type = other DNA
ggcctaccgctaaggctagcctagt

SEQ ID NO: 674 - protein, 1411 aa, synthetic construct
  REGION 1..1411: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVG

SEQ ID NO: 675 - protein, 1483 aa, synthetic construct; identical to SEQ ID NO: 1
  REGION 1..1483: note = Description of Artificial Sequence: Synthetic polypeptide

SEQ ID NO: 676 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 677-678 - skipped (2 entries)

SEQ ID NO: 679 - DNA, 20 bp, synthetic construct
  misc_feature 1..20: note = Description of Artificial Sequence: Synthetic oligonucleotide
  source 1..20: mol_type = other DNA
gtgtagagtaagtcagccta

SEQ ID NO: 680 - DNA, 20 bp, synthetic construct
  misc_feature 1..20: note = Description of Artificial Sequence: Synthetic oligonucleotide
  source 1..20: mol_type = other DNA
gttggacagaacttaccgac

SEQ ID NO: 681 - DNA, 20 bp, synthetic construct
  misc_feature 1..20: note = Description of Artificial Sequence: Synthetic oligonucleotide
  source 1..20: mol_type = other DNA
gtttgcttcgctataaaacg

SEQ ID NO: 682 - DNA, 20 bp, synthetic construct
  misc_feature 1..20: note = Description of Artificial Sequence: Synthetic oligonucleotide
  source 1..20: mol_type = other DNA
gtctgaggatggggccgcaa

SEQ ID NO: 683 - DNA, 20 bp, synthetic construct
  misc_feature 1..20: note = Description of Artificial Sequence: Synthetic oligonucleotide
  source 1..20: mol_type = other DNA
ggatctgtcaaatcgcctgc

SEQ ID NO: 684 - DNA, 20 bp, synthetic construct
  misc_feature 1..20: note = Description of Artificial Sequence: Synthetic oligonucleotide
  source 1..20: mol_type = other DNA
gccaggatggcattgggcag

SEQ ID NO: 685 - DNA, 20 bp, synthetic construct
  misc_feature 1..20: note = Description of Artificial Sequence: Synthetic oligonucleotide
  source 1..20: mol_type = other DNA
gctgaatctgcggtggcagg

SEQ ID NO: 686 - DNA, 20 bp, synthetic construct
  misc_feature 1..20: note = Description of Artificial Sequence: Synthetic oligonucleotide
  source 1..20: mol_type = other DNA
gttcttttgttcttctagcc

SEQ ID NO: 687 - DNA, 20 bp, synthetic construct
  misc_feature 1..20: note = Description of Artificial Sequence: Synthetic oligonucleotide
  source 1..20: mol_type = other DNA
ggaaaagcttgagcaagtca

SEQ ID NO: 688 - DNA, 20 bp, synthetic construct
  misc_feature 1..20: note = Description of Artificial Sequence: Synthetic oligonucleotide
  source 1..20: mol_type = other DNA
ggaagagttgcccctgcgcc

SEQ ID NO: 689 - DNA, 20 bp, synthetic construct
  misc_feature 1..20: note = Description of Artificial Sequence: Synthetic oligonucleotide
  source 1..20: mol_type = other DNA
gacaaatctccagtggataa

SEQ ID NO: 690 - DNA, 20 bp, synthetic construct
  misc_feature 1..20: note = Description of Artificial Sequence: Synthetic oligonucleotide
  source 1..20: mol_type = other DNA
gtgtttctcaggtaaagctc

SEQ ID NO: 691 - DNA, 20 bp, synthetic construct
  misc_feature 1..20: note = Description of Artificial Sequence: Synthetic oligonucleotide
  source 1..20: mol_type = other DNA
ggaaggaccatttgacgtta

SEQ ID NO: 692 - DNA, 20 bp, synthetic construct
  misc_feature 1..20: note = Description of Artificial Sequence: Synthetic oligonucleotide
  source 1..20: mol_type = other DNA
gaactgctatttcagtttcc

SEQ ID NO: 693 - DNA, 20 bp, synthetic construct
  misc_feature 1..20: note = Description of Artificial Sequence: Synthetic oligonucleotide
  source 1..20: mol_type = other DNA
gccagccactcagccagtga

SEQ ID NO: 694 - DNA, 20 bp, synthetic construct
  misc_feature 1..20: note = Description of Artificial Sequence: Synthetic oligonucleotide
  source 1..20: mol_type = other DNA
ggtatgcttttctgttaaag

SEQ ID NO: 695 - DNA, 20 bp, synthetic construct
  misc_feature 1..20: note = Description of Artificial Sequence: Synthetic oligonucleotide
  source 1..20: mol_type = other DNA
gctcctggactgaccactat

SEQ ID NO: 696 - DNA, 23 bp, synthetic construct
  misc_feature 1..23: note = Description of Artificial Sequence: Synthetic oligonucleotide
  source 1..23: mol_type = other DNA
ggaacagaggcgtccccagttgg

SEQ ID NO: 697 - DNA, 20 bp, synthetic construct
  misc_feature 1..20: note = Description of Artificial Sequence: Synthetic oligonucleotide
  source 1..20: mol_type = other DNA
ggaggctagaacaatcatta

SEQ ID NO: 698 - DNA, 20 bp, synthetic construct
  misc_feature 1..20: note = Description of Artificial Sequence: Synthetic oligonucleotide
  source 1..20: mol_type = other DNA
gacaagaacaccttcagaac

SEQ ID NO: 699 - DNA, 20 bp, synthetic construct
  misc_feature 1..20: note = Description of Artificial Sequence: Synthetic oligonucleotide
  source 1..20: mol_type = other DNA
gggtttctgtgattttcttt

SEQ ID NO: 700 - DNA, 20 bp, synthetic construct
  misc_feature 1..20: note = Description of Artificial Sequence: Synthetic oligonucleotide
  source 1..20: mol_type = other DNA
gggccaaagacctccgccag

SEQ ID NO: 701 - DNA, 20 bp, synthetic construct
  misc_feature 1..20: note = Description of Artificial Sequence: Synthetic oligonucleotide
  source 1..20: mol_type = other DNA
gttggagaagcattcataaa

SEQ ID NO: 702 - DNA, 20 bp, synthetic construct
  misc_feature 1..20: note = Description of Artificial Sequence: Synthetic oligonucleotide
  source 1..20: mol_type = other DNA
gtcgctcactcaccctgcaa

SEQ ID NO: 703 - DNA, 20 bp, synthetic construct
  misc_feature 1..20: note = Description of Artificial Sequence: Synthetic oligonucleotide
  source 1..20: mol_type = other DNA
gaaaagagctgatgaaacaa

SEQ ID NO: 704 - DNA, 20 bp, synthetic construct
  misc_feature 1..20: note = Description of Artificial Sequence: Synthetic oligonucleotide
  source 1..20: mol_type = other DNA
gtacacttttcaaaatgctt

SEQ ID NO: 705 - DNA, 20 bp, synthetic construct
  misc_feature 1..20: note = Description of Artificial Sequence: Synthetic oligonucleotide
  source 1..20: mol_type = other DNA
ggagatgatcatcaagcaga

SEQ ID NO: 706 - DNA, 20 bp, synthetic construct
  misc_feature 1..20: note = Description of Artificial Sequence: Synthetic oligonucleotide
  source 1..20: mol_type = other DNA
gctttgaaagagcaataaaa

SEQ ID NO: 707 - DNA, 20 bp, synthetic construct
  misc_feature 1..20: note = Description of Artificial Sequence: Synthetic oligonucleotide
  source 1..20: mol_type = other DNA
gcacaaaagtcaaatcggaa

SEQ ID NO: 708 - DNA, 20 bp, synthetic construct
  misc_feature 1..20: note = Description of Artificial Sequence: Synthetic oligonucleotide
  source 1..20: mol_type = other DNA
gatttcaatataagattcgg

SEQ ID NO: 709 - DNA, 20 bp, synthetic construct
  misc_feature 1..20: note = Description of Artificial Sequence: Synthetic oligonucleotide
  source 1..20: mol_type = other DNA
gcttaagcaatcccgaactc

SEQ ID NO: 710 - DNA, 20 bp, synthetic construct
  misc_feature 1..20: note = Description of Artificial Sequence: Synthetic oligonucleotide
  source 1..20: mol_type = other DNA
gaggccaaacctcggcttac

SEQ ID NO: 711 - DNA, 20 bp, synthetic construct
  misc_feature 1..20: note = Description of Artificial Sequence: Synthetic oligonucleotide
  source 1..20: mol_type = other DNA
gttcgaaaatttcaggtaag

SEQ ID NO: 712 - DNA, 20 bp, synthetic construct
  misc_feature 1..20: note = Description of Artificial Sequence: Synthetic oligonucleotide
  source 1..20: mol_type = other DNA
ggcagaacaggagataacag

SEQ ID NO: 713 - DNA, 20 bp, synthetic construct
  misc_feature 1..20: note = Description of Artificial Sequence: Synthetic oligonucleotide
  source 1..20: mol_type = other DNA
ggcggccctcgcccttctct

SEQ ID NO: 714 - DNA, 20 bp, synthetic construct
  misc_feature 1..20: note = Description of Artificial Sequence: Synthetic oligonucleotide
  source 1..20: mol_type = other DNA
gtagtgatcgtggatacgag

SEQ ID NO: 715 - DNA, 20 bp, synthetic construct
  misc_feature 1..20: note = Description of Artificial Sequence: Synthetic oligonucleotide
  source 1..20: mol_type = other DNA
gtacagccctcggtgtatat

SEQ ID NO: 716 - DNA, 20 bp, synthetic construct
  misc_feature 1..20: note = Description of Artificial Sequence: Synthetic oligonucleotide
  source 1..20: mol_type = other DNA
gggaaggaattaagcccgaa

SEQ ID NO: 717 - DNA, 20 bp, synthetic construct
  misc_feature 1..20: note = Description of Artificial Sequence: Synthetic oligonucleotide
  source 1..20: mol_type = other DNA
gggaacagctttcgtagttg

SEQ ID NO: 718 - DNA, 20 bp, synthetic construct
  misc_feature 1..20: note = Description of Artificial Sequence: Synthetic oligonucleotide
  source 1..20: mol_type = other DNA
gataaagtccagtgtcgatc

SEQ ID NO: 719 - DNA, 20 bp, synthetic construct
  misc_feature 1..20: note = Description of Artificial Sequence: Synthetic oligonucleotide
  source 1..20: mol_type = other DNA
gaaaaccagagcttcggtca

SEQ ID NO: 720 - DNA, 31 bp, synthetic construct
  misc_feature 1..31: note = Description of Artificial Sequence: Synthetic oligonucleotide
  source 1..31: mol_type = other DNA
ggagtcttctgggcaggcttaaaggctaacc

SEQ ID NO: 721 - DNA, 31 bp, synthetic construct
  misc_feature 1..31: note = Description of Artificial Sequence: Synthetic oligonucleotide
  source 1..31: mol_type = other DNA
gtcgggtgagcatgtctttaatctacctcga

SEQ ID NO: 722 - DNA, 20 bp, synthetic construct
  misc_feature 1..20: note = Description of Artificial Sequence: Synthetic oligonucleotide
  source 1..20: mol_type = other DNA
ggtgtcaccagagtaacagt

SEQ ID NO: 723 - DNA, 20 bp, synthetic construct
  misc_feature 1..20: note = Description of Artificial Sequence: Synthetic oligonucleotide
  source 1..20: mol_type = other DNA
gtgatcatcaagcagaaggt

SEQ ID NO: 724 - DNA, 21 bp, synthetic construct
  misc_feature 1..21: note = Description of Artificial Sequence: Synthetic oligonucleotide
  source 1..21: mol_type = other DNA
gaacttcgaaaatttcaggta

SEQ ID NO: 725 - DNA, 21 bp, synthetic construct
  misc_feature 1..21: note = Description of Artificial Sequence: Synthetic oligonucleotide
  source 1..21: mol_type = other DNA
ggaaactcatcaaatatgcgt

SEQ ID NO: 726 - DNA, 21 bp, synthetic construct
  misc_feature 1..21: note = Description of Artificial Sequence: Synthetic oligonucleotide
  source 1..21: mol_type = other DNA
gtcatttacactaacacgcat

SEQ ID NO: 727 - DNA, 21 bp, synthetic construct
  misc_feature 1..21: note = Description of Artificial Sequence: Synthetic oligonucleotide
  source 1..21: mol_type = other DNA
ggaatgaaactcatcaaatat

SEQ ID NO: 728 - DNA, 21 bp, synthetic construct
  misc_feature 1..21: note = Description of Artificial Sequence: Synthetic oligonucleotide
  source 1..21: mol_type = other DNA
gtcatcaatatctttgaagga

SEQ ID NO: 729 - DNA, 21 bp, synthetic construct
  misc_feature 1..21: note = Description of Artificial Sequence: Synthetic oligonucleotide
  source 1..21: mol_type = other DNA
gtgttttcataggaaaaatag

SEQ ID NO: 730 - DNA, 21 bp, synthetic construct
  misc_feature 1..21: note = Description of Artificial Sequence: Synthetic oligonucleotide
  source 1..21: mol_type = other DNA
gaattggaaaatgtgatggga

SEQ ID NO: 731 - DNA, 21 bp, synthetic construct
  misc_feature 1..21: note = Description of Artificial Sequence: Synthetic oligonucleotide
  source 1..21: mol_type = other DNA
gatgatcatcaagcagaaggt

SEQ ID NO: 732 - DNA, 21 bp, synthetic construct
  misc_feature 1..21: note = Description of Artificial Sequence: Synthetic oligonucleotide
  source 1..21: mol_type = other DNA
gagatgatcatcaagcagaag

SEQ ID NO: 733 - DNA, 21 bp, synthetic construct
  misc_feature 1..21: note = Description of Artificial Sequence: Synthetic oligonucleotide
  source 1..21: mol_type = other DNA
gcattttttctcataccttct

SEQ ID NO: 734 - DNA, 21 bp, synthetic construct
  misc_feature 1..21: note = Description of Artificial Sequence: Synthetic oligonucleotide
  source 1..21: mol_type = other DNA
gtcctactcagactgttactc

SEQ ID NO: 735 - DNA, 21 bp, synthetic construct
  misc_feature 1..21: note = Description of Artificial Sequence: Synthetic oligonucleotide
  source 1..21: mol_type = other DNA
gacaggttgtgtcaccagagt

SEQ ID NO: 736 - DNA, 21 bp, synthetic construct
  misc_feature 1..21: note = Description of Artificial Sequence: Synthetic oligonucleotide
  source 1..21: mol_type = other DNA
gttatcattttttctcatacc

SEQ ID NO: 737 - DNA, 21 bp, synthetic construct
  misc_feature 1..21: note = Description of Artificial Sequence: Synthetic oligonucleotide
  source 1..21: mol_type = other DNA
gttgcctaagaactggtggga

SEQ ID NO: 738 - DNA, 21 bp, synthetic construct
  misc_feature 1..21: note = Description of Artificial Sequence: Synthetic oligonucleotide
  source 1..21: mol_type = other DNA
gaaacagttgcctaagaactg

SEQ ID NO: 739 - DNA, 21 bp, synthetic construct
  misc_feature 1..21: note = Description of Artificial Sequence: Synthetic oligonucleotide
  source 1..21: mol_type = other DNA
gtttcccaccagttcttaggc

SEQ ID NO: 740 - DNA, 21 bp, synthetic construct
  misc_feature 1..21: note = Description of Artificial Sequence: Synthetic oligonucleotide
  source 1..21: mol_type = other DNA
gtggctttgatttccctaggg

SEQ ID NO: 741 - DNA, 21 bp, synthetic construct
  misc_feature 1..21: note = Description of Artificial Sequence: Synthetic oligonucleotide
  source 1..21: mol_type = other DNA
gtagggaaatcaaagccaatg

SEQ ID NO: 742 - DNA, 21 bp, synthetic construct
  misc_feature 1..21: note = Description of Artificial Sequence: Synthetic oligonucleotide
  source 1..21: mol_type = other DNA
ggaccctagggaaatcaaagc

SEQ ID NO: 743 - DNA, 21 bp, synthetic construct
  misc_feature 1..21: note = Description of Artificial Sequence: Synthetic oligonucleotide
  source 1..21: mol_type = other DNA
gtgagggctccaccctcacga

SEQ ID NO: 744 - DNA, 21 bp, synthetic construct
  misc_feature 1..21: note = Description of Artificial Sequence: Synthetic oligonucleotide
  source 1..21: mol_type = other DNA
gaaggattgagggctccaccc

SEQ ID NO: 745 - DNA, 21 bp, synthetic construct
  misc_feature 1..21: note = Description of Artificial Sequence: Synthetic oligonucleotide
  source 1..21: mol_type = other DNA
ggctccaccctcacgagtggg

SEQ ID NO: 746 - DNA, 21 bp, synthetic construct
  misc_feature 1..21: note = Description of Artificial Sequence: Synthetic oligonucleotide
  source 1..21: mol_type = other DNA
gtatcccctatcgaggaaacc

SEQ ID NO: 747 - DNA, 21 bp, synthetic construct
  misc_feature 1..21: note = Description of Artificial Sequence: Synthetic oligonucleotide
  source 1..21: mol_type = other DNA
ggataaagaaggcctatttca

SEQ ID NO: 748 - DNA, 21 bp, synthetic construct
  misc_feature 1..21: note = Description of Artificial Sequence: Synthetic oligonucleotide
  source 1..21: mol_type = other DNA
gaggccttctttatcccctat

SEQ ID NO: 749 - DNA, 21 bp, synthetic construct
  misc_feature 1..21: note = Description of Artificial Sequence: Synthetic oligonucleotide
  source 1..21: mol_type = other DNA
gtgagggctccaccctcacga

SEQ ID NO: 750 - DNA, 21 bp, synthetic construct
  misc_feature 1..21: note = Description of Artificial Sequence: Synthetic oligonucleotide
  source 1..21: mol_type = other DNA
ggataaagaaggcctatttca

(19) United States
(12) Patent Application Publication
(10) Pub. No.: US 2026/0209791 A1
(43) Pub. Date: Jul. 23, 2026

(54) RNA-GUIDED GENE EDITING AND GENE REGULATION

(71) Applicant: Duke University, Durham, NC (US)

(72) Inventors: Charles A. Gersbach, Chapel Hill, NC (US); Isaac B. Hilton, Houston, TX (US); Pablo Perez-Pinera, Urbana, IL (US); Ami M. Kabadi, Greensboro, NC (US); Pratiksha I. Thakore, Durham, IL (US); David G. Ousterout, Raleigh, NC (US); Joshua B. Black, Durham, NC (US)

(21) Appl. No.: 19/570,060

(22) Filed: Mar. 17, 2026

Related U.S. Application Data
(63) Continuation of application No. 16/927,679, filed on Jul. 13, 2020.
Application No. 16/927,679 is a continuation of application No. 15/991,333, filed on May 29, 2018, now Pat. No. 10,745,714.
Application No. 15/991,333 is a division of application No. 14/895,316, filed on Jun. 5, 2014, now Pat. No. 10,704,060.
(60) Provisional application No. 61/831,481, filed on Jun. 5, 2013.
(60) Provisional application No. 61/839,127, filed on Jun. 25, 2013.
(60) Provisional application No. 61/904,911, filed on Nov. 15, 2013.
(60) Provisional application No. 61/967,466, filed on Mar. 19, 2014.
(60) Provisional application No. 61/981,575, filed on Apr. 18, 2014.

(51) Int. Cl. C12N 15/85; A61K 38/46; A61K 48/00; C07K 14/47; C12N 9/22; C12N 9/96; C12N 15/90

(52) U.S. Cl. CPC C12N 15/85; A61K 38/465; A61K 48/0058; C07K 14/4708; C12N 9/22; C12N 9/96; C12N 15/907; A61K 48/005; C07K 2319/71; C12N 2740/16043; C12N 2800/40; C12N 2840/20; C12Y 301/00

(57) ABSTRACT

Disclosed herein are Clustered Regularly Interspaced Short Palindromic Repeats (CRISPR)/CRISPR-associated (Cas) 9-based system related compositions and methods of using said CRISPR/Cas9-based system related compositions for altering gene expression and genome engineering. Also disclosed herein are compositions and methods of using said compositions for altering gene expression and genome engineering in muscle, such as skeletal muscle and cardiac muscle.

### CROSS REFERENCE TO RELATED APPLICATIONS

This application is a continuation of U.S. patent application Ser. No. 16/927,679, filed Jul. 13, 2020, which is a continuation of U.S. patent application Ser. No. 15/991,333, filed May 29, 2018, which is a divisional of U.S. patent application Ser. No. 14/895,316 filed Dec. 2, 2015, that issued as U.S. Pat. No. 10,704,060, which is a national stage filing under 35 U.S.C. 371 of International Patent Application No. PCT/US2014/041190, filed Jun. 5, 2014, which claims the benefit of priority to U.S. Provisional Application No. 61/831,481, filed Jun. 5, 2013, U.S. Provisional Application No. 61/839,127, filed Jun. 25, 2013, U.S. Provisional Application No. 61/904,911, filed Nov. 15, 2013, U.S. Provisional Application No. 61/967,466, filed Mar. 19, 2014, and U.S. Provisional Application No. 61/981,575, filed Apr. 18, 2014, all of which are incorporated herein by reference in their entirety.

### STATEMENT OF GOVERNMENT INTEREST

This invention was made with government support under federal grant numbers DP2-OD008586 and R01DA036865 awarded by National Institutes of Health and CBET-1151035 awarded by the National Science Foundation. The U.S. Government has certain rights to this invention.

### REFERENCE TO AN ELECTRONIC SEQUENCE LISTING

The sequence listing is filed with the application and is incorporated by reference herein. The electronic sequence listing file “028193-9164-US07_Sequence_Listing.xml” was created on Mar. 16, 2026, and is 1,020,967 bytes in size.

### TECHNICAL FIELD

The present disclosure relates to the field of gene expression alteration, genome engineering and genomic alteration of genes using Clustered Regularly Interspaced Short Palindromic Repeats (CRISPR)/CRISPR-associated (Cas) 9-based systems and viral delivery systems. The present disclosure also relates to the field of genome engineering and genomic alteration of genes in muscle, such as skeletal muscle and cardiac muscle.

### BACKGROUND

Synthetic transcription factors have been engineered to control gene expression for many different medical and scientific applications in mammalian systems, including stimulating tissue regeneration, drug screening, compensating for genetic defects, activating silenced tumor suppressors, controlling stem cell differentiation, performing genetic screens, and creating synthetic gene circuits. These transcription factors can target promoters or enhancers of endogenous genes, or be purposefully designed to recognize sequences orthogonal to mammalian genomes for transgene regulation. The most common strategies for engineering novel transcription factors targeted to user-defined sequences have been based on the programmable DNA-binding domains of zinc finger proteins and transcription-activator like effectors (TALEs). Both of these approaches involve applying the principles of protein-DNA interactions of these domains to engineer new proteins with unique DNA-binding specificity. Although these methods have been widely successful for many applications, the protein engineering necessary for manipulating protein-DNA interactions can be laborious and require specialized expertise.

Additionally, these new proteins are not always effective. The reasons for this are not yet known but may be related to the effects of epigenetic modifications and chromatin state on protein binding to the genomic target site. In addition, there are challenges in ensuring that these new proteins, as well as other components, are delivered to each cell. Existing methods for delivering these new proteins and their multiple components include delivery to cells on separate plasmids or vectors which leads to highly variable expression levels in each cell due to differences in copy number. Additionally, gene activation following transfection is transient due to dilution of plasmid DNA, and temporary gene expression may not be sufficient for inducing therapeutic effects. Furthermore, this approach is not amenable to cell types that are not easily transfected. Thus another limitation of these new proteins is the potency of transcriptional activation.

Site-specific nucleases can be used to introduce site-specific double strand breaks at targeted genomic loci. This DNA cleavage stimulates the natural DNA-repair machinery, leading to one of two possible repair pathways. In the absence of a donor template, the break will be repaired by non-homologous end joining (NHEJ), an error-prone repair pathway that leads to small insertions or deletions of DNA. This method can be used to intentionally disrupt, delete, or alter the reading frame of targeted gene sequences. However, if a donor template is provided along with the nucleases, then the cellular machinery will repair the break by homologous recombination, which is enhanced several orders of magnitude in the presence of DNA cleavage. This method can be used to introduce specific changes in the DNA sequence at target sites. Engineered nucleases have been used for gene editing in a variety of human stem cells and cell lines, and for gene editing in the mouse liver. However, the major hurdle for implementation of these technologies is delivery to particular tissues in vivo in a way that is effective, efficient, and facilitates successful genome modification.

Hereditary genetic diseases have devastating effects on children in the United States. These diseases currently have no cure and can only be managed by attempts to alleviate the symptoms. For decades, the field of gene therapy has promised a cure to these diseases. However technical hurdles regarding the safe and efficient delivery of therapeutic genes to cells and patients have limited this approach. Duchenne Muscular Dystrophy (DMD) is the most common hereditary monogenic disease and occurs in 1 in 3500 males. DMD is the result of inherited or spontaneous mutations in the dystrophin gene. Dystrophin is a key component of a protein complex that is responsible for regulating muscle cell integrity and function. DMD patients typically lose the ability to physically support themselves during childhood, become progressively weaker during the teenage years, and die in their twenties. Current experimental gene therapy strategies for DMD require repeated administration of transient gene delivery vehicles or rely on permanent integration of foreign genetic material into the genomic DNA. Both of these methods have serious safety concerns. Furthermore, these strategies have been limited by an inability to deliver the large and complex dystrophin gene sequence.

### SUMMARY

The present invention is directed to a fusion protein comprising two heterologous polypeptide domains. The first polypeptide domain comprises a Clustered Regularly Interspaced Short Palindromic Repeats associated (Cas) protein and the second polypeptide domain has an activity selected from the group consisting of transcription activation activity, transcription repression activity, transcription release factor activity, histone modification activity, nuclease activity, nucleic acid association activity, methylase activity, and demethylase activity. The Cas protein may comprise Cas9. The Cas9 may comprise at least one amino acid mutation which knocks out nuclease activity of Cas9. The at least one amino acid mutation may be at least one of D10A and H840A. The Cas protein may comprise iCas9 (amino acids 36-1403 of SEQ ID NO: 1). The second polypeptide domain may have transcription activation activity. The second polypeptide domain may comprise at least one VP16 transcription activation domain repeat. The second polypeptide domain may comprise a VP16 tetramer (“VP64”) or a p65 activation domain. The fusion protein may further comprise a linker connecting the first polypeptide domain to the second polypeptide domain. The fusion protein may comprise iCas9-VP64.

The present invention is directed to a DNA targeting system comprising said fusion protein and at least one guide RNA (gRNA). The at least one gRNA may comprise a 12-22 base pair complementary polynucleotide sequence of the target DNA sequence followed by a protospacer-adjacent motif. The at least one gRNA may target a promoter region of a gene, an enhancer region of a gene, or a transcribed region of a gene. The at least one gRNA may target an intron of a gene. The at least one gRNA may target an exon of a gene. The at least one gRNA may target a the promoter region of a gene selected from the group consisting of ASCL1, BRN2, MYT1L, NANOG, VEGFA, TERT, IL1B, IL1R2, IL1RN, HBG1, HBG2, and MYOD1. The at least one gRNA may comprise at least one of SEQ ID NOs: 5-40, 65-144, 492-515, 540-563, and 585-625.

The present invention is directed to a DNA targeting system that binds to a dystrophin gene comprising Cas9 and at least one guide RNA (gRNA). The at least one gRNA may target an intron of the dystrophin gene. The at least one gRNA may target an exon of the dystrophin gene. The at least one guide RNA may comprise at least one of SEQ ID NOs: 5-40, 65-144, 492-515, 540-563, and 585-625. The DNA targeting system may comprise between one and ten different gRNAs.

The present invention is directed to an isolated polynucleotide encoding said fusion protein or said DNA targeting system.

The present invention is directed to a vector comprising said isolated polynucleotide.

The present invention is directed to a cell comprising said isolated polynucleotide or said vector.

The present invention is directed to a method of modulating mammalian gene expression in a cell. The method comprises contacting the cell with said fusion protein, said DNA targeting system, said isolated polynucleotide, or said vector. The gene expression may be induced.

The present invention is directed to a method of transdifferentiating or inducing differentiation of a cell. The method comprises contacting the cell with said fusion protein, said DNA targeting system, said isolated polynucleotide, or said vector. The cell may be a fibroblast cell or an induced pluripotent stem cells. The fibroblast cell may be trandifferentiated into a neuronal cell or a myogenic cell. The DNA targeting system may be contacted with the cell and at least one gRNA targets a promoter region of at least one gene selected from the group consisting of ASCL1, BRN2, MYOD1, and MYT1L. The DNA targeting system may comprise at least one gRNA that targets the promoter region of the ASCL1 gene and at least one gRNA that targets the promoter region of the BRN2 gene. The DNA targeting system may comprise between one and twenty different gRNAs. The DNA targeting system may comprise 8 or 16 different gRNAs. The DNA targeting system may comprise dCas9-VP64. The DNA targeting system may be delivered to the cell virally or non-virally.

The present invention is directed to a method of correcting a mutant gene in a cell. The method comprises administering to a cell containing said DNA targeting system, said isolated polynucleotide, or said vector. The correction of the mutant gene may comprise homology-directed repair. The method may further comprise administering to the cell a donor DNA. The mutant gene may comprise a frameshift mutation which causes a premature stop codon and a truncated gene product. The correction of the mutant gene may comprise nuclease mediated non-homologous end joining. The correction of the mutant gene may comprise a deletion of a premature stop codon, a disruption of a splice acceptor site, a deletion of one or more exons, or disruption of a splice donor sequence. The deletion of one or more exons may result in the correction of the reading frame.

The present invention is directed to a method of treating a subject in need thereof having a mutant dystrophin gene. The method comprises administering to the subject said DNA targeting system, said isolated polynucleotide, or said vector. The subject may be suffering from Duchenne muscular dystrophy.

The present invention is directed to a method of correcting a mutant dystrophin gene in a cell. The method comprises administering to a cell containing a mutant dystrophin gene said DNA targeting system, said isolated polynucleotide, said vector, or said cell. The mutant dystrophin gene may comprise a premature stop codon, disrupted reading frame via gene deletion, an aberrant splice acceptor site, or an aberrant splice donor site, and wherein the target region is upstream or downstream of the premature stop codon, disrupted reading frame, aberrant splice acceptor site, or the aberrant splice donor site. The correction of the mutant dystrophin gene may comprise homology-directed repair. The method may further comprise administering to the cell a donor DNA. The mutant dystrophin gene may comprise a frameshift mutation which causes a premature stop codon and a truncated gene product. The correction of the mutant dystrophin gene may comprise nuclease mediated non-homologous end joining. The correction of the mutant dystrophin gene may comprise a deletion of a premature stop codon, correction of a disrupted reading frame, or modulation of splicing by disruption of a splice acceptor site or disruption of a splice donor sequence. The correction of the mutant dystrophin gene may comprise a deletion of exons 45-55 or exon 51.

The present invention is directed to a kit comprising said fusion protein, said DNA targeting system, said isolated polynucleotide, said vector, or said cell.

The present invention is directed to a method of modulating mammalian gene expression in a cell. The method comprises contacting the cell with a polynucleotide encoding a DNA targeting system. The DNA targeting system comprises said fusion protein and at least one guide RNA (gRNA). The DNA targeting system may comprise between one and ten different gRNAs. The different gRNAs may bind to different target regions within the target gene. The target regions may be separated by at least one nucleotide. The target regions may be separated by about 15 to about 700 base pairs. Each of the different gRNAs may bind to at least one different target genes. The different target genes may be located on same chromosome. The different target genes may be located on different chromosomes. The at least one target region may be within a non-open chromatin region, an open chromatin region, a promoter region of the target gene, an enhancer region of the target gene, a transcribed region of the target gene, or a region upstream of a transcription start site of the target gene. The at least one target region may be located between about 1 to about 1000 base pairs upstream of a transcription start site of a target gene. The at least one target region may be located between about 1 to about 600 base pairs upstream of a transcription start site of a target gene. The gene expression may be induced. The DNA targeting system may comprise two different gRNAs, three different gRNAs, four different gRNAs, five different gRNAs, six different gRNAs, seven different gRNAs, eight different gRNAs, nine different gRNAs, or ten different gRNAs. The at least one guide RNA may target a promoter region of a gene selected from the group consisting of ASCL1, BRN2, MYT1L, NANOG, VEGFA, TERT, IL1B, IL1R2, IL1RN, HBG1, HBG2, and MYOD1. The at least one guide RNA may comprise at least one of SEQ ID NOs: 5-40, 65-144, 492-515, 540-563, and 585-625. The at least one target region may be within an intron or an exon of a target gene.

The present invention is directed to a composition for inducing mammalian gene expression in a cell. The composition comprises said fusion protein and at least one guide RNA (gRNA).

The present invention is directed to a composition for inducing mammalian gene expression in a cell. The composition comprises an isolated polynucleotide sequence encoding said fusion protein and at least one guide RNA (gRNA). The at least one guide RNA may target a promoter region of a gene selected from the group consisting of ASCL1, BRN2, MYT1L, NANOG, VEGFA, TERT, IL1B, IL1R2, IL1RN, HBG1, HBG2, and MYOD1. The at least one guide RNA may comprise at least one of SEQ ID NOs 5-40, 65-144, 492-515, 540-563, and 585-625.

The present invention is directed to a cell comprising said composition for inducing mammalian gene expression in a cell.

The present invention is directed to a kit comprising said composition for inducing mammalian gene expression in a cell or said cell comprising said composition for inducing mammalian gene expression in a cell.

The present invention is directed to a kit for inducing mammalian gene expression in a cell. The kit comprises said composition for inducing mammalian gene expression in a cell or said cell comprising said composition for inducing mammalian gene expression in a cell.

The present invention is directed to a composition for genome editing in a muscle of a subject. The composition comprises a modified adeno-associated virus (AAV) vector and a nucleotide sequence encoding a site-specific nuclease. The muscle is skeletal muscle or cardiac muscle. The modified AAV vector may have enhanced cardiac and skeletal muscle tissue tropism. The site-specific nuclease may comprise a zinc finger nuclease, a TAL effector nuclease, or a CRISPR/Cas9 system. The site-specific nuclease may bind a gene or locus in the cell of the muscle. The gene or locus may be dystrophin gene. The composition may further comprise a donor DNA or transgene.

The present invention is directed to a kit comprising said composition for genome editing in a muscle of a subject.

The present invention is directed to a method of genome editing in a muscle of a subject. The method comprises administering to the muscle said composition for genome editing in a muscle of a subject, wherein the muscle is skeletal muscle or cardiac muscle. The genome editing may comprise correcting a mutant gene or inserting a transgene. Correcting a mutant gene may comprise deleting, rearranging, or replacing the mutant gene. Correcting the mutant gene may comprise nuclease-mediated non-homologous end joining or homology-directed repair.

The present invention is directed to a method of treating a subject. The method comprises administering said composition for genome editing in a muscle of a subject to a muscle of the subject, wherein the muscle is skeletal muscle or cardiac muscle. The subject may be suffering from a skeletal muscle condition or a genetic disease. The subject may be suffering from Duchenne muscular dystrophy.

The present invention is directed to a method of correcting a mutant gene in a subject, the method comprises administering said composition for genome editing in a muscle of a subject. The muscle is skeletal muscle or cardiac muscle. The composition may be injected into the skeletal muscle of the subject. The composition may be injected systemically to the subject. The skeletal muscle may be tibialis anterior muscle.

The present invention is directed to a modified lentiviral vector for genome editing in a subject comprising a first polynucleotide sequence encoding said fusion protein and a second polynucleotide sequence encoding at least one sgRNA. The first polynucleotide sequence may be operably linked to a first promoter. The first promoter may be a constitutive promoter, an inducible promoter, a repressible promoter, or a regulatable promoter. The second polynucleotide sequence may encode between one and ten different sgRNAs. The second polynucleotide sequence may encode two different sgRNAs, three different sgRNAs, four different sgRNAs, five different sgRNAs, six different sgRNAs, seven different sgRNAs, eight different sgRNAs, nine different sgRNAs, or ten different sgRNAs. Each of the polynucleotide sequences encoding the different sgRNAs may be operably linked to a promoter. Each of the promoters operably linked to the different sgRNAs may be the same promoter. Each of the promoters operably linked to the different sgRNAs may be different promoters. The promoter may be a constitutive promoter, an inducible promoter, a repressible promoter, or a regulatable promoter. The sgRNA may bind to a target gene. Each of the sgRNA may bind to a different target region within one target loci. Each of the sgRNA may bind to a different target region within different gene loci. The fusion protein may comprise Cas9 protein or iCas9-VP64 protein. The fusion protein may comprise a VP64 domain, a p300 domain, or a KRAB domain. The two or more endogenous genes may be transcriptionally activated. The two or more endogenous genes may be repressed.

The present invention is directed to a method of activating an endogenous gene in a cell. The method comprises contacting a cell with said modified lentiviral vector. The endogenous gene may be transiently activated. The endogenous gene may be stably activated. The endogenous gene may be transiently repressed. The endogenous gene may be stably repressed. The fusion protein may be expressed at similar levels to the sgRNAs. The fusion protein may be expressed at different levels to the sgRNAs. The cell may be a primary human cell.

The present invention is directed to a method of multiplex gene editing in a cell. The method comprises contacting a cell with said modified lentiviral vector. The multiplex gene editing may comprise correcting at least one mutant gene or inserting a transgene. Correcting a mutant gene may comprise deleting, rearranging, or replacing the at least one mutant gene. Correcting the at least one mutant gene may comprise nuclease-mediated non-homologous end joining or homology-directed repair. The multiplex gene editing may comprise deleting at least one gene, wherein the gene is an endogenous normal gene or a mutant gene. The multiplex gene editing may comprise deleting at least two genes. The multiplex gene editing may comprise deleting between two and ten genes.

The present invention is directed to a method of modulating gene expression of at least one target gene in a cell. The method comprises contacting a cell with said modified lentiviral vector. The gene expression of at least two genes may be modulated. The gene expression of between two genes and ten genes may be modulated. The gene expression of the at least one target gene may be modulated when gene expression levels of the at least one target gene are increased or decreased compared to normal gene expression levels for the at least one target gene.

### BRIEF DESCRIPTION OF THE DRAWINGS

FIGS. 1A-1F show RNA-guided activation of the human IL1RN gene by iCas9-VP64. FIG. 1A and FIG. 1B show an RNA-guided transcriptional activator was created by fusing the inactivated Cas9 (iCas9, D10A/H840A) to the VP64 transactivation domain. iCas9-VP64 recognizes genomic target sites through the hybridization of a guide RNA (gRNA) to a 20 bp target sequence. FIG. 1C shows expression plasmids for four gRNAs or crRNA/tracrRNAs targeted to sequences in the IL1RN promoter were co-transfected with the iCas9-VP64 expression plasmid into HEK293T cells. Activation of IL1RN expression was assessed by qRT-PCR. FIG. 1D shows four gRNA expression plasmids were co-transfected with iCas9-VP64 individually or in combination. Robust gene activation was observed by qRT-PCR only in response to the combination of gRNAs. FIG. 1E shows activation of IL1RN expression was confirmed by assessing secretion of the IL-1ra gene product into the media by ELISA. IL-1ra was only detected in three of the six samples treated with the combination of gRNAs. For FIG. 1C and FIG. 1E, data are shown as the mean±s.e.m. (n=3 independent experiments). Treatment with the combination of gRNAs was statistically different than all other treatments (*P≤0.02) by Tukey's test. FIG. 1F shows RNA-seq was performed on samples treated with empty expression vector (n=2) or co-transfected with the expression plasmids for iCas9-VP64 and the four gRNAs targeting IL1RN (n=2). The only statistically significant changes in gene expression between these treatments were an increase in the four IL1RN isoforms (false discovery rate≤3×10-4) and a decrease in IL32 (false discovery rate=0.03).

FIGS. 2A-2H show RNA-guided activation of human genes relevant to cell and gene therapy, genetic reprogramming, and regenerative medicine. HEK293T cells were transfected with the iCas9-VP64 expression plasmid and four gRNAs individually or in combination. Target gene expression was measured by qRT-PCR and normalized to GAPDH mRNA levels. Data are shown as the mean±s.e.m. (n=3 independent experiments). Treatment with the combination of gRNAs was statistically different than all other treatments (*P<0.05) by Tukey's test.

FIG. 3 shows expression of iCas9-VP64. Expression of iCas9-VP64 in transfected HEK293 cells was confirmed by western blot for the N-terminal Flag epitope tag. The wt Cas9 expression plasmid does not contain the epitope tag.

FIGS. 4A-4D show positions of gRNA target sites and DNAse hypersensitivity of human target genes. The four gRNA target sites for each locus are designated as custom tracks above each gene and DNase-seq data indicating DNAse-hypersensitive open chromatin regions is shown below each gene. DNase-seq was performed in HEK293T cells to identify DNase hypersensitive regions, as previously described (Song et al., Cold Spring Harbor protocols 2010, pdb prot5384 (2010); Song et al. Genome Res 21, 1757-1767 (2011)). The results show that open chromatin was not a requirement for gene activation by combinations of gRNAs with iCas9-VP64.

FIG. 5 shows the absence of nuclease activity by iCas9-VP64. Wild-type Cas9 or inactivated (D10A, H840A) iCas9-VP64 expression plasmids were co-transfected with expression plasmids for four different guide RNAs targeting the IL1RN promoter. Nuclease activity was determined by the Surveyor assay (Guschin et al., Methods Mol Biol 649, 247-256 (2010)). The lower molecular weight bands indicative of nuclease activity and DNA repair by non-homologous end joining are only present following treatment with wild-type Cas9, supporting abrogation of nuclease activity by iCas9-VP64.

FIG. 6 shows RNA-seq for samples treated with gRNAs targeting HBG1 and HBG2. RNA-seq was performed on samples treated with a control empty expression vector (n=3) or cotransfected with the expression plasmids for iCas9-VP64 and the four gRNAs targeting HBG1 (n=2). Three of these gRNAs also target HBG2. Increases in both HBG1 and HBG2 relative to control were observed but were not statistically significant due to low expression levels. The only statistically significant changes in gene expression between these treatments were decreases in IL32 (false discovery rate=0.0007) and TNFRS9 (false discovery rate=0.002).

FIG. 7 shows upregulation of Ascl1 and γ-globin by iCas9-VP64. HEK293T cells were transfected with iCas9-VP64 and four gRNAs targeting the ASCL1 or HBG1 promoter. Levels of corresponding Ascl1 and γ-globin protein production were assessed by western blot. Low levels of these proteins were detectable in HEK293T cells and increases in expression were detectable following iCas9-VP64 treatment in two independent experiments.

FIGS. 8A-8H show activation of downstream targets of Ascl1 in iCas9-VP64-treated murine embryonic fibroblasts. Mouse embryonic fibroblasts (MEFs) were transfected with a control GFP expression plasmid or the iCas9-VP64 expression plasmid and a combination of four gRNA expression plasmids targeting ASCL1 at a ratio of 50:50 or 75:25. FIG. 8A shows gRNA target sites in the human ASCL1 promoter (SEQ ID NO: 3) are conserved in the mouse ASCL1 promoter (SEQ ID NO: 4). Target sites are indicated by solid lines and the transcribed region is indicated by dashed line. FIG. 8B shows ASCL1 expression in MEFs increased at two days after iCas9-VP64/gRNA treatment as determined by qRT-PCR. FIGS. 8C-8H show that after 10 days in neural induction media, cells were stained for Ascl1 and Tuj1, an early marker of neuronal differentiation (FIGS. 8C-8D), or for Tuj1 and MAP2, a marker of more mature neuronal differentiation (FIGS. 8E-8F). FIGS. 8F-8G show that some Tuj1-positive cells adopted neuronal morphologies. FIG. 8G shows that a single cell was found to be positive for Tuj1 and MAP2. FIG. 8H shows Tuj 1-positive cells were readily identified in the iCas9-VP64/gRNA-treated cultures (~0.05%) but were absent in controls. n=3 independent samples and data are represented as mean±standard error of the mean. gRNA 75/25 is significantly different than gRNA 50/50 and control (*P<0.01, Tukey's test).

FIG. 9A shows the iCas9-VP64 protein sequence (SEQ ID NO: 1) and FIG. 9B shows the sequence of the gRNA expression cassette with U6 promoter (SEQ ID NO: 2).

FIGS. 10A-10B show the standard curves for qRT-PCR. For each gene, the experimental sample with the highest expression level was diluted to create a standard curve that was assayed by qRT-PCR to ensure efficient amplification over an appropriate dynamic range. The efficiencies of all amplification reactions were within 90-115%.

FIG. 11A and FIG. 11B show the validation of RNA-guided repair. FIG. 11A shows the Surveyor assay results of genomic DNA harvested from HEK 293T cells two days after Cas9 was co-transfected into the cells with empty vector (negative control) or gRNA. FIG. 11B shows the location of the gRNA target. FIG. 11C shows the expected cleavage sizes for each gRNA.

FIG. 12 shows RNA-guided repair in DMD 8036 (del48-50) cells as shown by Surveyor assay.

FIG. 13 shows RNA-guided repair in DMD 8036 (del48-50) cells as shown by PCR across the entire locus. The PCR of a wild-type dystrophin gene generates a fragment of 1447 bp in size, whereas PCR of the mutant gene in the DMD 8036 cell line shows a deletion of approximately 817 bp. The deletion band after introduction of the CRISPR/Cas9-based system was approximately 630 bp.

FIG. 14 shows RNA-guided repair in DMD 8036 (del48-50) cells as shown by Western blot with MANDYS8 (anti-dystrophin antibody) and GAPDH antibody (positive control).

FIG. 15 shows ChIP sequencing data illustrating the specific binding of iCas9-VP64 targeting the IL1RN promoter. HEK 293T cells were transfected with iCas9-VP64 targeting the IL1RN promoter.

FIGS. 16A-16D show CRISPR/Cas9 targeting of the dystrophin gene. FIG. 16A shows sgRNA sequences were designed to bind sequences in the exon 45-55 mutational hotspot region of the dystrophin gene, such that gene editing could restore dystrophin expression from a wide variety of patient-specific mutations. Arrows within introns indicate sgRNA targets designed to delete entire exons from the genome. Arrows within exons indicate sgRNA targets designed to create targeted frameshifts in the dystrophin gene. FIG. 16B shows an example of frame correction following introduction of small insertions or deletions by NHEJ DNA repair in exon 51 using the CR3 sgRNA. FIG. 16B discloses SEQ ID NOS: 633-636, respectively, in order of appearance. FIG. 16C shows a schematic of multiplex sgRNA targets designed to delete exon 51 and restore the dystrophin reading frame in a patient mutation with the deletion of exons 48-50. FIG. 16D shows a schematic of multiplex sgRNA targets designed to delete the entire exon 45-55 region to address a variety of DMD patient mutations.

FIG. 17 shows images of TBE-PAGE gels used to quantify Surveyor assay results to measure day 3 gene modification in Table 7. Asterisks mark expected sizes of bands indicative of nuclease activity.

FIG. 18 shows images of TBE-PAGE gels used to quantify Surveyor assay results to measure day 10 gene modification in Table 7. Asterisks mark expected sizes of bands indicative of nuclease activity.

FIGS. 19A-19D show fluorescence-activated flow sorting to enrich genetically modified DMD myoblasts. FIG. 19A shows a plasmid expressing a human-codon optimized SpCas9 protein linked to a GFP marker using a T2A ribosomal skipping peptide sequence was co-electroporated into human DMD myoblasts with one or two plasmids carrying sgRNA expression cassettes. FIG. 19B shows the indicated sgRNA expression cassettes were independently co-transfected into HEK293 Ts with a separate plasmid expressing SpCas9 with (bottom) or without (top) a GFP marker linked to SpCas9 by a T2A ribosomal skipping peptide sequence. Gene modification frequencies were assessed at 3 days post-transfection by the Surveyor assay. FIG. 19C shows DMD myoblasts with deletions of exons 48-50 in the dystrophin gene were treated with sgRNAs that correct the dystrophin reading frame in these patient cells. Gene modification was assessed at 20 days post-electroporation in unsorted (bulk) or GFP+ sorted cells. FIG. 19D shows GFP expression in DMD myoblasts 3 days after electroporation with indicated expression plasmids. Transfection efficiencies and sorted cell populations are indicated by the gated region.

FIGS. 20A-20D show targeted frameshifts to restore the dystrophin reading frame using CRISPR/Cas9. FIG. 20A shows the 5′ region of exon 51 was targeted using a sgRNA (SEQ ID NO: 637), CR3, that binds immediately upstream of the first out-of-frame stop codon. PAM: protospacer-adjacent motif. FIG. 20B shows the exon 51 locus was PCR amplified from HEK293T cells treated with SpCas9 and CR3 expression cassettes. Sequences of individual clones were determined by Sanger sequencing. The top sequence (bolded, exon in red) is the native, unmodified sequence. The number of clones for each sequence is indicated in parentheses. FIG. 20C shows summary of total gene editing efficiency and reading frame conversions resulting from gene modification shown in FIG. 20B. FIG. 20D shows western blot for dystrophin expression in human DMD myoblasts treated with SpCas9 and the CR3 sgRNA expression cassette (FIG. 19C) to create targeted frameshifts to restore the dystrophin reading frame. Dystrophin expression was probed using an antibody against the rod-domain of the dystrophin protein after 6 days of differentiation.

FIGS. 21A-21D show deletion of exon 51 from the human genome using multiplex CRISPR/Cas9 gene editing. FIG. 21A shows end-point genomic PCR across the exon 51 locus in human DMD myoblasts with a deletion of exons 48-50. The top arrow indicates the expected position of full-length PCR amplicons and the two lower arrows indicate the expected position of PCR amplicons with deletions caused by the indicated sgRNA combinations. FIG. 21B shows PCR products from FIG. 21A were cloned and individual clones were sequenced to determine insertions and deletions present at the targeted locus (SEQ ID NOS: 424, 638, 425-428, 639 and 429-431, respectively, in order of appearance). The top row shows the wild-type unmodified sequence and the triangles indicate SpCas9 cleavage sites. At the right are representative chromatograms showing the sequences of the expected deletion junctions (SEQ ID NOS: 640-642, respectively, in order of appearance). FIG. 21C shows end-point RT-PCR analysis of dystrophin mRNA transcripts in CRISPR/Cas9-modified human Δ48-50 DMD myoblasts treated with the indicated sgRNAs. A representative chromatogram of the expected deletion PCR product is shown at the right. Asterisk: band resulting from hybridization of the deletion product strand to the unmodified strand. FIG. 21D shows rescue of dystrophin protein expression by CRISPR/Cas9 genome editing was assessed by western blot for the dystrophin protein with GAPDH as a loading control. The arrow indicates the expected restored dystrophin protein band.

FIGS. 22A-22D show deletion of the entire exon 45-55 region in human DMD myoblasts by multiplex CRISPR/Cas9 gene editing. FIG. 22A shows end-point genomic PCR of genomic DNA to detect deletion of the region between intron 44 and intron 55 after treating HEK293 Ts or DMD myoblasts with the indicated sgRNAs. FIG. 22B shows individual clones of PCR products (SEQ ID NOS: 432, 643 and 433, respectively, in order of appearance) of the expected size for the deletions from DMD myoblasts in FIG. 22A were analyzed by Sanger sequencing to determine the sequences of genomic deletions present at the targeted locus. Below is a representative chromatograms showing the sequence of the expected deletion junctions (SEQ ID NO: 644). FIG. 22C shows end-point RT-PCR analysis of dystrophin mRNA transcripts in CRISPR/Cas9-modified human Δ48-50 DMD myoblasts treated with the indicated sgRNAs. A representative chromatogram of the expected deletion PCR product is shown at the right (SEQ ID NO: 645). FIG. 22D shows analysis of restored dystrophin protein expression by western blot following electroporation of DMD myoblasts with sgRNAs targeted to intron 44 and/or intron 55.

FIG. 23 shows verification of flow cytometry-based enrichment of gene-modified DMD myoblasts used for in vivo cell transplantation experiment. DMD myoblasts were treated with Cas9 with or without sgRNA expression vectors for CR1 and CR5 and sorted for GFP+ cells by flow cytometry. Deletions at the exon 51 locus were detected by end-point PCR using primers flanking the locus. Neg ctrl: DMD myoblasts treated with Cas9 only and sorted for GFP+ cells.

FIG. 24 shows expression of restored human dystrophin in vivo following transplantation of CRISPR/Cas9-treated human DMD myoblasts into immunodeficient mice. Human Δ48-50 DMD myoblasts were treated with SpCas9, CR1, and CR5 to delete exon 51 and sorted for GFP expression as shown in FIG. 19. These sorted cells and untreated control cells were injected into the hind limbs of immunodeficient mice and assessed for human-specific protein expression in muscle fibers after 4 weeks post-transplantation. Cryosections were stained with anti-human spectrin, which is expressed by both uncorrected and corrected myoblasts that have fused into mouse myofibers, or anti-human dystrophin antibodies as indicated. White arrows indicate muscle fibers positive for human dystrophin.

FIGS. 25A-25F show additional immunofluorescence images probing human dystrophin expression. Serial sections from regions stained with anti-human spectrin are shown inset in top left. FIGS. 25A-25C show sections from muscles injected with untreated human DMD myoblasts. FIGS. 25D-25F show sections from muscles injected with CR1/5 treated human DMD myoblasts enriched by flow cytometry. White arrows indicate dystrophin positive fibers.

FIGS. 26A-26D show evaluation of CRISPR/Cas9 toxicity and off-target effects for CR1/CR5-mediated deletion of exon 51 in human cells. FIG. 26A shows results of a cytotoxicity assay in HEK293T cells treated with human-optimized SpCas9 and the indicated sgRNA constructs. Cytotoxicity is based on survival of GFP-positive cells that are co-transfected with the indicated nuclease. I-SceI is a well-characterized non-toxic meganuclease and GZF3 is a known toxic zinc finger nuclease. FIG. 26B shows surveyor analysis at off-target sites in sorted hDMD cells treated with expression cassettes encoding Cas9 the indicated sgRNAs. These three off-target sites tested in hDMD cells were identified from a panel of 50 predicted sites tested in HEK293T cells (FIG. 27 and Table 4). TGT: on-target locus for indicated sgRNA. OT: off-target locus. FIGS. 26C-26D show end-point nested PCR to detect chromosomal translocations in HEK293T cells treated with Cas9 and CR1 (FIG. 26C) or sorted hDMD cells treated with Cas9, CR1, and CR5 (FIG. 26D). The schematic depicts the relative location of nested primer pairs customized for each translocation event. The expected size of each band was estimated based on the primer size and the location of the predicted sgRNA cut site at each locus. Asterisks indicate bands detected at the expected size. The identities of the bands in FIG. 26C were verified by Sanger sequencing from each end (FIG. 30). A representative chromatogram for the P2/P5 translocation in HEK293T cells is shown (SEQ ID NO: 646).

FIG. 27 shows images of TBE-PAGE gels used to quantify Surveyor assay results to measure on-target and off-target gene modification in Table 4. Asterisks mark expected sizes of bands indicative of nuclease activity.

FIGS. 28A-28C show end-point nested PCR to detect chromosomal translocations caused by CRISPR/Cas9 off-target activity for CR3 and CR6/CR36 in human cells. Nested end-point PCR analysis was used to detect translocations in HEK293Tor sorted hDMD cells treated with Cas9 and CR3 as indicated (FIG. 28A), HEK293T cells treated with Cas9 and CR36 alone (FIG. 28B), or sorted hDMD cells treated with Cas9, CR6, and CR36 expression cassettes (FIG. 28C). The second nested PCR reaction for translocation was amplified using custom primers for each predicted translocation locus to maximize specificity (See Table 4). The schematic depicts the relative location of nested primer pairs used to probe for the presence of translocations. Each possible translocation event was first amplified from genomic DNA isolated from cells treated with or without the indicated sgRNA(s). A second nested PCR reaction was performed using primers within the predicted PCR amplicons that would result from translocations. Expected size was estimated based on the indicated primer binding site and the predicted sgRNA cut site at each locus. *indicates bands detected at the expected size and verified by Sanger sequencing from each end. #indicates amplicons in which Sanger sequencing showed sequences other than the predicted translocation, likely a result of mispriming during the nested PCR.

FIG. 29 shows Sanger sequencing chromatograms (SEQ ID NOS: 647-654, respectively, in order of appearance) for bands detected in FIGS. 28A-28C resulting from translocations between CR3 and CR3-OT1, on chromosomes X and 1, respectively, in HEK293T cells treated with Cas9 and CR3 gene cassettes. Arrows show regions of homology to the indicated chromosome nearby the expected break points caused by the appropriate sgRNAs. Note that sequencing reads become out of phase near the break point due to the error-prone nature of DNA repair by non-homologous end-joining.

FIG. 30 shows Sanger sequencing chromatograms (SEQ ID NOS: 655-660, respectively, in order of appearance) for bands detected in FIG. 26C resulting from translocations between CR1 and CR1-OT1, on chromosomes X and 16, respectively, in HEK293T cells treated with Cas9 and CR1 gene cassettes. Arrows show regions of homology to the indicated chromosome nearby the expected break points caused by the appropriate sgRNAs. Note that sequencing reads become out of phase near the break point due to the error-prone nature of DNA repair by non-homologous end-joining.

FIG. 31 shows an overview of in vivo AAV injections and tissue harvest.

FIGS. 32A-32C show Surveyor analysis of Rosa26 ZFN activities in skeletal muscle in vitro and in vivo following delivery of AAV-SASTG-ROSA. Arrows indicate expected bands resulting from Surveyor cleavage. n.d.: not detected. FIG. 32A shows proliferating C2C12s were transduced with the indicated amount of virus and harvested at 4 days post-infection. Arrows indicate expected bands sizes resulting from Surveyor cleavage. FIG. 32B shows C2C12s were incubated in differentiation medium for 5 days and then transduced with the indicated amount of AAV-SASTG-ROSA virus in 24 well plates. Samples were collected at 10 days post-transduction. FIG. 32C shows the indicated amount of AAV-SASTG-ROSA was injected directly into the tibialis anterior of C57BL/6J mice and muscles were harvested 4 weeks post-infection. The harvested TA muscles were partitioned into 8 separate pieces for genomic DNA analysis, each shown in a separate lane.

FIG. 33 shows Rosa T2A opt DNA sequence (SEQ ID NO: 434) and Rosa T2A opt protein sequence (SEQ ID NO: 435).

FIGS. 34A and 34B show SASTG capsid DNA sequence (SEQ ID NO:436) and SASTG capsid peptide sequence (SEQ ID NO: 437).

FIG. 35 shows DZF16 ZFN target site sequence (SEQ ID NO: 442), DZF16-L6 left full amino acid sequence (SEQ ID NO: 443) and DZF16-R6 right full amino acid sequence (SEQ ID NO: 444).

FIG. 36 shows E51C3 ZFN target site sequence (SEQ ID NO: 445), E51C3-3L left full amino acid sequence (SEQ ID NO: 446) and E51C3-3R right full amino acid sequence (SEQ ID NO: 447).

FIG. 37 shows DZF15 ZFN target site sequence (SEQ ID NO: 448), DZF15-L6 left full amino acid sequence (SEQ ID NO: 449), DZF15-R6 right full amino acid sequence (SEQ ID NO: 450), DZF15-L5 left full amino acid sequence (SEQ ID NO: 451), DZF15-R5 right full amino acid sequence (SEQ ID NO: 452).

FIG. 38 shows E51C4 ZFN target site sequence (SEQ ID NO: 453), E51C4-4L left full amino acid sequence (SEQ ID NO: 454) and E51C4-4R right full amino acid sequence (SEQ ID NO: 455).

FIG. 39 shows schematic diagrams of a “Single vector, multiplex CRISPR system,” Dual vector, multiplex CRISPR system,” and “Single vector, single gRNA system.”

FIG. 40 shows the nucleotide sequences of SaCas9-NLS (with the NLS underlined) (SEQ ID NO: 459) and SaCas9 gRNA (SEQ ID NO: 460).

FIG. 41 shows the nucleotide sequences of NmCas9 (with the NLS 1 underlined, the NLS 2 underlined and bolded, and the HA tag bolded; SEQ ID NO: 461), NmCas9 short hairpin from Thomson PNAS 2013 (SEQ ID NO: 462), and NmCas9 long hairpin from Church Nature Biotech 2013 (SEQ ID NO: 463).

FIGS. 42A-42C show validation of sgRNA and lentiviral Cas9 expression constructs. FIG. 42A shows constructs encoding unique Pol III promoters expressing sgRNAs targeting the AAVS1 locus or a construct containing the hU6 promoter immediately followed by poly-thymidine to terminate expression (“PolyT”) were transfected into HEK293T cells. End-point RT-PCR was used to probe for expression of each indicated promoter/sgRNA construct two days post-transfection.—RT: no reverse transcriptase control. FIG. 42B shows HEK293 Ts were transfected with expression vectors encoding the AAVS1 zinc-finger nuclease or Cas9-T2A-GFP and the indicated promoter/sgRNA expression cassettes and assessed for gene modification levels 3 days post-transfection using the Surveyor assay. FIG. 42C shows HEEK293T cells were transduced with lentiviral constructs encoding the indicated Cas9-T2A-GFP constructs without sgRNAs and assessed for Cas9 expression by western blot 7 days post-transduction by probing for a FLAG epitope tag on the N-terminus of the Cas9 protein.

FIG. 43 shows Golden Gate assembly of single lentiviral CRISPR/Cas9 expression cassettes. FIG. 43 discloses SEQ ID NOS: 661-672, respectively, in order of appearance.

FIGS. 44A-44B show single lentiviral delivery of a multiplex CRISPR/Cas9 system. FIG. 44A shows four sgRNAs targeting distinct genomic loci were cloned into a lentiviral vector expressing the active Cas9 nuclease. FIG. 44B shows HEK293 Ts and primary human dermal fibroblasts were transduced with lentivirus expressing the indicated sgRNAs and assayed for cleavage events using the Surveyor assay. HEK293 Ts were assayed 7 days post transduction. The human fibroblasts were assayed 10 days post transduction.

FIGS. 45A-45D show transient gene activation in HEK293 Ts stably expressing dCas9-VP64. HEK293 Ts were transduced with lentivirus to stably express dCas9-VP64 and were subsequently transfected with plasmid expressing the indicated sgRNA combinations. By varying the number of sgRNAs delivered, tunable endogenous gene activation of the endogenous IL1RN (FIG. 45A) and HBG1 (FIG. 45B) loci was achieved 3 days post transfection. Peak levels of endogenous IL1RN (FIG. 45C) and HBG1 (FIG. 45D) were observed 3-6 days post transfection and the level of activation returned to background levels between days 15-20. Importantly, the cell lines were able to reactive following a second transfection on day 20 albeit at a lower level than previously observed.

FIGS. 46A-46D show stable gene activation in HEK293 Ts using a single lentiviral multiplex dCas9-VP64 vector. HEK293 Ts were transduced with lentivirus to stably express dCas9-VP64 and the indicated combinations of gRNAs. By varying the number of sgRNAs delivered, tunable endogenous gene activation of the endogenous IL1RN (FIG. 46A) and HBG1 (FIG. 46B) loci was achieved 7 days post transduction. Peak levels of endogenous IL1RN (FIG. 46C) and HBG1 (FIG. 46D) were observed 6 days post transduction and the level of activation was sustained out to day 21.

FIG. 47 shows IL1RN mRNA expression levels.

FIG. 48 shows a schematic representing the direct conversion of fibroblasts to neurons through ectopic expression of the BAM neuronal transcription factors.

FIG. 49A shows a schematic of the dCas9-VP64 construct. dCas9-VP64 is a catalytically inactive form of the Cas9 protein fused to a tetramer of the VP16 transcriptional activation domain. FIG. 49B is a schematic showing the mechanism of RNA-guided recruitment of dCas9-VP64 to a genomic target. FIG. 49C is a schematic of the experimental protocol to generate iNs with CRISPR/Cas9 transcription factors.

FIGS. 50A-50B shows endogenous ASCL1 expression at day 3 determined by qRT-PCR (FIG. 50A) or total ASCL1 protein detected by immunofluorescence in MEFs transduced with dCas9-VP64 and transfected with either gRNAs targeted to the ASCL1 promoter, ASCL1 cDNA, or luciferase (FIG. 50B). Asterisk (*) indicates significant (p<0.05) increase in ASCL1 expression with the co-delivery of 8 gRNAs compared to 4 gRNAs. Ectopic expression of ASCL1 produced more protein than induced by dCas9-VP64 and 8 gRNAs targeted to the Ascl1 promoter, but did not activate the endogenous locus by day 3 in culture.

FIG. 51A shows TUJ1 and MAP2-postive cells generated by ectopic BAM factors or by dCas9-VP64 and gRNAs targeted to the BRN2 and ASCL1 promoters. FIG. 51B shows cells with neuronal morphology expressing a hSyn-RFP reporter at day 11 in N3 medium.

FIG. 52A shows a cell with neuronal morphology positive for the GCaMP5 calcium indicator in the presence (bottom) or absence (top) of KCl in the culture medium. FIG. 52B shows a trace of normalized fluorescent intensity over time showing depolarization of the cell in response to KCl addition.

FIGS. 52A-52B show activation of downstream targets of Ascl1 and Brn2, i.e., master regulatory genes, in iCas9-VP64-treated murine embryonic fibroblasts using dCas9-VP64 transcription factors to convert the fibroblasts to neurons. Mouse embryonic fibroblasts (MEFs) were transfected with a control GFP expression plasmid or the iCas9-VP64 expression plasmid and a combination of eight gRNA expression plasmids targeting ASCL1 and BRN2. The dCas9 transcription factors were delivered virally. After 10 days in neural induction media, cells were stained for Tuj1, an early marker of neuronal differentiation and MAP2, a marker of more mature neuronal differentiation. The conversion to neurons was efficient.

FIGS. 53A-53C show the CRISPR/Cas9 platform for control of mammalian gene regulation. FIG. 53A shows Cas9-based effectors bind genomic sequences in the presence of a chimeric gRNA molecule consisting of a constant region that complexes with Cas9 preceded by an exchangeable 20 bp protospacer that confers target site specificity. FIG. 53A discloses SEQ ID NO: 673. FIG. 53B shows Cas9-based synthetic transcription factors repress transcription of a target gene by interfering with RNA polymerase activity or by binding within the promoter and blocking the binding sites of endogenous transcription factors. FIG. 53C shows targeting regulatory elements such as enhancers could also potentially block the expression of multiple distal genes.

FIG. 54 shows targeting the HS2 enhancer using CRISPR/dCas9-KRAB. The HS2 region is a potent enhancer that distally regulates the expression of globin genes>10 kb downstream. A panel of single gRNAs was designed to target sites along the enhancer region.

FIGS. 55A-55E that single gRNAs targeting the HS2 enhancer effect potent transcriptional repression of globin genes. FIG. 55A shows dCas9 and dCas9-KRAB repressors were delivered on a lentiviral vector. Single gRNAs were transiently transfected for screening. When assayed by quantitative RT-PCR at 3 days post-transfection, K562s expressing dCas9-KRAB achieve up to 80% repression of γ-globin (FIG. 55B), ε-globin (FIG. 55C), and β-globin (FIG. 55D) genes, as compared to control cells that received no gRNA treatment. FIG. 55E shows protein expression in cells expressing dCas9 or dCas9-KRAB and treated with Cr4 or Cr8 show mild repression of γ-globin expression at day 3, compared to β-actin controls.

FIGS. 56A-56C expression of globin locus genes with varying doses of gRNA plasmid delivered to cells treated with no lentivirus (FIG. 56A), dCas9 lentivirus (FIG. 56B), or dCas9-KRAB lentivirus (FIG. 56C). Increasing the dose of Cr4 gRNA plasmid delivered enhanced repression in dCas9-KRAB treated cells, indicating that both the dCas9-KRAB effector and targeted gRNA play a role in achieving repression.

FIGS. 57A-57D show that stably delivering single gRNAs with dCas9-KRAB silences expression of the globin genes. FIG. 57A shows dCas9 and dCas9-KRAB repressors were co-expressed on a lentiviral vector with single gRNAs. When assayed by quantitative RT-PCR at 7 days post-transduction, K562s expressing dCas9-KRAB achieve up to 95% repression of γ-globin (FIG. 57B), ε-globin (FIG. 57C), and β-globin genes (FIG. 57D), as compared to control cells that received no lentiviral treatment.

FIG. 58 shows that isolating the p300 HAT “Core” for targeted epigenetic modification of histones only via dCas9 fusion.

FIG. 59 shows a simplified schematic of S. pyogenes dCas9-VP64 fusion (top) and dCas9-p300 core fusion (bottom). The Protospacer Adjacent Motifs (PAM) are shown with arrows at target gene loci and synthetic guide RNA (gRNA) is shown with hatched arrows.

FIGS. 60A-60C show representative data at three human loci demonstrating the efficacy of activation using dCas9-p300 in relation to dCas9-VP64 and dCas9 without any fused effector domain in the human 293T cell culture line.

FIGS. 61A-61C show the amino acid sequences of the dCas9 constructs. The legend for all FIGS. 61A-61C is shown in FIG. 61A. FIGS. 61A-61C disclose SEQ ID NOS: 674-676, respectively, in order of appearance.

FIG. 62 shows that HAT-dCase9-p300 fusion proteins fail to activate gene expression.

FIGS. 63A-63B show that gRNA's also act synergistically with dCas9-p300 Core.

FIGS. 64A-64C show TALEN mediated integration of minidystrophin at the 5′UTR of the Dp427m skeletal muscle isoform of dystrophin in skeletal myoblast cell lines derived from human DMD patients carrying different deletions in the dystrophin gene. DMD patient cells were electroporated with constructs encoding a TALEN pair active at the 5′UTR locus and a donor template carrying the minidystrophin gene. FIG. 64A is a schematic showing how minidystrophin is integrated into the 5′UTR. FIG. 64B shows hygromycin-resistant clonal cell lines were isolated and screened by PCR for successful site-specific integrations at the 5′UTR using the primers shown in FIG. 64A. Asterisks indicate clones selected for further analysis in FIG. 64C. FIG. 64C shows clonally isolated DMD myoblasts with detected integration events were differentiated for 6 days and assessed for expression of an HA tag fused to the C terminus of minidystrophin.

### DETAILED DESCRIPTION

As described herein, certain methods and engineered CRISPR/CRISPR-associated (Cas) 9-based system compositions have been discovered to be useful for altering the expression of genes, genome engineering, and correcting or reducing the effects of mutations in genes involved in genetic diseases. The CRISPR/Cas9-based system involves a Cas9 protein and at least one guide RNA, which provide the DNA targeting specificity for the system. In particular, the present disclosure describes a Cas9 fusion protein that combines the DNA sequence targeting function of the CRISPR/Cas9-based system with an additional activity, thus allowing changes in gene expression and/or epigenetic status. The system may also be used in genome engineering and correcting or reducing the effects of gene mutations.

The present disclosure also provides certain compositions and methods for delivering CRISPR/CRISPR-associated (Cas) 9-based system and multiple gRNAs to target one or more endogenous genes. Co-transfection of multiple sgRNAs targeted to a single promoter allow for synergistic activation, however, co-transfection of multiple plasmids leads to variable expression levels in each cell due to differences in copy number. Additionally, gene activation following transfection is transient due to dilution of plasmid DNA over time. Moreover, many cell types are not easily transfected and transient gene expression may not be sufficient for inducing a therapeutic effect. To address these limitations, a single lentiviral system was developed to express Cas9 and up to four sgRNAs from independent promoters. A platform is disclosed that expresses Cas9 or dCas9 fusion proteins and up to four gRNAs from a single lentiviral vector. The lentiviral vector expresses a constitutive or inducible Cas9 or dCas9-VP64 in addition to one, two, three, or four gRNAs expressed from independent promoters. This system enables control of both the magnitude and timing of CRISPR/Cas9-based gene regulation. Furthermore, the lentiviral platform provides the potent and sustained levels of gene expression that will facilitate therapeutic applications of the CRISPR/Cas9 system in primary cells. Finally, this system may be used for editing multiple genes simultaneously, such as the concurrent knockout of several oncogenes.

The present disclosure also provides certain compositions and methods for delivering site-specific nucleases to skeletal muscle and cardiac muscle using modified adeno-associated virus (AAV) vectors. The site-specific nucleases, which may be engineered, are useful for altering the expression of genes, genome engineering, correcting or reducing the effects of mutations in genes involved in genetic diseases, or manipulating genes involved in other conditions affecting skeletal muscle or cardiac muscle or muscle regeneration. The engineered site-specific nucleases may include a zinc finger nuclease (ZFN), a TAL effector nuclease (TALEN), and/or a CRISPR/Cas9 system for genome editing. As described herein, genes in skeletal muscle tissue were successfully edited in vivo using this unique delivery system. The disclosed invention provides a means to rewrite the human genome for therapeutic applications and target model species for basic science applications.

Gene editing is highly dependent on cell cycle and complex DNA repair pathways that vary from tissue to tissue. Skeletal muscle is a very complex environment, consisting of large myofibers with more than 100 nuclei per cell. Gene therapy and biologics in general have been limited for decades by in vivo delivery hurdles. These challenges include stability of the carrier in vivo, targeting the right tissue, getting sufficient gene expression and active gene product, and avoiding toxicity that might overcome activity, which is common with gene editing tools. Other delivery vehicles, such as direct injection of plasmid DNA, work to express genes in skeletal muscle and cardiac muscle in other contexts, but do not work well with these site-specific nucleases for achieving detectable levels of genome editing.

While many gene sequences are unstable in AAV vectors and therefore undeliverable, these site-specific nucleases are surprisingly stable in the AAV vectors. When these site-specific nucleases are delivered and expressed, they remained active in the skeletal muscle tissue. The protein stability and activity of the site-specific nucleases are highly tissue type- and cell type-dependent. These active and stable nucleases are able to modify gene sequences in the complex environment of skeletal muscle. The current invention describes a way to deliver active forms of this class of therapeutics to skeletal muscle or cardiac muscle that is effective, efficient and facilitates successful genome modification.

The present disclosure also provides certain fusion epigenetic effector molecules, a dCas9-p300 fusion protein, which provides a robust and potentially more widely applicable tool for synthetic transcriptional modulation compared to the dCas9-VP64 fusion. The activated target genes to a substantially greater extent than the dCas9-VP64 fusion protein at all loci tested. In addition, the p300 has intrinsic endogenous activity at enhancers within the human genome. The dCas9-p300 fusion protein may be able to activate endogenous target gene promoters and enhancer regions.

The dCas9-p300 fusion protein can be used in human tissue culture cell lines to activate gene expression. This fusion protein may be used to direct the epigenetic state of target loci within human cells with precision and predictability in order to control differentiation, modulate cellular regulation, and apply innovative potential therapies. Current technologies are limited in the strength of activation and the extent and sustainability of epigenetic modulation; obstacles which may be obviated via utilization of this new fusion protein.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Binding region” as used herein refers to the region within a nuclease target region that is recognized and bound by the nuclease.

“Cardiac muscle” or “heart muscle” as used interchangeably herein means a type of involuntary striated muscle found in the walls and histological foundation of the heart, the myocardium. Cardiac muscle is made of cardiomyocytes or myocardiocytes. Myocardiocytes show striations similar to those on skeletal muscle cells but contain only one, unique nucleus, unlike the multinucleated skeletal cells.

“Cardiac muscle condition” as used herein refers to a condition related to the cardiac muscle, such as cardiomyopathy, heart failure, arrhythmia, and inflammatory heart disease.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template” and “repair template” as used interchangeably herein refers to a double-stranded DNA fragment or molecule that includes at least a portion of the gene of interest. The donor DNA may encode a full-functional protein or a partially-functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Exon 51” as used herein refers to the 51^{st} exon of the dystrophin gene. Exon 51 is frequently adjacent to frame-disrupting deletions in DMD patients and has been targeted in clinical trials for oligonucleotide-based exon skipping. A clinical trial for the exon 51 skipping compound eteplirsen recently reported a significant functional benefit across 48 weeks, with an average of 47% dystrophin positive fibers compared to baseline. Mutations in exon 51 are ideally suited for permanent correction by NHEJ-based genome editing.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, non-homologous end joining may take place instead.

“Genome editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression.

“Nuclease mediated NHEJ” as used herein refers to NHEJ that is initiated after a nuclease, such as a cas9, cuts double stranded DNA.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

“Site-specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas9-based systems.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells”, sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Skeletal muscle condition” as used herein refers to a condition related to the skeletal muscle, such as muscular dystrophies, aging, muscle degeneration, wound healing, and muscle weakness or atrophy.

“Spacers” and “spacer region” as used interchangeably herein refers to the region within a TALEN or ZFN target region that is between, but not a part of, the binding regions for two TALENs or ZFNs.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Target gene” as used herein refers to any nucleotide sequence encoding a known or putative gene product. The target gene may be a mutated gene involved in a genetic disease.

“Target region” as used herein refers to the region of the target gene to which the site-specific nuclease is designed to bind and cleave.

“Transcription activator-like effector” or “TALE” as used herein refers to a protein structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules may be arranged in any order to assemble an array that recognizes a defined sequence.

A binding specificity of a TALE DNA-binding domain is determined by the RVD array followed by a single truncated repeat of 20 amino acids. A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors, methyltransferases, integrases, nucleases, and recombinases.

“Transcription activator-like effector nucleases” or “TALENs” as used interchangeably herein refers to engineered fusion proteins of the catalytic domain of a nuclease, such as endonuclease FokI, and a designed TALE DNA-binding domain that may be targeted to a custom DNA sequence. A “TALEN monomer” refers to an engineered fusion protein with a catalytic nuclease domain and a designed TALE DNA-binding domain. Two TALEN monomers may be designed to target and cleave a TALEN target region.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode an iCas9-VP64 fusion protein comprising the amino acid sequence of SEQ ID NO: 1 or at least one gRNA nucleotide sequence of any one of SEQ ID NOs: 5-40, 65-144, 492-515, 540-563, and 585-625. Alternatively, the vector may encode Cas9 and at least one gRNA nucleotide sequence of any one of SEQ ID NOs: 5-40, 65-144, 492-515, 540-563, and 585-625.

“Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

“Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Compositions for Genome Editing

The present invention is directed to compositions for genome editing, genomic alteration or altering gene expression of a target gene. The compositions may include a may include viral vector and fusion protein such as a site-specific nuclease or CRISPR/Cas9-system with at least one gRNA.

a. Compositions for Genome Editing in Muscle

The present invention is directed to a composition for genome editing a target gene in skeletal muscle or cardiac muscle of a subject. The composition includes a modified AAV vector and a nucleotide sequence encoding a site-specific nuclease. The composition delivers active forms of site-specific nucleases to skeletal muscle or cardiac muscle. The composition may further comprise a donor DNA or a transgene. These compositions may be used in genome editing, genome engineering, and correcting or reducing the effects of mutations in genes involved in genetic diseases and/or other skeletal or cardiac muscle conditions.

The target gene may be involved in differentiation of a cell or any other process in which activation, repression, or disruption of a gene may be desired, or may have a mutation such as a deletion, frameshift mutation, or a nonsense mutation. If the target gene has a mutation that causes a premature stop codon, an aberrant splice acceptor site or an aberrant splice donor site, the site-specific nucleases may be designed to recognize and bind a nucleotide sequence upstream or downstream from the premature stop codon, the aberrant splice acceptor site or the aberrant splice donor site. The site-specific nucleases may also be used to disrupt normal gene splicing by targeting splice acceptors and donors to induce skipping of premature stop codons or restore a disrupted reading frame. The site-specific nucleases may or may not mediate off-target changes to protein-coding regions of the genome.

### 3. CRISPR System

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the sgRNA, and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system naturally prefers to use an “NGG” sequence, where “N” can be any nucleotide, but also accepts other PAM sequences, such as “NAG” in engineered systems (Hsu et al., Nature Biotechnology (2013) doi: 10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi: 10.1038/nmeth.2681).

### 4. CRISPR/Cas9-Based System

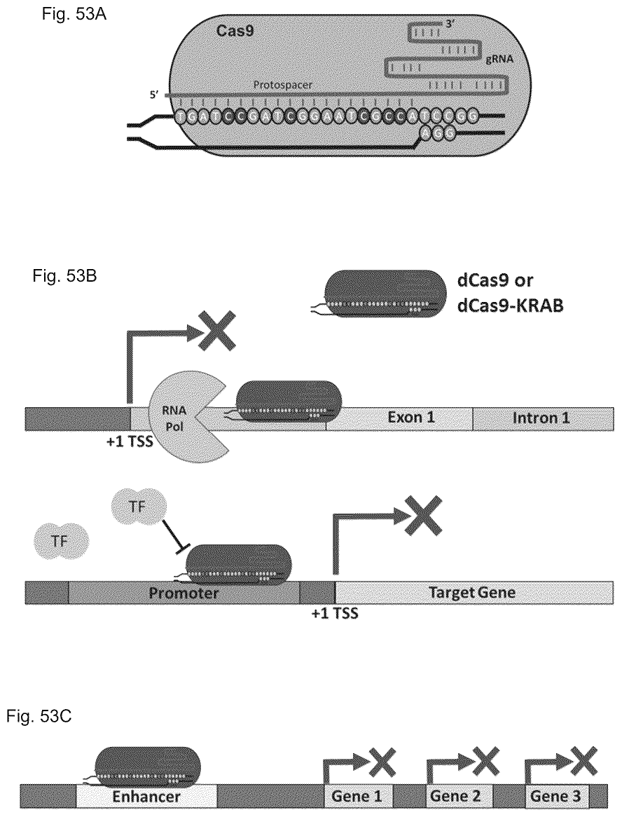

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general (see FIG. 53A). Provided herein are CRISPR/Cas9-based engineered systems for use in genome editing and treating genetic diseases. The CRISPR/Cas9-based engineered systems may be designed to target any gene, including genes involved in a genetic disease, aging, tissue regeneration, or wound healing. The CRISPR/Cas9-based systems may include a Cas9 protein or Cas9 fusion protein and at least one gRNA. The Cas9 fusion protein may, for example, include a domain that has a different activity that what is endogenous to Cas9, such as a transactivation domain.

The target gene may be involved in differentiation of a cell or any other process in which activation of a gene may be desired, or may have a mutation such as a frameshift mutation or a nonsense mutation. If the target gene has a mutation that causes a premature stop codon, an aberrant splice acceptor site or an aberrant splice donor site, the CRISPR/Cas9-based system may be designed to recognize and bind a nucleotide sequence upstream or downstream from the premature stop codon, the aberrant splice acceptor site or the aberrant splice donor site. The CRISPR-Cas9-based system may also be used to disrupt normal gene splicing by targeting splice acceptors and donors to induce skipping of premature stop codons or restore a disrupted reading frame. The CRISPR/Cas9-based system may or may not mediate off-target changes to protein-coding regions of the genome.

a. Cas9

The CRISPR/Cas9-based system may include a Cas9 protein or a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. The Cas9 protein may be mutated so that the nuclease activity is inactivated. An inactivated Cas9 protein from Streptococcus pyogenes (iCas9, also referred to as “dCas9”) with no endonuclease activity has been recently targeted to genes in bacteria, yeast, and human cells by gRNAs to silence gene expression through steric hindrance. As used herein, “iCas9” and “dCas9” both refer to a Cas9 protein that has the amino acid substitutions D10A and H840A and has its nuclease activity inactivated. For example, the CRISPR/Cas9-based system may include a Cas9 of SEQ ID NO: 459 or 461.

b. Cas9 Fusion Protein

The CRISPR/Cas9-based system may include a fusion protein. The fusion protein may comprise two heterologous polypeptide domains, wherein the first polypeptide domain comprises a Cas protein and the second polypeptide domain has an activity such as transcription activation activity, transcription repression activity, transcription release factor activity, histone modification activity, nuclease activity, nucleic acid association activity, methylase activity, or demethylase activity. The fusion protein may include a Cas9 protein or a mutated Cas9 protein, as described above, fused to a second polypeptide domain that has an activity such as transcription activation activity, transcription repression activity, transcription release factor activity, histone modification activity, nuclease activity, nucleic acid association activity, methylase activity, or demethylase activity.

### (1) Transcription Activation Activity

The second polypeptide domain may have transcription activation activity, i.e., a transactivation domain. For example, gene expression of endogenous mammalian genes, such as human genes, may be achieved by targeting a fusion protein of iCas9 and a transactivation domain to mammalian promoters via combinations of gRNAs. The transactivation domain may include a VP16 protein, multiple VP16 proteins, such as a VP48 domain or VP64 domain, or p65 domain of NF kappa B transcription activator activity. For example, the fusion protein may be iCas9-VP64.

### (2) Transcription Repression Activity

The second polypeptide domain may have transcription repression activity. The second polypeptide domain may have a Kruppel associated box activity, such as a KRAB domain, ERF repressor domain activity, Mxi1 repressor domain activity, SID4X repressor domain activity, Mad-SID repressor domain activity or TATA box binding protein activity. For example, the fusion protein may be dCas9-KRAB.

### (3) Transcription Release Factor Activity

The second polypeptide domain may have transcription release factor activity. The second polypeptide domain may have eukaryotic release factor 1 (ERF1) activity or eukaryotic release factor 3 (ERF3) activity.

### (4) Histone Modification Activity

The second polypeptide domain may have histone modification activity. The second polypeptide domain may have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300.

### (5) Nuclease Activity

The second polypeptide domain may have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases are deoxyribonuclease and ribonuclease.

### (6) Nucleic Acid Association Activity

The second polypeptide domain may have nucleic acid association activity or nucleic acid binding protein-DNA-binding domain (DBD) is an independently folded protein domain that contains at least one motif that recognizes double- or single-stranded DNA. A DBD can recognize a specific DNA sequence (a recognition sequence) or have a general affinity to DNA. nucleic acid association region selected from the group consisting of helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

### (7) Methylase Activity

The second polypeptide domain may have methylase activity, which involves transferring a methyl group to DNA, RNA, protein, small molecule, cytosine or adenine. The second polypeptide domain may include a DNA methyltransferase.

### (8) Demethylase Activity

The second polypeptide domain may have demethylase activity. The second polypeptide domain may include an enzyme that remove methyl (CH3-) groups from nucleic acids, proteins (in particular histones), and other molecules. Alternatively, the second polypeptide may covert the methyl group to hydroxymethylcytosine in a mechanism for demethylating DNA. The second polypeptide may catalyze this reaction. For example, the second polypeptide that catalyzes this reaction may be Tet1.

c. gRNA

The gRNA provides the targeting of the CRISPR/Cas9-based system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide.

The number of gRNA administered to the cell may be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

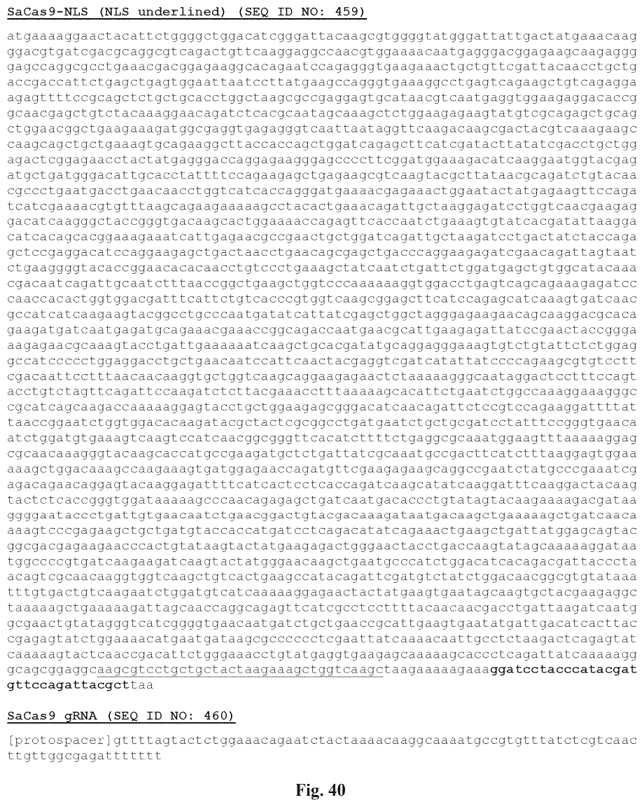

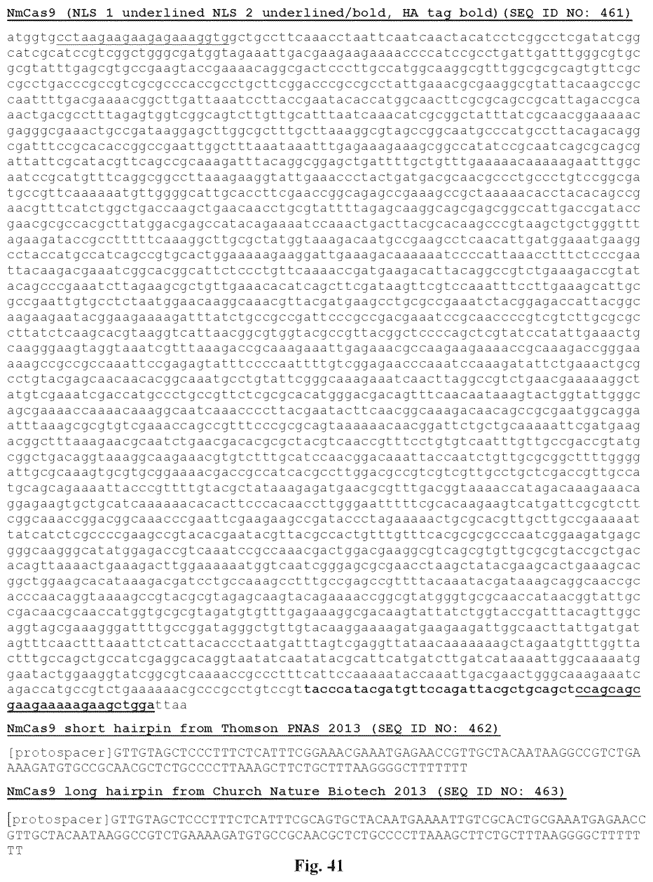

The gRNA may comprise a complementary polynucleotide sequence of the target DNA sequence followed by a PAM sequence. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. The PAM sequence may be “NGG”, where “N” can be any nucleotide. The gRNA may target at least one of the promoter region, the enhancer region or the transcribed region of the target gene. The gRNA may include a nucleic acid sequence of at least one of SEQ ID NOs: 5-40, 65-144, 492-515, 540-563, 585-625, 462 (FIG. 40), 464 (FIG. 41), and 465 (FIG. 41).

The gRNA may target any nucleic acid sequence. The nucleic acid sequence target may be DNA. The DNA may be any gene. For example, the gRNA may target a gene, such as BRN2, MYT1L, ASCL1, NANOG, VEGFA, TERT, IL1B, IL1R2, IL1RN, HBG1, HBG2, MYOD1, OCT4, and DMD.

### (1) Dystrophin

Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons code for the protein which is over 3500 amino acids. Normal skeleton muscle tissue contains only small amounts of dystrophin but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. Naturally occurring mutations and their consequences are relatively well understood for DMD. It is known that in-frame deletions that occur in the exon 45-55 region contained within the rod domain can produce highly functional dystrophin proteins, and many carriers are asymptomatic or display mild symptoms. Furthermore, more than 60% of patients may theoretically be treated by targeting exons in this region of the dystrophin gene. Efforts have been made to restore the disrupted dystrophin reading frame in DMD patients by skipping non-essential exons during mRNA splicing to produce internally deleted but functional dystrophin proteins. The deletion of internal dystrophin exons retain the proper reading frame but cause the less severe Becker muscular dystrophy.

### (2) CRISPR/Cas9-Based System for Targeting Dystrophin

A CRISPR/Cas9-based system specific for dystrophin gene are disclosed herein. The CRISPR/Cas9-based system may include Cas9 and at least one gRNA to target the dystrophin gene. The CRISPR/Cas9-based system may bind and recognize a target region. The target regions may be chosen immediately upstream of possible out-of-frame stop codons such that insertions or deletions during the repair process restore the dystrophin reading frame by frame conversion. Target regions may also be splice acceptor sites or splice donor sites, such that insertions or deletions during the repair process disrupt splicing and restore the dystrophin reading frame by splice site disruption and exon exclusion. Target regions may also be aberrant stop codons such that insertions or deletions during the repair process restore the dystrophin reading frame by eliminating or disrupting the stop codon.

Single or multiplexed sgRNAs may be designed to restore the dystrophin reading frame by targeting the mutational hotspot at exons 45-55 and introducing either intraexonic small insertions and deletions, or large deletions of one or more exons. Following treatment with Cas9 and one or more sgRNAs, dystrophin expression may be restored in Duchenne patient muscle cells in vitro. Human dystrophin was detected in vivo following transplantation of genetically corrected patient cells into immunodeficient mice. Significantly, the unique multiplex gene editing capabilities of the CRISPR/Cas9 system enable efficiently generating large deletions of this mutational hotspot region that can correct up to 62% of patient mutations by universal or patient-specific gene editing approaches.

The CRISPR/Cas9-based system may use gRNA of varying sequences and lengths. Examples of gRNAs may be found in Table 6. The CRISPR/Cas9-based system may target a nucleic acid sequence of SEQ ID NOs: 65-144, or a complement thereof. The gRNA may include a nucleotide sequence selected from the group consisting of SEQ ID NO: 65-144, or a complement thereof. For example, the disclosed CRISPR/Cas9-based systems were engineered to mediate highly efficient gene editing at exon 51 of the dystrophin gene. These CRISPR/Cas9-based systems restored dystrophin protein expression in cells from DMD patients.

### (a) Exons 51 and 45-55

Exon 51 is frequently adjacent to frame-disrupting deletions in DMD. Elimination of exon 51 from the dystrophin transcript by exon skipping can be used to treat approximately 15% of all DMD patients. This class of DMD mutations is ideally suited for permanent correction by NHEJ-based genome editing and HDR. The CRISPR/Cas9-based systems described herein have been developed for targeted modification of exon 51 in the human dystrophin gene. These CRISPR/Cas9-based systems were transfected into human DMD cells and mediated efficient gene modification and conversion to the correct reading frame. Protein restoration was concomitant with frame restoration and detected in a bulk population of CRISPR/Cas9-based system-treated cells. Similarly, the elimination of exons 45-55 of the dystrophin transcript can be used to treat approximately 62% of all DMD patients.

### (3) AAV/CRISPR Constructs

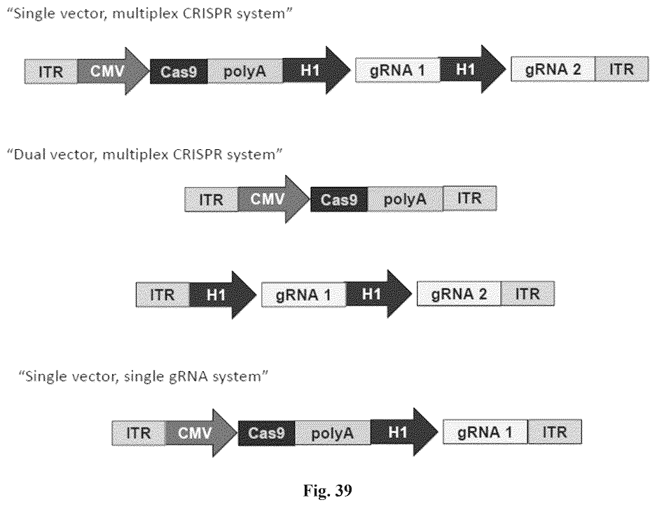

AAV may be used to deliver CRISPRs using various construct configurations (see FIG. 39). For example, AAV may deliver Cas9 and gRNA expression cassettes on separate vectors. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit (see FIG. 39).

### 5. Multiplex CRISPR/Cas9-Based System

The present disclosure is directed to a multiplex CRISPR/Cas9-Based System which includes a CRISPR/CRISPR-associated (Cas) 9-based system, such as Cas9 or dCas9, and multiple gRNAs to target one or more endogenous genes. This platform utilizes a convenient Golden Gate cloning method to rapidly incorporate up to four independent sgRNA expression cassettes into a single lentiviral vector. Each sgRNA was efficiently expressed and could mediate multiplex gene editing at diverse loci in immortalized and primary human cell lines. Transient transcriptional activation in cell lines stably expressing dCas9-VP64 was demonstrated to be tunable by synergistic activation with one to four sgRNAs. Furthermore, the single lentiviral vector can induce sustained and long-term endogenous gene expression in immortalized and primary human cells. This system allows for rapid assembly of a single lentiviral vector that enables efficient multiplex gene editing or activation in model and primary cell lines.

The multiplex CRISPR/Cas9-Based System provides potency of transcriptional activation and tunable induction of transcriptional activation. Readily generated by Golden Gate assembly, the final vector expresses a constitutive Cas9 or dCas9-VP64 in addition to one, two, three, or four sgRNAs expressed from independent promoters. Each promoter is capable of efficiently expressing sgRNAs that direct similar levels of Cas9 nuclease activity. Furthermore, lentiviral delivery of a single vector expressing Cas9 and four sgRNAs targeting independent loci resulted in simultaneous multiplex gene editing of all four loci. Tunable transcriptional activation at two endogenous genes in both transient and stable contexts was achieved using lentiviral delivery of Cas9 with or without sgRNAs. Highly efficient and long-term gene activation in primary human cells is accomplished. This system is therefore an attractive and efficient method to generate multiplex gene editing and long-term transcriptional activation in human cells.

The multiplex CRISPR/Cas9-Based System allows efficient multiplex gene editing for simultaneously inactivating multiple genes. The CRISPR/Cas9 system can simultaneously target multiple distinct genomic loci by co-expressing a single Cas9 protein with two or more sgRNAs, making this system uniquely suited for multiplex gene editing or synergistic activation applications. The CRISPR/Cas9 system greatly expedites the process of molecular targeting to new sites by simply modifying the expressed sgRNA molecule. The single lentiviral vector may be combined with methods for achieving inducible control of these components, either by chemical or optogenetic regulation, to facilitate investigation of the dynamics of gene regulation in both time and space.

The multiplex CRISPR/Cas9-based systems may transcriptionally activate two or more endogenous genes. The multiplex CRISPR/Cas9-based systems may transcriptionally repress two or more endogenous genes. For example, at least two endogenous genes, at least three endogenous genes, at least four endogenous genes, at least five endogenous genes, or at least ten endogenous genes may be activated or repressed by the multiplex CRISPR/Cas9-based system. Between two and fifteen genes, between two and ten genes, between two and five genes, between five and fifteen genes, or between five and ten genes may be activated or repressed by the multiplex CRISPR/Cas9-based system.

### (1) Modified Lentiviral Vector

The multiplex CRISPR/Cas9-based system includes a modified lentiviral vector. The modified lentiviral vector includes a first polynucleotide sequence encoding a fusion protein and a second polynucleotide sequence encoding at least one sgRNA. The fusion protein may be the fusion protein of the CRISPR/Cas9-based system, as described above. The first polynucleotide sequence may be operably linked to a promoter. The promoter may be a constitutive promoter, an inducible promoter, a repressible promoter, or a regulatable promoter.

The second polynucleotide sequence encodes at least 1 sgRNA. For example, the second polynucleotide sequence may encode at least 1 sgRNA, at least 2 sgRNAs, at least 3 sgRNAs, at least 4 sgRNAs, at least 5 sgRNAs, at least 6 sgRNAs, at least 7 sgRNAs, at least 8 sgRNAs, at least 9 sgRNAs, at least 10 sgRNAs, at least 11 sgRNA, at least 12 sgRNAs, at least 13 sgRNAs, at least 14 sgRNAs, at least 15 sgRNAs, at least 16 sgRNAs, at least 17 sgRNAs, at least 18 sgRNAs, at least 19 sgRNAs, at least 20 sgRNAs, at least 25 sgRNA, at least 30 sgRNAs, at least 35 sgRNAs, at least 40 sgRNAs, at least 45 sgRNAs, or at least 50 sgRNAs. The second polynucleotide sequence may encode between 1 sgRNA and 50 sgRNAs, between 1 sgRNA and 45 sgRNAs, between 1 sgRNA and 40 sgRNAs, between 1 sgRNA and 35 sgRNAs, between 1 sgRNA and 30 sgRNAs, between 1 sgRNA and 25 different sgRNAs, between 1 sgRNA and 20 sgRNAs, between 1 sgRNA and 16 sgRNAs, between 1 sgRNA and 8 different sgRNAs, between 4 different sgRNAs and 50 different sgRNAs, between 4 different sgRNAs and 45 different sgRNAs, between 4 different sgRNAs and 40 different sgRNAs, between 4 different sgRNAs and 35 different sgRNAs, between 4 different sgRNAs and 30 different sgRNAs, between 4 different sgRNAs and 25 different sgRNAs, between 4 different sgRNAs and 20 different sgRNAs, between 4 different sgRNAs and 16 different sgRNAs, between 4 different sgRNAs and 8 different sgRNAs, between 8 different sgRNAs and 50 different sgRNAs, between 8 different sgRNAs and 45 different sgRNAs, between 8 different sgRNAs and 40 different sgRNAs, between 8 different sgRNAs and 35 different sgRNAs, between 8 different sgRNAs and 30 different sgRNAs, between 8 different sgRNAs and 25 different sgRNAs, between 8 different sgRNAs and 20 different sgRNAs, between 8 different sgRNAs and 16 different sgRNAs, between 16 different sgRNAs and 50 different sgRNAs, between 16 different sgRNAs and 45 different sgRNAs, between 16 different sgRNAs and 40 different sgRNAs, between 16 different sgRNAs and 35 different sgRNAs, between 16 different sgRNAs and 30 different sgRNAs, between 16 different sgRNAs and 25 different sgRNAs, or between 16 different sgRNAs and 20 different sgRNAs. Each of the polynucleotide sequences encoding the different sgRNAs may be operably linked to a promoter. The promoters that are operably linked to the different sgRNAs may be the same promoter. The promoters that are operably linked to the different sgRNAs may be different promoters. The promoter may be a constitutive promoter, an inducible promoter, a repressible promoter, or a regulatable promoter.

The at least one sgRNA may bind to a target gene or loci. If more than one sgRNA is included, each of the sgRNAs binds to a different target region within one target loci or each of the sgRNA binds to a different target region within different gene loci. The fusion protein may include Cas9 protein or iCas9-VP64 protein. The fusion protein may include a VP64 domain, a p300 domain, or a KRAB domain.

### 6. Site-Specific Nucleases

The composition, as described above, includes a nucleotide sequence encoding a site-specific nuclease that binds and cleaves a target region. The site-specific nuclease may be engineered. For example, an engineered site-specific nuclease may be a CRISPR/Cas9-based system, a ZFN, or a TALEN. The site-specific nuclease may bind and cleave a gene or locus in the genome of a cell in the skeletal muscle or cardiac muscle. For example, the gene or locus may be the Rosa26 locus or the dystrophin gene.

a. CRISPR/Cas9-Based System

The CRISPR/Cas9-based system, as described above, may be used to introduce site-specific double strand breaks at targeted genomic loci.

b. Zinc Finger Nucleases (ZFN)

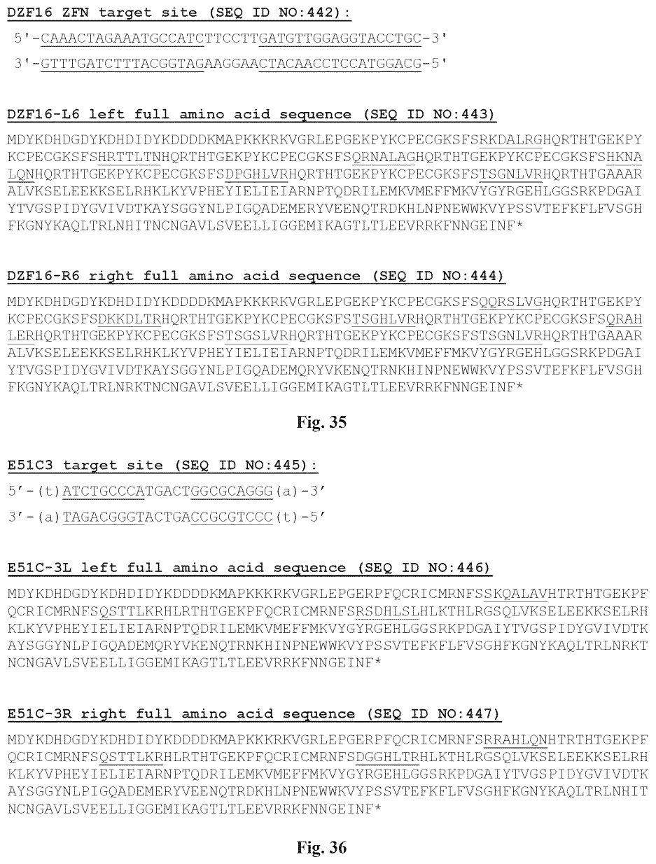

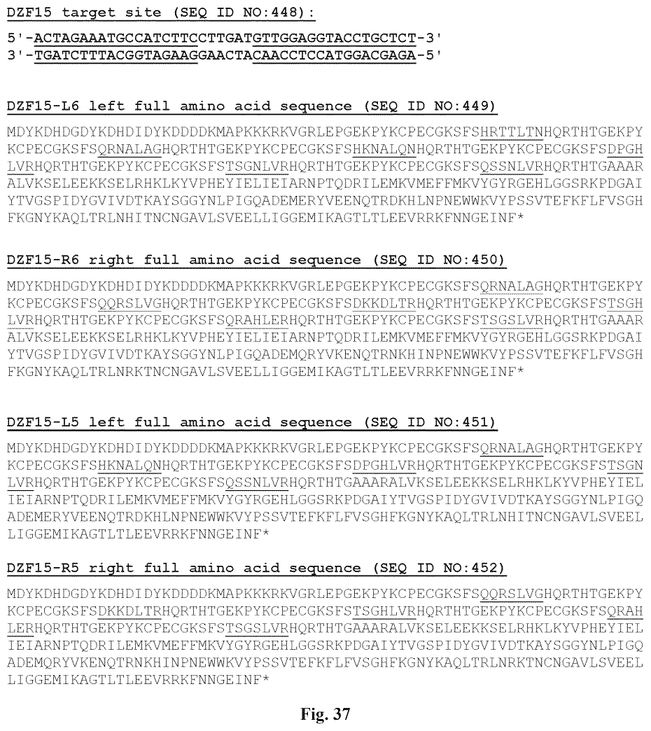

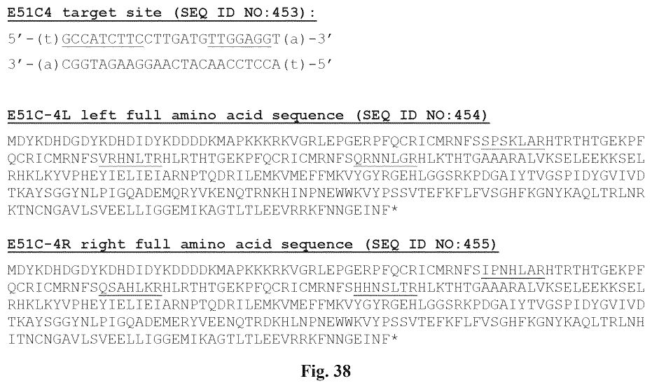

The site-specific nuclease may be a ZFN. A single zinc finger contains approximately 30 amino acids and the domain functions by binding 3 consecutive base pairs of DNA via interactions of a single amino acid side chain per base pair. The modular structure of the zinc finger motif permits the conjunction of several domains in series, allowing for the recognition and targeting of extended sequences in multiples of 3 nucleotides. These targeted DNA-binding domains can be combined with a nuclease domain, such as FokI, to generate a site-specific nuclease, called a “zinc finger nuclease” (ZFNs) that can be used to introduce site-specific double strand breaks at targeted genomic loci. This DNA cleavage stimulates the natural DNA-repair machinery, leading to one of two possible repair pathways, NHEJ and HDR. For example, the ZFN may target the Rosa26 locus (Perez-Pinera et al. Nucleic Acids Research (2012) 40:3741-3752) or a dystrophin gene. Examples of ZFNs are shown in Table 1 and FIGS. 35-38. In Table 1, the DNA recognition helices are underlined and “Fok ELD-S” and “Fok KKR-S” refers to the FokI nuclease domain that is fused to the zinc finger protein DNA-binding domains. In FIGS. 35-38, the target DNA sequence in the target sites (i.e., in SEQ ID NOs: 442, 445, 448, and 453) and the DNA recognition helices in the ZFN amino acid sequences (i.e., in SEQ ID NOs: 443, 444, 446, 447, 449-452, 454, and 455) are underlined, respectively.

TABLE 1
Full amino acid sequences of identified ZFNs.

ZFN B left Fok ELD-S full amino acid sequence
(SEQ ID NO: 438)
MDYKDHDGDYKDHDIDYKDDDDKMAPKKKRKVGRLEPGEKPYKCPECGKS
FSRKDALRGHQRTHTGEKPYKCPECGKSFSHRTTLTNHQRTHTGEKPYKC
PECGKSFSQRNALAGHQRTHTGEKPYKCPECGKSFSHKNALQNHQRTHTG
EKPYKCPECGKSFSDPGHLVRHQRTHTGEKPYKCPECGKSFSTSGNLVRH
QRTHTGAAARALVKSELEEKKSELRHKLKYVPHEYIELIEIARNPTQDRI
LEMKVMEFFMKVYGYRGEHLGGSRKPDGAIYTVGSPIDYGVIVDTKAYSG
GYNLPIGQADEMERYVEENQTRDKHLNPNEWWKVYPSSVTEFKFLFVSGH
FKGNYKAQLTRLNHITNCNGAVLSVEELLIGGEMIKAGTLTLEEVRRKFN
NGEINF*
ZFN B right Fok KKR-S full amino acid sequence
(SEQ ID NO: 439)
MDYKDHDGDYKDHDIDYKDDDDKMAPKKKRKVGRLEPGEKPYKCPECGKS
FSQQRSLVGHQRTHTGEKPYKCPECGKSFSDKKDLTRHQRTHTGEKPYKC
PECGKSFSTSGHLVRHQRTHTGEKPYKCPECGKSFSQRAHLERHQRTHTG
EKPYKCPECGKSFSTSGSLVRHQRTHTGEKPYKCPECGKSFSTSGNLVRH
QRTHTGAAARALVKSELEEKKSELRHKLKYVPHEYIELIEIARNPTQDRI
LEMKVMEFFMKVYGYRGEHLGGSRKPDGAIYTVGSPIDYGVIVDTKAYSG
GYNLPIGQADEMQRYVKENQTRNKHINPNEWWKVYPSSVTEFKFLEVSGH
FKGNYKAQLTRLNRKTNCNGAVLSVEELLIGGEMIKAGTLTLEEVRRKFN
NGEINF*
ZFN J left Fok KKR-S full amino acid sequence
(SEQ ID NO: 440)
MDYKDHDGDYKDHDIDYKDDDDKMAPKKKRKVGRLEPGERPFQCRICMRN
FSSKQALAVHTRTHTGEKPFQCRICMRNFSQSTTLKRHLRTHTGEKPFQC
RICMRNFSRSDHLSLHLKTHLRGSQLVKSELEEKKSELRHKLKYVPHEYI
ELIEIARNPTQDRILEMKVMEFFMKVYGYRGEHLGGSRKPDGAIYTVGSP
IDYGVIVDTKAYSGGYNLPIGQADEMQRYVKENQTRNKHINPNEWWKVYP
SSVTEFKFLEVSGHFKGNYKAQLTRLNRKTNCNGAVLSVEELLIGGEMIK
AGTLTLEEVRRKFNNGEINF*
ZFN J right Fok ELD-S full amino acid sequence
(SEQ ID NO: 441)
MDYKDHDGDYKDHDIDYKDDDDKMAPKKKRKVGRLEPGERPFQCRICMRN
FSRRAHLQNHTRTHTGEKPFQCRICMRNFSQSTTLKRHLRTHTGEKPFQC
RICMRNFSDGGHLTRHLKTHLRGSQLVKSELEEKKSELRHKLKYVPHEYI
ELIEIARNPTQDRILEMKVMEFFMKVYGYRGEHLGGSRKPDGAIYTVGSP
IDYGVIVDTKAYSGGYNLPIGQADEMERYVEENQTRDKHLNPNEWWKVYP
SSVTEFKFLEVSGHFKGNYKAQLTRLNHITNCNGAVLSVEELLIGGEMIK
AGTLTLEEVRRKFNNGEINF*

c. TAL effector Nucleases (TALENs)

TALENs may be used to introduce site-specific double strand breaks at targeted genomic loci. Site-specific double-strand breaks are created when two independent TALENs bind to nearby DNA sequences, thereby permitting dimerization of FokI and cleavage of the target DNA. TALENs have advanced genome editing due to their high rate of successful and efficient genetic modification. This DNA cleavage may stimulate the natural DNA-repair machinery, leading to one of two possible repair pathways: homology-directed repair (HDR) or the non-homologous end joining (NHEJ) pathway. The TALENs may be designed to target any gene involved in a genetic disease.

The TALENs may include a nuclease and a TALE DNA-binding domain that binds to the target gene in a TALEN target region. The target gene may have a mutation such as a frameshift mutation or a nonsense mutation. If the target gene has a mutation that causes a premature stop codon, the TALEN may be designed to recognize and bind a nucleotide sequence upstream or downstream from the premature stop codon. A “TALEN target region” includes the binding regions for two TALENs and the spacer region, which occurs between the binding regions. The two TALENs bind to different binding regions within the TALEN target region, after which the TALEN target region is cleaved. Examples of TALENs are described in International Patent Application No. PCT/US2013/038536, which is incorporated by reference in its entirety.

### 7. Transcriptional Activators

The composition, as described above, includes a nucleotide sequence encoding a transcriptional activator that activates a target gene. The transcriptional activator may be engineered. For example, an engineered transcriptional activator may be a CRISPR/Cas9-based system, a zinc finger fusion protein, or a TALE fusion protein.

a. CRISPR/Cas9-Based System

The CRISPR/Cas9-based system, as described above, may be used to activate transcription of a target gene with RNA. The CRISPR/Cas9-based system may include a fusion protein, as described above, wherein the second polypeptide domain has transcription activation activity or histone modification activity. For example, the second polypeptide domain may include VP64 or p300.

b. Zinc Finger Fusion Proteins

The transcriptional activator may be a zinc finger fusion protein. The zinc finger targeted DNA-binding domains, as described above, can be combined with a domain that has transcription activation activity or histone modification activity. For example, the domain may include VP64 or p300.

c. TALE Fusion Proteins

TALE fusion proteins may be used to activate transcription of a target gene. The TALE fusion protein may include a TALE DNA-binding domain and a domain that has transcription activation activity or histone modification activity. For example, the domain may include VP64 or p300.

### 8. Compositions

The present invention is directed to a composition for altering gene expression and engineering or altering genomic DNA in a cell or subject. The composition may also include a viral delivery system.

a. Compositions for Genome Editing in Muscle

The present invention is directed to a composition for genome editing a target gene in skeletal muscle or cardiac muscle of a subject. The composition includes a modified AAV vector and a nucleotide sequence encoding a site-specific nuclease. The composition delivers active forms of site-specific nucleases to skeletal muscle or cardiac muscle. The composition may further comprise a donor DNA or a transgene. These compositions may be used in genome editing, genome engineering, and correcting or reducing the effects of mutations in genes involved in genetic diseases and/or other skeletal or cardiac muscle conditions.

The target gene may be involved in differentiation of a cell or any other process in which activation, repression, or disruption of a gene may be desired, or may have a mutation such as a deletion, frameshift mutation, or a nonsense mutation. If the target gene has a mutation that causes a premature stop codon, an aberrant splice acceptor site or an aberrant splice donor site, the site-specific nucleases may be designed to recognize and bind a nucleotide sequence upstream or downstream from the premature stop codon, the aberrant splice acceptor site or the aberrant splice donor site. The site-specific nucleases may also be used to disrupt normal gene splicing by targeting splice acceptors and donors to induce skipping of premature stop codons or restore a disrupted reading frame. The site-specific nucleases may or may not mediate off-target changes to protein-coding regions of the genome.

b. Adeno-Associated Virus Vectors

The composition, as described above, includes a modified adeno-associated virus (AAV) vector. The modified AAV vector may have enhanced cardiac and skeletal muscle tissue tropism. The modified AAV vector may be capable of delivering and expressing the site-specific nuclease in the cell of a mammal. For example, the modified AAV vector may be an AAV-SASTG vector (Piacentino et al. (2012) Human Gene Therapy 23:635-646). The modified AAV vector may deliver nucleases to skeletal and cardiac muscle in vivo. The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy (2012) 12:139-151).

c. CRISPR/Cas9-Based System

The present disclosure also provides DNA targeting systems or compositions of at least one CRISPR/Cas9-based system, as described above. These compositions may be used in genome editing, genome engineering, and correcting or reducing the effects of mutations in genes involved in genetic diseases. The composition includes a CRISPR/Cas9-based system that includes a Cas9 protein or Cas9 fusion protein, as described above. The CRISPR/Cas9-based system may also include at least one gRNA, as described above.

d. Multiplex CRISPR/Cas9-Based System

The present disclosure also provides multiplex CRISPR/Cas9-based systems, as described above. These compositions may be used in genome editing, genome engineering, and correcting or reducing the effects of mutations in genes involved in genetic diseases. These compositions may be used to target more than one gene. The composition includes a modified lentiviral vector comprising a CRISPR/Cas9-based system that includes a Cas9 protein or Cas9 fusion protein, as described above and more than one gRNA, as described above.

### 9. Methods of Uses

Potential applications of the compositions are diverse across many areas of science and biotechnology. The disclosed compositions may be used to repair genetic mutations that cause disease. The disclosed compositions may be used to disrupt genes such that gene disruption leads to increases in muscle regeneration or muscle strength, or decreases in muscle aging. The disclosed compositions may be used to introduce therapeutic genes to be expressed systemically from skeletal muscle or cardiac muscle, such as clotting factors or monoclonal antibodies. The disclosed compositions may be used to modulate mammalian gene expression. The disclosed compositions may be used to transdifferentiate or induce the differentiation of a cell or correct a mutant gene in a cell. Examples of activation of genes related to cell and gene therapy, genetic reprogramming, and regenerative medicine are provided. RNA-guided transcriptional activators may be used to reprogram cell lineage specification. Activation of endogenous genes encoding the key regulators of cell fate, rather than forced overexpression of these factors, may potentially lead to more rapid, efficient, stable, or specific methods for genetic reprogramming, transdifferentiation, and/or induced differentiation.

### 10. Methods of Genome Editing in Muscle

The present disclosure is directed to a method of genome editing in a skeletal muscle or cardiac muscle of a subject. The method comprises administering to the skeletal muscle or cardiac muscle of the subject the composition for genome editing in skeletal muscle or cardiac muscle, as described above. The genome editing may include correcting a mutant gene or inserting a transgene. Correcting the mutant gene may include deleting, rearranging, or replacing the mutant gene. Correcting the mutant gene may include nuclease-mediated NHEJ or HDR.

### 11. Methods of Using CRISPR/Cas9-Based System

Potential applications of the CRISPR/Cas9-based system are diverse across many areas of science and biotechnology. The disclosed CRISPR/Cas9-based systems may be used to modulate mammalian gene expression. The disclosed CRISPR/Cas9-based systems may be used to transdifferentiate or induce differentiation of a cell or correct a mutant gene in a cell. Examples of activation of genes related to cell and gene therapy, genetic reprogramming, and regenerative medicine are provided. RNA-guided transcriptional activators may be used to reprogram cell lineage specification. Although reprogramming was incomplete and inefficient in these experiments, there are many ways by which this method could be improved, including repeated transfections of iCas9-VP64/gRNA combinations, stable expression of these factors, and targeting multiple genes, such as Brn2 and Mytl1 in addition to Ascl1 for transdifferentiation into a neuronal phenotype. Activation of endogenous genes encoding the key regulators of cell fate, rather than forced overexpression of these factors, may potentially lead to more rapid, efficient, stable, or specific methods for genetic reprogramming and transdifferentiation or induced differentiation of a cell. Finally, Cas9 fusions to other domains, including repressive and epigenetic-modifying domains, could provide a greater diversity of RNA-guided transcriptional regulators to complement other RNA-based tools for mammalian cell engineering.

a. Methods of Activating Gene Expression

The present disclosure provides a mechanism for activating the expression of endogenous genes, such as mammalian genes, based on targeting a transcriptional activator to promoters via RNA using a CRISPR/Cas9 based system, as described above. This is fundamentally different from previously described methods based on engineering sequence-specific DNA-binding proteins and may provide opportunities for targeted gene regulation. Because the generation of gRNA expression plasmids simply involves synthesizing two short custom oligonucleotides and one cloning step, it is possible to generate many new gene activators quickly and economically. The gRNAs can also be transfected directly to cells following in vitro transcription. Multiple gRNAs targeted to single promoters were shown, but simultaneous targeting of multiple promoters could also be possible. Recognition of genomic target sites with RNAs, rather than proteins, may also circumvent limitations of targeting epigenetically modified sites, such as methylated DNA.

In contrast to current methods based on engineering DNA-binding proteins, Cas9 fused to a transcriptional activation domain can be targeted by combinations of guide RNA molecules to induce the expression of endogenous human genes. This straightforward and versatile approach for targeted gene activation circumvents the need for engineering new proteins and allows for widespread synthetic gene regulation.

The method may include administering to a cell or subject a CRISPR/Cas9-based system, a polynucleotide or vector encoding said CRISPR/Cas9-based system, or DNA targeting systems or compositions of at least one CRISPR/Cas9-based system, as described above. The method may include administering a CRISPR/Cas9-based system, such as administering a Cas9 fusion protein containing transcription activation domain or a nucleotide sequence encoding said Cas9 fusion protein. The Cas9 fusion protein may include a transcription activation domain such as aVP16 protein or a transcription co-activator such as a p300 protein.

(1) dCas9-VP16

The Cas9 fusion protein may include a transcription activation domain such as aVP16 protein. The transcription activation domain may contain at least 1 VP16 protein, at least 2 VP16 proteins, at least 3 VP16 proteins, at least 4 VP16 proteins (i.e., a VP64 activator domain), at least 5 VP16 proteins, at least 6 VP16 proteins, at least 6 VP16 proteins, or at least 10 VP16 proteins. The Cas9 protein may be a Cas9 protein in which the nuclease activity is inactivated. For example, the Cas9 protein in the fusion protein may be iCas9 (amino acids 36-1403 of SEQ ID NO: 1), which includes the amino acid substitutions of D10A and H840A. The Cas9 fusion protein may be iCas9-VP64.

(2) dCas9-p300

The Cas9 fusion protein may include a transcription co-activation domain such as a p300 protein. The p300 protein (also known as EP300 or E1A binding protein p300) is encoded by the EP300 gene and regulates the activity of many genes in tissues throughout the body. The p300 protein plays a role in regulating cell growth and division, prompting cells to mature and assume specialized functions (differentiate) and preventing the growth of cancerous tumors. The p300 protein activates transcription by connecting transcription factors with a complex of proteins that carry out transcription in the cell's nucleus. The p300 interaction with transcription factors is managed by one or more of p300 domains: the nuclear receptor interaction domain (RID), the CREB and MYB interaction domain (KIX), the cysteine/histidine regions (TAZ1/CH1 and TAZ2/CH3) and the interferon response binding domain (IBID). The last four domains, KIX, TAZ1, TAZ2 and IBiD of p300, each bind tightly to a sequence spanning both transactivation domains 9aaTADs of transcription factor p53. The protein functions as histone acetyltransferase that regulates transcription via chromatin remodeling, and is important in the processes of cell proliferation and differentiation. It mediates cAMP-gene regulation by binding specifically to phosphorylated CREB protein.

The p300 protein may activate Mothers against decapentaplegic homolog 7, MAF, TSG101, Peroxisome proliferator-activated receptor alpha, NPAS2, PAX6, DDX5, MYBL2, Mothers against decapentaplegic homolog 1, Mothers against decapentaplegic homolog 2, Lymphoid enhancer-binding factor 1, SNIP1, TRERF1, STAT3, EID1, RAR-related orphan receptor alpha, ELK1, HIF1A, ING5, Peroxisome proliferator-activated receptor gamma, SS18, TCF3, Zif268, Estrogen receptor alpha, GPS2, MyoD, YY1, ING4, PROX1, CITED1, HNF1A, MEF2C, MEF2D, MAML1, Twist transcription factor, PTMA, IRF2, DTX1, Flap structure-specific endonuclease 1, Myocyte-specific enhancer factor 2A, CDX2, BRCA1, HNRPU, STAT6, CITED2, RELA, TGS1, CEBPB, Mdm2, NCOA6, NFATC2, Thyroid hormone receptor alpha, BCL3, TFAP2A, PCNA, P53 and TAL1.

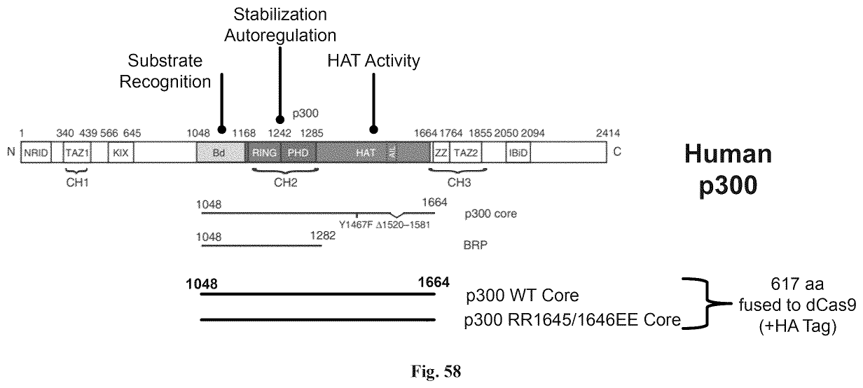

The transcription co-activation domain may include a human p300 protein or a fragment thereof. The transcription co-activation domain may include a wild-type human p300 protein or a mutant human p300 protein, or fragments thereof. The transcription co-activation domain may include the core lysine-acetyltranserase domain of the human p300 protein, i.e., the p300 HAT Core (also known as “p300 WT Core”; see FIG. 58). The Cas9 protein may be a Cas9 protein in which the nuclease activity is inactivated. For example, the Cas9 protein in the fusion protein may be iCas9 (amino acids 36-1403 of SEQ ID NO: 1), which includes the amino acid substitutions of D10A and H840A. The Cas9 fusion protein may be iCas9-p300 WT Core.

(3) gRNA

The method may also include administering to a cell or subject a CRISPR/Cas9-based system at least one gRNA, wherein the gRNAs target different DNA sequences. The target DNA sequences may be overlapping. The number of gRNA administered to the cell may be at least 1 gRNA, at least 2 different gRNAs, at least 3 different gRNAs at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, 8 different gRNAs to at least 12 different gRNAs, at least 8 different gRNAs to at least 8 different gRNAs,

The gRNA may comprise a complementary polynucleotide sequence of the target DNA sequence followed by NGG. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may target at least one of the promoter region, the enhancer region or the transcribed region of the target gene. The gRNA may include a nucleic acid sequence of at least one of SEQ ID NOs: 5-40, 65-144, 492-515, 540-563, and 585-625.

b. Methods of Repressing Gene Expression

The present disclosure provides a mechanism for repressing the expression of endogenous genes, such as mammalian genes, based on targeting genomic regulatory elements, such as distal enhancers, via RNA using a CRISPR/Cas9 based system, as described above. The Cas9 fusion protein may include a transcriptional repressor, such as the KRAB repressor. The Cas9 fusion protein may be dCas9-KRAB. The dCas9-KRAB may additionally affect epigenetic gene regulation by recruiting heterochromatin-forming factors to the targeted locus. The CRISPR/dCas9-KRAB system may be used to repress the transcription of genes, but can also be used to target genomic regulatory elements which were previously inaccessible by traditional repression methods such as RNA interference (FIG. 53B). Delivering dCas9-KRAB with gRNAs targeted to a distal enhancer may disrupt expression of multiple genes regulated by the targeted enhancer (see FIG. 53C). The targeted enhancer may be any enhancer for a gene such as the HS2 enhancer.

a. Methods of Transdifferentiation or Induced Differentiation

The present disclosure provides a mechanism for transdifferentiating or inducing differentiation of cells by activating endogenous genes via RNA using a CRISPR/Cas9-based system, as described above.

### (1) Transdifferentiation

The CRISPR/Cas9-based system may be used to transdifferentiate cells. Transdifferentiation, also known as lineage reprogramming or direct conversion, is a process where cells convert from one differentiated cell type to another without undergoing an intermediate pluripotent state or progenitor cell type. It is a type of metaplasia, which includes all cell fate switches, including the interconversion of stem cells. Transdifferentiation of cells has potential uses for disease modeling, drug discovery, gene therapy and regenerative medicine. Activation of endogenous genes, such as BRN2, MYT1L, ASCL1, NANOG, and/or MYOD1, using the CRISPR/Cas9 based system described above may lead to transdifferentiation of several cell types, such as fibroblasts, cardiomyocytes, hepatocytes, chondrocytes, mesenchymal progenitor cells, hematopoetic stem cells, or smooth muscle cells, into neuronal and myogenic phenotypes, respectively.

### (2) Inducing Differentiation

The CRISPR/Cas9-based system may be used to induce differentiation of cells, such as stem cells, cardiomyocytes, hepatocytes, chondrocytes, mesenchymal progenitor cells, hematopoetic stem cells, or smooth muscle cells. For example, stem cells, such as embryonic stem cells or pluripotent stem cells, may be induced to differentiate into muscle cells or vascular endothelial cell, i.e., induce neuronal or myogenic differentiation.

### 12. Uses of Multiplex CRISPR/Cas9-Based System

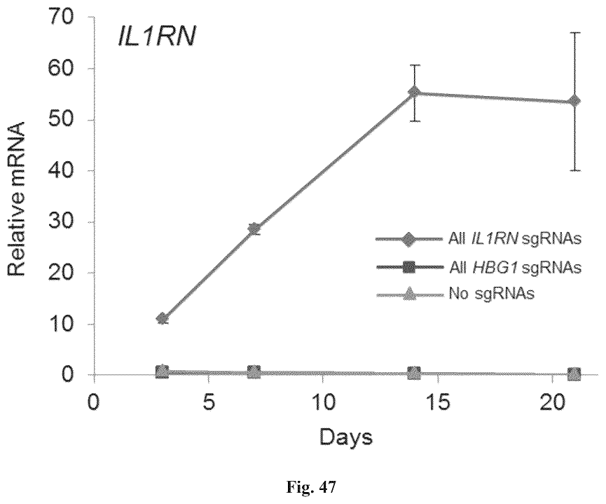

The multiplex CRISPR/Cas9-Based System takes advantage of the simplicity and low cost of sgRNA design and may be helpful in exploiting advances in high-throughput genomic research using CRISPR/Cas9 technology. For example, the single lentiviral vectors described here are useful in expressing Cas9 and numerous sgRNAs in difficult cell lines, such as primary fibroblasts described here (FIG. 47). The multiplex CRISPR/Cas9-Based System may be used in the same ways as the CRISPR/Cas9-Based System described above.

In addition to the described transcriptional activation and nuclease functionality, this system will be useful for expressing other novel Cas9-based effectors that control epigenetic modifications for diverse purposes, including interrogation of genome architecture and pathways of endogenous gene regulation. As endogenous gene regulation is a delicate balance between multiple enzymes, multiplexing Cas9 systems with different functionalities will allow for examining the complex interplay among different regulatory signals. The vector described here should be compatible with aptamer-modified sgRNAs and orthogonal Cas9s to enable independent genetic manipulations using a single set of sgRNAs.

The multiplex CRISPR/Cas9-Based System may be used to activate at least one endogenous gene in a cell. The method includes contacting a cell with the modified lentiviral vector. The endogenous gene may be transiently activated or stably activated. The endogenous gene may be transiently repressed or stably repressed. The fusion protein may be expressed at similar levels to the sgRNAs. The fusion protein may be expressed at different levels compared to the sgRNAs. The cell may be a primary human cell.

The multiplex CRISPR/Cas9-Based System may be used in a method of multiplex gene editing in a cell. The method includes contacting a cell with the modified lentiviral vector. The multiplex gene editing may include correcting a mutant gene or inserting a transgene. Correcting a mutant gene may include deleting, rearranging or replacing the mutant gene. Correcting the mutant gene may include nuclease-mediated non-homologous end joining or homology-directed repair. The multiplex gene editing may include deleting or correcting at least one gene, wherein the gene is an endogenous normal gene or a mutant gene. The multiplex gene editing may include deleting or correcting at least two genes. For example, at least two genes, at least three genes, at least four genes, at least five genes, at least six genes, at least seven genes, at least eight genes, at least nine genes, or at least ten genes may be deleted or corrected.

The multiplex CRISPR/Cas9-Based System may be used in a method of multiplex modulation of gene expression in a cell. The method includes contacting a cell with the modified lentiviral vector. The method may include modulating the gene expression levels of at least one gene. The gene expression of the at least one target gene is modulated when gene expression levels of the at least one target gene are increased or decreased compared to normal gene expression levels for the at least one target gene. The gene expression levels may be RNA or protein levels.

### 13. Methods of Correcting a Mutant Gene and Treating a Subject

The present disclosure is also directed to a method of correcting a mutant gene in a subject. The method comprises administering to the skeletal muscle or cardiac muscle of the subject the composition for genome editing in skeletal muscle or cardiac muscle, as described above. Use of the composition to deliver the site-specific nuclease to the skeletal muscle or cardiac muscle may restore the expression of a full-functional or partially-functional protein with a repair template or donor DNA, which can replace the entire gene or the region containing the mutation. The site-specific nuclease may be used to introduce site-specific double strand breaks at targeted genomic loci. Site-specific double-strand breaks are created when the site-specific nuclease binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

The present disclosure is directed to genome editing with a site-specific nuclease without a repair template, which can efficiently correct the reading frame and restore the expression of a functional protein involved in a genetic disease. The disclosed site-specific nucleases may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active site-specific nucleases with an efficient gene editing method for the treatment of genetic diseases caused by mutations in nonessential coding regions that cause frameshifts, premature stop codons, aberrant splice donor sites or aberrant splice acceptor sites.

a. Nuclease Mediated Non-Homologous End Joining

Restoration of protein expression from an endogenous mutated gene may be through template-free NHEJ-mediated DNA repair. In contrast to a transient method targeting the target gene RNA, the correction of the target gene reading frame in the genome by a transiently expressed site-specific nuclease may lead to permanently restored target gene expression by each modified cell and all of its progeny.

Nuclease mediated NHEJ gene correction may correct the mutated target gene and offers several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based gene correction using a CRISPR/Cas9-based system, as well as other engineered nucleases including meganucleases and zinc finger nucleases, may be combined with other existing ex vivo and in vivo platforms for cell- and gene-based therapies, in addition to the plasmid electroporation approach described here. For example, delivery of a CRISPR/Cas9-based system by mRNA-based gene transfer or as purified cell permeable proteins could enable a DNA-free genome editing approach that would circumvent any possibility of insertional mutagenesis.

b. Homology-Directed Repair

Restoration of protein expression from an endogenous mutated gene may involve homology-directed repair. The method as described above further includes administrating a donor template to the cell. The donor template may include a nucleotide sequence encoding a full-functional protein or a partially-functional protein. For example, the donor template may include a miniaturized dystrophin construct, termed minidystrophin (“minidys”), a full-functional dystrophin construct for restoring a mutant dystrophin gene, or a fragment of the dystrophin gene that after homology-directed repair leads to restoration of the mutant dystrophin gene.

c. Methods of Correcting a Mutant Gene and Treating a Subject Using CRISPR/Cas9

The present disclosure is also directed to genome editing with the CRISPR/Cas9-based system to restore the expression of a full-functional or partially-functional protein with a repair template or donor DNA, which can replace the entire gene or the region containing the mutation. The CRISPR/Cas9-based system may be used to introduce site-specific double strand breaks at targeted genomic loci. Site-specific double-strand breaks are created when the CRISPR/Cas9-based system binds to a target DNA sequences using the gRNA, thereby permitting cleavage of the target DNA. The CRISPR/Cas9-based system has the advantage of advanced genome editing due to their high rate of successful and efficient genetic modification. This DNA cleavage may stimulate the natural DNA-repair machinery, leading to one of two possible repair pathways: homology-directed repair (HDR) or the non-homologous end joining (NHEJ) pathway. For example, a CRISPR/Cas9-based system directed towards the dystrophin gene may include a gRNA having a nucleic acid sequence of any one of SEQ ID NOs: 65-115.

The present disclosure is directed to genome editing with CRISPR/Cas9-based system without a repair template, which can efficiently correct the reading frame and restore the expression of a functional protein involved in a genetic disease. The disclosed CRISPR/Cas9-based system and methods may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based system with an efficient gene editing method for the treatment of genetic diseases caused by mutations in nonessential coding regions that cause frameshifts, premature stop codons, aberrant splice donor sites or aberrant splice acceptor sites.

The present disclosure provides methods of correcting a mutant gene in a cell and treating a subject suffering from a genetic disease, such as DMD. The method may include administering to a cell or subject a CRISPR/Cas9-based system, a polynucleotide or vector encoding said CRISPR/Cas9-based system, or composition of said CRISPR/Cas9-based system as described above. The method may include administering a CRISPR/Cas9-based system, such as administering a Cas9 protein or Cas9 fusion protein containing a second domain having nuclease activity, a nucleotide sequence encoding said Cas9 protein or Cas9 fusion protein, and/or at least one gRNA, wherein the gRNAs target different DNA sequences. The target DNA sequences may be overlapping. The number of gRNA administered to the cell may be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNA, at least 15 different gRNA, at least 20 different gRNA, at least 30 different gRNA, or at least 50 different gRNA, as described above. The gRNA may include a nucleic acid sequence of at least one of SEQ ID NOs: 65-115. The method may involve homology-directed repair or non-homologous end joining.

### 14. Methods of Treating a Disease

The present disclosure is directed to a method of treating a subject in need thereof. The method comprises administering to a tissue of a subject the composition for altering gene expression and engineering or altering genomic DNA in a cell or subject genome editing, as described above. The method may comprises administering to the skeletal muscle or cardiac muscle of the subject the composition for genome editing in skeletal muscle or cardiac muscle, as described above. The subject may be suffering from a skeletal muscle or cardiac muscle condition causing degeneration or weakness or a genetic disease. For example, the subject may be suffering from Duchenne muscular dystrophy, as described above.

a. Duchenne Muscular Dystrophy

The method, as described above, may be used for correcting the dystrophin gene and recovering full-functional or partially-functional protein expression of said mutated dystrophin gene. In some aspects and embodiments the disclosure provides a method for reducing the effects (e.g., clinical symptoms/indications) of DMD in a patient. In some aspects and embodiments the disclosure provides a method for treating DMD in a patient. In some aspects and embodiments the disclosure provides a method for preventing DMD in a patient. In some aspects and embodiments the disclosure provides a method for preventing further progression of DMD in a patient.

### 15. Constructs and Plasmids

The compositions, as described above, may comprise genetic constructs that encodes the CRISPR/Cas9-based system, as disclosed herein. The genetic construct, such as a plasmid, may comprise a nucleic acid that encodes the CRISPR/Cas9-based system, such as the Cas9 protein and Cas9 fusion proteins and/or at least one of the gRNAs. The compositions, as described above, may comprise genetic constructs that encodes the modified AAV vector and a nucleic acid sequence that encodes the site-specific nuclease, as disclosed herein. The genetic construct, such as a plasmid, may comprise a nucleic acid that encodes the site-specific nuclease. The compositions, as described above, may comprise genetic constructs that encodes the modified lentiviral vector, as disclosed herein. The genetic construct, such as a plasmid, may comprise a nucleic acid that encodes the Cas9-fusion protein and at least one sgRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the fusion protein, such as the Cas9-fusion protein or site-specific nuclease, in the cell of a mammal. The vector may be recombinant. The vector may comprise heterologous nucleic acid encoding the fusion protein, such as the Cas9-fusion protein or site-specific nuclease. The vector may be a plasmid. The vector may be useful for transfecting cells with nucleic acid encoding the Cas9-fusion protein or site-specific nuclease, which the transformed host cell is cultured and maintained under conditions wherein expression of the Cas9-fusion protein or the site-specific nuclease system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas9-based system or the site-specific nuclease and may further comprise an initiation codon, which may be upstream of the CRISPR/Cas9-based system or the site-specific nuclease coding sequence, and a stop codon, which may be downstream of the CRISPR/Cas9-based system or the site-specific nuclease coding sequence. The initiation and termination codon may be in frame with the CRISPR/Cas9-based system or the site-specific nuclease coding sequence. The vector may also comprise a promoter that is operably linked to the CRISPR/Cas9-based system or the site-specific nuclease coding sequence. The promoter operably linked to the CRISPR/Cas9-based system or the site-specific nuclease coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication No. US20040175727, the contents of which are incorporated herein in its entirety.

The vector may also comprise a polyadenylation signal, which may be downstream of the CRISPR/Cas9-based system or the site-specific nuclease. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

The vector may also comprise an enhancer upstream of the CRISPR/Cas9-based system, i.e., the Cas9 protein or Cas9 fusion protein coding sequence or sgRNAs, or the site-specific nuclease. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cas9-based system, including the nucleic acid sequence encoding the Cas9 protein or Cas9 fusion protein and the nucleic acid sequence encoding the at least one gRNA comprising the nucleic acid sequence of at least one of SEQ ID NOs: 5-40, 65-144, 492-515, 540-563, and 585-625.

### 16. Pharmaceutical Compositions

The composition may be in a pharmaceutical composition. The pharmaceutical composition may comprise about 1 ng to about 10 mg of DNA encoding the CRISPR/Cas9-based system or CRISPR/Cas9-based system protein component, i.e., the Cas9 protein or Cas9 fusion protein. The pharmaceutical composition may comprise about 1 ng to about 10 mg of the DNA of the modified AAV vector and nucleotide sequence encoding the site-specific nuclease. The pharmaceutical composition may comprise about 1 ng to about 10 mg of the DNA of the modified lentiviral vector. The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 17. Methods of Delivery

Provided herein is a method for delivering the pharmaceutical formulations, preferably compositions described above, for providing genetic constructs. The delivery of the compositions may be the transfection or electroporation of the composition as a nucleic acid molecule that is expressed in the cell and delivered to the surface of the cell. The nucleic acid molecules may be electroporated using BioRad Gene Pulser Xcell or Amaxa Nucleofector IIb devices. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

Upon delivery of the composition to the tissue, and thereupon the vector into the cells of the mammal, the transfected cells will express the fusion protein, such as a CRISPR/Cas9-based system and/or a site-specific nuclease. The composition may be administered to a mammal to alter gene expression or to re-engineer or alter the genome. For example, the composition may be administered to a mammal to correct the dystrophin gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

a. CRISPR/Cas9-Based System

The vector encoding a CRISPR/Cas9-based system protein component, i.e., the Cas9 protein or Cas9 fusion protein, may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The nucleotide encoding a CRISPR/Cas9-based system protein component, i.e., the Cas9 protein or Cas9 fusion protein, may be introduced into a cell to genetically correct the target gene or alter gene expression of a gene, such as activate or repress endogenous genes. For example, a nucleotide encoding a CRISPR/Cas9-based system protein component, i.e., the Cas9 protein or Cas9 fusion protein, directed towards a mutant dystrophin gene by the gRNA may be introduced into a myoblast cell from a DMD patient. Alternatively, they may be introduced into a fibroblast cell from a DMD patient, and the genetically corrected fibroblast cell may be treated with MyoD to induce differentiation into myoblasts, which may be implanted into subjects, such as the damaged muscles of a subject to verify that the corrected dystrophin protein was functional and/or to treat the subject. The modified cells may also be stem cells, such as induced pluripotent stem cells, bone marrow-derived progenitors, skeletal muscle progenitors, human skeletal myoblasts from DMD patients, CD133+ cells, mesoangioblasts, and MyoD- or Pax7-transduced cells, or other myogenic progenitor cells. For example, the CRISPR/Cas9-based system may cause neuronal or myogenic differentiation of an induced pluripotent stem cell.

### 18. Routes of Administration

The compositions may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle.

### 19. Cell Types

Any of these delivery methods and/or routes of administration could be utilized with a myriad of cell types, for example, those cell types currently under investigation for cell-based therapies. Cell types may be fibroblasts, pluripotent stem cells, cardiomyocytes, hepatocytes, chondrocytes, mesenchymal progenitor cells, hematopoetic stem cells, smooth muscle cells, or K562 human erythroid leukemia cell line.

a. DMD

Cell types currently under investigation for cell-based therapies of DMD include immortalized myoblast cells, such as wild-type and DMD patient derived lines, for example Δ48-50 DMD, DMD 8036 (del48-50), C25C14 and DMD-7796 cell lines, primal DMD dermal fibroblasts, induced pluripotent stem cells, bone marrow-derived progenitors, skeletal muscle progenitors, human skeletal myoblasts from DMD patients, CD133+ cells, mesoangioblasts, cardiomyocytes, hepatocytes, chondrocytes, mesenchymal progenitor cells, hematopoetic stem cells, smooth muscle cells, and MyoD- or Pax7-transduced cells, or other myogenic progenitor cells. Immortalization of human myogenic cells may be used for clonal derivation of genetically corrected myogenic cells. Cells may be modified ex vivo to isolate and expand clonal populations of immortalized DMD myoblasts that contain a genetically corrected dystrophin gene and are free of other nuclease-introduced mutations in protein coding regions of the genome. Alternatively, transient in vivo delivery of nucleases by non-viral or non-integrating viral gene transfer, or by direct delivery of purified proteins and gRNAs containing cell-penetrating motifs may enable highly specific correction in situ with minimal or no risk of exogenous DNA integration.

### 20. Kits

Provided herein is a kit, which may be used to edit a genome in skeletal muscle or cardiac muscle, such as correcting a mutant gene. The kit comprises a composition for genome editing in skeletal muscle or cardiac muscle, as described above, and instructions for using said composition. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The composition for genome editing in skeletal muscle or cardiac muscle may include a modified AAV vector and a nucleotide sequence encoding a site-specific nuclease, as described above. The site-specific nuclease may include a ZFN, a TALEN, or CRISPR/Cas9-based system, as described above, that specifically binds and cleaves a mutated gene. The site-specific nuclease, as described above, may be included in the kit to specifically bind and target a particular region in the mutated gene. The site-specific nuclease may be specific for a mutated dystrophin gene, as described above. The kit may further include donor DNA, a gRNA, or a transgene, as described above.

a. CRISPR/Cas9-Based System

Provided herein is a kit, which may be used to correct a mutated gene. The kit comprises at least one component for correcting a mutated gene and instructions for using the CRISPR/Cas9-based system. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

At least one component may include at least one CRISPR/Cas9-based system, as described above, which specifically targets a gene. The kit may include a Cas9 protein or Cas9 fusion protein, a nucleotide sequence encoding said Cas9 protein or Cas9 fusion protein, and/or at least one gRNA. The CRISPR/Cas9-based system, as described above, may be included in the kit to specifically bind and target a particular target region upstream, within or downstream of the coding region of the target gene. For example, a CRISPR/Cas9-based system may be specific for a promoter region of a target gene or a CRISPR/Cas9-based system may be specific for a mutated gene, for example a mutated dystrophin gene, as described above. The kit may include donor DNA, as described above.

### 21. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present invention has multiple aspects, illustrated by the following non-limiting examples.

### Example 1

### Materials and Methods

Cell culture and transfection. HEK293T cells were obtained from the American Tissue Collection Center (ATCC) through the Duke University Cancer Center Facilities and were maintained in DMEM supplemented with 10% fetal bovine serum and 1% penicillin/streptomycin at 37° C. with 5% CO₂. HEK293T cells were transfected with Lipofectamine 2000 (Invitrogen) according to manufacturer's instructions. Transfection efficiencies were routinely higher than 80% as determined by fluorescence microscopy following delivery of a control eGFP expression plasmid. Cas9 expression plasmid was transfected at a mass ratio of 3:1 to either the individual gRNA expression plasmids or the identical amount of gRNA expression plasmid consisting of a mixture of equal amounts of the four gRNAs.

Primary mouse embryonic fibroblasts (PMEF-HL, Millipore, Billerica, MA) were seeded (75,000 per well) in 24-well TCPS plates (BD, Franklin Lakes, NJ) and maintained at 37° C. and 5% CO₂ in complete MEF medium consisting of high glucose DMEM supplemented with 10% Premium Select FBS (Atlanta Biologicals, Lawrenceville, GA), 25 μg mL⁻¹ gentamicin (Invitrogen), 1×GlutaMAX, non-essential amino acids, sodium pyruvate, and β-mercaptoethanol (Invitrogen). MEF transfections were performed with a single 1 μg cm⁻² dose of total plasmid DNA, delivered as cationic nanocomplexes following electrostatic condensation with poly (CBA-ABOL) in serum- and antibiotic-free OptiMEM, as described previously (Adler et al. Molecular therapy. Nucleic acids 1, e32 (2012)). OptiMEM was replaced with complete MEF medium 4 hrs after transfection. 48 hrs after transfection, MEFs were processed for qRT-PCR, or the complete MEF medium was replaced with N3 neural induction medium containing: DMEM/F-12 (Invitrogen), 1×N-2 Supplement (Invitrogen), 10 ng mL⁻¹ human bFGF2 (Stemgent, Cambridge, MA), and 25 μg mL⁻¹ gentamicin (Invitrogen). A GFP reporter vector (pmax-GFP, 3486 bp, Amaxa, Cologne, Germany) was used to optimize transfection conditions. Cas9 expression plasmid was transfected at a mass ratio of 3:1 or 1:1 to an equal mixture of four gRNA expression plasmids.

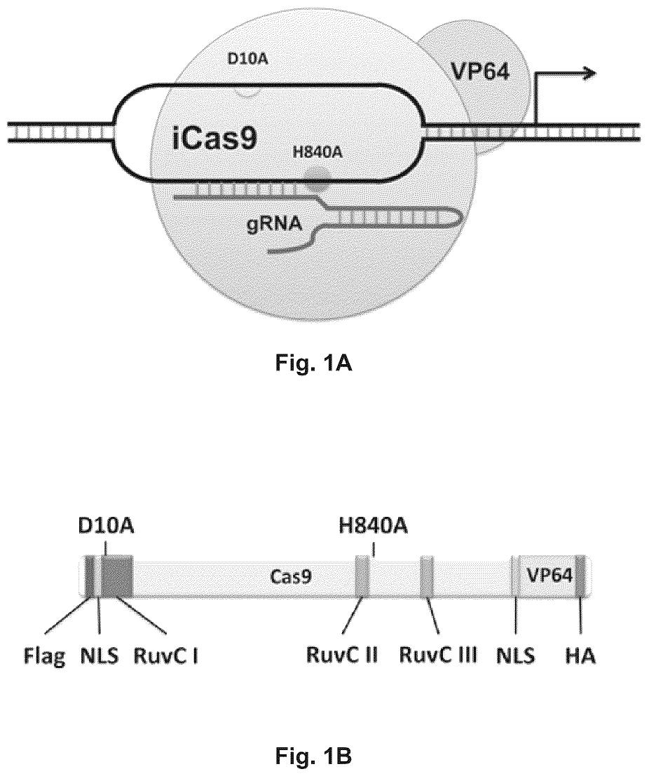

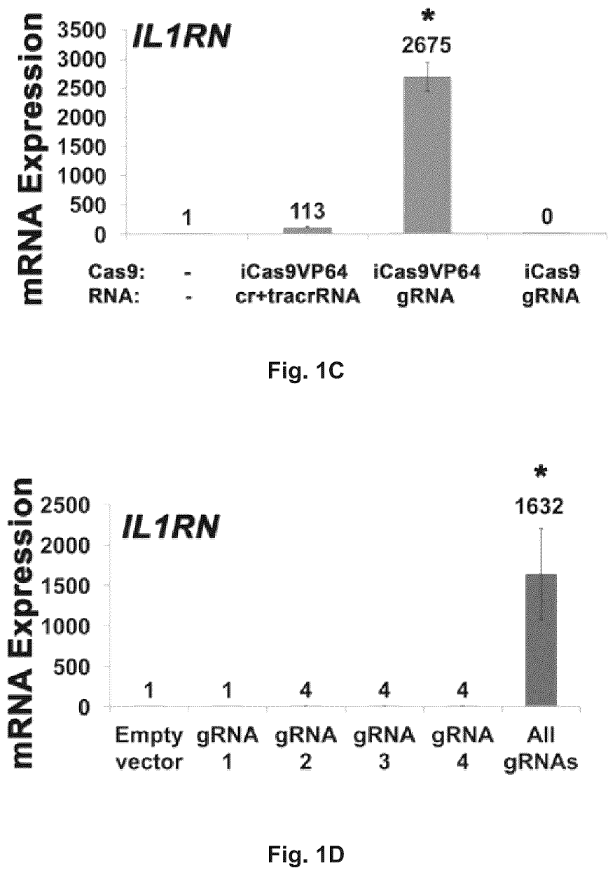

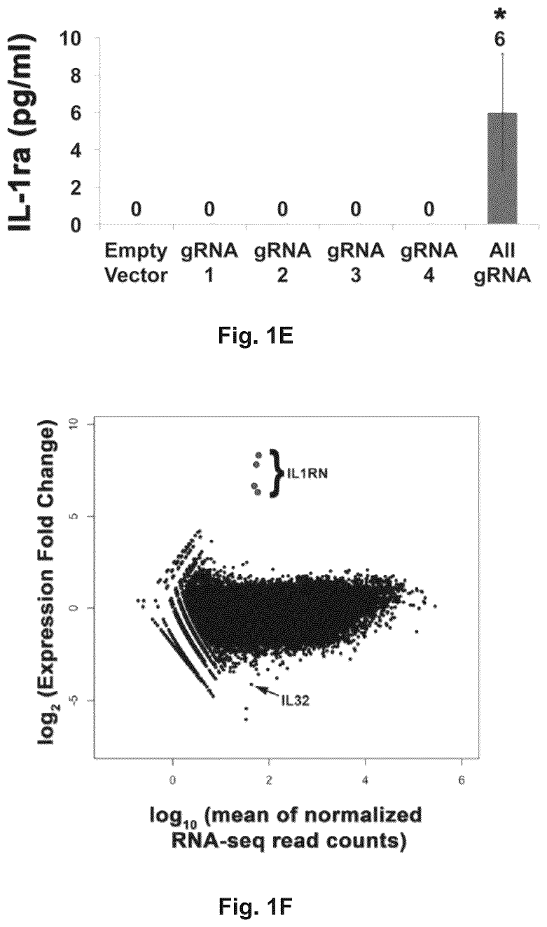

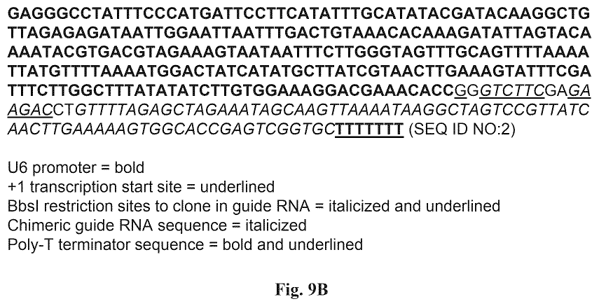

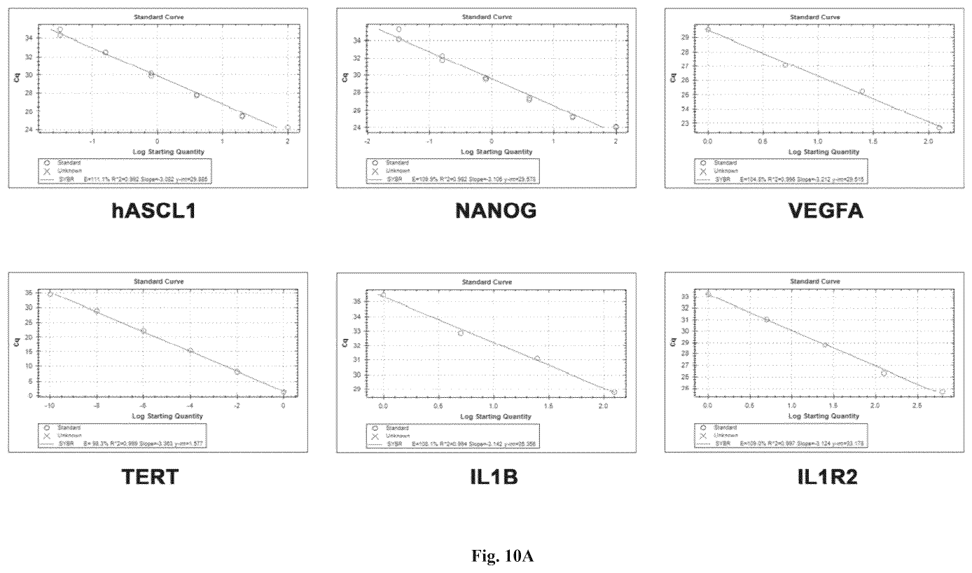

Plasmids. The plasmids encoding wild-type and H840A Cas9 were obtained from Addgene (Plasmid #39312 and Plasmid #39316; Jinek, et al. Science 337, 816-821 (2012)). H840A Cas9 was cloned into the vector pcDNA3.1 in frame with a FLAG epitope tag and a nuclear localization sequence (NLS) at the N-terminus with a primer pair that introduced the D10A mutation. The VP64 domain, an NLS, and an HA epitope tag were cloned in frame with the Cas9 ORF at the C-terminus (FIG. 1A, FIG. 9A). The tracrRNA and crRNA expression cassettes (Cong et al. Science 339, 819-823 (2013)) were ordered as gBlocks (Integrated DNA Technologies (IDT)) and cloned into a pZDonor plasmid (Sigma) with KpnI and SacII sites. A chimeric guide RNA expression cassette (Mali et al. Science 339, 823-826 (2013)) was also ordered as gBlocks with modifications to include a BbsI restriction site to facilitate rapid cloning of new guide RNA spacer sequences (FIG. 9B). The oligonucleotides containing the target sequences were obtained from IDT, hybridized, phosphorylated, and cloned in the appropriate plasmids using BbsI sites. The target sequences are provided in Table 2.

TABLE 2
Target sequences of gRNAs

| Target | Name | Sequence | SEQ ID NO |
|---|---|---|---|
| ASCL1 | CR1 | GCTGGGTGTCCCATTGAAA | 5 |
| | CR2 | CAGCCGCTCGCTGCAGCAG | 6 |
| | CR3 | TGGAGAGTTTGCAAGGAGC | 7 |
| | CR4 | GTTTATTCAGCCGGGAGTC | 8 |
| NANOG | CR1 | CGCCAGGAGGGGTGGGTCTA | 9 |
| | CR2 | CCTTGGTGAGACTGGTAGA | 10 |
| | CR3 | GTCTTCAGGTTCTGTTGCT | 11 |
| | CR4 | ATATTCCTGATTTAAAAGT | 12 |
| VEGFA | CR1 | TTAAAAGTCGGCTGGTAGC | 13 |
| | CR2 | CGGGCCGGGGGCGGGGTCC | 14 |
| | CR3 | GCCCGAGCCGCGTGTGGAA | 15 |
| | CR4 | CCTTCATTGCGGCGGGCTG | 16 |
| TERT | CR1 | CCGACCCCTCCCGGGTCCC | 17 |
| | CR2 | CAGGACCGCGCTTCCCACG | 18 |
| | CR3 | TGCACCCTGGGAGCGCGAG | 19 |
| | CR4 | CCGCACGCACCTGTTCCCA | 20 |
| IL1B | CR1 | AAAACAGCGAGGGAGAAAC | 21 |
| | CR2 | TTAACTTGATTGTGAAATC | 22 |
| | CR3 | AAAACAATGCATATTTGCA | 23 |
| | CR4 | AAAATCCAGTATTTTAATG | 24 |
| IL1R2 | CR1 | ACCCAGCACTGCAGCCTGG | 25 |
| | CR2 | AACTTATGCGGCGTTTCCT | 26 |
| | CR3 | TCACTTTAAAACCACCTCT | 27 |
| | CR4 | GCATCTTTTTCTCTTTAAT | 28 |
| IL1RN | CR1 | TGTACTCTCTGAGGTGCTC | 29 |
| | CR2 | ACGCAGATAAGAACCAGTT | 30 |
| | CR3 | CATCAAGTCAGCCATCAGC | 31 |
| | CR4 | GAGTCACCCTCCTGGAAAC | 32 |
| HBG1/2 | CR1 | GCTAGGGATGAAGAATAAA | 33 |
| | CR2 | TTGACCAATAGCCTTGACA | 34 |
| | CR3 | TGCAAATATCTGTCTGAAA | 35 |
| | CR4 | AAATTAGCAGTATCCTCTT | 36 |
| MYOD1 | CR1 | CCTGGGCTCCGGGGCGTTT | 37 |
| | CR2 | GGCCCCTGCGGCCACCCCG | 38 |
| | CR3 | CTCCCTCCCTGCCCGGTAG | 39 |
| | CR4 | AGGTTTGGAAAGGGCGTGC | 40 |

Western blot. Cells were lysed in 50 mM Tris-Cl (pH 7.4), 150 mM NaCl, 0.5% Triton X-100, and 0.1% SDS. Lysates were mixed with loading buffer, boiled for 5 min, and equal volumes of protein were run in NuPAGE® Novex 4-12% or 10% Bis-Tris Gel polyacrylamide gels and transferred to nitrocellulose membranes. Non-specific antibody binding was blocked with 50 mM Tris/150 mM NaCl/0.1% Tween-20 (TBS-T) with 5% nonfat milk for 30 min. The membranes were incubated with primary antibodies (HRP-conjugated anti-Flag (Cell Signaling, Cat #2044) in 5% BSA in TBS-T diluted 1:1000 overnight; anti-GAPDH (Cell Signaling, clone 14C10) in 5% milk in TBS-T diluted 1:5000 for 30 min; anti-ASCL1 (Santa Cruz, clone sc-48449) in 5% BSA diluted 1:500; or anti-g-globin (Santa Cruz, clone 51-7) in 5% milk diluted 1:500 and the membranes were washed with TBS-T for 30 min. Membranes labeled with primary antibodies were incubated with anti-rabbit HRP-conjugated antibody (Sigma-Aldrich) diluted 1:5000 for 30 min, anti-goat (1:3000) or anti-mouse (1:5000) and washed with TBS-T for 30 min. Membranes were visualized using the Immun-Star WesternC™ Chemiluminescence Kit (Bio-Rad) and images were captured using a ChemiDoc™ XRS+ System and processed using ImageLab software (Bio-Rad).

ELISA. Serum-free culture media (OPTI-MEM) was collected and frozen at −80° C. Human IL-1ra secretion into culture media was quantified via enzyme-linked immunosorbent assay (ELISA), according to the manufacturer's protocols (R&D Systems, Cat. No. DY280). The standard curve was prepared by diluting recombinant human IL-1ra in OPTI-MEM and the IL-1ra in culture media was measured undiluted. The samples were concentrated about 8 fold via centrifugation through 3 kDa MWCO filters for 20 min (Amicon Ultra, Cat #UFC500396). Reported values were corrected by the concentration factor for each sample.

Optical density was measured at 450 nm, with a wavelength correction at 540 nm. Each standard and sample was assayed in duplicate. The duplicate readings were averaged and normalized by subtracting the average zero standard optical density. A standard curve was generated by log-transforming the data and performing a linear regression of the IL-1ra concentration versus the optical density. Reported values are the mean and standard error of the mean from three independent experiments (n=3) that were performed on different days with technical duplicates that were averaged for each experiment.

qRT-PCR. Total RNA was isolated using the RNeasy Plus RNA isolation kit (Qiagen). cDNA synthesis was performed using the SuperScript® VILO™ CDNA Synthesis Kit (Invitrogen). Real-time PCR using PerfeCTa® SYBR® Green FastMix was performed with the CFX96 Real-Time PCR Detection System (Bio-Rad) with oligonucleotide primers reported in Table 3 that were designed using Primer3Plus software and purchased from IDT.

TABLE 3
Sequences of primers used for qRT-PCR.

| | | SEQ ID | | SEQ ID |
|---|---|---|---|---|
| Target | Forward Primer | NO | Reverse Primer | NO |
| HASCL1 | GGAGCTTCTCGACTTCACCA | 41 | AACGCCACTGACAAGAAAGC | 53 |
| NANOG | GATTTGTGGGCCTGAAGAAA | 42 | CAGATCCATGGAGGAAGGAA | 54 |
| VEGFA | AAGGAGGAGGGCAGAATCAT | 43 | GGGTACTCCTGGAAGATGTCC | 55 |
| TERT | AAACCTTCCTCAGCTATGCCC | 44 | GTTTGCGACGCATGTTCCTC | 56 |
| IL1B | AGCTGATGGCCCTAAACAGA | 45 | AAGCCCTTGCTGTAGTGGTG | 57 |
| IL1R2 | CAGGAGGACTCTGGCACCTA | 46 | CGGCAGGAAAGCATCTGTAT | 58 |
| IL1RN | GGAATCCATGGAGGGAAGAT | 47 | TGTTCTCGCTCCAGGTCAGTG | 59 |
| HBG1/2 | GCTGAGTGAACTGCACTGTGA | 48 | GAATTCTTTGCCGAAATGGA | 60 |
| MYOD1 | CTCTCTGCTCCTTTGCCACA | 49 | GTGCTCTTCGGGTTTCAGGA | 61 |
| GAPDH | CAATGACCCCTTCATTGACC | 50 | TTGATTTTGGAGGGATCTCG | 62 |
| mASCL1 | GGAACAAGAGCTGCTGGACT | 51 | GTTTTTCTGCCTCCCCCATTT | 63 |
| mGAPDH | AACTTTGGCATTGTGGAAGG | 52 | GGATGCAGGGATGATGTTCT | 64 |

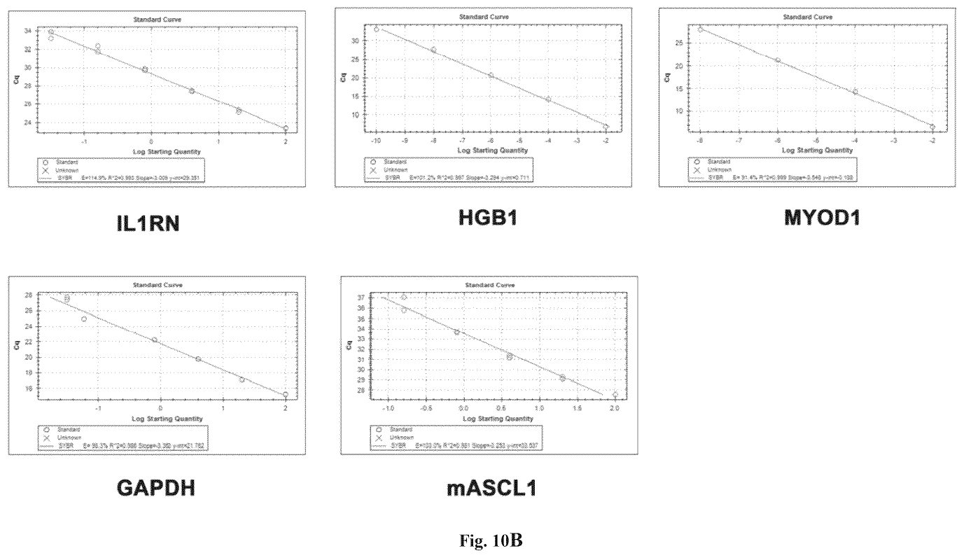

Primer specificity was confirmed by agarose gel electrophoresis and melting curve analysis. Reaction efficiencies over the appropriate dynamic range were calculated to ensure linearity of the standard curve (FIG. 10). The results are expressed as fold-increase mRNA expression of the gene of interest normalized to GAPDH expression by the ΔΔC_{T} method. Reported values are the mean and standard error of the mean from three independent experiments performed on different days (n=3) with technical duplicates that were averaged for each experiment.

RNA-Seq. RNA seq libraries were constructed. Briefly, first strand cDNA was synthesized from oligo dT Dynabead® (Invitrogen) captured mRNA using SuperScript® VILO™ cDNA Synthesis Kit (Invitrogen). Second strand cDNA was synthesized using DNA Polymerase I (New England Biolabs). cDNA was purified using Agencourt AMPure XP beads (Beckman Coulter) and Nextera transposase (Illumina; 5 min at 55° C.) was used to simultaneously fragment and insert sequencing primers into the double-stranded cDNA. Transposition reactions were halted using QG buffer (Qiagen) and fragmented cDNA was purified on AMPure XP beads. Indexed sequencing libraries were generated by 6 cycles of PCR.

Libraries were sequenced using 50-bp single end reads on two lanes of an Illumina HiSeq 2000 instrument, generating between 29 million and 74 million reads per library. Reads were aligned to human RefSeq transcripts using Bowtie (Langmeadet al. Genome biology 10, R25 (2009)). The statistical significance of differential expression, including correction for multiple hypothesis testing, was calculated using DESeq (Anders et al. Genome biology 11, R106 (2010)). Raw RNA-seq reads and the number of reads aligned to each RefSeq transcript have been deposited for public access in the Gene Expression Omnibus (GEO), with accession number currently pending.

Immunofluorescence staining. For detection of Tuj1 and MAP2 expression, transfected MEFs were fixed at day 10 of culture in N3 medium with 4% PFA (EMS, Hatfield, PA) at room temperature (RT) for 20 min. Cells were then incubated in blocking buffer containing 0.2% Triton X-100, 3% w/v BSA, and 10% goat serum (Sigma-Aldrich, Saint Louis, MO) for two hrs at room temperature with rabbit anti-Tuj1 (Covance, Princeton, New Jersey, clone TUJ1 Jan. 15, 1979, 1:500) and mouse anti-MAP2 (BD, clone Ap20, 1:500), or for an additional 24 hrs at 4° C. with mouse anti-Ascl1 (BD clone 24B72D11.1, 1:100). The cells were then washed three times with PBS, incubated for 1 hr at room temperature in blocking buffer with Alexa Fluor 488 goat anti-mouse IgG and Alexa Fluor 594 goat anti-rabbit IgG (Invitrogen, 1:200), and washed three times with PBS. Stained MEFs were then scanned with a Nikon Eclipse TE2000-U inverted fluorescence microscope with a ProScanII motorized stage (Prior Scientific, Rockland, MA) to produce large mosaic images of each complete culture area. These mosaics were processed with a FIJI macro to automatically and uniformly threshold each image according to local contrast, exclude small debris, and to count the number of Tuj1+ cells in each well.

Statistics. Statistical analysis was performed by Tukey's test with alpha equal to 0.05 in JMP 10 Pro.

### Example 2

### Results

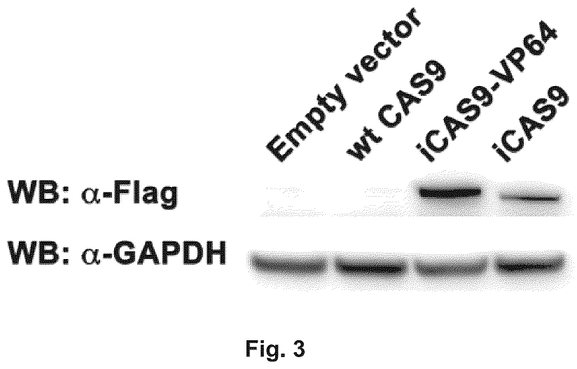

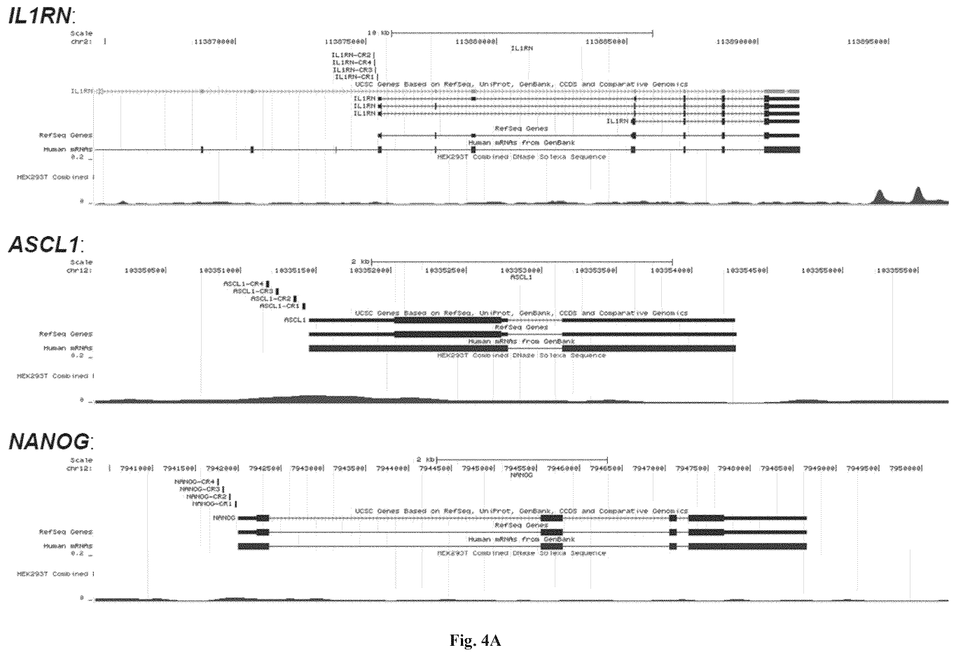

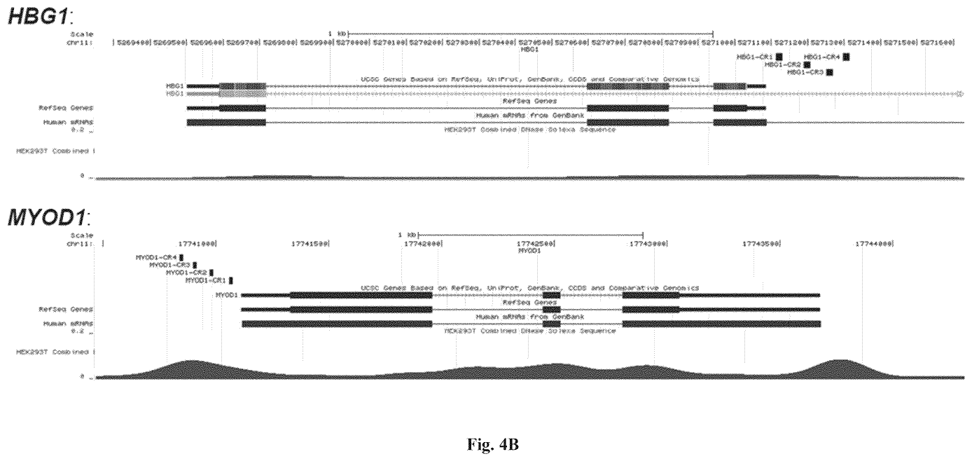

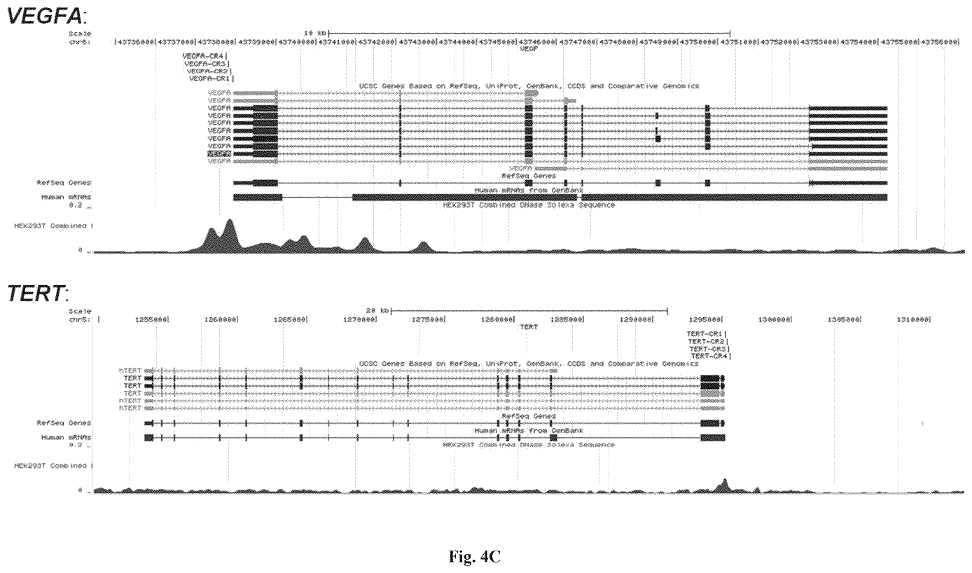

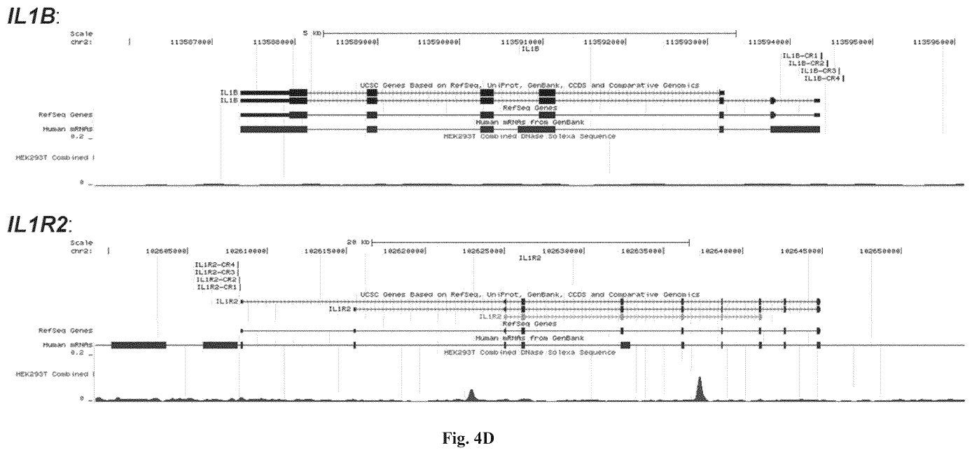

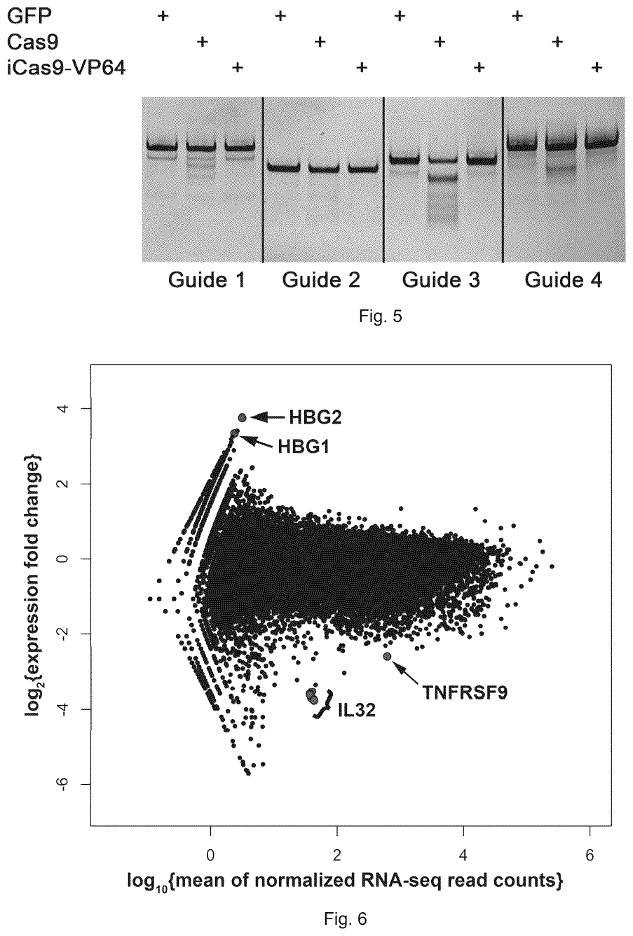

To create a CRISPR/Cas9-based transcriptional activation system, catalytic residues of Cas9 (D10A, H840A) were mutated to create iCas9 and genetically fused with a C-terminal VP64 acidic transactivation domain (FIGS. 1A and 1B). Robust expression of iCas9-VP64 was observed from the transfected plasmid in human embryonic kidney (HEK) 293T cells by western blot of the N-terminal Flag epitope tag (FIG. 3). The CRISPR system recognizes its target through base pairing of a 20 bp sequence in the gRNA to a complementary DNA target, which is followed by the NGG protospacer-adjacent motif (PAM) sequence, where N is any base pair. Combinations of synthetic transcription factors targeted to endogenous human promoters result in synergistic and robust activation of gene expression. Therefore four gRNA target sites followed by the NGG PAM sequence were identified in the promoter of the IL1RN gene within 500 bp of the transcriptional start site (FIG. 4, Table 2). To compare crRNA- and gRNA-based targeting strategies, the four target site sequences were introduced into crRNA and gRNA expression plasmids17 and co-transfected with the iCas9-VP64 expression plasmid into HEK293T cells. Although substantial induction of IL1RN expression was observed by qRT-PCR in samples treated with the combination of crRNAs, much higher levels were achieved with the combination of gRNAs (FIG. 1C). No changes to gene expression were observed in cells treated with gRNAs and an expression plasmid for iCas9 without VP64, demonstrating the critical role of the activation domain in modulating gene expression (FIG. 1C). Nuclease activity at these target sites was confirmed to have been abrogated in the iCas9-VP64 system by performing the Surveyor assay to detect DNA repair events in samples treated with iCas9-VP64 and wild-type Cas9 (FIG. 5). By transfecting each of the four gRNAs individually or in combination, targeting multiple sites in the promoter with combinations of gRNAs showed robust increases in gene expression (FIG. 1D). High levels of IL1RN expression were observed only when the gRNA combinations were co-transfected with iCas9-VP64 (FIG. 1D), as seen with other classes of engineered transcription factors. Similarly, production of the IL-1 receptor antagonist (IL-1ra) protein, encoded by the IL1RN gene, was only observed in three of the six samples treated with the combination of gRNAs across three different experiments, whereas it was never detected in samples treated with single gRNAs or control plasmid (FIG. 1E). To examine the specificity of gene activation by iCas9-VP64, global gene expression of HEK293T cells treated with the combination of four gRNAs by RNA-seq was assessed (FIG. 1F). Notably, the only genes with significantly increased expression relative to control (false discovery rate≤3×10⁻⁴) were the four isoforms expressed from the IL1RN locus (FIG. 4), indicating a high level of specificity of gene activation.

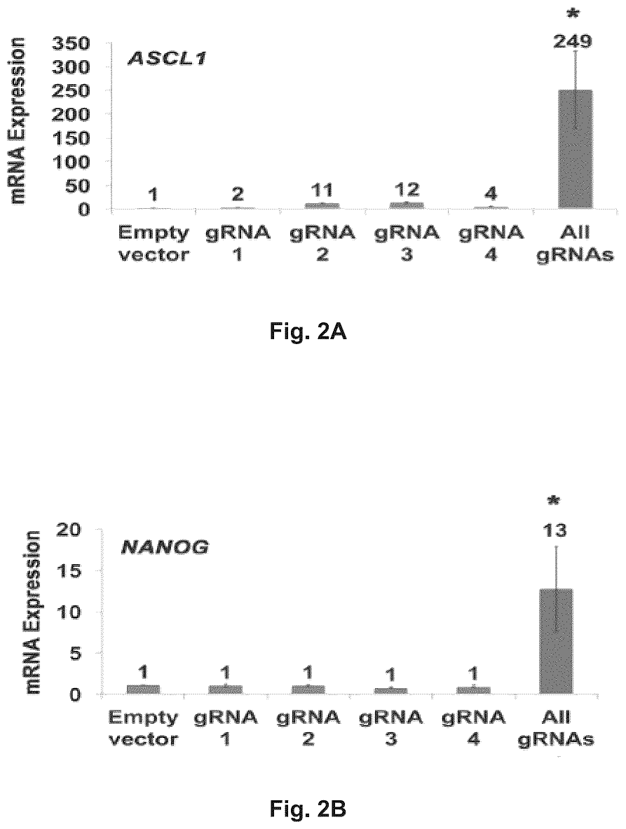

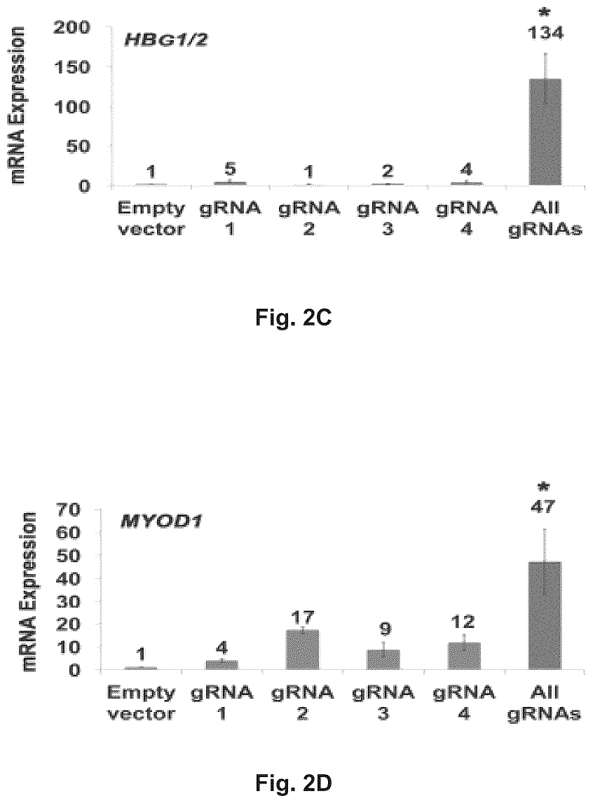

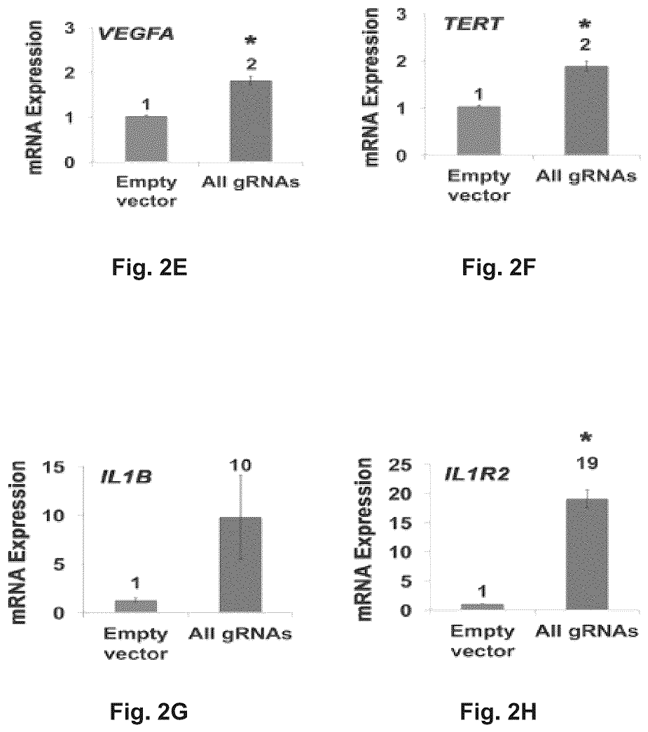

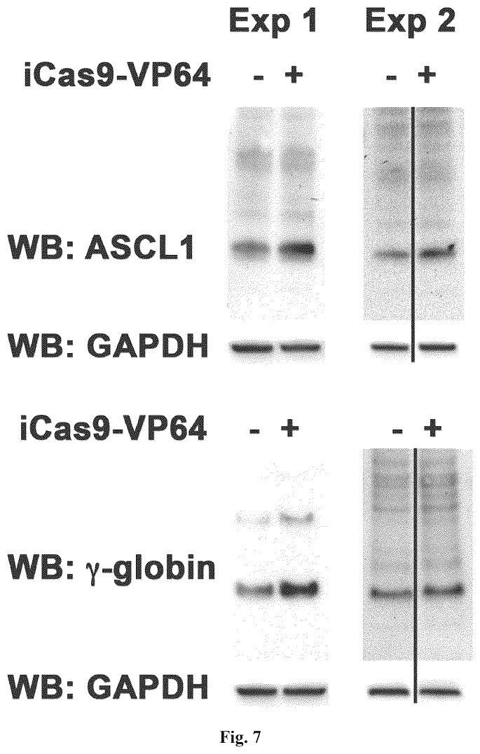

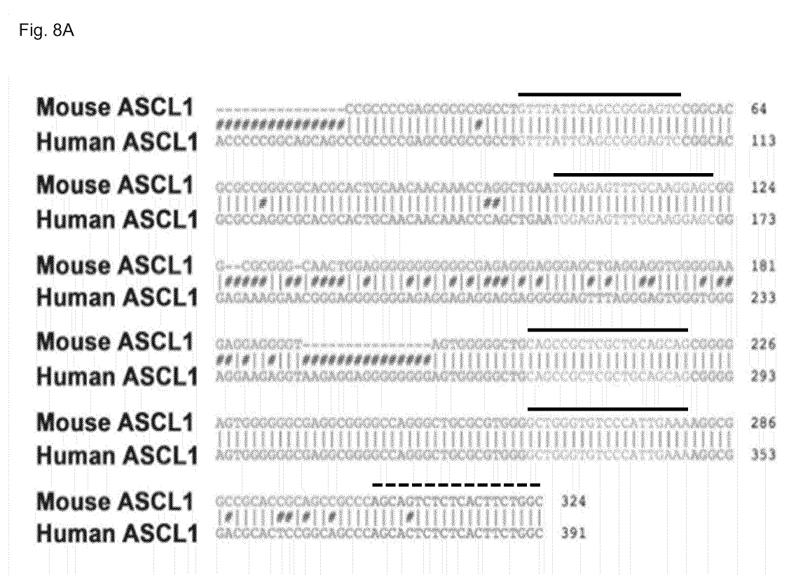

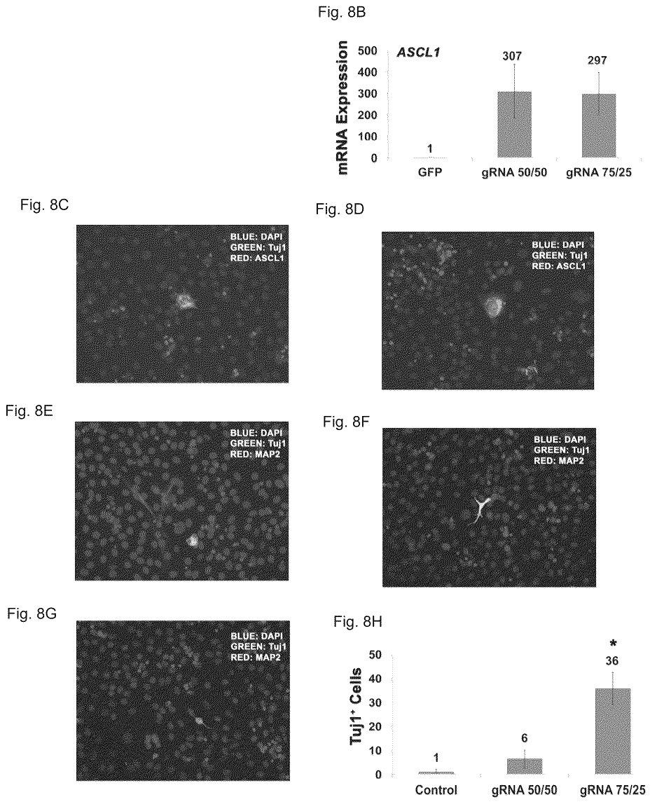

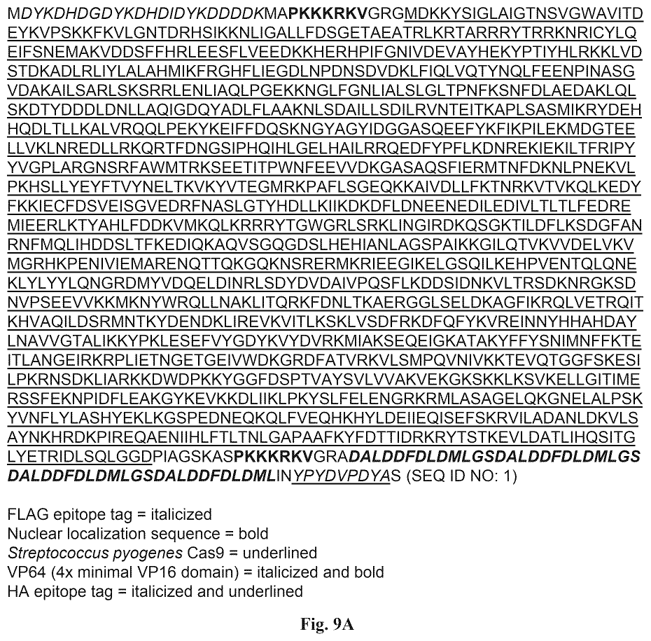

To demonstrate the general applicability of this system, four gRNAs were designed to target each of the promoters of eight other genes relevant to medicine and biotechnology, including ASCL1, NANOG, HBG1/2, MYOD1, VEGFA, TERT, IL1B, and IL1R2 (FIG. 4, Table 2). Forced expression of ASCL1 and MYOD1 leads to transdifferentiation of several cell types into neuronal and myogenic phenotypes, respectively. NANOG is a marker of pluripotency and that is also used in genetic reprogramming strategies. Activation of the homologs HBG1 and HBG2, which encode γ-globin during fetal development, can be used as a therapeutic strategy to compensate for β-globin mutations in sickle cell disease. Up-regulation of VEGFA by synthetic transcription factors has been explored as a strategy to enhance tissue regeneration and wound healing. The forced expression of telomerase, encoded by the TERT gene, can be used to immortalize cell lines. IL1B encodes the IL-1β cytokine that mediates inflammation and autoimmunity. IL-1β signaling can be blocked by expression of IL-1ra or the decoy receptor encoded by IL1R2. Expression of each of these genes was enhanced by co-transfection of expression plasmids for iCas9-VP64 and the four gRNAs into HEK293T cells, as determined by qRT-PCR (FIGS. 2A-2H). In some cases expression of a single gRNA was sufficient to induce gene expression, but in all cases co-transfection of the four gRNAs led to synergistic effects (FIGS. 2A-2D). Notably, chromatin accessibility, as determined by DNase-seq, was not a predictor of successful gene activation (FIG. 4). RNA-seq was performed on cells transfected with iCas9-VP64 and the four gRNAs targeting HBG1, three of which also target HBG2. This revealed specific and reproducible increases in expression of both HBG1 and HBG2, which cannot be distinguished by RNA-seq, although statistical significance was not achieved due to low total expression levels (FIG. 6). Increases in protein expression of Ascl1 and γ-globin following treatment with iCas9-VP64 and the four gRNAs were detected by western blot (FIG. 7), corroborating higher mRNA levels observed by qRT-PCR (FIGS. 2A-2H). Low baseline levels of Ascl1 and γ-globin protein expression were detectable in empty vector controls. As preliminary evidence that the activation of gene targets by iCas9-VP64 can lead to secondary changes in gene networks and cell phenotypes, expression plasmids for iCas9-VP64 and the four gRNAs targeting ASCL1 were co-transfected into murine embryonic fibroblasts (MEFs) (FIGS. 8A-8H). Forced expression of Ascl1 in MEFs has been shown to partially activate the neuronal gene network, including the downstream target Tuj1. Because the gRNA target sites are conserved in the human and mouse ASCL1 promoters (FIG. 8A), activation of ASCL1 expression was also observed in MEFs treated with plasmids encoding iCas9-VP64 and the four gRNAs (FIG. 8B). Furthermore, cells expressing Ascl1 and the neuronal marker Tuj1 were readily detected by immunofluorescence staining 12 days after transfection in the iCas9-VP64/gRNA-treated samples (FIGS. 8C-8H). No Tuj1-positive cells were observed in the cells treated with the control plasmid.

Thus far there has not been any comprehensive survey of the specificity of Cas9/CRISPR activity in mammalian cells. Using RNA-seq, targeted gene activation was shown to be exquisitely specific with no detectable off-target gene activation (FIG. 1F, FIG. 6). IL1RN and HBG1/2 were chosen for this specificity analysis as the gene products, IL-1ra and γ-globin, may not generate secondary effects on gene expression in HEK293T cells. Exploiting the synergistic activity of multiple weak transcriptional activators, in contrast to using a single strong activator, may increase specific gene regulation since it is unlikely that multiple adjacent off-target sites would exist at another locus. Interestingly, the IL32 gene was moderately downregulated (false discovery rate<0.03) in both the samples treated with iCas9-VP64 and either the IL1RN- or HBG1/2-targeted gRNAs compared to control samples treated with only an empty expression plasmid (FIG. 1F, FIG. 6). Because both the IL1RN and HBG1/2-targeted samples were similarly affected, it is unlikely that this is the result of off-target iCas9-VP64 activity related to the identity of the target sequences.

To evaluate the specificity with which iCas9-VP64 binds the genome, ChIP sequencing was performed using an anti-HA antibody on cells treated with iCas9-VP64 and four gRNAs targeting the IL1RN promoter. The experiment revealed that iCas9 targets the IL1RN promoter (FIG. 15). Moreover, the experiment revealed an extremely high level of specificity. The iCas9 had only 10 potential off-target binding sites (FDR<5%). To further query the specificity, RNA sequencing experiments were performed with iCas9 EGEMs and found that only IL1RN gene isoforms increased in expression relative to control (FDR≤3×10.4).

### Example 3

### CRISPRs Targeting the Dystrophin Gene—Methods and Materials

Plasmid constructs. The expression cassettes for the S. pyogenes sgRNA and human codon optimized Cas9 (hCas9) nuclease were used, as previously described (Perez-Pinera et al., Nat Methods 10:973-976 (2013)). In order to create a fluorescent reporter system to enrich CRISPR/Cas9-modified cells, a GeneBlock (IDT) was synthesized containing a portion of the 3′ end of the Cas9 coding sequence fused to a T2A skipping peptide immediately upstream of a multiple cloning site and subsequently cloned into the hCas9 expression vector. An eGFP reporter gene was then cloned into the T2A vector to allow co-translation of Cas9 and eGFP proteins from the same expression vector (hCas9-T2A-GFP, SEQ ID NO: 116).

Cell culture and transfection. HEK293T cells were obtained from the American Tissue Collection Center (ATCC) through the Duke Cell Culture Facility and were maintained in DMEM supplemented with 10% bovine calf serum and 1% penicillin/streptomycin. Immortalized myoblasts (Mamchaoui, K. et al. Skelet Muscle 1, 1-11 (2011)) (one from a wild-type donor, and two Δ48-50 DMD patient derived lines) were maintained in skeletal muscle media (PromoCell) supplemented with 20% bovine calf serum (Sigma), 50 μg/ml fetuin, 10 ng/ml human epidermal growth factor (Sigma), 1 ng/ml human basic fibroblast growth factor (Sigma), 10 μg/ml human insulin (Sigma), 1% GlutaMAX (Invitrogen), and 1% penicillin/streptomycin (Invitrogen). Primary DMD dermal fibroblasts were obtained from the Coriell Cell repository (GM05162A, Δ46-50) and maintained in DMEM supplemented with 10% fetal bovine serum, 1 ng/mL human basic fibroblast growth factor, and 1% penicillin/streptomycin. All cell lines were maintained at 37° C. and 5% CO₂.

HEK293T cells were transfected with Lipofectamine 2000 (Invitrogen) with 400 ng of each expression vector according to the manufacturer's protocol in 24 well plates. Immortalized myoblasts and primary fibroblasts were transfected with 5 μg of each expression vector by electroporation using the Gene Pulser XCell (BioRad) with PBS as an electroporation buffer using optimized conditions for each line (FIGS. 1A-1F) (Ousterout et al. Mol Ther 21:1718-1726 (2013)). Transfection efficiencies were measured by delivering an eGFP expression plasmid (pmaxGFP, Clontech) and using flow cytometry. These efficiencies were routinely ≥95% for HEK293T and ≥70% for the primary fibroblasts and immortalized myoblasts. For all experiments, the indicated mass of electroporated plasmid corresponds to the amount used for each CRISPR/Cas9-based system.

### Cel-I Quantification of Endogenous Gene Modification (Surveyor Assay).

CRISPR/Cas9-based system-induced lesions at the endogenous target site were quantified using the Surveyor nuclease assay (Guschin, D. Y. et al. Meth Mol Biol 649, 247-256 (2010)), which can detect mutations characteristic of nuclease-mediated NHEJ. After transfection, cells were incubated for 3 or 10 days at 37° C. and genomic DNA was extracted using the DNeasy Blood and Tissue kit (QIAGEN). The target locus was amplified by 35 cycles of PCR with the AccuPrime High Fidelity PCR kit (Invitrogen) using primers specific to each locus (see Table 4), such as 5′-GAGTTTGGCTCAAATTGTTACTCTT-3′ (SEQ ID NO: 626) and 5′-GGGAAATGGTCTAGGAGAGTAAAGT-3′ (SEQ ID NO: 627).

TABLE 4
Summary of top 10 off target sites predicted in silico and activity at each site
as detected by the Surveyor assay in HEK293T cells transfected with Cas9 and the
indicated sgRNA expression cassettes. n.d.: not detected.
SEQ

| ID | | | | | | | | Intron/ | | % |
|---|---|---|---|---|---|---|---|---|---|---|
| NO. | Guide | Target | Sequence | PAM | Score | Chr | Gene | Exon | #MMs | indels |
| 67 | CR3 | Guide | GCCTACTCAGACTGTTACTC | — | — | — | — | — | — | — |
| 150 | | Target | tCCTACTCAGACTGTTACTC | TGG | — | X | DMD | Exon | 1 | 13.0 |
| 151 | | OT1 | tCCTACTCAcACTGTTACTC | AGG | 7.4 | 1 | STRIP1 | Intron | 2 | 9.3 |
| 152 | | OT2 | aCCTgCTCAcACTGTTACTC | CAG | 2.5 | 2 | ARHGAP25 | Intron | 3 | n.d. |
| 153 | | OT3 | GCaTtCTCAaACTGTTACTC | AGG | 2.4 | 13 | None | None | 3 | n.d. |
| 154 | | OT4 | GgaTtCTCAcACTGTTACTC | GGG | 1.3 | 14 | PGPEP1 | Exon | 4 | n.d. |
| 155 | | OT5 | aCaTACTtAtACTGTTACTC | TAG | 1.3 | 19 | MDGA2 | Intron | 4 | n.d. |
| 156 | | OT6 | tatTcCTaAGACTGTTACTC | AAG | 0.9 | 8 | LPPR1 | Intron | 5 | n.d. |
| 157 | | OT7 | aaggACTaAGACTGTTACTC | GGG | 0.9 | 9 | RNF122 | Intron | 5 | n.d. |
| 158 | | OT8 | GagctCTCAtACTGTTACTC | TAG | 0.8 | 3 | DNMBP | Exon | 5 | n.d. |
| 159 | | OT9 | GCaaAaTgAGACTGTTACTC | CAG | 0.8 | 5 | SLC12A2 | Intron | 4 | n.d. |
| 160 | | OT10 | cCtcAtTCAGACTGTTACTC | AAG | 0.8 | 4 | KCNIP4 | Intron | 4 | n.d. |
| 65 | CR1 | Guide | GATTGGCTTTGATTTCCCTA | — | — | — | — | — | — | — |
| 161 | | Target | cATTGGCTTTGATTTCCCTA | GGG | — | X | DMD | Intron | 1 | 8.3 |
| 162 | | OT1 | aATTGGCATTGATTTCCCTA | GAG | 7.1 | 16 | None | None | 2 | 0.8 |
| 163 | | OT2 | cATTGGCTTTaATTTCCCTA | TAG | 4.8 | 4 | None | None | 2 | n.d. |
| 164 | | OT3 | GATaGGCTgTGATTTCCCTA | GAG | 3.9 | 9 | None | None | 2 | n.d. |
| 165 | | OT4 | GAaTaGCcTTGATTTCCCTA | AAG | 2.4 | 1 | None | None | 3 | n.d. |
| 166 | | OT5 | aATTtGCTTTGATTTCCCTg | AGG | 1.5 | 1 | TIMM17A | Intron | 3 | n.d. |
| 167 | | OT6 | GATgtGCTTTGATTTCCCTt | GGG | 1.4 | 17 | MYO1D | Intron | 3 | n.d. |
| 168 | | OT7 | aATTGGtTTTaATTTCCCTA | AAG | 1.1 | 8 | PIK1A | Intron | 3 | n.d. |
| 169 | | OT8 | aATTGGgTTTGATTTCCCTt | TGG | 1.1 | 11 | MS4A1 | Intron | 3 | n.d. |
| 170 | | OT9 | GATgGGtTTTtATTTCCCTA | GAG | 1.0 | 11 | None | None | 3 | n.d. |
| 171 | | OT10 | GAaTGGtTTTGATTTCCCTg | GAG | 1.0 | 11 | None | None | 3 | n.d. |
| 69 | CR5 | Guide | GCAGTTGCCTAAGAACTGGT | — | — | — | — | — | — | — |
| 172 | | Target | aCAGTTGCCTAAGAACTGGT | GGG | — | X | DMD | Intron | 1 | 14.0 |
| 173 | | OT1 | cCAGTTGtCTAAGAACTGGg | GAG | 1.5 | 5 | NRG1 | Intron | 3 | n.d. |
| 174 | | OT2 | GCAGTTGCCTgtGAACTGGT | AGG | 1.4 | X | None | None | 2 | n.d. |
| 175 | | OT3 | GCAGaTGCagAAGAACTGGT | GAG | 1.4 | 19 | SMIM7 | Intron | 3 | n.d. |
| 176 | | OT4 | GCAGTTcCagAAGAACTGGT | GAG | 0.9 | 11 | GLB1L2 | Intron | 3 | n.d. |
| 177 | | OT5 | caAcTTGCCTAtGAACTGGT | AGG | 0.7 | 8 | ASAP1 | Intron | 4 | n.d. |
| 178 | | OT6 | aCAccTGCCTAAGAACTGGa | GGG | 0.7 | 11 | None | None | 4 | n.d. |
| 179 | | OT7 | tCAGgTGgCTAAGAACTGGg | TGG | 0.7 | 14 | NIN | Intron | 4 | n.d. |
| 180 | | OT8 | GaAGTTGgCcAAGAACTGGa | GAG | 0.6 | 7 | None | None | 4 | n.d. |
| 181 | | OT9 | GCtGcTGCCcAAGAACTGGc | AGG | 0.6 | 11 | AMOTL1 | Intron | 4 | n.d. |
| 182 | | OT10 | tCAGcTGgCTAAGAACgGGT | AAG | 0.6 | 7 | ACTR3C | Intron | 4 | n.d. |
| 70 | CR6 | Guide | GGGGCTCCACCCTCACGAGT | — | — | — | — | — | — | — |
| 183 | | Target | aGGGCTCCACCCTCACGAGT | GGG | — | X | DMD | Intron | 1 | 19.9 |
| 184 | | OT1 | GcaGCTCagCCCTCACGAGT | CAG | 0.8 | 3 | None | None | 4 | n.d. |
| 185 | | OT2 | GGGGCTtCAgCaTCACGAGT | GAG | 0.8 | 8 | None | None | 3 | n.d. |
| 186 | | OT3 | GGGGCTCtcCCCTCACtAGT | GAG | 0.6 | 8 | None | None | 3 | n.d. |
| 187 | | OT4 | GGGGaTCCACCtTCACcAGT | CAG | 0.6 | 2 | None | None | 3 | n.d. |
| 188 | | OT5 | aGGGCTggACCCTCACaAGT | AAG | 0.4 | 16 | AXIN1 | Intron | 4 | n.d. |
| 189 | | OT6 | tGGtCTCCtCCCcCACGAGT | GGG | 0.4 | 2 | None | None | 4 | n.d. |
| 190 | | OT7 | aGGGCTCCcaCCcCACGAGT | GAG | 0.3 | 5 | None | None | 4 | n.d. |
| 191 | | OT8 | GaGGCTCCAtaCTCACcAGT | GAG | 0.3 | 11 | None | None | 4 | n.d. |
| 192 | | OT9 | GGaGCTgCcCCtTCACGAGT | GGG | 0.3 | 3 | None | None | 4 | n.d. |
| 193 | | OT10 | atGaCTCCACCCTCAaGAGT | AAG | 0.3 | 8 | AGPAT5 | None | 4 | n.d. |
| 100 | CR36 | Guide | GCCTTCTTTATCCCCTATCG | — | — | — | — | — | — | — |
| 194 | | Target | GCCTTCTTTATCCCCTATCG | AGG | — | X | DMD | Intron | 0 | 20.6 |
| 195 | | OT1 | GtCTgCTgTgTCCCCTATCG | GGG | 1.3 | 21 | None | None | 4 | n.d. |
| 196 | | OT2 | cCCTTCTcTATCCCCTgTCG | TGG | 1.3 | 8 | None | None | 3 | n.d. |
| 197 | | OT3 | GCCTTCTTTATCCCCTcTCt | TGG | 0.9 | 10 | None | None | 2 | 0.5 |
| 198 | | OT4 | GCgcTCTTTtTCCCCTATCt | TAG | 0.6 | 16 | None | None | 4 | n.d. |
| 199 | | OT5 | GCCcTCTgTcTCCCCTgTCG | CAG | 0.5 | 1 | NFASC | None | 4 | n.d. |
| 200 | | OT6 | tCCATCTtTgTCCCCTATtG | AGG | 0.5 | 10 | None | None | 4 | n.d. |
| 201 | | OT7 | aCCtTCTCTcTCCCCTATaG | AGG | 0.5 | 5 | LOC100996485 | Intron | 4 | n.d. |
| 202 | | OT8 | GttTTCTTTtTCCCCTATgG | GAG | 0.5 | 3 | None | None | 4 | n.d. |
| 203 | | OT9 | tgCTTCTTaATCCCCTATCa | AAG | 0.4 | 7 | None | None | 4 | n.d. |
| 204 | | OT10 | aCCTTCTTacTCCCCTATCc | GGG | 0.4 | 10 | ADARB2 | None | 4 | n.d. |

The resulting PCR products were randomly melted and reannealed in a thermal cycler with the program: 95° C. for 240 s, followed by 85° C. for 60 s, 75° C. for 60 s, 65° C. for 60 s, 55° C. for 60 s, 45° C. for 60 s, 35° C. for 60 s, and 25° C. for 60 s with a −0.3° C./s rate between steps. Following reannealing, 8 μL of PCR product was mixed with 1 μL of Surveyor Nuclease S and 1 μL of Enhancer S (Transgenomic) and incubated at 42° C. for 1 hr. After incubation, 6 μL of digestion product was loaded onto a 10% TBE polyacrylamide gel and run at 200V for 30 min. The gels were stained with ethidium bromide and quantified using ImageLab (Bio-Rad) by densitometry as previously described (Guschin, et al. Meth Mol Biol 649, 247-256 (2010)).

Fluorescence-activated cell sorting of myoblasts. DMD myoblasts were electroporated with 5 micrograms each of hCas9-T2A-GFP and sgRNA expression vectors and incubated at 37° C. and 5% CO₂. Three days after electroporation, cells were trypsinized and collected for FACS sorting using a FACSvantage II sorting machine. GFP-positive cells were collected and grown for analysis.

PCR-based assay to detect genomic deletions. The exon 51 or exon 45-55 loci were amplified from genomic DNA by PCR (Invitrogen AccuPrime High Fidelity PCR kit) using primers flanking each locus. The flanking primers were CelI-CR1/2-F and CelI-CR5-R for exon 51 or CelI-CR6-F and CelI-CR36-R for exon 45-55 analysis (Table 4). PCR products were separated on TAE-agarose gels and stained with ethidium bromide for analysis.

PCR-based detection of translocations. Loci with predicted possible translocations were amplified by a two-step nested PCR (Invitrogen AccuPrime High Fidelity PCR kit for each step) of genomic DNA from cells transfected with Cas9 alone (control) or Cas9 with sgRNA. In the first step, translocations that may occur at each on-target and off-target sgRNA target site were amplified by 35 cycles of PCR using combinations of Surveyor primers for each locus that were modified to include restriction sites to facilitate cloning and sequencing analysis (Table 4). One microliter of each PCR reaction was subjected to a second round of amplification by 35 rounds of PCR using nested primer sets custom designed for each individual predicted translocation (Table 4). Each second nested PCR primer binds within the same approximate region within the primary amplicon; however, each pair was optimized using Primer3 online bioinformatics software to ensure specific detection of each translocation. PCR amplicons corresponding to the expected length of predicted translocations and only present in cells treated with sgRNA were purified (QIAGEN Gel Extraction kit) and analyzed by Sanger sequencing.

mRNA analysis. Immortalized myoblasts were differentiated into myofibers by replacing the growth medium with DMEM supplemented with 1% insulin-transferrin-selenium (Invitrogen #51500056) and 1% penicillin/streptomycin (Invitrogen #15140) for 5 days before the cells were trypsinized and collected. Total RNA was isolated from these cells using the RNeasy Plus Mini Kit (QIAGEN) according to the manufacturer's instructions. RNA was reverse transcribed to cDNA using the VILO cDNA synthesis kit (Life Technologies #11754) and 1.5 micrograms of RNA for 2 hrs at 42° C. according to the manufacturer's instructions. The target loci were amplified by 35 cycles of PCR with the AccuPrime High Fidelity PCR kit (Invitrogen) using primers annealing to exons 44 and 52 to detect exon 51 deletion by CR1/5 or CR2/5 or primers annealing to exons 44 and 60 to detect exon 45-55 deletion by CR6/36 (Table 4). PCR products were run on TAE-agarose gels and stained with ethidium bromide for analysis. The resolved PCR bands were cloned and analyzed by Sanger sequencing to verify the expected exon junctions. Table 5 lists the sequences of primers used in Example 4.

TABLE 5

| SEQ | | | |
|---|---|---|---|
| ID | | | |
| NO. | Primer name | Primer sequence | Notes |
| 205 | CelI-CR1/2-F | GAGAGGTTATGTGGCTTTACCA | Forward Surveyor primer for CR1/2 |
| 206 | CelI-CR1-R | AAAAATGCTTCCCACTTTGC | Reverse Surveyor primer for CR1 |
| 207 | CelI-CR2-R | CTCATTCTCATGCCTGGACA | Reverse Surveyor primer for CR2 |
| 208 | CelI-CR3-F | GAGTTTGGCTCAAATTGTTACTCTT | Forward Surveyor primer for CR3 |
| 209 | CelI-CR3-R | GGGAAATGGTCTAGGAGAGTAAAG | Reverse Surveyor primer for CR3 |
| | | T | |
| 210 | CelI-CR4/31-F | GTTTGGCTCAAATTGTTACTCTTCA | Forward Surveyor primer for CR4 or |
| | | | CR31 |
| 211 | CelI-CR4/31-R | GTGAGAGTAATGTGTTTGCTGAGAG | Reverse Surveyor primer for CR4 or |
| | | | CR31 |
| 212 | CelI-CRS-F | CGGGCTTGGACAGAACTTAC | Forward Surveyor primer for CR5 |
| 213 | CelI-CR5-R | CTGCGTAGTGCCAAAACAAA | Reverse Surveyor primer for CR5 |
| 214 | CelI-CR6-F | TAATTTCATTGAAGAGTGGCTGAA | Forward Surveyor primer for CR6 |
| 215 | CelI-CR6-R | AAGCCCTGTGTGGTAGTAGTCAGT | Reverse Surveyor primer for CR6 |
| 216 | CelI-CR7-F | TGAGTCATGTTGGATAACCAGTCT | Forward Surveyor primer for CR7 |
| 217 | CelI-CR7-R | GAAGGTCAGGAACATACAATTCAA | Reverse Surveyor primer for CR7 |
| 218 | CelI-CR10/11-F | GATATGGGCATGTCAGTTTCATAG | Forward Surveyor primer for CR10 or |
| | | | CR11 |
| 219 | CelI-CR10/11-R | TGCTGTTGATTAATGGTTGATAGG | Reverse Surveyor primer for CR10 or |
| | | | CR11 |
| 220 | CelI-CR12/13-F | TTTTAAATTGCCATGTTTGTGTC | Forward Surveyor primer for CR12 or |
| | | | CR13 |
| 221 | CelI-CR12/13-R | ATGAATAACCTAATGGGCAGAAAA | Reverse Surveyor primer for CR12 or |
| | | | CR13 |
| 222 | CelI-CR14/15-F | TCAAGTCGCTTCATTTTGATAGAC | Forward Surveyor primer for CR14 or |
| | | | CR15 |
| 223 | CelI-CR14/15-R | CACAACAAAACATATAGCCAAAGC | Reverse Surveyor primer for CR14 or |
| | | | CR15 |
| 224 | CelI-CR16/17-F | TGCTGCTAAAATAACACAAATCAGT | Forward Surveyor primer for CR16 or |
| | | | CR17 |
| 225 | CelI-CR16/17-R | CTGTGCCTATTGTGGTTATCCTG | Reverse Surveyor primer for CR16 or |
| | | | CR17 |
| 226 | CelI-CR18/19-F | ATTGATCTGCAATACATGTGGAGT | Forward Surveyor primer for CR18 or |
| | | | CR19 |
| 227 | CelI-CR18/19-R | TTTGCCTCTGCTATTACAGTATGG | Reverse Surveyor primer for CR18 or |
| | | | CR19 |
| 228 | CelI-CR20/21-F | TGTAGGGTGGTTGGCTAAAATAAT | Forward Surveyor primer for CR20 or |
| | | | CR21 |
| 229 | CelI-CR20/21-R | TTTTTGCACAGTCAATAACACAAA | Reverse Surveyor primer for CR20 or |
| | | | CR21 |
| 230 | CelI-CR22/23-F | GGCTGGTCTCACAATTGTACTTTA | Forward Surveyor primer for CR22 or |
| | | | CR23 |
| 231 | CelI-CR22/23-R | CATTATGGACTGAAAATCTCAGCA | Reverse Surveyor primer for CR22 or |
| | | | CR23 |
| 232 | CelI-CR24/25-F | ATCATCCTAGCCATAACACAATGA | Forward Surveyor primer for CR24 or |
| | | | CR25 |
| 233 | CelI-CR24/25-R | TTCAGCTTTAACGTGATTTTCTGT | Reverse Surveyor primer for CR24 or |
| | | | CR25 |
| 234 | CelI-CR26/27-F | GGATTCAGAAGCTGTTTACGAAGT | Forward Surveyor primer for CR26 or |
| | | | CR27 |
| 235 | CelI-CR26/27-R | TTTAGCTGGATTGGAAAAACAAAT | Reverse Surveyor primer for CR26 or |
| | | | CR27 |
| 236 | CelI-CR28/29-F | AACTCACCCCATTGTTGGTATATT | Forward Surveyor primer for CR28 or |
| | | | CR29 |
| 237 | CelI-CR28/29-R | CCTTGTCCAAATACCGAAATACAT | Reverse Surveyor primer for CR28 or |
| | | | CR29 |
| 238 | CelI-CR33-F | CACATAATTCATGAACTTGGCTTC | Forward Surveyor primer for CR33 |
| 239 | CelI-CR33-R | TAGTAGCTGGGGAGGAAGATACAG | Reverse Surveyor primer for CR33 |
| 240 | CelI-CR34-F | TTTTTGTTTTAATTGCGACTGTGT | Forward Surveyor primer for CR34 |
| 241 | CelI-CR34-R | AGAAAAGGGGTTTTCTTTTGACTT | Reverse Surveyor primer for CR34 |
| 242 | CelI-CR35-F | CATTGTGACTGGATGAGAAGAAAC | Forward Surveyor primer for CR35 |
| 243 | CelI-CR35-R | AACGGCTGTTATTAAAGTCCTCAG | Reverse Surveyor primer for CR35 |
| 244 | CelI-CR36-F | CAAGTCAGAAGTCACTTGCTTTGT | Forward Surveyor primer for CR36 |
| 245 | CelI-CR36-R | TTTTATGTGCAGGAATCAGTCTGT | Reverse Surveyor primer for CR36 |
| 246 | Dys-E44-F | TGGCGGCGTTTTCATTAT | Forward RT-PCR primer binding in |
| | | | exon 44 |
| 247 | Dys-E52-R | TTCGATCCGTAATGATTGTTCTAGCC | Reverse RT-PCR primer binding in |
| | | | exon 52 |
| 248 | Dys-E60-R | GGTCTTCCAGAGTGCTGAGG | Reverse RT-PCR primer binding in |
| | | | exon 60 |
| 249 | CR3-CelI-OT1-F | TGTGTGCTTCTGTACACATCATCT | Forward Surveyor primer for CR3 off- |
| | | | target 1 |
| 250 | CR3-CelI-OT1-R | AGATTTCAACCCTCAAAAACTGAG | Reverse Surveyor primer for CR3 off- |
| | | | target 1 |
| 251 | CR3-CelI-OT2-F | TAAACTCTTTCTTTTCCGCAATTC | Forward Surveyor primer for CR3 off- |
| | | | target 2 |
| 252 | CR3-CelI-OT2-R | CAAGGTGACCTGCTACCTAAAAAT | Reverse Surveyor primer for CR3 off- |
| | | | target 2 |
| 253 | CR3-CelI-OT3-F | TATGACCAAGGCTATGTGTTCACT | Forward Surveyor primer for CR3 off- |
| | | | target 3 |
| 254 | CR3-CelI-OT3-R | ACAGCCTCTCTCCAGTAACATTCT | Reverse Surveyor primer for CR3 off- |
| | | | target 3 |
| 255 | CR3-CelI-OT4-F | TATTCTTGCAGTGGTTTCACATTT | Forward Surveyor primer for CR3 off- |
| | | | target 4 |
| 256 | CR3-CelI-OT4-R | ATATTTTAAGCCAAGACCCAACAA | Reverse Surveyor primer for CR3 off- |
| | | | target 4 |
| 257 | CR3-CelI-OT5-F | CTTTCAACTGTCTGTCTGATTGCT | Forward Surveyor primer for CR3 off- |
| | | | target 5 |
| 258 | CR3-CelI-OT5-R | AACAGCCTCTCTTCATTGTTCTCT | Reverse Surveyor primer for CR3 off- |
| | | | target 5 |
| 259 | CR3-CelI-OT6-F | CTCTGGAACTTGTCTCTGTCTTGA | Forward Surveyor primer for CR3 off- |
| | | | target 6 |
| 260 | CR3-CelI-OT6-R | CTTTCCTGCGTTCTCATGTTACTA | Reverse Surveyor primer for CR3 off- |
| | | | target 6 |
| 261 | CR3-CelI-OT7-F | CCTTATATCCGTATCGCTCACTCT | Forward Surveyor primer for CR3 off- |
| | | | target 7 |
| 262 | CR3-CelI-OT7-R | CATATCTGTCTAACTTCCGCACAC | Reverse Surveyor primer for CR3 off- |
| | | | target 7 |
| 263 | CR3-CelI-OT8-F | ACAGGTGTTATGTTGTCTGCATCT | Forward Surveyor primer for CR3 off- |
| | | | target 8 |
| 264 | CR3-CelI-OT8-R | ACTCCATTCCCAGATTAGTTATGC | Reverse Surveyor primer for CR3 off- |
| | | | target 8 |
| 265 | CR3-CelI-OT9-F | CTGTTTTCTTTGTGAGAGTGGAGA | Forward Surveyor primer for CR3 off- |
| | | | target 9 |
| 266 | CR3-CelI-OT9-R | TGTAAGGTGGTCAAACTTGCTCTA | Reverse Surveyor primer for CR3 off- |
| | | | target 9 |
| 267 | CR3-CelI-OT10-F | TTTTTCCTAGTACCCACAGATTTTT | Forward Surveyor primer for CR3 off- |
| | | | target 10 |
| 268 | CR3-CelI-OT10-R | TCCCTGATTCTCTCATTTGTGTTA | Reverse Surveyor primer for CR3 off- |
| | | | target 10 |
| 269 | CR1-CelI-OT1-F | TTGGGAACATCAGAGAAAGTATGA | Forward Surveyor primer for CR1 off- |
| | | | target 1 |
| 270 | CR1-CelI-OT1-R | ACAAATTACAGTCTCCTGGGAAAG | Reverse Surveyor primer for CR1 off- |
| | | | target 1 |
| 271 | CR1-CelI-OT2-F | AGTAGCTTACCTTGGCAGAGAAAA | Forward Surveyor primer for CR1 off- |
| | | | target 2 |
| 272 | CR1-CelI-OT2-R | TGACATACTGTTACCCTTTGCAGT | Reverse Surveyor primer for CR1 off- |
| | | | target 2 |
| 273 | CR1-CelI-OT3-F | GAAAGGCTCAGTGAATGTTTGTT | Forward Surveyor primer for CR1 off- |
| | | | target 3 |
| 274 | CR1-CelI-OT3-R | CACTGCATCATCTCATTAAATCAA | Reverse Surveyor primer for CR1 off- |
| | | | target 3 |
| 275 | CR1-CelI-OT4-F | CCCATATATTCATGATTACCCACA | Forward Surveyor primer for CR1 off- |
| | | | target 4 |
| 276 | CR1-CelI-OT4-R | TATCAGAACGAGCACTAAAAGCAC | Reverse Surveyor primer for CR1 off- |
| | | | target 4 |
| 277 | CR1-CelI-OT5-F | TTGGGAGGCTGAGGTACAAG | Forward Surveyor primer for CR1 off- |
| | | | target 5 |
| 278 | CR1-CelI-OT5-R | GAATGAAAAACAAACAGAAGGTGA | Reverse Surveyor primer for CR1 off- |
| | | | target 5 |
| 279 | CR1-CelI-OT6-F | CTCCTCATCTGTACCCTTCAATCT | Forward Surveyor primer for CR1 off- |
| | | | target 6 |
| 280 | CR1-CelI-OT6-R | AGAGTGGCATCTAGTGTCAGTGAG | Reverse Surveyor primer for CR1 off- |
| | | | target 6 |
| 281 | CR1-CelI-OT7-F | TACCAAAAGCTTCTCCTGTTTACC | Forward Surveyor primer for CR1 off- |
| | | | target 7 |
| 282 | CR1-CelI-OT7-R | GTAAGTTGGATGGCCTATTCTTTG | Reverse Surveyor primer for CR1 off- |
| | | | target 7 |
| 283 | CR1-CelI-OT8-F | GAAGGAAATGCAAGGATACAAGAT | Forward Surveyor primer for CR1 off- |
| | | | target 8 |
| 284 | CR1-CelI-OT8-R | TGATTGAAAGAATCATTCCAGAAA | Reverse Surveyor primer for CR1 off- |
| | | | target 8 |
| 285 | CR1-CelI-OT9-F | TCAGAAGGAAAATTGAAATTGGTT | Forward Surveyor primer for CR1 off- |
| | | | target 9 |
| 286 | CR1-CelI-OT9-R | CAGATGTGTTCTTCATCATTCCTC | Reverse Surveyor primer for CR1 off- |
| | | | target 9 |
| 287 | CR1-CelI-OT10-F | TTCTCTTTAGGGAAAGCTCTCAAA | Forward Surveyor primer for CR1 off- |
| | | | target 10 |
| 288 | CR1-CelI-OT10-R | GGGTATAGATCATATGGAGGGAAG | Reverse Surveyor primer for CR1 off- |
| | | | target 10 |
| 289 | CR5-CelI-OT1-F | AGATGATCTGCCCACCTCAG | Forward Surveyor primer for CR5 off- |
| | | | target 1 |
| 290 | CR5-CelI-OT1-R | CTTTCTTCCTCATTTAGTGGCAAT | Reverse Surveyor primer for CR5 off- |
| | | | target 1 |
| 291 | CR5-CelI-OT2-F | ATGAATTGCAGATTGATGGTACTG | Forward Surveyor primer for CR5 off- |
| | | | target 2 |
| 292 | CR5-CelI-OT2-R | TCTCACCAAGAACCAAATTGTCTA | Reverse Surveyor primer for CR5 off- |
| | | | target 2 |
| 293 | CR5-CelI-OT3-F | GTAGGATACCTTGGCAACAGTCTT | Forward Surveyor primer for CR5 off- |
| | | | target 3 |
| 294 | CR5-CelI-OT3-R | TTAACGAATTGTGAGATTTGCTGT | Reverse Surveyor primer for CR5 off- |
| | | | target 3 |
| 295 | CR5-CelI-OT4-F | TCAGAAAGTCAAGTAGCACACACA | Forward Surveyor primer for CR5 off- |
| | | | target 4 |
| 296 | CR5-CelI-OT4-R | AGAAGCACACACTCAGGTAAAGC | Reverse Surveyor primer for CR5 off- |
| | | | target 4 |
| 297 | CR5-CelI-OT5-F | TCTTTGGGGGAATAATGACTAAAA | Forward Surveyor primer for CR5 off- |
| | | | target 5 |
| 298 | CR5-CelI-OT5-R | TTTGGCATTTATGGGAATAAAACT | Reverse Surveyor primer for CR5 off- |
| | | | target 5 |
| 299 | CR5-CelI-OT6-F | ACTAATTCTGGTCAAGCCCATCA | Forward Surveyor primer for CR5 off- |
| | | | target 6 |
| 300 | CR5-CelI-OT6-R | TTAAGACATCGGATGAACAGAAAG | Reverse Surveyor primer for CR5 off- |
| | | | target 6 |
| 301 | CR5-CelI-OT7-F | AGAAGCTTTCTGACATGATCTGC | Forward Surveyor primer for CR5 off- |
| | | | target 7 |
| 302 | CR5-CelI-OT7-R | TCAATTGCATTAGGACTTAGACCA | Reverse Surveyor primer for CR5 off- |
| | | | target 7 |
| 303 | CR5-CelI-OT8-F | GTTAAATTACCTGTGAAGCCCTTG | Forward Surveyor primer for CR5 off- |
| | | | target 8 |
| 304 | CR5-CelI-OT8-R | CGGAAAACAGATCCACTTTATGAT | Reverse Surveyor primer for CR5 off- |
| | | | target 8 |
| 305 | CR5-CelI-OT9-F | AAATCCACTGGAAACATCTTGAGT | Forward Surveyor primer for CR5 off- |
| | | | target 9 |
| 306 | CR5-CelI-OT9-R | AGTCTCTTCAGAATCATGCCCTAT | Reverse Surveyor primer for CR5 off- |
| | | | target 9 |
| 307 | CR5-CelI-OT10-F | GCTTGGTGGCACATACCTGTAG | Forward Surveyor primer for CR5 off- |
| | | | target 10 |
| 308 | CR5-CelI-OT10-R | GGTAGGTAGATTTGCTTGCTTGTT | Reverse Surveyor primer for CR5 off- |
| | | | target 10 |
| 309 | CR6-CelI-OT1-F | AGCTCTCAGCAGAGTAGGGATTTA | Forward Surveyor primer for CR6 off- |
| | | | target 1 |
| 310 | CR6-CelI-OT1-R | GTGAGTCTACTGCACCCCATC | Reverse Surveyor primer for CR6 off- |
| | | | target 1 |
| 311 | CR6-CelI-OT2-F | TGACACTGTGAAGTCAATTCTGTC | Forward Surveyor primer for CR6 off- |
| | | | target 2 |
| 312 | CR6-CelI-OT2-R | TCAAGAACTTGACAATGAGCAAAT | Reverse Surveyor primer for CR6 off- |
| | | | target 2 |
| 313 | CR6-CelI-OT3-F | TATCCGATCCACTGTTGTGTGT | Forward Surveyor primer for CR6 off- |
| | | | target 3 |
| 314 | CR6-CelI-OT3-R | CAGGAGACCCAAAACCACTCTAC | Reverse Surveyor primer for CR6 off- |
| | | | target 3 |
| 315 | CR6-CelI-OT4-F | TTGTTCTACAAATAGGGCTTCCTT | Forward Surveyor primer for CR6 off- |
| | | | target 4 |
| 316 | CR6-CelI-OT4-R | TGTTAAGTTTGGGCTTATGTTCCT | Reverse Surveyor primer for CR6 off- |
| | | | target 4 |
| 317 | CR6-CelI-OT5-F | CACAAGTCTCACTGCACAAACAT | Forward Surveyor primer for CR6 off- |
| | | | target 5 |
| 318 | CR6-CelI-OT5-R | TGACCCATGATTATCTCTCTTTGA | Reverse Surveyor primer for CR6 off- |
| | | | target 5 |
| 319 | CR6-CelI-OT6-F | TTCAGCTTCTGATTGGTTTTAATG | Forward Surveyor primer for CR6 off- |
| | | | target 6 |
| 320 | CR6-CelI-OT6-R | CCAATTCCTTAATTTTCCCTACAG | Reverse Surveyor primer for CR6 off- |
| | | | target 6 |
| 321 | CR6-CelI-OT7-F | ATCTCAGACCAGGAGGGAGAC | Forward Surveyor primer for CR6 off- |
| | | | target 7 |
| 322 | CR6-CelI-OT7-R | CCTCAGGGTCAGTACATTTTTCAG | Reverse Surveyor primer for CR6 off- |
| | | | target 7 |
| 323 | CR6-CelI-OT8-F | TTCTTAGGACATTGCTCCACATAC | Forward Surveyor primer for CR6 off- |
| | | | target 8 |
| 324 | CR6-CelI-OT8-R | GCAAACATAATGCAACTCGTAATC | Reverse Surveyor primer for CR6 off- |
| | | | target 8 |
| 325 | CR6-CelI-OT9-F | GCAAGGGAGTCTGTGTCTTTG | Forward Surveyor primer for CR6 off- |
| | | | target 9 |
| 326 | CR6-CelI-OT9-R | TCATTTAAGTGGCTGTTCTGTGTT | Reverse Surveyor primer for CR6 off- |
| | | | target 9 |
| 327 | CR6-CelI-OT10-F | ACAAAACAGAGAGAAAAGGCAGAG | Forward Surveyor primer for CR6 off- |
| | | | target 10 |
| 328 | CR6-CelI-OT10-R | GTTTTGATTTCTGGTGCCTACAG | Reverse Surveyor primer for CR6 off- |
| | | | target 10 |
| 329 | CR36-CelI-OT1-F | ACTGAAGCTGAAGCCCAGTC | Forward Surveyor primer for CR36 |
| | | | off-target 1 |
| 330 | CR36-CelI-OT1-R | ACATGAGCTCTCAGGTTTCTGAC | Reverse Surveyor primer for CR36 |
| | | | off-target 1 |
| 331 | CR36-CelI-OT2-F | TCAAACTTAGATGGTTCCCTATGTT | Forward Surveyor primer for CR36 |
| | | | off-target 2 |
| 332 | CR36-CelI-OT2-R | GTACCCTGAAAATGTAGGGTGACT | Reverse Surveyor primer for CR36 |
| | | | off-target 2 |
| 333 | CR36-CelI-OT3-F | CACTTCCCAAGTGAGGCAAT | Forward Surveyor primer for CR36 |
| | | | off-target 3 |
| 334 | CR36-CelI-OT3-R | CTATACTTGGGGCTGACTTGCTAC | Reverse Surveyor primer for CR36 |
| | | | off-target 3 |
| 335 | CR36-CelI-OT4-F | TCGTATAGGTTACTTTGGCTCACA | Forward Surveyor primer for CR36 |
| | | | off-target 4 |
| 336 | CR36-CelI-OT4-R | AGGGATCTTTACTCCTCAGTGTGT | Reverse Surveyor primer for CR36 |
| | | | off-target 4 |
| 337 | CR36-CelI-OT5-F | TGTAGAAGTTGGAATATCCTGCTG | Forward Surveyor primer for CR36 |
| | | | off-target 5 |
| 338 | CR36-CelI-OT5-R | GTCAACAATTTGATCTCAGGCTTC | Reverse Surveyor primer for CR36 |
| | | | off-target 5 |
| 339 | CR36-CelI-OT6-F | CTCAGTACTAAAGATGGACGCTTG | Forward Surveyor primer for CR36 |
| | | | off-target 6 |
| 340 | CR36-CelI-OT6-R | AATCATTTCAGTCTTCCCAACAAT | Reverse Surveyor primer for CR36 |
| | | | off-target 6 |
| 341 | CR36-CelI-OT7-F | GGGAATCACAGTAGATGTTTGTCA | Forward Surveyor primer for CR36 |
| | | | off-target 7 |
| 342 | CR36-CelI-OT7-R | AGACCAGGAGGTAAGAACATTTTG | Reverse Surveyor primer for CR36 |
| | | | off-target 7 |
| 343 | CR36-CelI-OT8-F | CCACATAGAAAGAGACTTGCAGAA | Forward Surveyor primer for CR36 |
| | | | off-target 8 |
| 344 | CR36-CelI-OT8-R | AGAGATGCCAAAAGAACAGTCAAT | Reverse Surveyor primer for CR36 |
| | | | off-target 8 |
| 345 | CR36-CelI-OT9-F | TGTGCCTTAGGCTATGTAAACTGT | Forward Surveyor primer for CR36 |
| | | | off-target 9 |
| 346 | CR36-CelI-OT9-R | AAACCCTTGTAACCAAAATTACCA | Reverse Surveyor primer for CR36 |
| | | | off-target 9 |
| 347 | CR36-CelI-OT10- | TAACTGCATCAGAAGTCCTTGCTA | Forward Surveyor primer for CR36 |
| | F | | off-target 10 |
| 348 | CR36-CelI-OT10- | GGAGACCAAGCTGCTAAAGTCA | Reverse Surveyor primer for CR36 |
| | R | | off-target 10 |
| 349 | CelI-CR3-F-nested | GTGGTGccgcggGAGTTTGGCTCAAAT | Nested PCR first round primers |
| | | TGTTACTCTT | |
| 350 | CelI-CR3-R-nested | GTGGTGccgcggGGGAAATGGTCTAG | Nested PCR first round primers |
| | | GAGAGTAAAGT | |
| 351 | CelI-CR1-F-nested | GTGGTGccgcggGAGAGGTTATGTGGC | Nested PCR first round primers |
| | | TTTACCA | |
| 352 | CelI-CR1-R-nested | GTGGTGccgcggCTCATTCTCATGCCT | Nested PCR first round primers |
| | | GGACA | |
| 353 | CelI-CR5-F-nested | GTGGTGccgcggCGGGCTTGGACAGA | Nested PCR first round primers |
| | | ACTTAC | |
| 354 | CelI-CR5-R-nested | GTGGTGccgcggCTGCGTAGTGCCAAA | Nested PCR first round primers |
| | | ACAAA | |
| 355 | CelI-CR6-F-nested | GTGGTGccgcggTAATTTCATTGAAGA | Nested PCR first round primers |
| | | GTGGCTGAA | |
| 356 | CelI-CR6-R-nested | GTGGTGccgcggAAGCCCTGTGTGGTA | Nested PCR first round primers |
| | | GTAGTCAGT | |
| 357 | CelI-CR36-F- | GTGGTGccgcggCAAGTCAGAAGTCAC | Nested PCR first round primers |
| | nested | TTGCTTTGT | |
| 358 | CelI-CR36-R- | GTGGTGccgcggTTTTATGTGCAGGAA | Nested PCR first round primers |
| | nested | TCAGTCTGT | |
| 359 | CR3-CelI-OT1-F- | GTGGTGccgcggTGTGTGCTTCTGTAC | Nested PCR first round primers |
| | nested | ACATCATCT | |
| 360 | CR3-CelI-OT1-R- | GTGGTGccgcggAGATTTCAACCCTCA | Nested PCR first round primers |
| | nested | AAAACTGAG | |
| 361 | CR1-CelI-OT1-F- | GTGGTGccgcggTTGGGAACATCAGAG | Nested PCR first round primers |
| | nested | AAAGTATGA | |
| 362 | CR1-CelI-OT1-R- | GTGGTGccgcggACAAATTACAGTCTC | Nested PCR first round primers |
| | nested | CTGGGAAAG | |
| 363 | CR36-CelI-OT3-F- | GTGGTGccgcggCACTTCCCAAGTGAG | Nested PCR first round primers |
| | nested | GCAAT | |
| 364 | CR36-CelI-OT3-R- | GTGGTGccgcggCTATACTTGGGGCTG | Nested PCR first round primers |
| | nested | ACTTGCTAC | |
| 365 | CR3-P1/P3-F | GTGGTGccgcggTTGGCTCTTTAGCTT | Nested PCR second round primers |
| | | GTGTTTC | |
| 366 | CR3-P1/P3-R | GTGGTGccgcggTGAGACTCCCAAAGG | Nested PCR second round primers |
| | | CAATC | |
| 367 | CR3-P1/P4-F | GTGGTGccgcggTTGGCTCTTTAGCTT | Nested PCR second round primers |
| | | GTGTTTC | |
| 368 | CR3-P1/P4-R | GTGGTGccgcggACTGAGGGGTGATCT | Nested PCR second round primers |
| | | TGGTG | |
| 369 | CR3-P2/P3-F | GTGGTGccgcggGCAGAGAAAGCCAG | Nested PCR second round primers |
| | | TCGGTA | |
| 370 | CR3-P2/P3-R | GTGGTGccgcggTGAGACTCCCAAAGG | Nested PCR second round primers |
| | | CAATC | |
| 371 | CR3-P2/P4-F | GTGGTGccgcggGCAGAGAAAGCCAG | Nested PCR second round primers |
| | | TCGGTA | |
| 372 | CR3-P2/P4-R | GTGGTGccgcggACTGAGGGGTGATCT | Nested PCR second round primers |
| | | TGGTG | |
| 373 | CR1-P1/P5-F | GTGGTGccgcggCCAGAGTTCCTAGGG | Nested PCR second round primers |
| | | CAGAG | |
| 374 | CR1-P1/P5-R | GTGGTGccgcggAGCTAGTCCCCACAT | Nested PCR second round primers |
| | | TCCAC | |
| 375 | CR1-P1/P6-F | GTGGTGccgcggCCAGAGTTCCTAGGG | Nested PCR second round primers |
| | | CAGAG | |
| 376 | CR1-P1/P6-R | GTGGTGccgcggGGTGGAGGGAAACT | Nested PCR second round primers |
| | | TTAGGC | |
| 377 | CR1-P2/P5-F | GTGGTGccgcggCTCATTCTCATGCCT | Nested PCR second round primers |
| | | GGACA | |
| 378 | CR1-P2/P5-R | GTGGTGccgcggAGCTAGTCCCCACAT | Nested PCR second round primers |
| | | TCCAC | |
| 379 | CR1-P2/P6-F | GTGGTGccgcggTCTCATGCCTGGACA | Nested PCR second round primers |
| | | AGTAACT | |
| 380 | CR1-P2/P6-R | GTGGTGccgcggGGTGGAGGGAAACT | Nested PCR second round primers |
| | | TTAGGC | |
| 381 | CR5-P3/P5-F | GTGGTGccgcggGGCTTGGACAGAACT | Nested PCR second round primers |
| | | TACCG | |
| 382 | CR5-P3/P5-R | GTGGTGccgcggCACCACTGTCTGCCT | Nested PCR second round primers |
| | | AAGGA | |
| 383 | CR5-P4/P6-F | GTGGTGccgcggGGCTTGGACAGAACT | Nested PCR second round primers |
| | | TACCG | |
| 384 | CR5-P4/P6-R | GTGGTGccgcggGGTGGAGGGAAACT | Nested PCR second round primers |
| | | TTAGGC | |
| 385 | CR5-P3/P5-F | GTGGTGccgcggCGTAGTGCCAAAACA | Nested PCR second round primers |
| | | AACAGT | |
| 386 | CR5-P3/P5-R | GTGGTGccgcggCACCACTGTCTGCCT | Nested PCR second round primers |
| | | AAGGA | |
| 387 | CR5-P4/P6-F | GTGGTGccgcggCGTAGTGCCAAAACA | Nested PCR second round primers |
| | | AACAGT | |
| 388 | CRS-P4/P6-R | GTGGTGccgcggGGTGGAGGGAAACT | Nested PCR second round primers |
| | | TTAGGC | |
| 389 | CR6-P1/P5-F | GTGGTGccgcggGCGAGGGCCTACTTG | Nested PCR second round primers |
| | | ATATG | |
| 390 | CR6-P1/P5-R | GTGGTGccgcggCTTCCCAAGTGAGGC | Nested PCR second round primers |
| | | AATGC | |
| 391 | CR6-P1/P6-F | GTGGTGccgcggACGTTTTGTGCTGCT | Nested PCR second round primers |
| | | GTAACA | |
| 392 | CR6-P1/P6-R | GTGGTGccgcggCTGCAGGCACATTCT | Nested PCR second round primers |
| | | CTTCC | |
| 393 | CR6-P2/P5-F | GTGGTGccgcggGCCCTGTGTGGTAGT | Nested PCR second round primers |
| | | AGTCA | |
| 394 | CR6-P2/P5-R | GTGGTGccgcggCTTCCCAAGTGAGGC | Nested PCR second round primers |
| | | AATGC | |
| 395 | CR6-P2/P6-F | GTGGTGccgcggCAGTATTAAGGGGTG | Nested PCR second round primers |
| | | GGAGCT | |
| 396 | CR6-P2/P6-R | GTGGTGccgcggTCTCTTCCTCACACA | Nested PCR second round primers |
| | | GCTGA | |
| 397 | CR36-P3/P5-F | GTGGTGccgcggGGAGCTTGGAGGGA | Nested PCR second round primers |
| | | AGAGAA | |
| 398 | CR36-P3/P5-R | GTGGTGccgcggCTTCCCAAGTGAGGC | Nested PCR second round primers |
| | | AATGC | |
| 399 | CR36-P4/P6-F | GTGGTGccgcggATGGATGGGGAAGA | Nested PCR second round primers |
| | | CACTGG | |
| 400 | CR36-P4/P6-R | GTGGTGccgcggCTGCAGGCACATTCT | Nested PCR second round primers |
| | | CTTCC | |
| 401 | CR36-P3/P5-F | GTGGTGccgcggGGATGAAACAGGGC | Nested PCR second round primers |
| | | AGGAAC | |
| 402 | CR36-P3/P5-R | GTGGTGccgcggTTCCCAAGTGAGGCA | Nested PCR second round primers |
| | | ATGC | |
| 403 | CR36-P4/P6-F | GTGGTGccgcggTTTGCAGAGCCATGA | Nested PCR second round primers |
| | | TGAGG | |
| 404 | CR36-P4/P6-R | GTGGTGccgcggCGACAGCCAAAACA | Nested PCR second round primers |
| | | GCCG | |

Western blot analysis. To assess dystrophin protein expression, immortalized myoblasts were differentiated into myofibers by replacing the growth medium with DMEM supplemented with 1% insulin-transferrin-selenium (Invitrogen) and 1% antibiotic/antimycotic (Invitrogen) for 4-7 days, such as 6 or 7 days. Fibroblasts were transdifferentiated into myoblasts by inducing MyoD overexpression and incubating the cells in DMEM supplemented with 1% insulin-transferrin-selenium (Invitrogen), 1% antibiotic/antimycotic (Invitrogen) and 3 μg/mL doxycycline for 15 days. Dystrophin expression was assessed at 3 days after transfecting HEK293T cells. Cells were trypsinized, collected and lysed in RIPA buffer (Sigma) supplemented with a protease inhibitor cocktail (Sigma) and the total protein amount was quantified using the bicinchoninic acid assay according to the manufacturer's instructions (Pierce). Samples were then mixed with NuPAGE loading buffer (Invitrogen) and 5% β-mercaptoethanol and heated to 85° C. for 10 minutes. Twenty-five micrograms of protein were separated on 4-12% NuPAGE Bis-Tris gels (Invitrogen) with MES buffer (Invitrogen). Proteins were transferred to nitrocellulose membranes for 1-2 hrs in transfer buffer containing 10-20% methanol, such as 10% methanol, and 0.01% SDS. The blot was then blocked for 1 hr with 5% milk-TBST at room temperature. Blots were probed with the following primary antibodies: MANDYS8 to detect dystrophin (1:100, Sigma D8168) and rabbit anti-GAPDH (1:5000, Cell Signaling 2118S). Blots were then incubated with mouse or rabbit horseradish peroxidase-conjugated secondary antibodies (Santa Cruz) and visualized using the ChemiDoc chemilumescent system (BioRad) and Western-C ECL substrate (BioRad).

Transplantation into immunodeficient mice. All animal experiments were conducted under protocols approved by the Duke Institutional Animal Care & Use Committee. Cells were trypsinized, collected and washed in 1×Hank's Balanced Salt Solution (HBSS, Sigma). Two million cells were pelleted and resuspended in five μL 1×HBSS (Sigma) supplemented with cardiotoxin (Sigma #C9759) immediately prior to injection. These cells were transplanted into the hind limb tibialis anterior (TA) muscle of NOD.SCID.gamma (NSG) mice (Duke CCIF Breeding Core) by intramuscular injection. Four weeks after injection, mice were euthanized and the TA muscles were harvested.

Immunofluorescence staining. Harvested TA muscles were incubated in 30% glycerol overnight at 4° C. before mounting and freezing in Optimal Cutting Temperature compound. Serial 10 micron sections were obtained by cryosectioning of the embedded muscle tissue at −20° C. Cryosections were then washed in PBS to remove the OCT compound and subsequently blocked for 30-60 minutes at room temperature in PBS containing 10% heat-inactivated fetal bovine serum for spectrin detection or 5% heat-inactivated fetal bovine serum for dystrophin detection. Cryosections were incubated overnight at 4° C. with the following primary antibodies that are specific to human epitopes only: anti-spectrin (1:20, Leica NCL-SPEC1) or anti-dystrophin (1:2, Leica NCL-DYS3). After primary staining, spectrin or dystrophin expression was detected using a tyramide-based immunofluorescence signal amplification detection kit (Life Technologies, TSA Kit #22, catalog #T-20932). Briefly, cryosections were incubated with 1:200 goat anti-mouse biotin-XX secondary (Life Technologies #B2763) in blocking buffer for 1 hr at room temperature. The signal was then amplified using streptavidin-HRP conjugates (1:100, from TSA Kit) in blocking buffer for 1 hr at room temperature. Finally, cryosections were incubated with tyramide-AlexaFluor488 conjugates (1:100, TSA kit) in manufacturer-provided amplification buffer for 10 minutes at room temperature. Stained cryosections were then mounted in ProLong AntiFade (Life Technologies #P36934) and visualized with conventional fluorescence microscopy.

Cytotoxicity assay. To quantitatively assess potential sgRNA or SpCas9 nuclease-associated cytotoxicity, HEK293T cells were transfected with 10 ng of a GFP reporter and 100 ng SpCas9 expression vector and 100 ng sgRNA expression vector using Lipofectamine 2000 according to the manufacturer's instructions (Invitrogen). The percentage of GFP positive cells was assessed at 2 and 5 days by flow cytometry. The survival rate was calculated as the decrease in GFP positive cells from days 2 to 5 and normalized to cells transfected with an empty nuclease expression vector as described (Cornu et al., Meth Mol Biol 649:237-245 (2010)).

### Example 4

### CRISPRs Targeting the Dystrophin Gene—Results

The CRISPR/Cas9-based system was designed to target the dystrophin gene. Various gRNAs were chosen to target different regions of the human and mouse dystrophin gene based on NNNNNNNNNN NNNNN NNNNN NGG (SEQ ID NO: 677) and GNNNN NNNNN NNNNN NNNNN NGG (SEQ ID NO: 678) (see Tables 6, 7 and 8).

TABLE 6

| Name | | | | | | |
|---|---|---|---|---|---|---|
| (SEQ | | | | | 19 bp for chimeric | |
| ID NO) | Species | Gene | Target | Strand | (add G on 5′ end) | PAM |
| DCR1 | Human | DMD | Intron 50 | + | attggctttgatttcccta (SEQ ID NO: 65) | GGG |
| (628) | | | | | | |
| DCR2 | Human | DMD | Intron 50 | − | tgtagagtaagtcagccta (SEQ ID | TGG |
| (66) | | | | | NO: 679) | |
| DCR3 | Human | DMD | Exon 51-55′ | + | cctactcagactgttactc (SEQ ID NO: 67) | TGG |
| (629) | | | | | | |
| DCR4 | Human | DMD | Exon 51-53 | + | ttggacagaacttaccgac (SEQ ID | TGG |
| (68) | | | | | NO: 680) | |
| DCR5 | Human | DMD | Intron 51 | − | cagttgcctaagaactggt (SEQ ID | GGG |
| (630) | | | | | NO: 69) | |
| DCR6 | Human | DMD | Intron 44 | − | GGGCTCCACCCTCACGAGT | GGG |
| (631) | | | | | (SEQ ID NO: 70) | |
| DCR7 | Human | DMD | Intron 55 | + | TTTGCTTCGCTATAAAACG | AGG |
| (71) | | | | | (SEQ ID NO: 681) | |
| DCR8 | Human | DMD | Exon 41 | + | TCTGAGGATGGGGCCGCAA | TGG |
| (72) | | | | | (SEQ ID NO: 682) | |
| DCR9 | Human | DMD | Exon 44 | − | GATCTGTCAAATCGCCTGC | AGG |
| (73) | | | | | (SEQ ID NO: 683) | |
| DCR10 | Human | DMD | Exon 45 | + | CCAGGATGGCATTGGGCAG | CGG |
| (74) | | | | | (SEQ ID NO: 684) | |
| DCR11 | Human | DMD | Exon 45 | + | CTGAATCTGCGGTGGCAGG | AGG |
| (75) | | | | | (SEQ ID NO: 685) | |
| DCR12 | Human | DMD | Exon 46 | − | TTCTTTTGTTCTTCTAGCc (SEQ | TGG |
| (76) | | | | | ID NO: 686) | |
| DCR13 | Human | DMD | Exon 46 | + | GAAAAGCTTGAGCAAGTCA | AGG |
| (77) | | | | | (SEQ ID NO: 687) | |
| DCR14 | Human | DMD | Exon 47 | + | GAAGAGTTGCCCCTGCGCC | AGG |
| (78) | | | | | (SEQ ID NO: 688) | |
| DCR15 | Human | DMD | Exon 47 | + | ACAAATCTCCAGTGGATAA | AGG |
| (79) | | | | | (SEQ ID NO: 689) | |
| DCR16 | Human | DMD | Exon 48 | − | TGTTTCTCAGGTAAAGCTC | TGG |
| (80) | | | | | (SEQ ID NO: 690) | |
| DCR17 | Human | DMD | Exon 48 | + | GAAGGACCATTTGACGTTa | AGG |
| (81) | | | | | (SEQ ID NO: 691) | |
| DCR18 | Human | DMD | Exon 49 | − | AACTGCTATTTCAGTTTCc | TGG |
| (82) | | | | | (SEQ ID NO: 692) | |
| DCR19 | Human | DMD | Exon 49 | + | CCAGCCACTCAGCCAGTGA | AGG |
| (83) | | | | | (SEQ ID NO: 693) | |
| DCR20 | Human | DMD | Exon 50 | + | gtatgcttttctgttaaag (SEQ ID | AGG |
| (84) | | | | | NO: 694) | |
| DCR21 | Human | DMD | Exon 50 | + | CTCCTGGACTGACCACTAT | TGG |
| (85) | | | | | (SEQ ID NO: 695) | |
| DCR22 | Human | DMD | Exon 52 | + | GAACAGAGGCGTCCCCAGT | TGG |
| (86) | | | | | (SEQ ID NO: 696) | |
| DCR23 | Human | DMD | Exon 52 | + | GAGGCTAGAACAATCATTA | CGG |
| (87) | | | | | (SEQ ID NO: 697) | |
| DCR24 | Human | DMD | Exon 53 | + | ACAAGAACACCTTCAGAAC | CGG |
| (88) | | | | | (SEQ ID NO: 698) | |
| DCR25 | Human | DMD | Exon 53 | − | GGTTTCTGTGATTTTCTTT | TGG |
| (89) | | | | | (SEQ ID NO: 699) | |
| DCR26 | Human | DMD | Exon 54 | + | GGCCAAAGACCTCCGCCAG | TGG |
| (90) | | | | | (SEQ ID NO: 700) | |
| DCR27 | Human | DMD | Exon 54 | + | TTGGAGAAGCATTCATAAA | AGG |
| (91) | | | | | (SEQ ID NO: 701) | |
| DCR28 | Human | DMD | Exon 55 | − | TCGCTCACTCACCctgcaa (SEQ | AGG |
| (92) | | | | | ID NO: 702) | |
| DCR29 | Human | DMD | Exon 55 | + | AAAAGAGCTGATGAAACAA | TGG |
| (93) | | | | | (SEQ ID NO: 703) | |
| DCR30 | Human | DMD | 5′UTR/Exon | + | TAcACTTTTCaAAATGCTT (SEQ | TGG |
| (94) | | | 1 | | ID NO: 704) | |
| DCR31 | Human | DMD | Exon 51 | + | gagatgatcatcaagcaga (SEQ ID | AGG |
| (95) | | | | | NO: 705) | |
| DCR32 | Mouse | DMD | mdx | + | ccttgaaagagcaaTaaaa (SEQ ID | TGG |
| (96) | | | mutation | | NO: 706) | |
| DCR33 | Human | DMD | Intron 44 | − | CACAAAAGTCAAATCGGAA | TGG |
| (97) | | | | | (SEQ ID NO: 707) | |
| DCR34 | Human | DMD | Intron 44 | − | ATTTCAATATAAGATTCGG | AGG |
| (98) | | | | | (SEQ ID NO: 708) | |
| DCR35 | Human | DMD | Intron 55 | − | CTTAAGCAATCCCGAACTC | TGG |
| (99) | | | | | (SEQ ID NO: 709) | |
| DCR36 | Human | DMD | Intron 55 | − | CCTTCTTTATCCCCTATCG | AGG |
| (632) | | | | | (SEQ ID NO: 100) | |
| DCR40 | Mouse | DMD | Exon 23 | − | aggccaaacctcggcttac (SEQ ID | NNGRR |
| (104) | | | | | NO: 710) | |
| DCR41 | Mouse | DMD | Exon 23 | + | TTCGAAAATTTCAGgtaag (SEQ | NNGRR |
| (105) | | | | | ID NO: 711) | |
| DCR42 | Mouse | DMD | Exon 23 | + | gcagaacaggagataacag (SEQ ID | NNGRRT |
| (106) | | | | | NO: 712) | |
| DCR43 | Mouse | ACVR2B | Exon 1 | + | gcggccctcgcccttctct (SEQ ID | ggggat |
| (107) | | | | | NO: 713) | |
| DCR48 | Human | DMD | Intron 45 | − | TAGTGATCGTGGATACGAG | AGG |
| (108) | | | | | (SEQ ID NO: 714) | |
| DCR49 | Human | DMD | Intron 45 | − | TACAGCCCTCGGTGTATAT | TGG |
| (109) | | | | | (SEQ ID NO: 715) | |
| DCR50 | Human | DMD | Intron 52 | − | GGAAGGAATTAAGCCCGAA | TGG |
| (110) | | | | | (SEQ ID NO: 716) | |
| DCR51 | Human | DMD | Intron 53 | − | GGAACAGCTTTCGTAGTTG | AGG |
| (111) | | | | | (SEQ ID NO: 718) | |
| DCR52 | Human | DMD | Intron 54 | + | ATAAAGTCCAGTGTCGATC | AGG |
| (112) | | | | | (SEQ ID NO: 719) | |
| DCR53 | | | Intron 54 | + | AAAACCAGAGCTTCGGTCA | AGG |
| (113) | | | | | (SEQ ID NO: 720) | |
| DCR54 | Mouse | Rosa26 | ZFN region | + | GAGTCTTCTGGGCAGGCTTAA | TGG |
| (114) | | | | | AGGCTAACC (SEQ ID NO: 720) | |
| DCR55 | Mouse | Rosa26 | mRNA | − | TCGGGTGAGCATGTCTTTAAT | TGG |
| (115) | | | | | CTACCTCGA (SEQ ID NO: 721) | |
| DCR49 | Human | DMD | Ex 51 | − | gtgtcaccagagtaacagt (SEQ ID | ctgagt |
| (116) | | | | | NO: 722) | |
| DCR50 | Human | DMD | Ex 51 | + | tgatcatcaagcagaaggt (SEQ ID | atgag |
| (117) | | | | | NO: 723) | |
| DCR60 | Mouse | DMD | Exon 23 | + | AACTTCGAAAATTTCAGgta | agccgagg |
| (118) | | | | | (SEQ ID NO: 724) | |
| DCR61 | Mouse | DMD | Intron 22 | + | gaaactcatcaaatatgcgt (SEQ ID | gttagtgt |
| (119) | | | | | NO: 725) | |
| DCR62 | Mouse | DMD | Intron 22 | − | tcatttacactaacacgcat (SEQ ID | atttgatg |
| (120) | | | | | NO: 726) | |
| DCR63 | Mouse | DMD | Intron 22 | + | gaatgaaactcatcaaatat (SEQ ID | gcgtgtta |
| (121) | | | | | NO: 727) | |
| DCR64 | Mouse | DMD | Intron 23 | − | tcatcaatatctttgaagga (SEQ ID | ctctgggt |
| (122) | | | | | NO: 728) | |
| DCR65 | Mouse | DMD | Intron 23 | − | tgttttcataggaaaaatag (SEQ ID | gcaagttg |
| (123) | | | | | NO: 729) | |
| DCR66 | Mouse | DMD | Intron 23 | + | aattggaaaatgtgatggga (SEQ ID | aacagata |
| (124) | | | | | NO: 730) | |
| DCR67 | Human | DMD | Exon 51 | + | atgatcatcaagcagaaggt (SEQ ID | atgagaaa |
| (125) | | | | | NO: 731) | |
| DCR68 | Human | DMD | Exon 51 | + | agatgatcatcaagcagaag (SEQ ID | gtatgaga |
| (126) | | | | | NO: 732) | |
| DCR69 | Human | DMD | Exon 51 | − | cattttctctcataccttct (SEQ ID NO: | gcttgatg |
| (127) | | | | | 733) | |
| DCR70 | Human | DMD | Exon 51 | + | tcctactcagactgttactc (SEQ ID | tggtgaca |
| (128) | | | | | NO: 734) | |
| DCR71 | Human | DMD | Exon 51 | − | acaggttgtgtcaccagagt (SEQ ID | aacagtct |
| (129) | | | | | NO: 735) | |
| DCR72 | Human | DMD | Exon 51 | − | ttatcattttttctcatacc (SEQ ID NO: | ttctgctt |
| (130) | | | | | 736) | |
| DCR73 | Human | DMD | Intron 51 | − | ttgcctaagaactggtggga (SEQ ID | aatggtct |
| (131) | | | | | NO: 737) | |
| DCR74 | Human | DMD | Intron 51 | − | aaacagttgcctaagaactg (SEQ ID | gtgggaaa |
| (132) | | | | | NO: 738) | |
| DCR75 | Human | DMD | Intron 51 | + | tttcccaccagttcttaggc (SEQ ID | aactgttt |
| (133) | | | | | NO: 739) | |
| DCR76 | Human | DMD | Intron 50 | + | tggctttgatttccctaggg (SEQ ID | tccagctt |
| (134) | | | | | NO: 740) | |
| DCR77 | Human | DMD | Intron 50 | − | tagggaaatcaaagccaatg (SEQ ID | aaacgttc |
| (135) | | | | | NO: 741) | |
| DCR78 | Human | DMD | Intron 50 | − | gaccctagggaaatcaaagc (SEQ ID | caatgaaa |
| (136) | | | | | NO: 742) | |
| DCR79 | Human | DMD | Intron 44 | − | TGAGGGCTCCACCCTCACGA | GTGGGTTT |
| (137) | | | | | (SEQ ID NO: 743) | |
| DCR80 | Human | DMD | Intron 44 | − | AAGGATTGAGGGCTCCACCC | TCACGAGT |
| (138) | | | | | (SEQ ID NO: 744) | |
| DCR81 | Human | DMD | Intron 44 | − | GCTCCACCCTCACGAGTGGG | TTTGGTTC |
| (139) | | | | | (SEQ ID NO: 745) | |
| DCR82 | Human | DMD | Intron 55 | − | TATCCCCTATCGAGGAAACC | ACGAGTTT |
| (140) | | | | | (SEQ ID NO: 746) | |
| DCR83 | Human | DMD | Intron 55 | + | GATAAAGAAGGCCTATTTCA | TAGAGTTG |
| (141) | | | | | (SEQ ID NO: 747) | |
| DCR84 | Human | DMD | Intron 55 | − | AGGCCTTCTTTATCCCCTAT | CGAGGAAA |
| (142) | | | | | (SEQ ID NO: 748) | |
| DCR85 | Human | DMD | Intron 44 | − | TGAGGGCTCCACCCTCACGA | GTGGGT |
| (143) | | | | | (SEQ ID NO: 749) | |
| DCR86 | Human | DMD | Intron 55 | + | GATAAAGAAGGCCTATTTCA | TAGAGT |
| (144) | | | | | (SEQ ID NO: 750) | |

TABLE 7

| Name | Notes | % Mod |
|---|---|---|
| DCR1 | Delete exon 51 | 6.6 |
| DCR2 | Delete exon 51 | 10.3 |
| DCR3 | Frameshift | 13 |
| DCR4 | Delete exon 51 | 11.9 |
| DCR5 | Delete exon 51 | 12.4 |
| DCR6 | As close to exon 44 as possible in intron 44 | 16.1 |
| | (in case of patient deletions) | |
| DCR7 | As close to exon 56 as possible in intron 55 | 6.8 |
| | (in case of patient deletions) | |
| DCR8 | Can correct exon 42-43 deletion (−1/+2) | 17.3 |
| | only, (−2/+1) is not correctable by this | |
| DCR9 | Skip exon 44 (5′) | 14.4 |
| DCR10 | Frameshift | 14.9 |
| DCR11 | Correct downstream of exon 45 | <1 |
| DCR12 | 5′ splice acceptor/frameshift | <1 |
| DCR13 | Correct downstream of exon 46 | 16.9 |
| DCR14 | Frameshift | 17.2 |
| DCR15 | Correct downstream of exon 47 | 15.4 |
| DCR16 | Frameshift | 11.5 |
| DCR17 | Correct downstream of exon 48 | <1 |
| DCR18 | 5′ splice acceptor/frameshift | 1.8 |
| DCR19 | Correct downstream of exon 49 | 33.7 |
| DCR20 | 5′ splice acceptor | 14.9 |
| DCR21 | Correct downstream of exon 50 | 24.1 |
| DCR22 | Frameshift | 25.9 |
| DCR23 | Correct downstream of exon 52 | 25.2 |
| DCR24 | Frameshift (can only correct +1 frame) | 24.8 |
| DCR25 | Correct downstream of exon 53 | 2.6 |
| DCR26 | Frameshift | 24.5 |
| DCR27 | Correct downstream of exon 54 | 13.4 |
| DCR28 | 5′ splice acceptor | 21.6 |
| DCR29 | Correct downstream of exon 55 | 19.2 |
| DCR30 | Integrate minidys in exon 1 | not |
| | | tested |
| DCR31 | Correct downstream of exon 51 | 18.9 |
| DCR32 | Delete stop codon | not |
| | | tested |
| DCR33 | Alternative to CR6 | 1.3 |
| DCR34 | Alternative to CR6 | 13.2 |
| DCR35 | Alternative to CR7 | 22.5 |
| DCR36 | Alternative to CR7 | 26.4 |
| DCR40 | Disrupt exon 23 5′ splice donor | |
| | (correct mdx mutation) | |
| DCR41 | Disrupt exon 23 5′ splice donor | |
| | (correct mdx mutation) | |
| DCR42 | Delete exon 53 mdx4cv mutation | |
| DCR43 | Disrupt myostatin receptor | |

TABLE 8

| Name | Cas9 | | Notes | Cas9 used |
|---|---|---|---|---|
| DCR49 | S. Aureus | | Frameshift in exon 51 | SaCas9 (from Zhang |
| | | | | pX441) (NNGRRT |
| | | | | PAM) |
| DCR50 | S. Aureus | | Disrupt 5′ end of exon 51 | SaCas9 (from Zhang |
| | | | | pX441) (NNGRR PAM) |
| DCR60 | N. | NNNNGANN | Target 3′ splice donor of | NmCas9 (NNNNGANN |
| | Meningitidis | | exon 23 to bypass mdx | PAM) |
| | | | mutation | |
| DCR61 | N. | NNNNGNNT | Delete exon 23 and | NmCas9 (NNNNGNNT |
| | Meningitidis | | bypass mdx mutation | PAM) |
| DCR62 | N. | NNNNGANN | Delete exon 23 and | NmCas9 (NNNNGANN |
| | Meningitidis | | bypass mdx mutation | PAM) |
| DCR63 | N. | NNNNGTTN | Delete exon 23 and | NmCas9 (NNNNGTTN |
| | Meningitidis | | bypass mdx mutation | PAM) |
| DCR64 | N. | NNNNGNNT | Delete exon 23 and | NmCas9 (NNNNGNNT |
| | Meningitidis | | bypass mdx mutation | PAM) |
| DCR65 | N. | NNNNGTTN | Delete exon 23 and | NmCas9 (NNNNGTTN |
| | Meningitidis | | bypass mdx mutation | PAM) |
| DCR66 | N. | NNNNGANN | Delete exon 23 and | NmCas9 (NNNNGANN |
| | Meningitidis | | bypass mdx mutation | PAM) |
| DCR67 | N. | NNNNGANN | Target 3′ splice donor of | NmCas9 (NNNNGANN |
| | Meningitidis | | exon 51 to skip exon | PAM) |
| DCR68 | N. | NNNNGANN | Target 3′ splice donor of | NmCas9 (NNNNGANN |
| | Meningitidis | | exon 51 to skip exon | PAM) |
| DCR69 | N. | NNNNGANN | Target 3′ splice donor of | NmCas9 (NNNNGANN |
| | Meningitidis | | exon 51 to skip exon | PAM) |
| DCR70 | N. | NNNNGANN | Frameshift in exon 51 | NmCas9 (NNNNGANN |
| | Meningitidis | | | PAM) |
| DCR71 | N. | NNNNGNNT | Frameshift in exon 51 | NmCas9 (NNNNGNNT |
| | Meningitidis | | | PAM) |
| DCR72 | N. | NNNNGNNT | Target 3′ splice donor of | NmCas9 (NNNNGNNT |
| | Meningitidis | | exon 51 to skip exon | PAM) |
| DCR73 | N. | NNNNGNNT | Delete exon 51 (bind as | NmCas9 (NNNNGNNT |
| | Meningitidis | | close to DCR5 as | PAM) |
| | | | possible) | |
| DCR74 | N. | NNNNGANN | Delete exon 51 (bind as | NmCas9 (NNNNGANN |
| | Meningitidis | | close to DCR5 as | PAM) |
| | | | possible) | |
| DCR75 | N. | NNNNGTTN | Delete exon 51 (bind as | NmCas9 (NNNNGTTN |
| | Meningitidis | | close to DCR5 as | PAM) |
| | | | possible) | |
| DCR76 | N. | NNNNGNNT | Delete exon 51 (bind as | NmCas9 (NNNNGNNT |
| | Meningitidis | | close to DCR1/2 as | PAM) |
| | | | possible) | |
| DCR77 | N. | NNNNGTTN | Delete exon 51 (bind as | NmCas9 (NNNNGTTN |
| | Meningitidis | | close to DCR1/2 as | PAM) |
| | | | possible) | |
| DCR78 | N. | NNNNGANN | Delete exon 51 (bind as | NmCas9 (NNNNGANN |
| | Meningitidis | | close to DCR1/2 as | PAM) |
| | | | possible) | |
| DCR79 | N. | NNNNGNNT | Delete exons 45-55- | NmCas9 (NNNNGNNT |
| | Meningitidis | | overlaps NNNNGTTN | PAM) |
| | | | PAM, bind as close to | |
| | | | DCR6 as possible | |
| DCR80 | N. | NNNNGANN | Delete exons 45-55, bind | NmCas9 (NNNNGANN |
| | Meningitidis | | as close to DCR6 as | PAM) |
| | | | possible | |
| DCR81 | N. | NNNNGTTN | Delete exons 45-55, bind | NmCas9 (NNNNGTTN |
| | Meningitidis | | as close to DCR6 as | PAM) |
| | | | possible | |
| DCR82 | N. | NNNNGNNT | Delete exons 45-55, bind | NmCas9 (NNNNGNNT |
| | Meningitidis | | as close to DCR36 as | PAM) |
| | | | possible-overlaps | |
| | | | NNNNGTTN PAM | |
| DCR83 | N. | NNNNGTTN | Delete exons 45-55, bind | NmCas9 (NNNNGTTN |
| | Meningitidis | | as close to DCR36 as | PAM) |
| | | | possible | |
| DCR84 | N. | NNNNGANN | Delete exons 45-55, bind | NmCas9 (NNNNGANN |
| | Meningitidis | | as close to DCR36 as | PAM) |
| | | | possible | |
| DCR85 | S. Aureus | NNGRRT | Delete exons 45-55, bind | SaCas9 (from Zhang |
| | | | as close to DCR6 as | pX441) (NNGRRT |
| | | | possible | PAM) |
| DCR86 | S. Aureus | NNGRRT | Delete exons 45-55, bind | SaCas9 (from Zhang |
| | | | as close to DCR36 as | pX441) (NNGRRT |
| | | | possible | PAM) |

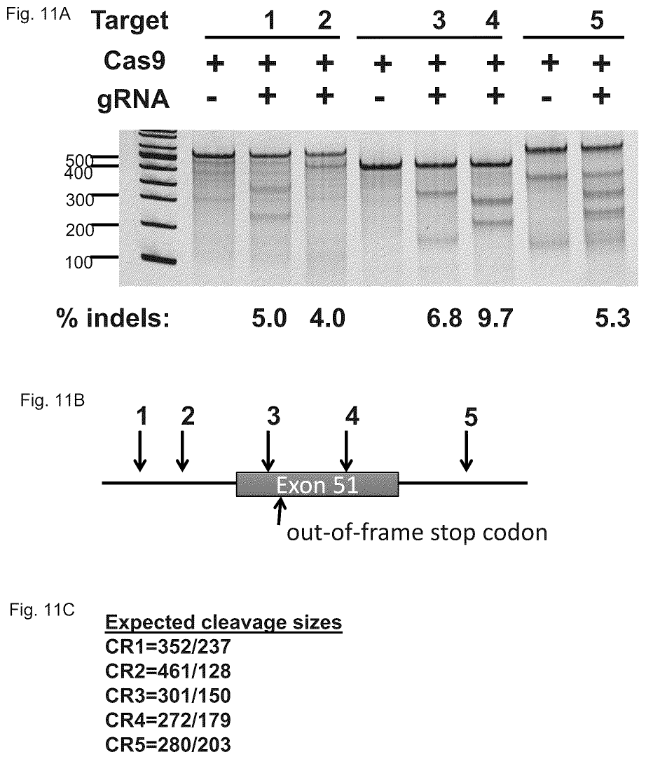

In particular, 400 ng of Cas9 was co-transfected into HEK 293T cells with either 400 ng of empty vector or gRNA that targets the region encompassing Exon 51, i.e., CR1, CR2, CR3, CR4, and CR5 (see FIG. 11B). Genomic DNA was harvested at 2 days post-transfection and analyzed using the Surveyor assay (see FIGS. 11A and 11C).

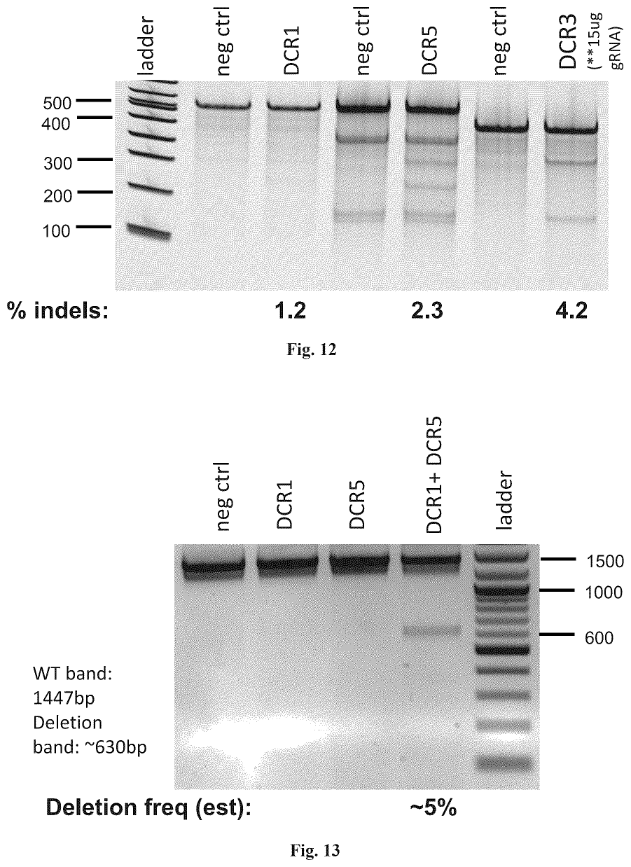

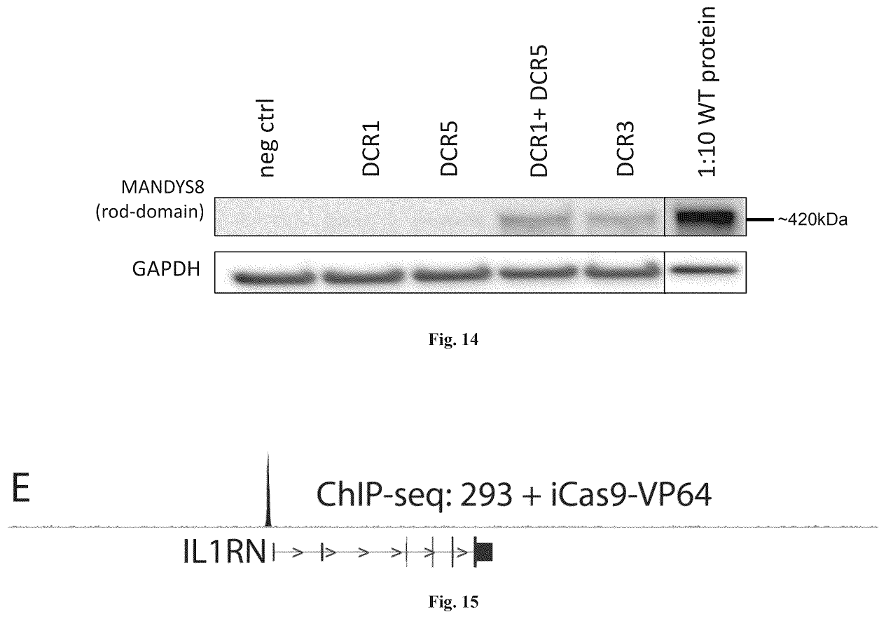

The CRISPR/Cas9-based system was used in DMD 8036 (del48-50) cells to determine if the system could repair a mutant dystrophin gene. 5 μg of Cas9 was co-transfected into DMD 8036 (del48-50) cells with either 7.5 μg of empty vector or gRNA. In particular, 7.5 μg of CR1 (“DCR1”), 7.5 μg of CR5 (“DCR5”), 15 μg of CR3 (“DCR3”) or 7.5 μg of a combination of CR1 and CR5 (DCR1+DCR5) were used. Genomic DNA was harvested at 3 days post-transfection and analyzed using the Surveyor assay (FIG. 12) or PCR analysis across the entire locus (FIG. 13). This locus was amplified by PCR using primers flanking the region containing the genomic targets for CR1 and CR5 (the forward primer: 5′-gagaggttatgtggctttacca (SEQ ID NO: 457), the reverse primer: 5′-ctgcgtagtgccaaaacaaa (SEQ ID NO:458)), resulting in a 1447 bp band for the wild-type locus or an expected size of approximately 630 bp for the deleted locus. After 7 days of differentiation, western blot of the treated cells shows expression of dystrophin protein (see FIG. 14).

### Example 5

### Targeting CRISPR/Cas9 to Hotspot Mutations in the Human Dystrophin Gene

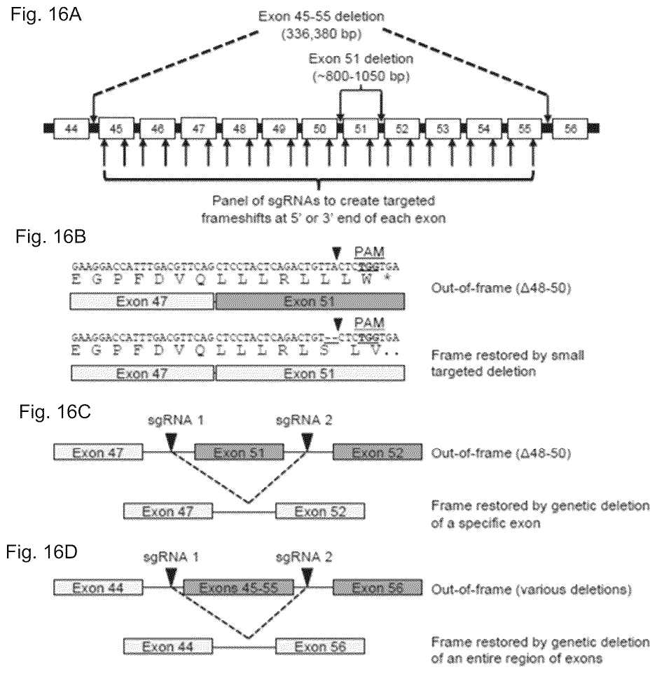

To utilize the CRISPR/Cas9 gene editing platform for correcting a wide range of dystrophin mutations, dozens of sgRNAs targeted to the hotspot mutation region between exons 45-55 were created (FIGS. 16A-16D). The S. pyogenes system that utilizes a human-codon optimized SpCas9 nuclease and a chimeric single-guide RNA (sgRNA) expression vector to guide efficient site-specific gene editing was used. Similar to Example 4 targeting exon 51 with TALENs, protospacers were selected to target the 5′ and 3′ ends of exons 45 through 55 which meet the 5′-NRG-3′ PAM requirement of SpCas9. Small insertions or deletions created by NHEJ-based DNA repair within these exons can generate targeted frameshift mutations that address various dystrophin mutations surrounding each exon (FIGS. 16A-16B). For example, CR3 was designed to correct dystrophin mutations or deletions surrounding exon 51 by introducing small insertions or deletions in the 5′ end of exon 51 to restore the downstream dystrophin reading frame (FIG. 16B). Additionally, sgRNAs were designed to employ the multiplex capability of the CRISPR/Cas9 system and specifically delete individual exons or a series of exons to restore the dystrophin reading frame, similar to the methods of oligonucleotide-based exon skipping. For this purpose, sgRNAs were targeted to the intronic regions surrounding exon 51 (FIG. 16C) or exons 45-55 (FIG. 16D). These sgRNAs were intentionally targeted to sites nearest to the downstream or upstream exon intended to be included in the resulting transcript to minimize the likelihood that the background patient deletion would include the intronic sgRNA target sites.

### Example 6

Screening of sgRNAs Targeted to the Dystrophin Gene in Human Cells

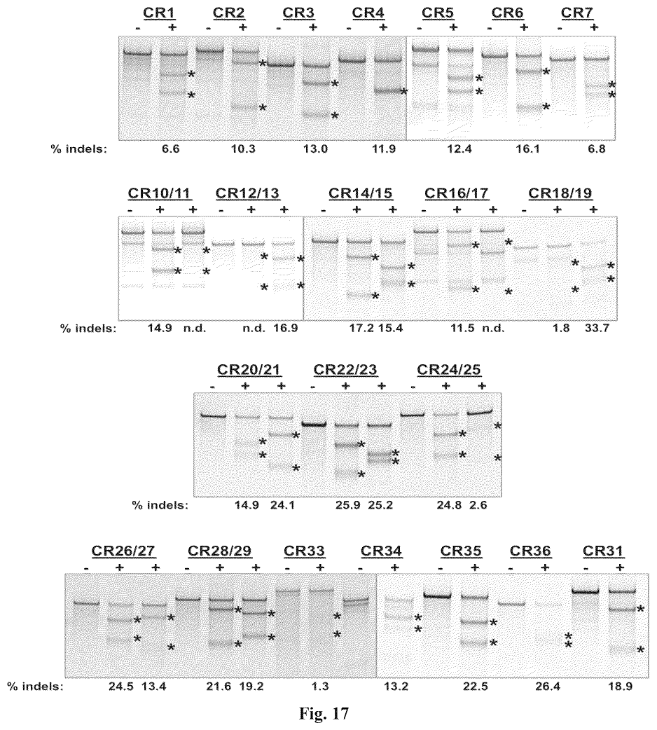

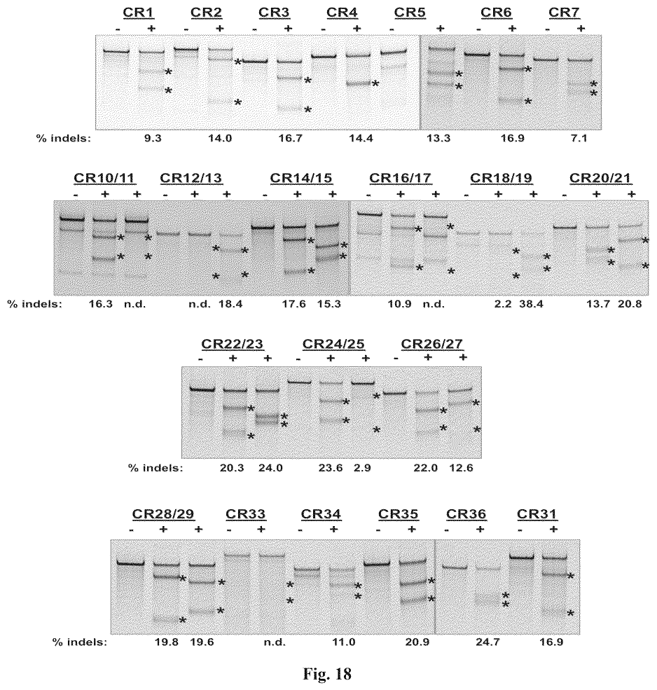

Gene editing frequency in the human HEK293T cell line was assessed to rapidly determine different sgRNA targeting efficiencies. HEK293 Ts were transfected with constructs encoding human codon-optimized SpCas9 and the indicated sgRNA. Each sgRNA was designed to modify the dystrophin gene as indicated. The frequency of gene modification at day 3 or day 10 post-transfection was determined by the Surveyor assay. The ratio of measured Surveyor signal at day 3 and day 10 was calculated to quantify the stability of gene editing frequencies for each sgRNA in human cells. As quantified by the Surveyor assay 3 days post-transfection, 29/32 (~90%) of the sgRNAs tested were able to mediate highly efficient gene modification at the intended locus (Table 9, FIG. 17). The gene editing frequencies were stable for almost all of the sgRNAs (<25% signal change from day 3 to day 10, Table 9, FIG. 18), indicating that gene editing mediated by each individual sgRNA was well-tolerated. A notable exception is CR33, which had no detectable activity at day 10, although activity may be below the sensitivity of the Surveyor assay (est. ~1%).

TABLE 9
Measured activity of sgRNAs in human cells

| | | | | |
|---|---|---|---|---|
| | | % modified | % modified | % change |
| | | alleles | alleles | day 10/ |
| Target | sgRNA # | at day 3 | at day 10 | day 3 |

Multiplex deletion of exon 51

| | | | | |
|---|---|---|---|---|
| Int 50 | CR1 | 6.6 | 9.3 | 41.8 |
| Int 50 | CR2 | 10.3 | 14.0 | 36.2 |
| Ex 51 | CR4 | 11.9 | 14.4 | 21.3 |
| Int 51 | CR5 | 12.4 | 13.3 | 7.8 |

Multiplex deletion of exons 45-55

| | | | | |
|---|---|---|---|---|
| Int 44 | CR6 | 16.1 | 16.9 | 4.3 |
| Int 44 | CR33 | 1.3 | <1 | n.d. |
| Int 44 | CR34 | 13.2 | 11.0 | −16.6 |
| Int 55 | CR7 | 6.8 | 7.1 | 5.3 |
| Int 55 | CR35 | 22.5 | 20.9 | −7.1 |
| Int 55 | CR36 | 26.4 | 24.7 | −6.4 |

Targeted frameshifts

| | | | | |
|---|---|---|---|---|
| Ex 45 | CR10 | 14.9 | 16.3 | 9.3 |
| Ex 45 | CR11 | <1 | <1 | n.d. |
| Ex 46 | CR12 | <1 | <1 | n.d. |
| Ex 46 | CR13 | 16.9 | 18.4 | 9.2 |
| Ex 47 | CR14 | 17.2 | 17.6 | 2.9 |
| Ex 47 | CR15 | 15.4 | 15.3 | −0.9 |
| Ex 48 | CR16 | 11.5 | 10.9 | −5.0 |
| Ex 48 | CR17 | <1 | <1 | n.d. |
| Ex 49 | CR18 | 1.8 | 2.2 | 20.1 |
| Ex 49 | CR19 | 33.7 | 38.4 | 13.9 |
| Ex 50 | CR20 | 14.9 | 13.7 | −7.6 |
| Ex 50 | CR21 | 24.1 | 20.8 | −13.5 |
| Ex 51 | CR3 | 13.0 | 16.7 | 28.0 |
| Ex 51 | CR31 | 18.9 | 16.9 | −10.2 |
| Ex 52 | CR22 | 25.9 | 20.3 | −21.6 |
| Ex 52 | CR23 | 25.2 | 24.0 | −4.8 |
| Ex 53 | CR24 | 24.8 | 23.6 | −4.6 |
| Ex 53 | CR25 | 2.6 | 2.9 | 9.5 |
| Ex 54 | CR26 | 24.5 | 22.0 | −10.1 |
| Ex 54 | CR27 | 13.4 | 12.6 | −5.9 |
| Ex 55 | CR28 | 21.6 | 19.8 | −8.4 |
| Ex 55 | CR29 | 19.2 | 19.6 | 2.2 |

### Example 7

### Enrichment of Gene-Edited Cells Using a Fluorescence-Based Reporter System

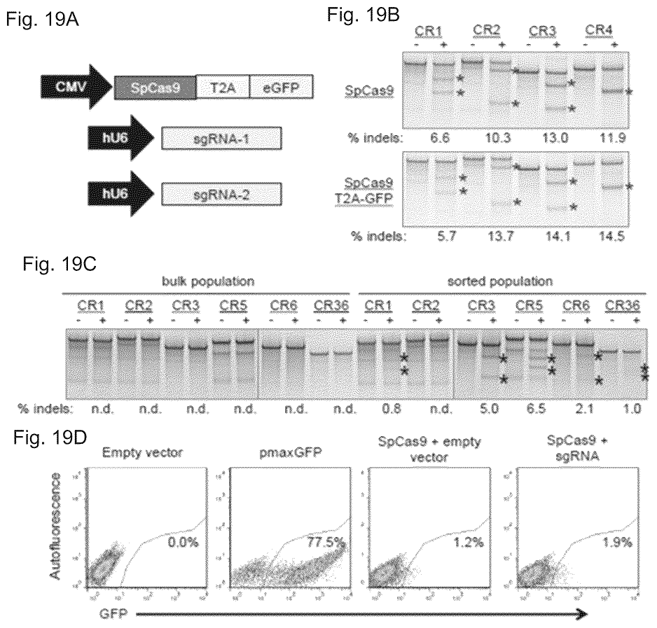

sgRNAs were selected to correct specific mutations in DMD patient myoblast cell lines. After transfection into DMD myoblasts, unexpectedly low or undetectable gene modification activity was observed as measured by the Surveyor assay (FIG. 19C, bulk population). Flow cytometry was used to select for transfected cells co-expressing GFP through a 2A ribosomal skipping peptide linked to the SpCas9 protein (FIG. 19A). The addition of this fluorescent reporter to the SpCas9 expression vector did not seem to significantly impact gene editing activity in HEK293T cells (FIG. 19B). A low percentage of transfected myoblasts (~0.5-2%) expressed the fluorescent reporter at 3 days after electroporation, despite high transfection efficiencies of control GFP expression plasmids (typically >70%, FIG. 19D, pmaxGFP). Given the high levels of CRISPR/Cas9 activity in the easily transfected HEK293T line, inefficient transgene expression after electroporation of SpCas9-T2A-GFP and sgRNA constructs into the DMD cells may explain the low observed gene editing efficiencies in unsorted cells. After sorting the GFP-positive DMD myoblasts, a substantial increase was observed in detectable activity at most sgRNA target loci (FIG. 19C). Therefore, all subsequent experiments used cells sorted for SpCas9 expression by expression of this fluorescent reporter.

### Example 8

### Restoration of Dystrophin Expression by Targeted Frameshifts

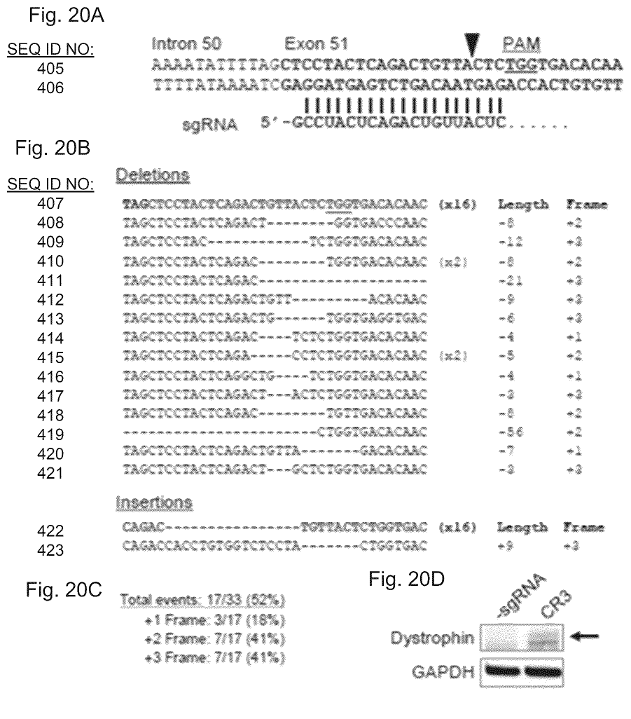

Small insertions and deletions created by NHEJ DNA repair may be used to create targeted frameshifts to correct aberrant reading frames. A sgRNA, CR3, was designed to restore the dystrophin reading frame by introducing small insertions and deletions within exon 51 (FIGS. 16B, 20A). The types of insertions and deletions generated by CRISPR/Cas9 at this locus were assessed by Sanger sequencing of alleles from the genomic DNA of HEK293T cells co-transfected with expression plasmids for SpCas9 and the CR3 sgRNA (FIG. 20B). Notably, the insertions and deletions resulted in conversion to all three reading frames (FIGS. 20B, 20C). To demonstrate genetic correction in a relevant patient cell line, expression plasmids for SpCas9 and the CR3 sgRNA were electroporated into a DMD myoblast line with a deletion of exons 48-50 that is correctable by creating frameshifts in exon 51. The treated cells were sorted, verified to have gene modification activity by the Surveyor assay (CR3, FIG. 19C sorted population), and differentiated into myotubes to test for restored dystrophin expression. Expression of dystrophin protein was observed concomitant with the detectable nuclease activity (FIG. 20D). The S. pyogenes CRISPR/Cas9 system presents a powerful method to quickly generate targeted frameshifts to address a variety of patient mutations and restore expression of the human dystrophin gene.

### Example 9

### Multiplex CRISPR/Cas9 Gene Editing Mediates Genomic Deletion of Exon 51 and Rescues Dystrophin Protein Expression

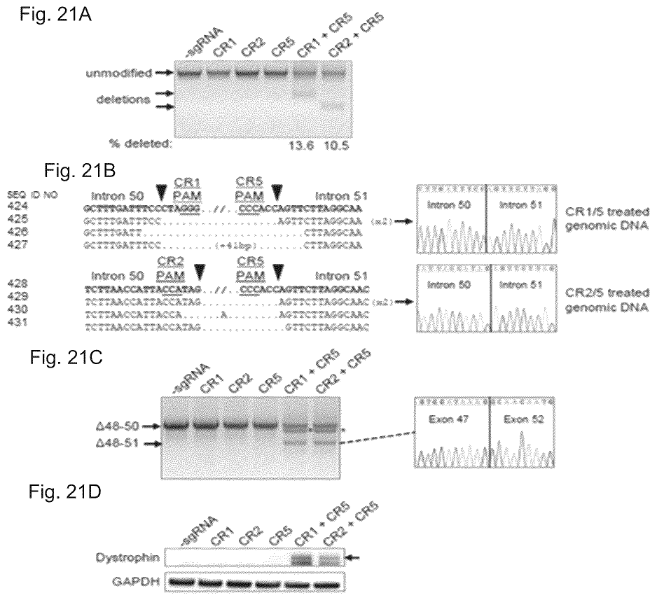

The multiplexing capability of the CRISPR/Cas9 system presents a novel method to efficiently generate genomic deletions of specific exons for targeted gene correction. DMD patient myoblasts with background deletions correctable by exon 51 skipping were treated with two combinations of sgRNAs flanking exon 51 (CR1/CR5 or CR2/CR5) and sorted to enrich for gene-edited cells as in FIGS. 19A-19D. As detected by end-point PCR of the genomic DNA from these treated cells, the expected genomic deletions were only present when both sgRNAs were electroporated into the cells with SpCas9 (FIG. 21A). Sanger sequencing confirmed the expected junction of the distal chromosomal segments (FIG. 21B) for both deletions. After differentiating the sorted myoblasts, a deletion of exon 51 from the mRNA transcript was detected only in the cells treated with both sgRNAs (FIG. 21C). Finally, restored dystrophin protein expression was detected in the treated cells concomitant with observed genome- and mRNA-level deletions of exon 51 (FIG. 21D).

### Example 10

### Dystrophin Rescue by a Multi-Exon Large Genomic Deletion

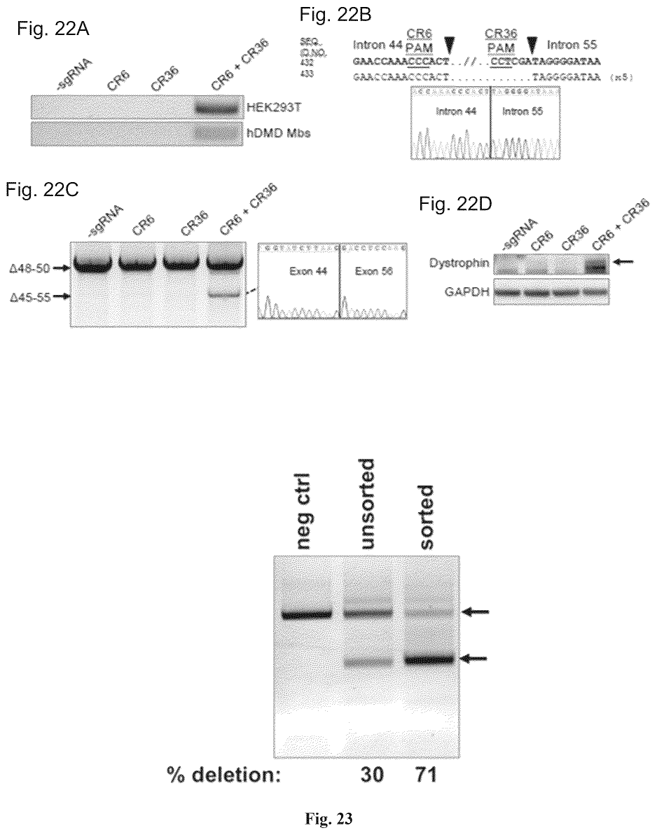

Although addressing patient-specific mutations is a powerful use of the CRISPR/Cas9 system, it would be advantageous to develop a single method that can address a myriad of common patient deletions. For example, a promising strategy is to exclude the entire exon 45-55 region as a method to correct up to 62% of known patient deletions. Multiplex CRISPR/Cas9-based gene editing was tested to determine if it may be able to generate efficient deletion of the exon 45-55 locus in human cells. After transfection into HEK293T cells, the expected deletion of ~336,000 bp was detected by PCR of the genomic DNA (FIG. 22A). Similarly, this deletion was detected by PCR of the genomic DNA from SpCas9/sgRNA-treated DMD patient cells harboring a background deletion of exons 48-50 of unknown length (FIG. 22A). Sanger sequencing of this deletion band from the genomic DNA of treated DMD cells revealed the expected junctions of intron 44 and intron 55 immediately adjacent to the sgRNA target sites (FIG. 22B). After differentiation of treated DMD cells, the expected deletion of exons 45-55 was detected in the dystrophin mRNA transcript and verified to be a fusion of exons 44 and 56 by Sanger sequencing (FIG. 22C). Restored protein expression was observed by western blot in the sorted cell populations containing the CRISPR/Cas9-induced deletion of exons 45-55 from the genome and resulting mRNA transcripts (FIG. 22D). These data demonstrate that multiplex CRISPR/Cas9 editing presents a single universal method to restore the dystrophin reading frame in more than 60% of DMD patient mutations.

### Example 11

Transplantation of Corrected Myoblasts into Immunodeficient Mice

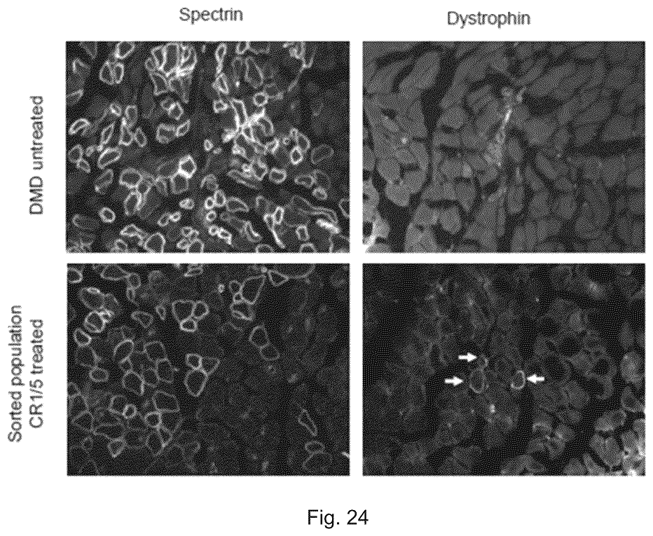

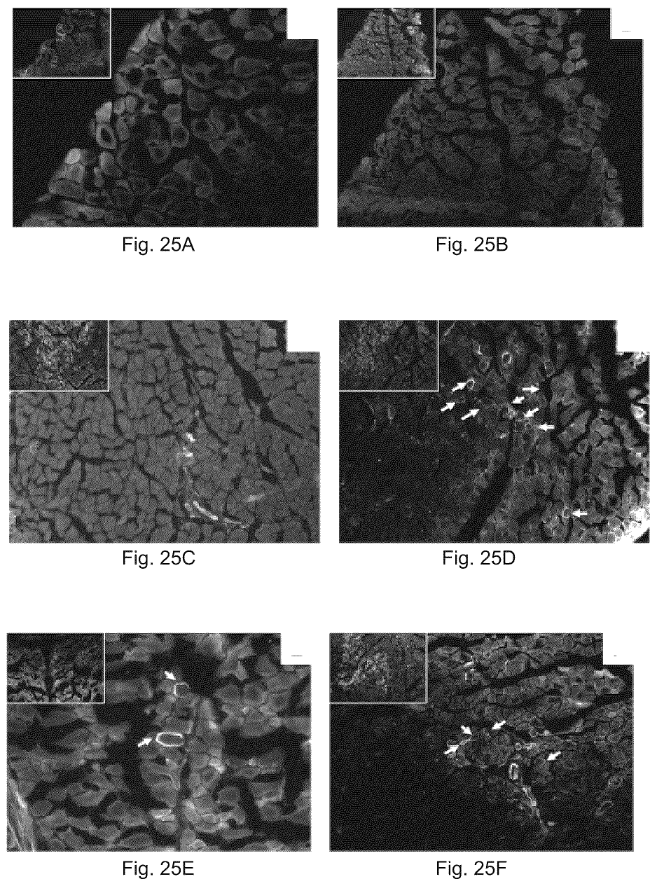

A promising method for DMD therapy is to correct a population of autologous patient muscle progenitor cells that can be engrafted into the patient's skeletal muscle tissue to rescue dystrophin expression. To demonstrate the ability of the corrected cells to express human dystrophin in vivo, a population of DMD myoblasts that were treated with sgRNAs CR1 and CR5, which flank exon 51, was transplanted and sorted for expression of GFP as before (FIGS. 19A-19D, FIG. 23). After 4 weeks, muscle fibers positive for human spectrin, which is expressed by both corrected and uncorrected cells, were detected in cryosections of injected muscle tissue (FIG. 24). A number of these fibers were also positive for human dystrophin with expression localized to the sarcolemma, demonstrating functional gene correction in these cells (FIG. 24, FIGS. 25A-25F). No fibers positive for human dystrophin were observed in sections from mice injected with the untreated DMD myoblasts (FIG. 24, FIGS. 25A-25F), indicating that the CRISPR/Cas9-modified cells were the source of human dystrophin expression.

### Example 12

### Off-Target and Cytotoxicity Analysis

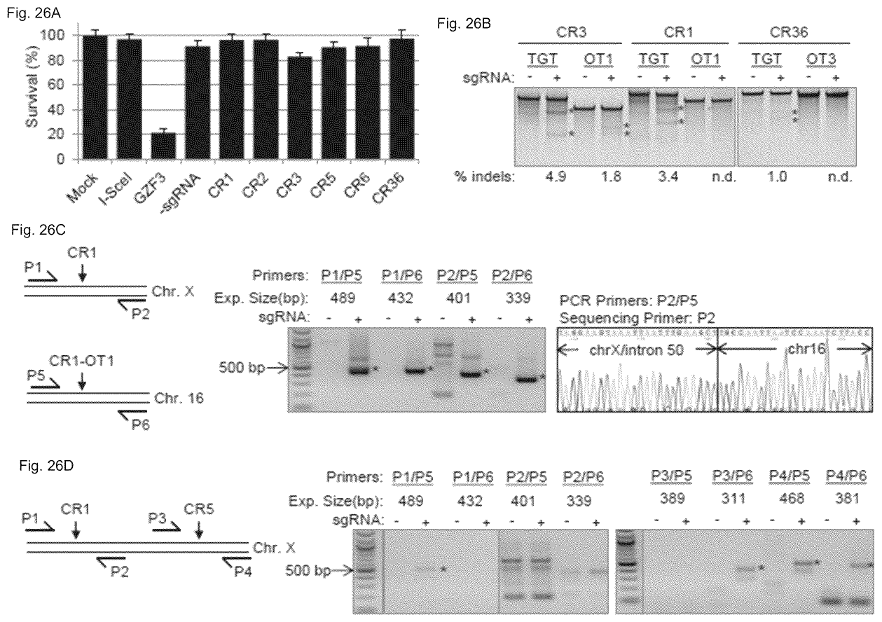

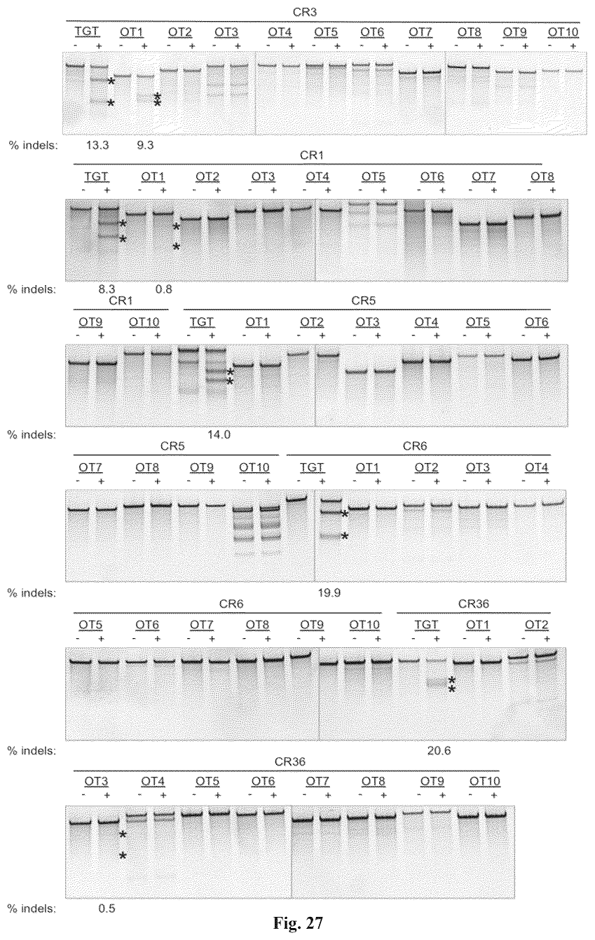

The relative cytotoxicity of the CRISPR/Cas9 system was assessed in human cells for select sgRNAs by adapting a flow cytometry-based GFP retention assay as previously described (Ousterout et al., Mol Ther 21:1718-1726 (2013)). Minimal cytotoxicity was observed for SpCas9 co-expressed with or without sgRNAs after transfection into human cells (FIG. 26A). Publicly available tools are available to assess and prioritize potential CRISPR/Cas9 activity at off-target loci based on predicted positional bias of a given mismatch in the sgRNA protospacer sequence and the total number of mismatches to the intended target site (Hsu et al., Nat Biotechnol 31:827-826 (2013)). This public webserver was used to predict the most likely off-target sites for the sgRNAs used to correct the dystrophin gene in this study (Table 4). The top ten potential off-target sites were assessed by the Surveyor assay in HEK293T cells treated with SpCas9 and individual sgRNA expression cassettes for CR1, CR3, CR5, CR6, or CR36. CR1, CR3 and CR36 each had one of these ten predicted off-target loci demonstrate significant levels of gene modification (Table 4 and FIG. 27). Interestingly, the CR3 off-target sequence had substantial homology and similar modification frequencies to the intended on-target (9.3% at OT-1 vs. 13.3% at intended site (Table 4 and FIG. 27). Notably, CR3-OT1 was the only one of these three off-target sites to show significant levels of activity in the sorted hDMD cells by the Surveyor assay (FIG. 26B).

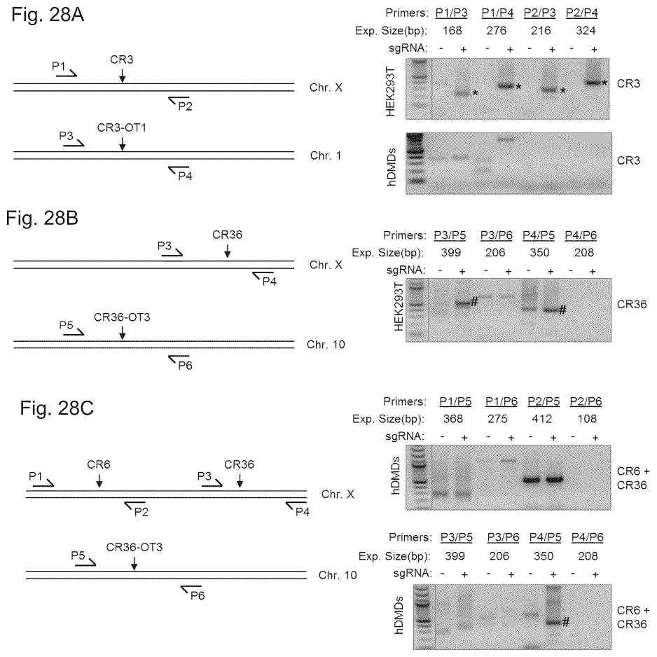

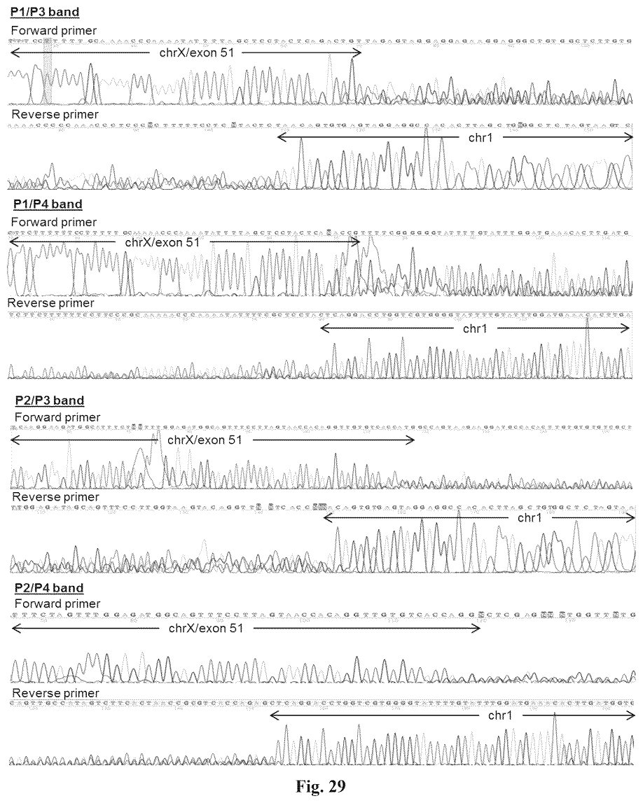

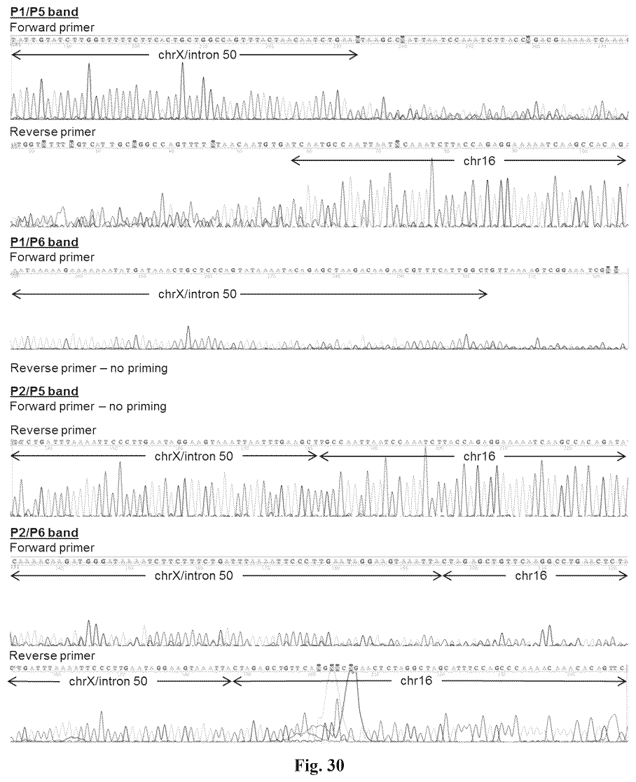

Nuclease activity at off-target sites may cause unintended chromosomal rearrangements by distal re-ligation between cleaved target and off-target loci on distinct chromosomes. This presents a significant concern for deletion-based gene correction strategies due to the increased potential for off-target activity by using two or more nucleases, such as in multiplex CRISPR/Cas9 gene editing. Potential translocations were probed for using a highly sensitive nested genomic PCR assay to detect translocations at the validated off-target loci (Table 4) during both single and multiplex CRISPR/Cas9 editing strategies. Using this assay, translocations were readily detected between on-target and off-target sites in the model HEK293T cell line that also shows high levels of off-target activity (FIG. 26C and FIG. 28A, 28B). Sanger sequencing of the PCR amplicons confirmed the identity of the predicted translocation event for each primer pair (FIGS. 29-30). A subset of the translocations detected in the HEK293T cells were also detectable by nested PCR in the sorted hDMD myoblasts, although the signal was considerably weaker and the sequence identity was not confirmed due to low yield of product (FIG. 26D and FIGS. 28A, 28C). Translocations were not detected using this assay in HEK293T or sorted hDMD cells treated with CR6 or CR6/CR36, respectively, (FIGS. 28A-28C) that had low levels of off-target activity at CR6-OT3 only in HEK293T cells (Table 4). These results underscore the importance of selecting highly specific sgRNAs, particularly for multiplex editing applications, and show that this approach can benefit from ongoing efforts to improve the specificity of the CRISPR/Cas9 system. These data suggest that the selected sgRNAs are able to correct the dystrophin gene without significant toxicity and with only a single strongly predicted off-target site with detectable levels of activity.

### Example 13

### Discussion

Genome editing is a powerful tool for correcting genetic disease and the recent development of the CRISPR/Cas9 system is dramatically accelerating progress in this area. The correction of DMD, the most common genetic disease that also currently has no approved therapeutic options, was demonstrated. Many gene- and cell-based therapies for DMD are in preclinical development and clinical trials, and genome editing methods are compatible with many of these approaches. For example, genome editing may be combined with patient-specific cell-based therapies for DMD. The CRISPR/Cas9 system may function in human pluripotent stem cells and other human cell lines, as well as human skeletal myoblasts, as shown. Importantly, gene editing with CRISPR/Cas9 did not abolish the myogenic capacity of these cells, as demonstrated by efficient dystrophin expression in vitro and in vivo after transplantation into immunodeficient mice. Thus, this strategy should be compatible with cell-based therapies for DMD.

Additionally, an enriched pool of gene-corrected cells demonstrated expression of human dystrophin in vivo following engraftment into immunodeficient mice. CRISPR/Cas9 gene editing did not have significant toxic effects in human myoblasts as observed by stable gene editing frequencies and minimal cytotoxicity of several sgRNAs. However, gene editing activity was confirmed at three out of 50 predicted off-target sites across five sgRNAs and CRISPR/Cas9-induced chromosomal translocations between on-target and off-target sites were detectable. The CRISPR/Cas9 technology is an efficient and versatile method for correcting a significant fraction of dystrophin mutations and can serve as a general platform for treating genetic disease.

Additionally, direct transfection of the sgRNA and Cas9 mRNA, in contrast to the plasmid-based delivery method used here, may be used to increase specificity and safety by reducing the duration of Cas9 expression and eliminating the possibility of random plasmid integration. Alternatively, delivery of the CRISPR/Cas9 system directly to skeletal and/or cardiac muscle by viral, plasmid, or RNA delivery vectors may be used for in vivo genome editing and translation of this approach. The large size of S. pyogenes Cas9 gene (~4.2 kilobases) presents a challenge to its use in size-restricted adeno-associated viral vectors. However, Cas9 genes from other species, such as N. meningitidis and S. thermophilus, are short enough to efficiently package both Cas9 and sgRNA expression cassettes into single AAV vectors for in vivo gene editing applications.

The CRISPR/Cas9 system enabled efficient modification of nearly 90% of tested targets, consistent with other reports of robust activity of this system at diverse loci. The robustness and versatility of this technology is a significant advancement towards at-will creation of patient-specific gene editing. Low levels of dystrophin, including as little as 4% of wild-type expression, may be sufficient to improve survival, motor function, and cardiac function in a mouse model. The levels of CRISPR/Cas9 activity may be sufficient for therapeutic benefit.

The use of multiplexing with CRISPR/Cas9 to delete exons also presents a unique set of opportunities and challenges. The deletion of complete exons from the genome to restore dystrophin expression was performed, in contrast to restoring the reading frame of the dystrophin gene with small indels generated by NHEJ-based DNA repair following the action of a single nuclease. The protein product of the edited gene is predictable and already characterized in Becker muscular dystrophy patients with the naturally occurring deletion, in contrast to the random indels created by intraexonic action of a single nuclease that will lead to the creation of novel epitopes from each DNA repair event. Furthermore, the product resulting from the exon deletions will lead to restored dystrophin for every successful gene editing event, whereas modifying the gene with random indels within exons will only restore the reading frame in the one-third of editing events that leads to the correct reading frame.

All of the sgRNAs tested were not associated with significant cytotoxic effects in human cells. Three potential off-target sites out of 50 total tested sites for the five sgRNAs used were identified to restore dystrophin expression. Furthermore, chromosomal translocations between the intended on-target sites and these off-target sites were detectable by highly sensitive nested PCR assays in HEK293T cells expressing high levels of Cas9 and sgRNAs. Notably, the off-target activity and translocations identified in HEK293T cells, which is an immortalized and aneuploid cell line that expresses very high levels of Cas9 and sgRNA, did not occur at as high a level and in some cases were undetectable in the hDMD myoblasts. Importantly, this level of specificity may be tolerable given the severity of DMD, the lack of an apparent cytotoxic effect in human cells

### Example 14

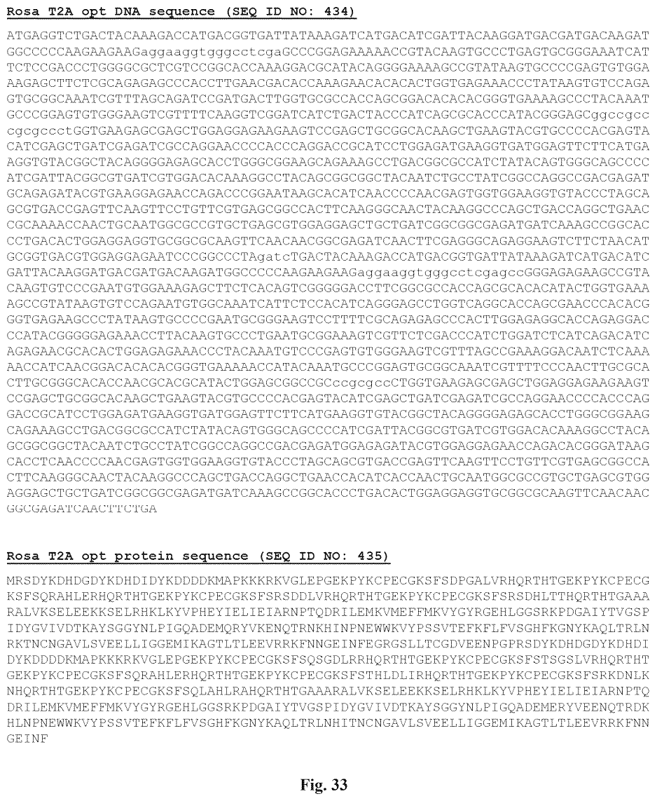

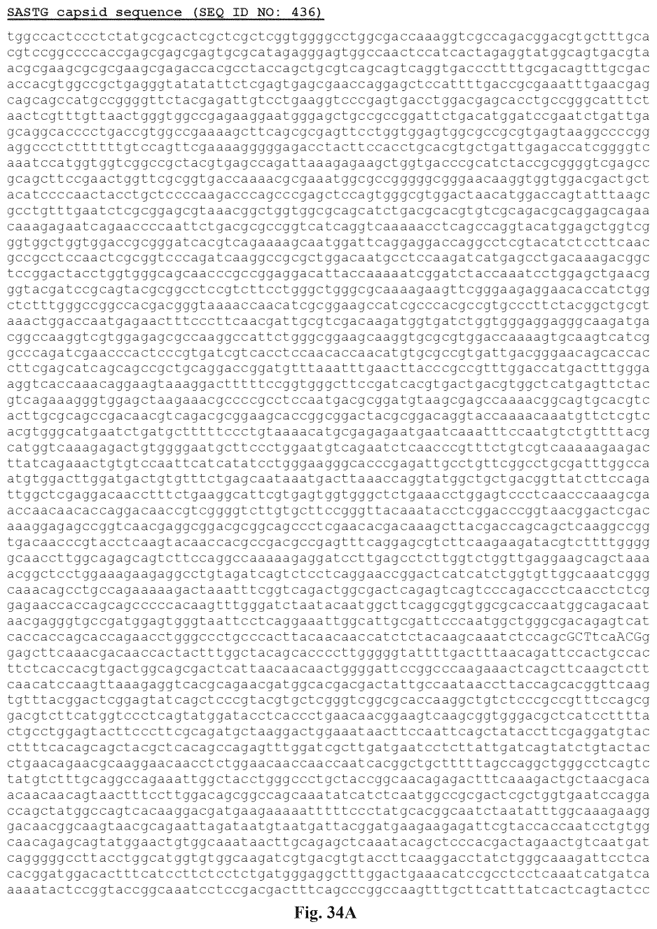

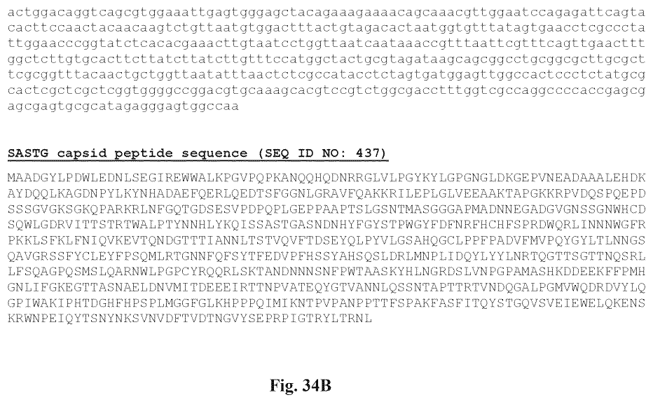

An engineered AAV capsid, termed SASTG (FIG. 34; SEQ ID NOs: 436 and 437), was developed for enhanced cardiac and skeletal muscle tissue tropism (Piacentino et al. (2012) Human Gene Therapy 23:635-646). A ZFN targeting the Rosa26 locus (“Rosa26 ZFN”; FIG. 33; SEQ ID NO: 434 and 435) was shown to be highly active in mouse cells (Perez-Pinera et al. Nucleic Acids Research (2012) 40:3741-3752). AAV-SASTG vectors encoding the Rosa26 ZEN protein were designed and subsequently generated and purified by the UNC Viral Vector Core. The Surveyor assay (Guschin et al., Methods Mol Biol 649, 247-256 (2010)) was used to demonstrate NHEJ mutagenesis at the Rosa26 locus following delivery of AAV-SASTG Rosa26 ZFNs in cultured C2C12 myoblasts that were actively cycling or forced into differentiation by serum removal (not shown).

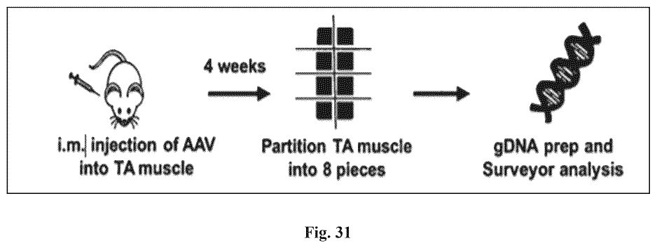

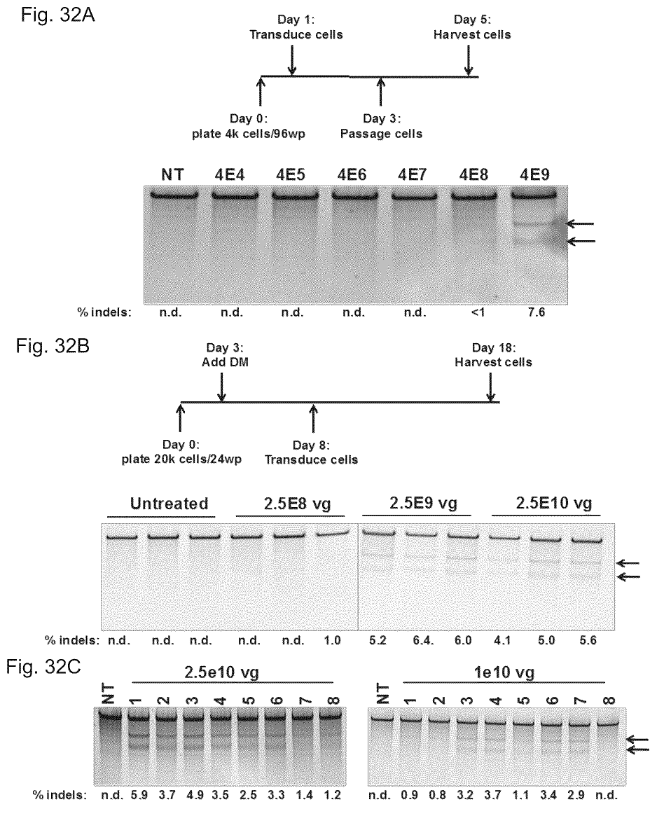

To verify that adult post-mitotic skeletal muscle were efficiently targeted by the Rosa26 ZFN following AAV transfer, AAV-SASTG vectors encoding the Rosa26 ZFN were injected directly into the tibialis anterior (TA) muscle of 6 week old C57BL6/J mice at titers of 1e10 vector genomes (vg) or 2.5e10 vg per muscle. Mice were sacrificed 4 weeks after injection and the TA muscles were harvested and partitioned into several fragments for genomic DNA extraction and analysis (FIG. 31). Genomic DNA was PCR amplified and subjected to the Surveyor assay to detect NHEJ mutations characteristic of ZFN mutagenesis at the Rosa26 target site (FIGS. 32A-32C). FIGS. 32A-32C show Surveyor analysis of Rosa26 ZFN activities in skeletal muscle in vitro and in vivo following delivery of AAV-SASTG-ROSA. Proliferating C2C12s were transduced with the indicated amount of virus and harvested at 4 days post-infection (FIG. 32A). C2C12s were incubated in differentiation medium for 5 days and then transduced with the indicated amount of AAV-SASTG-ROSA virus in 24 well plates (FIG. 32B). Samples were collected at 10 days post-transduction. The indicated amount of AAV-SASTG-ROSA was injected directly into the tibialis anterior of C57BL/6J mice and muscles were harvested 4 weeks post-infection. The harvested TA muscles were partitioned into 8 separate pieces for genomic DNA analysis, each shown in a separate lane (FIG. 32C). Notably, high levels of gene modification were detected in all fragments at the highest dose (2.5el0 vg).

### Example 15

### AAV-CRISPR Constructs for Targeting Mutant Dystrophin Genes

AAV constructs are designed to therapeutically correct mutations of the dystrophin gene that cause Duchenne muscular dystrophy and skeletal and cardiac muscle degeneration. CRISPR/Cas9 systems can be delivered using the AAV to restore the dystrophin reading frame by deleting exon 51, deleting exons 45-55, disrupting splice donor or acceptor sites, or creating frameshifts within exon 51 (Ousterout et al., Molecular Therapy 2013) to restore the dystrophin reading frame and protein expression. The CRISPR/Cas9 system will include a Cas9 having a sequence of SEQ ID NO: 64 or 114 (See FIGS. 40 and 41). gRNAs that could be combined with these Cas9s, targeting their respective PAM sequences, are provided (see FIGS. 40 and 41; see also Tables 2 and 3).

### Example 16

Generation of Induced Neurons (iNs)

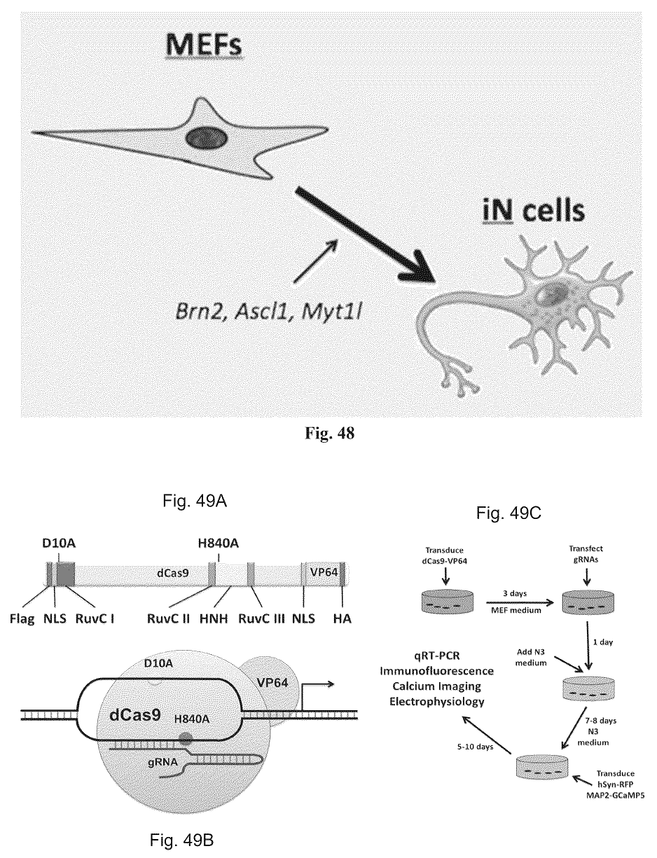

The generation of induced neurons (iNs) from other cell lineages has potential applications in regenerative medicine and the study of neurological diseases. The direct conversion of mouse embryonic fibroblasts (MEFs) to functional neuronal cells may occur through the delivery of a cocktail of three neuronal transcription factors-BRN2, ASCL1, and MYT1L (BAM factors, FIG. 48). Other methods may include additional factors to induce various neuronal subtypes. These experiments require transcription factors to be delivered ectopically, and the activation of the corresponding endogenous loci to sustain the neuronal phenotype. The CRISPR/Cas9 system was engineered as a versatile transcription factor to activate endogenous genes in mammalian cells with the capacity to target any promoter in the genome through an RNA-guided mechanism (FIGS. 49A,49B).

Materials & Methods. The CRISPR/Cas9-transcription factor was used to activate the endogenous genes encoding ASCL1 and BRN2 to directly reprogram MEFs to functional induced neurons.

Cell Culture: MEFs were seeded in either 24-well TCPS plates or on poly-D-lysine/laminin-coated coverslips. Following transduction of dCas9-VP64 and transfection of the gRNAs (see Tables 10 and 11 for sequences of gRNAs), the cells were cultured in MEF medium (Adler et al. Mol Ther Nucleic Acids 1: e32 (2012)) for 24 hrs and then transferred to N3 neural induction medium (Vierbuchen et al. Nature 463:1035-1041 (2010)) for the duration of the experiment (FIG. 49B).

TABLE 10
gRNAs for mouse ASCL1 (CR13):

| Oligo | 5′ | | | | SEQ ID |
|---|---|---|---|---|---|
| (5′ to 3′) | overhang | | ASCL1 Target Sequence | | NO |
| CR13-1_S: | cacc | G | CAGCCGCTCGCTGCAGCAG (SEQ ID NO: 468) | | 492 |
| CR13-1_AS: | AAAC | | CTGCTGCAGCGAGCGGCTG (SEQ ID NO: 469) | C | 493 |
| CR13-2_S: | cacc | G | GCTGGGTGTCCCATTGAAA (SEQ ID NO: 470) | | 494 |
| CR13-2_A5: | AAAC | | TTTCAATGGGACACCCAGC (SEQ ID NO: 471) | C | 495 |
| CR13-3_S: | cacc | G | GTTTATTCAGCCGGGAGTC (SEQ ID NO: 472) | | 496 |
| CR13-3_AS: | AAAC | | GACTCCCGGCTGAATAAAC (SEQ ID NO: 473) | C | 497 |
| CR13-4_S: | cacc | G | TGGAGAGTTTGCAAGGAGC (SEQ ID NO: 474) | | 498 |
| CR13-4_AS: | AAAC | | GCTCCTTGCAAACTCTCCA (SEQ ID NO: 475) | C | 499 |
| CR13-5_S: | cacc | G | CCCTCCAGACTTTCCACCT (SEQ ID NO: 476) | | 500 |
| CR13-5_AS: | AAAC | | AGGTGGAAAGTCTGGAGGG (SEQ ID NO: 477) | C | 501 |
| CR13-6_S: | cacc | G | AATTTTCTTCCAAGTTCTC (SEQ ID NO: 478) | | 502 |
| CR13-6_AS: | AAAC | | GAGAACTTGGAAGAAAATT (SEQ ID NO: 479) | C | 503 |
| CR13-7_S: | cacc | G | CTGCGGAGAGAAGAAAGGG (SEQ ID NO: 480) | | 504 |
| CR13-7_AS: | AAAC | | CCCTTTCTTCTCTCCGCAG (SEQ ID NO: 481) | C | 505 |
| CR13-8_S: | cacc | G | AGAGCCACCCCCTGGCTCC (SEQ ID NO: 482) | | 506 |
| CR13-8_AS: | AAAC | | GGAGCCAGGGGGTGGCTCT (SEQ ID NO: 483) | C | 507 |
| CR13-9_S: | cacc | G | cgaagccaaccgcggcggg (SEQ ID NO: 484) | | 508 |
| CR13-9_AS: | AAAC | | cccgccgcggttggcttcg (SEQ ID NO: 485) | C | 509 |
| CR13-10_S: | cacc | G | agagggaagacgatcgccc (SEQ ID NO: 486) | | 510 |
| CR13-10_AS: | AAAC | | gggcgatcgtcttccctct (SEQ ID NO: 487) | C | 511 |
| CR13-11_S: | cacc | G | cccctttaactttcctccg (SEQ ID NO: 488) | | 512 |
| CR13-11_AS: | AAAC | | cggaggaaagttaaagggg (SEQ ID NO: 489) | C | 513 |
| CR13-12_S: | cacc | G | gcagccccgcttccttcaa (SEQ ID NO: 490) | | 514 |
| CR13-12_AS: | AAAC | | ttgaaggaagcggggctgc (SEQ ID NO: 491) | C | 515 |

TABLE 11
gRNAs for mouse BRN2 (CR16):

| Oligo | 5′ | | | | SEQ ID |
|---|---|---|---|---|---|
| (5′ to 3′) | overhang | | ASCL1 Target Sequence | | NO |
| CR16-1_S: | cacc | G | CGAGAGCGAGAGGAGGGAG (SEQ ID NO: 516) | | 540 |
| CR16-1_AS: | AAAC | | CTCCCTCCTCTCGCTCTCG (SEQ ID NO: 517) | C | 541 |
| CR16-2_S: | cacc | G | GAGAGAGCTTGAGAGCGCG (SEQ ID NO: 518) | | 542 |
| CR16-2_AS: | AAAC | | CGCGCTCTCAAGCTCTCTC (SEQ ID NO: 519) | C | 543 |
| CR16-3_S: | cacc | G | GGTGGAGGGGGCGGGGCCC (SEQ ID NO: 520) | | 544 |
| CR16-3_AS: | AAAC | | GGGCCCCGCCCCCTCCACC (SEQ ID NO: 521) | C | 545 |
| CR16-4_S: | cacc | G | GGTATCCACGTAAATCAAA (SEQ ID NO: 522) | | 546 |
| CR16-4_AS: | AAAC | | TTTGATTTACGTGGATACC (SEQ ID NO: 523) | C | 547 |
| CR16-5_S: | cacc | G | CCAATCACTGGCTCCGGTC (SEQ ID NO: 524) | | 548 |
| CR16-5_AS: | AAAC | | GACCGGAGCCAGTGATTGG (SEQ ID NO: 525) | C | 549 |
| CR16-6_S: | cacc | G | GGCGCCCGAGGGAAGAAGA (SEQ ID NO: 526) | | 550 |
| CR16-6_AS: | AAAC | | TCTTCTTCCCTCGGGCGCC (SEQ ID NO: 527) | C | 551 |
| CR16-7_S: | cacc | G | GGGTGGGGGTACCAGAGGA (SEQ ID NO: 528) | | 552 |
| CR16-7_AS: | AAAC | | TCCTCTGGTACCCCCACCC (SEQ ID NO: 529) | C | 553 |
| CR16-8_S: | cacc | G | CCGGGGACAGAAGAGAGGG (SEQ ID NO: 530) | | 554 |
| CR16-8_AS: | AAAC | | CCCTCTCTTCTGTCCCCGG (SEQ ID NO: 531) | C | 555 |
| CR16-9_S: | cacc | G | gagagagagtgggagaagc (SEQ ID NO: 532) | | 556 |
| CR16-9_AS: | AAAC | | gcttctcccactctctctc (SEQ ID NO: 533) | C | 557 |
| CR16-10_S: | cacc | G | aaagtaactgtcaaatgcg (SEQ ID NO: 534) | | 558 |
| CR16-10_AS: | AAAC | | cgcatttgacagttacttt (SEQ ID NO: 535) | C | 559 |
| CR16-11_S: | cacc | G | ttaaccagagcgcccagtc (SEQ ID NO: 536) | | 560 |
| CR16-11_AS: | AAAC | | gactgggcgctctggttaa (SEQ ID NO: 537) | C | 561 |
| CR16-12_S: | cacc | G | cgtcggagctgcccgctag (SEQ ID NO: 538) | | 562 |
| CR16-12_AS: | AAAC | | ctagcgggcagctccgacg (SEQ ID NO: 539) | C | 563 |

qRT-PCR & IF: Activation of endogenous ASCL1 was assessed by qRT-PCR and immunofluorescence in MEFs on day 3 following delivery of either dCas9-VP64 and gRNAs, ASCL1 cDNA, or a negative control vector encoding luciferase. The generation of iNs was evaluated by TUJ1 and MAP2 co-staining and identification of cells with neuronal morphology and extended processes.

Live Cell Reporters: After 7-8 days in N3 medium, MEFs cultured on poly-D-lysine/laminin-coated coverslips were transduced with viruses carrying hSyn-RFP and MAP2-GCamP5 reporters to identify the most mature iNs for functional characterization via calcium imaging and electrophysiology (FIG. 49B).

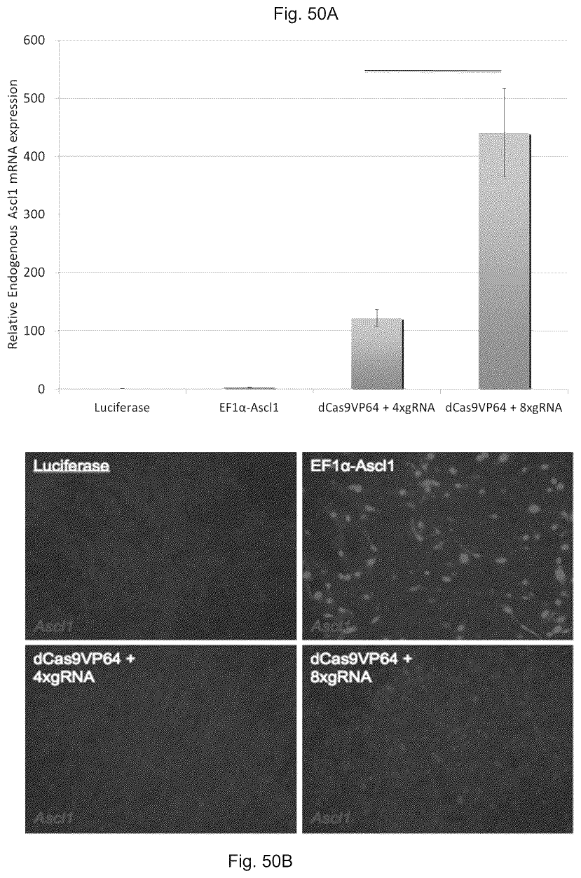

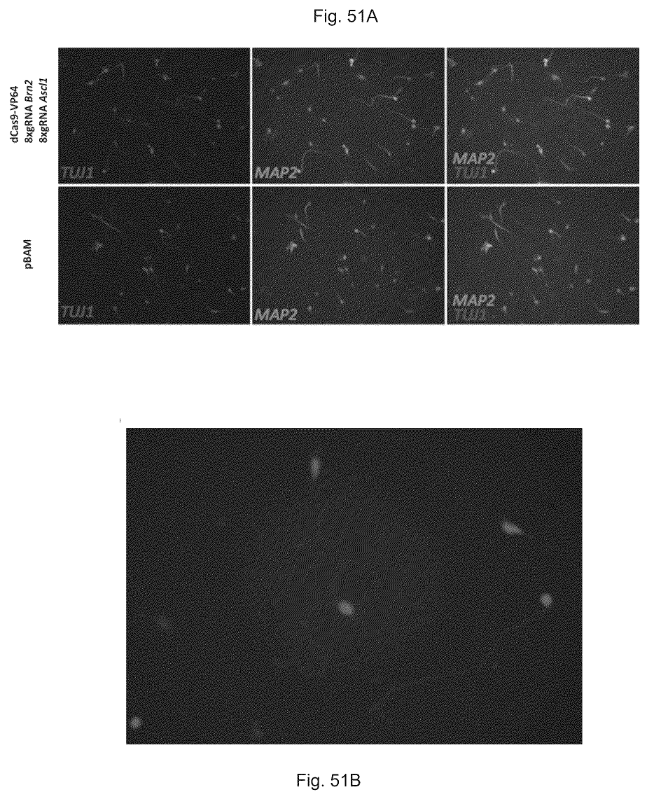

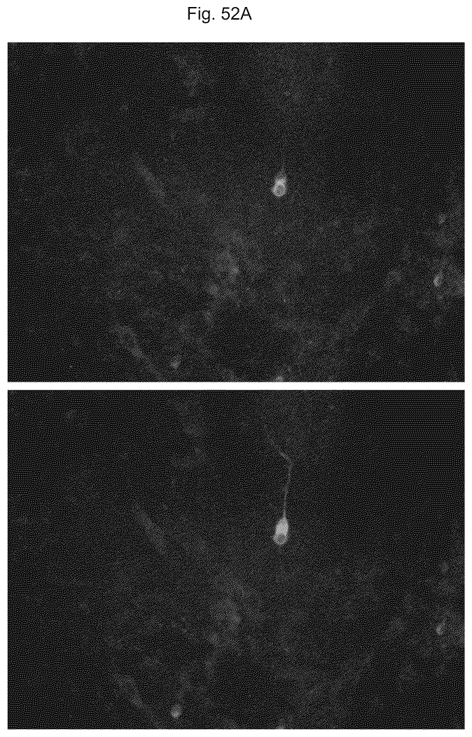

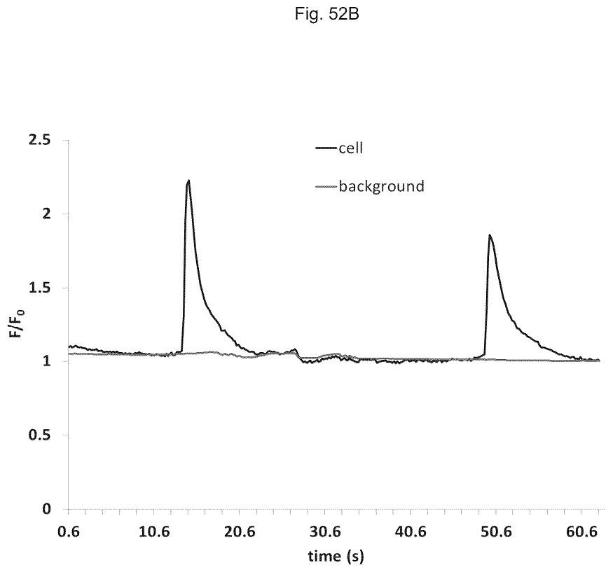

Results. dCas9-VP64 and gRNAs targeted to the ASCL1 promoter activated the endogenous gene in MEFs. Co-delivery of 8 gRNAs activated the endogenous gene 400-fold, a significant increase (p<0.05) over the 100-fold activation induced by the co-delivery of 4 gRNAs (FIG. 50A). Nuclear-localized Ascl1 protein was detected by immunofluorescence in MEFs. Ectopic Ascl1 expression produced more Ascl1 protein than dCas9-VP64 with either gRNA cocktail, but did not activate the endogenous locus by day 3 (FIG. 50A,50B). TUJ1 and MAP2 co-positive cells with extended processes were identified by immunofluorescence after 13 days in neurogenic medium following delivery of dCas9-VP64 and gRNAs targeting the ASCL1 and BRN2 promoters (FIG. 51A first row). A similar number of TUJ1 and MAP2 co-positive cells were identified with ectopic expression of the BAM factors (FIG. 51A second row). Cells with a neuronal morphology expressing the hSyn-RFP reporter were visible in culture as early as day 11 in neurogenic medium (FIG. 51B). A cell expressing the MAP2-GCaMP5 calcium indicator exhibited KCl-induced depolarization detected with a fluorescent microscope (FIG. 52A,52B).

The direct conversion of mouse embryonic fibroblasts to TUJ1 and MAP2 co-positive cells with a neuronal morphology was accomplished through activation of endogenous BRN2 and ASCL1 by CRISPR/Cas9-based transcription factors. Though dCas9-VP64 produces less protein than ectopic expression of ASCL1 (FIG. 50B), the generation of neuronal-like cells is similar. The activation of the endogenous loci may induce a reprogramming cascade of events that is not mechanistically identical to that generated with ectopic expression.

dCas9-VP64 was able to penetrate heterochromatin and activated stably silenced endogenous genes, a characteristic of only a subset of “pioneer” transcription factors. As a result, converting cell lineage with CRISPR/Cas9-transcription factors may better overcome epigenetic barriers to reprogramming than ectopic expression of transcription factors, particularly in hard-to-reprogram cell-types, such as adult human cells. This may have clinical importance in the field of regenerative medicine, as it is often desired to use autologous sources in cell replacement therapies.

### Example 17

### Multiplex CRISPR/Cas9-Based Genome Engineering—Materials and Methods

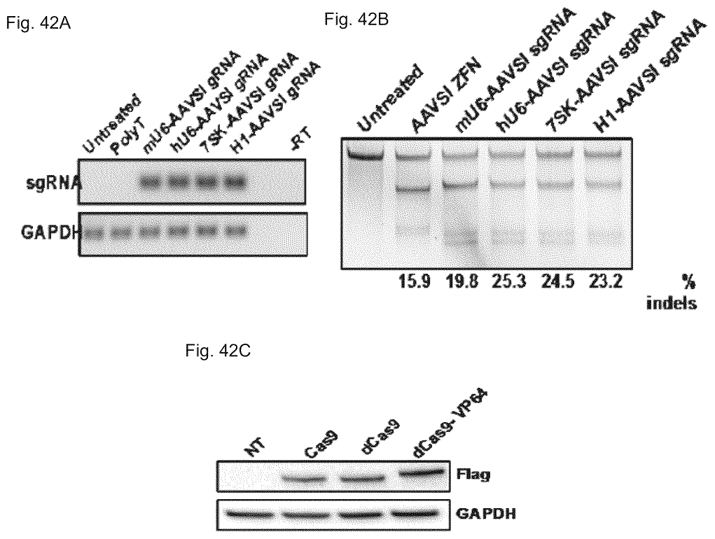

Plasmid constructs. The expression cassettes for the S. pyogenes sgRNA and human codon optimized Cas9 (hCas9) nuclease were used, as described above. Additional promoters for mU6 (Ohshima et al., Nucleic Acids Res 9:5145-5158 (1981)), H1 (Myslinski et al., Nucleic Acids Res 29:2502-2509 (2001)), and 7SK (Murphy et al., Cell 51:81-87 (1987)) pol-III promoters were synthesized using GeneBlocks (IDT) and cloned in place of the hU6 sgRNA expression cassette. A GeneBlock (IDT) was cloned onto the 3′ end of the Cas9 coding sequence to fuse a T2A skipping peptide and eGFP gene immediately after Cas9 to monitor vector expression. The coding region for hCas9-T2A-GFP (SEQ ID NO: 145) was then transferred into a lentiviral expression vector containing the human ubiquitin C (hUbC) promoter to drive expression of hCas9-T2A-GFP, as well as restriction sites to facilitate Golden Gate cloning of sgRNA expression cassettes immediately upstream of the hUbC promoter (FIG. 42A).

Protocol for assembly of custom lentiviral vectors. Assembly of custom lentiviral vectors expressing up to four sgRNAs of choice and active Cas9, dCas9, or dCas9-VP64 was accomplished in less than five days. The cloning method used the Golden Gate cloning and type IIS restriction enzymes that cleave outside their recognition sequence to create unique overhangs. Golden Gate assembly expedited cloning as all four expression cassettes were ligated into the final lentiviral vector in one step. The lentiviral vector expressed active Cas9, cCas9, or dCas9-VP64 in addition to one, two, three, or four sgRNAs expressed from independent promoters.

Step 1: Single stranded oligos containing each 20 bp protospacer were annealed in such a fashion to create sticky ends and were ligated into the desired pZDonor-promoter vector. Order two single stranded oligos for each desired genomic target. To anneal the complimentary oligos, mix 8 μL sense oligo+8 μL antisense oligo (both at 10 mM)+2 μL 10× ligase buffer. The oligos are melted and reannealed in a PCR machine with the program: 96° C. for 300 seconds, followed by 85° C. for 20 seconds, 75° C. for 20 seconds, 65° C. for 20 seconds, 55° C. for 20 seconds, 45° C. for 20 seconds, 35° C. for 20 seconds, and 25° C. for 20 seconds with a −0.3° C./second rate between steps. To phosphorylate the sticky ends, add 1 μL 25 mM ATP+1 microliter T4 Polynucleotide Kinase (NEB) and incubate at 37° C. for 60 minutes followed by 65° C. for 20 minutes to heat inactivate the enzyme. Each protospacer was ligated into the desired expression vector using T4 DNA ligase (NEB) incubated at 16° C. for 60 minutes using 50 ng of vector and 1 μL of annealed oligonucleotides in a 10 μL reaction volume according to manufacturer's instructions. Five microliters of each ligation was transformed into XL 1 blue chemically competent bacteria (Agilent) following the manufacturer's instructions. Plate transformation onto LB agar plates containing 50 μg/mL kanamycin (Sigma) and incubate overnight at 37° C. In our experience, >90% of the colonies will contain the desired ligation product. Sequencing using the M13 reverse standard sequencing primer was performed to validate each final sgRNA construct prior to moving onto step 2.

Step 2: Construct the four promoter-gRNA cassettes into a lentiviral destination vector using Golden Gate assembly. After completion of step 1, there are four independent plasmids each expressing a different sgRNA from a different promoter. To assemble the four different promoter-sgRNA constructs into the desired destination vector, combine 200 ng of each sgRNA expression plasmid and desired lentiviral destination vector with 1 μL of T4 DNA ligase (NEB), 1 μL BsmBI FastDigest (Fisher Scientific), and 2 μL 10× T4 ligase buffer (NEB) in a 20 μL reaction volume. Incubate the reaction as follows: 37° C. for 10 minutes, 16° C. for 15 minutes, 37° C. for 30 minutes, 80° C. for 5 minutes. Transform 5 μL of ligation reaction into SURE 2 chemically competent cells (Agilent) following the manufacturer's instructions. Plate transformations onto LB agar plates containing 100 μg/mL ampicillin and incubate overnight at 37° C. Optionally, colonies can be screened by lacZ-based blue/white screening using IPTG and X-gal; however, in our experience, >90% of the transformants contain the proper ligation product. Due to the inverted repeats formed by the opposing sgRNA expression cassettes, the final constructs may be unstable and thus we recommend maintaining these plasmids in the SURE 2 cell line and screening the final plasmid with a test PCR using the sense primer 5′-TCGGGTTTATTACAGGGACAGCAG-3′ (SEQ ID NO:464) and antisense primer 5′-TCTAAGGCCGAGTCTTATGAGCAG-3′ (SEQ ID NO:465). These primers amplify across the four promoter-gRNA region. Due to the repetitive nature, a distinct banding pattern should be observed with the largest product approximately 1800 bp in size.

Cell culture and transfection. HEK293T cells were obtained from the American Tissue Collection Center (ATCC, Manassas, VA) through the Duke University Cancer Center Facilities and were maintained in DMEM supplemented with 10% FBS and 1% penicillin streptomycin. Primary human dermal fibroblasts (Catalog ID: GM03348) were obtained from Coriell Institute (Camden, New Jersey) and were maintained in DMEM supplemented with 10% FBS and 1% penicillin streptomycin. All cells were cultured at 37° C. with 5% CO₂. HEK293T cells were transfected with Lipofectamine 2000 (Life Technologies) with 200 ng of each sgRNA expression vector (800 ng total pDNA) according to the manufacturer's protocol in 24 well plates.

Viral production and transduction. All lentiviral vectors used is this study are second generation and were produced using standard viral production methods. Briefly, 3.5 million HEK293 Ts were plated per 10 cm dish. The following day, cells were transfected by the calcium phosphate transfection method with 20 μg of transfer vector, 6 μg of pMD2G, and 10 μg psPAX2. The media was changed 12-14 hrs post transfection. The viral supernatant was collected 24 and 48 hrs after this media change, passed through a 0.45 micron filter and pooled. For transduction, the cell medium was replaced with viral supernatant supplemented with 4 μg/mL polybrene. The viral supernatant was exchanged for fresh medium 12-24 hrs later.

Reverse Transcription PCR. RNA was isolated using the miRNeasy Mini RNA isolation kit (Qiagen). DNAse digestion was performed using the DNA-free Kit (Applied Biosystems). cDNA synthesis was performed using the SuperScript VILO cDNA Synthesis Kit (Invitrogen). cDNA was PCR amplified using Taq DNA polymerase (NEB) and the resulting product was run on TAE agarose gels. Images were captured using a ChemiDoc XRS+ System and processed using ImageLab software (Bio-Rad).

Quantitative Real Time PCR. RNA was isolated using the RNeasy Plus RNA isolation kit (Qiagen). cDNA synthesis was performed using the SuperScript VILO cDNA Synthesis Kit (Invitrogen). Real-time PCR using PerfeCTa SYBR Green FastMix (Quanta Biosciences) was performed with the CFX96 Real-Time PCR Detection System (Bio-Rad). Primer specificity was confirmed by agarose gel electrophoresis and melting curve analysis. Reaction efficiencies over the appropriate dynamic range were calculated to ensure linearity of the standard curve. The results are expressed as fold-increase mRNA expression of the gene of interest normalized to β-Actin expression using the ΔΔCt method. Reported values are the mean and S.E.M. from two independent experiments (n=2) where technical replicates were averaged for each experiment.

Western Blot. Cells were lysed in RIPA Buffer (Sigma) supplemented with protease inhibitor cocktail (Sigma). Protein concentration was measured using BCA protein assay reagent (Thermo Scientific) and BioTek Synergy 2 Multi-Mode Microplate Reader. Lysates were mixed with loading buffer and boiled for 5 min; 25 μg of protein were run in NuPage 10% Bis-Tris Gel polyacrylamide gels (Bio-Rad) and transferred to nitrocellulose membranes. Nonspecific antibody binding was blocked with TBST (50 mM Tris, 150 mM NaCl and 0.1% Tween-20) with 5% nonfat milk for 1 hr at room temperature. The membranes were incubated with primary antibodies: (anti-MyoD (1:250, Santa Cruz Sc-32758) in 5% BSA in TBST overnight at 4° C.; anti-Myogenin (1:250, Santa Cruz Sc-12732) in 5% BSA overnight at 4° C.; anti-FLAG-HRP (1:1000, Cell Signaling 2044) in 5% milk in TBST for 60 min at room temperature; anti-GAPDH (1:5000, Cell Signaling, clone 14C10) in 5% milk in TBST for 30 min at room temperature. Membranes were then washed three times with TBST for 15 minutes total. Membranes were incubated with anti-rabbit HRP-conjugated antibody (Sigma, A 6154) or anti-mouse HRP-conjugated antibody (Santa Cruz, SC-2005) diluted 1:5,000 for 30 min and washed with TBST three times for 15 min each. Membranes were visualized using the ImmunStar WesternC Chemiluminescence Kit (Bio-Rad) and images were captured using a ChemiDoc XRS+ System and processed using ImageLab software (Bio-Rad).

Cel-I quantification of endogenous gene modification. CRISPR/Cas9 nuclease lesions at the endogenous target site were quantified using the Surveyor nuclease assay, which can detect mutations characteristic of nuclease-mediated NHEJ. After transfection or transduction, cells were incubated for 3-10 days at 37° C. and genomic DNA was extracted using the DNeasy Blood and Tissue kit (Qiagen). The target locus was amplified by 35 cycles of PCR with the AccuPrime High Fidelity PCR kit (Invitrogen). The resulting PCR products were randomly melted and reannealed in a PCR machine with the program: 95° C. for 240 seconds, followed by 85° C. for 60 seconds, 75° C. for 60 seconds, 65° C. for 60 seconds, 55° C. for 60 seconds, 45° C. for 60 seconds, 35° C. for 60 seconds, and 25° C. for 60 seconds with a −0.3° C./second rate between steps. Following re-annealing, 8 μL of PCR product was mixed with 1 μL of Surveyor Nuclease S and 1 μL of Enhancer S (Transgenomic, Omaha, NE) and incubated at 42° C. for 1 hr. After incubation, 6 μL of digestion product was loaded onto a 10% TBE polyacrylamide gel and run at 200 V for 30 minutes. The gels were stained with ethidium bromide and quantified using ImageLab (Bio-Rad) by densitometry (Perez-Pinera et al., Nucleic Acids Res 40:3741-3751 (2012)).

Statistical Analysis. At least two independent experiments were compiled as means and standard errors of the mean. Effects were evaluated with multivariate ANOVA and Dunnett's post hoc test using JMP 10 Pro.

### Example 18

### Development of a Single Lentiviral Vector for Multiplex CRISPR/Cas9 Applications

A limitation of current CRISPR/Cas9 gene editing systems, particularly transactivator systems, is the simultaneous and efficient delivery of multiple sgRNAs and Cas9 protein for multiplex gene editing and synergistic gene activation applications, especially in difficult to transfect cell types. To overcome this limitation, we developed a single lentiviral vector that efficiently expresses Cas9 and up to four sgRNAs. In order to maximize the expression efficiency of each sgRNA, this vector expresses four sgRNAs from four independent pol III promoters (human U6 promoter, mouse U6 promoter, 7SK, and H1). We validated sgRNA expression from each promoter using end-point RT-PCR to detect a sgRNA targeting the AAVS1 locus (FIG. 42A). To test the activity of each sgRNA expression construct, we co-transfected each promoter construct expressing an sgRNA targeting AAVS1 independently with an active Cas9 expression construct into human HEK293T cells. Notably, we detected consistent and high levels of gene modification at the target locus for each sgRNA that are comparable to a well-characterized zinc-finger nuclease with high activity at the AAVS1 locus (FIG. 42B). Furthermore, lentiviral delivery of different Cas9-based constructs, including an active Cas9 nuclease, dead Cas9, and dead Cas9 fused to the VP64 transactivator domain, resulted in expression of full-length Cas9 protein in HEK293T cells as determined by western blot (FIG. 42C).

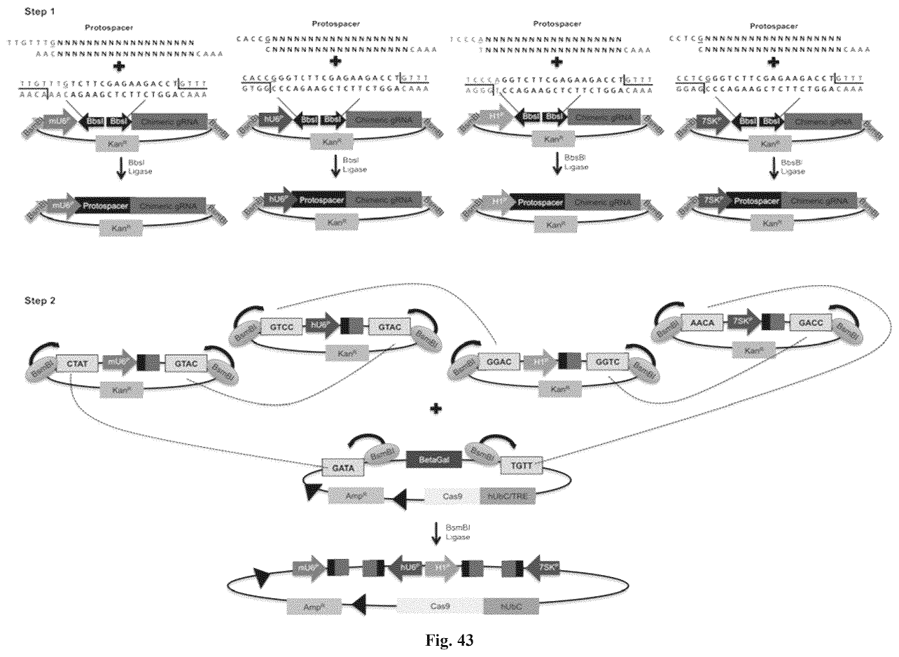

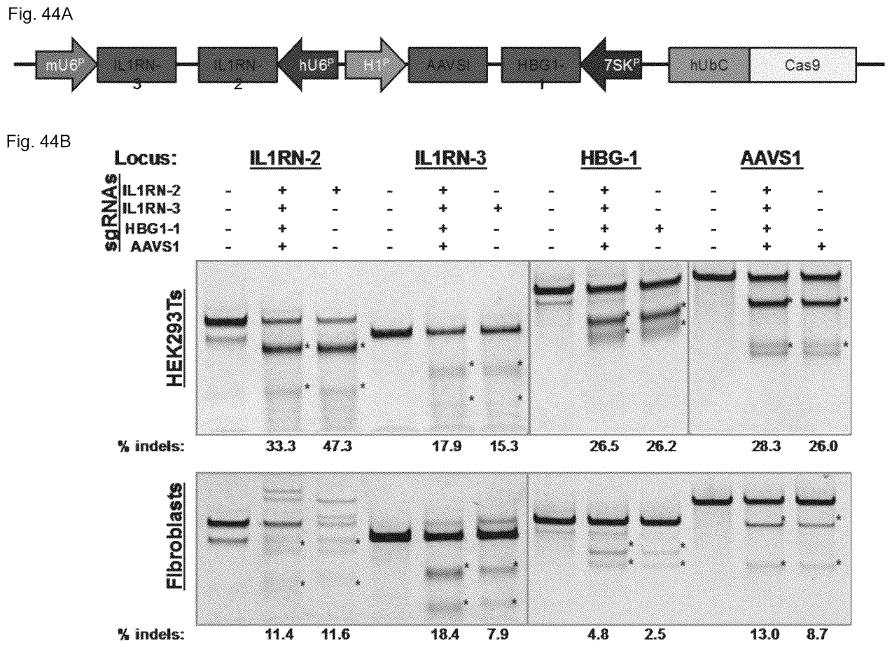

Using these components, we developed a Golden Gate cloning method to facilitate rapid and efficient cloning of multiple sgRNA expression cassettes into a single lentiviral vector expressing the desired Cas9 effector (FIG. 43). In the first step, oligonucleotides encoding sgRNA protospacer sequences are cloned independently into different expression vectors, each with a distinct promoter driving sgRNA expression. In the second step, each sgRNA expression construct is subcloned into a lentiviral Cas9 expression vector of choice by Golden Gate assembly. This strategy allows for robust and rapid cloning of up to four sgRNAs into a single lentiviral vector for gene editing or activation applications. To express less than four sgRNAs, a PolyT terminator sequence is cloned down-stream of unused promoters to prevent transcription from the unused promoters. Each vector co-expresses the choice Cas9 with eGFP via a 2A skipping peptide to enable fluorescence-activated flow sorting and enrichment of cells with a high multiplicity of infection. Finally, the entire region containing the sgRNA and Cas9 expression cassettes is flanked by loxP sites to mediate removal by Cre-lox excision.

### Example 19

Validation of a Single Lentiviral sgRNA/Cas9 Expression Vector for Multiplex Genome Engineering

To validate the independent activity of each sgRNA, we assembled a single lentiviral vector expressing active Cas9 and four sgRNAs, each targeting an independent loci (FIG. 44A). As control vectors, we assembled constructs expressing only one sgRNA along with polyT protospacers in the other three positions. We transduced HEK293 Ts and primary fibroblasts with lentiviral vectors expressing the indicated sgRNAs and monitored gene modification frequencies at 7 or 10 days post-transduction, respectively (FIG. 44B). In both cell types, the single lentiviral vector mediated highly efficient multiplex editing at all four loci (FIG. 44B). Interestingly, expression of all four sgRNAs together resulted in higher modification frequencies than a single sgRNA alone at 3 out of 4 loci in fibroblasts (FIG. 44B). We observed efficient multiplex gene editing in fibroblasts, which are conventionally a difficult to transfect cell type. These data demonstrate that a single lentivirus can express four active sgRNAs efficiently and that this lentiviral platform can be used to target four distinct loci for multiplex CRISPR/Cas9 gene editing.

### Example 20

### Transient RNA-Guided Gene Activation in Cell Lines Stably Expressing a Lentiviral Cas9-Based Transactivator

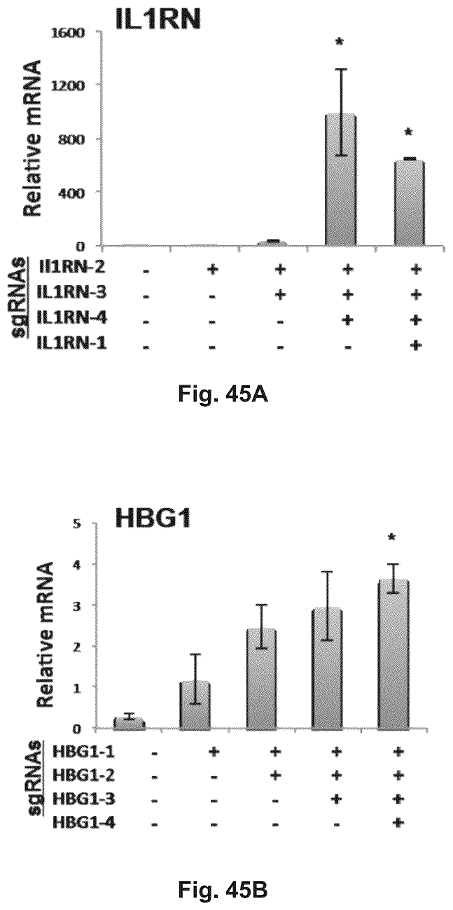

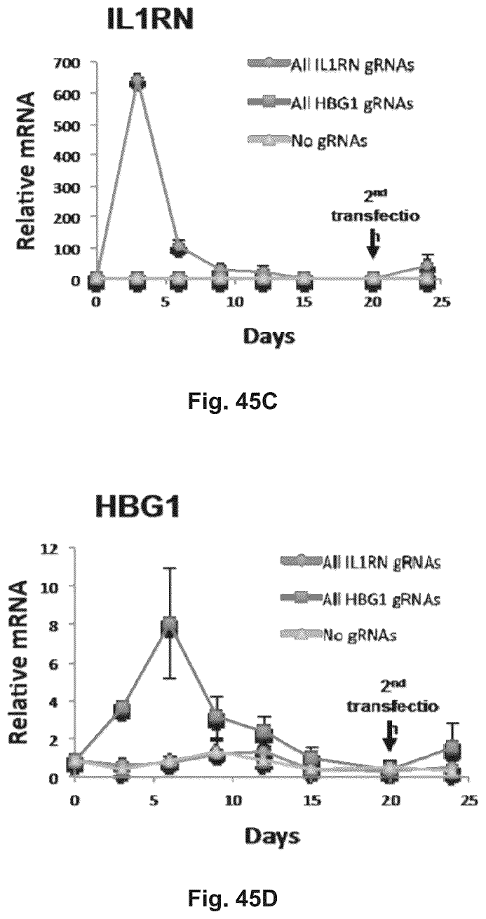

Next, we were interested in developing a system that enables transient gene activation by transfecting sgRNAs into model cell lines stably expressing Cas9. HEK293 Ts were transduced with different Cas9-T2A-GFP and GFP expression was monitored using flow cytometry. Following normal passaging every 2-3 days, each cell line exhibited stable GFP expression for up to 35 days post transduction. Transduced HEK293 Ts were then transfected with one to four separate sgRNA expression constructs targeting either the IL1RN or HBG1 promoter. Transient transfection of these sgRNA constructs in stable dCas9-VP64 expressing cells lines resulted in tunable endogenous gene activation (FIGS. 45A,45B). Gene activation following transient transfection of sgRNA constructs in cells expressing dCas9-VP64 reached a maximum level of activation approximately 3-6 post-transfection and fell to undetectable levels by 20 days post-transfection (FIGS. 45C,45D). Furthermore, we were able to re-activate each gene by a second transfection of all four sgRNA constructs targeting each promoter, although activation levels were significantly lower than observed from the first transfection (FIGS. 45C, 45D). This reduction in activity after the second transfection may be due to reduced vector expression or competitive growth of untransduced cells. Despite this, these data demonstrate that lentiviral Cas9 combined with transient sgRNA delivery can be used as a versatile system to tunably and transiently activate and re-activate target genes in a Cas9 stably tranduced cell line.

### Example 21

Stable Gene Activation in HEK293T Cells Using a Single Lentiviral sgRNA/Cas9 Transactivator Expression Vector

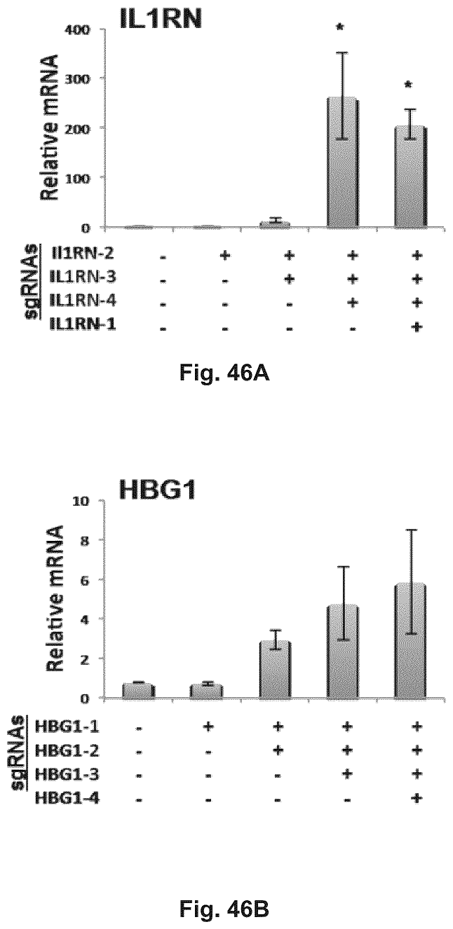

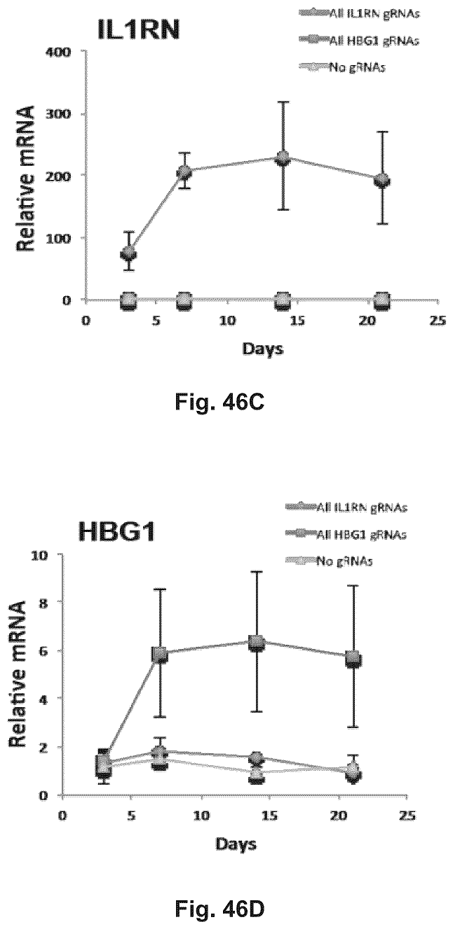

Lentiviral delivery may enable stable, long-term gene activation by CRISPR/Cas9 transactivation. To test this, HEK293 Ts were transduced using a single lentiviral vector encoding dCas9-VP64 and one to four sgRNA expression cassettes. Similar to our transient transfection results (FIGS. 45A-45D), we were able to tunably and robustly activate expression of endogenous IL1RN and HBG1 genes (FIGS. 46A,46B). Gene activation induced by co-transfection of HEK293T cells with dCas9-VP64 and four sgRNAs targeted to the IL1RN and HBG1 promoters peaked three-five days post-transfection and gene expression returned to background levels 15-20 days post-transfection FIGS. 45C and 45D. In contrast, lentiviral delivery of dCas9-VP64 and the same four IL1RN or HBG1-targeted sgRNAs induced sustained gene activation for more than 20 days post-transduction (FIGS. 46C,46D). Thus, single lentiviral delivery of multiplex dCas9-VP64 transactivators is a useful platform to efficiently and stably upregulate target endogenous genes.

### Example 22

dCas9-KRAB—Targeting the HS2 Enhancer

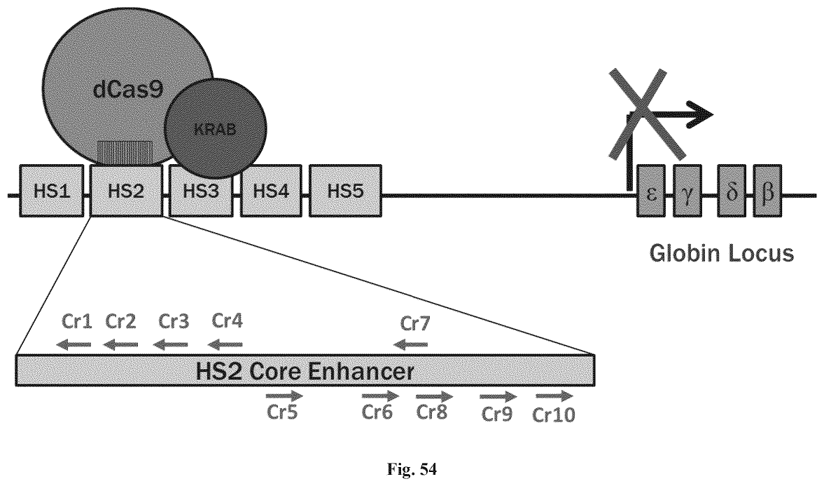

The HS2 enhancer is a well-characterized distal regulatory element necessary for activation of the globin gene locus. dCas9-KRAB with gRNAs targeted to the HS2 enhancer were delivered to determine if this system would repress γ-, ε-, and β-globin expression in the K562 human erythroid leukemia cell line (FIG. 54). A panel of gRNAs was created targeting different sites along the core region of the HS2 enhancer (SEQ ID NO: 467). See Table 12.

TABLE 12
HS2 gRNA Target Sequences

| Cr# | Protospacer | Complete gRNA Sequence |
|---|---|---|
| 1 | gagacacacagaaatgtaac | gagacacacagaaatgtaacgtaTAGAGCTAGAAATAGCAAGTTAAAATA |
| | (SEQ ID NO: 564) | AGGCTAGTCCGTTATCAACTTGAAAAAGTGGCACCGAGTCGG |
| | | TGCTTTTTTTCC (SEQ ID NO: 585) |
| 2 | ggtggggcactgaccccgac | ggtggggcactgaccccgacgtaTAGAGCTAGAAATAGCAAGTTAAAATA |
| | (SEQ ID NO: 565) | AGGCTAGTCCGTTATCAACTTGAAAAAGTGGCACCGAGTCGG |
| | | TGCTTTTTTTCC (SEQ ID NO: 586) |
| 3 | ctagagtgatgactcctatc | ctagagtgatgactcctatcgtttTAGAGCTAGAAATAGCAAGTTAAAATAA |
| | (SEQ ID NO: 566) | GGCTAGTCCGTTATCAACTTGAAAAAGTGGCACCGAGTCGGT |
| | | GCTTTTTTTCC (SEQ ID NO: 587) |
| 4 | gactaaaactccacctcaaa | gactaaaactccacctcaaagtaTAGAGCTAGAAATAGCAAGTTAAAATAA |
| | (SEQ ID NO: 567) | GGCTAGTCCGTTATCAACTTGAAAAAGTGGCACCGAGTCGGT |
| | | GCTTTTTTTCC (SEQ ID NO: 588) |
| 5 | aatatgtcacattctgtctc | aatatgtcacattctgtctcgtaTAGAGCTAGAAATAGCAAGTTAAAATAAG |
| | (SEQ ID NO: 568) | GCTAGTCCGTTATCAACTTGAAAAAGTGGCACCGAGTCGGTG |
| | | CTTTTTTTCC (SEQ ID NO: 589) |
| 6 | ggactatgggaggtcactaa | ggactatgggaggtcactaagtaTAGAGCTAGAAATAGCAAGTTAAAATA |
| | (SEQ ID NO: 569) | AGGCTAGTCCGTTATCAACTTGAAAAAGTGGCACCGAGTCGG |
| | | TGCTTTTTTTCC (SEQ ID NO: 590) |
| 7 | gctcatgcttggactatggg | gctcatgcttggactatggggtaTAGAGCTAGAAATAGCAAGTTAAAATAA |
| | (SEQ ID NO: 570) | GGCTAGTCCGTTATCAACTTGAAAAAGTGGCACCGAGTCGGT |
| | | GCTTTTTTTCC (SEQ ID NO: 591) |
| 8 | gttctggccaggcccctgtc | gttctggccaggcccctgtcgtaTAGAGCTAGAAATAGCAAGTTAAAATAA |
| | (SEQ ID NO: 571) | GGCTAGTCCGTTATCAACTTGAAAAAGTGGCACCGAGTCGGT |
| | | GCTTTTTTTCC (SEQ ID NO: 592) |
| 9 | agtgccccacccccgccttc | agtgccccacccccgccttcgtttTAGAGCTAGAAATAGCAAGTTAAAATAA |
| | (SEQ ID NO: 572) | GGCTAGTCCGTTATCAACTTGAAAAAGTGGCACCGAGTCGGT |
| | | GCTTTTTTTCC (SEQ ID NO: 593) |
| 10 | gtggggcactgaccccgaca | gtggggcactgaccccgacagtttTAGAGCTAGAAATAGCAAGTTAAAATA |
| | (SEQ ID NO: 573) | AGGCTAGTCCGTTATCAACTTGAAAAAGTGGCACCGAGTCGG |
| | | TGCTTTTTTTCC (SEQ ID NO: 594) |
| 11 | aaccttctaagcaaaccttc | aaccttctaagcaaaccttcgtaTAGAGCTAGAAATAGCAAGTTAAAATAA |
| | (SEQ ID NO: 574) | GGCTAGTCCGTTATCAACTTGAAAAAGTGGCACCGAGTCGGT |
| | | GCTTTTTTTCC (SEQ ID NO: 595) |
| 12 | gttacacagaaccagaaggc | gttacacagaaccagaaggcgtttTAGAGCTAGAAATAGCAAGTTAAAATA |
| | (SEQ ID NO: 575) | AGGCTAGTCCGTTATCAACTTGAAAAAGTGGCACCGAGTCGG |
| | | TGCTTTTTTTCC (SEQ ID NO: 596) |
| 13 | gaaggttacacagaaccaga | gaaggttacacagaaccagagtaTAGAGCTAGAAATAGCAAGTTAAAATA |
| | (SEQ ID NO: 576) | AGGCTAGTCCGTTATCAACTTGAAAAAGTGGCACCGAGTCGG |
| | | TGCTTTTTTTCC (SEQ ID NO: 597) |
| 14 | agtcatgatgagtcatgctg | agtcatgatgagtcatgctggtttTAGAGCTAGAAATAGCAAGTTAAAATAA |
| | (SEQ ID NO: 577) | GGCTAGTCCGTTATCAACTTGAAAAAGTGGCACCGAGTCGGT |
| | | GCTTTTTTTCC (SEQ ID NO: 598) |
| 15 | gatgagtcatgctgaggctt | gatgagtcatgctgaggcttgtaTAGAGCTAGAAATAGCAAGTTAAAATAA |
| | (SEQ ID NO: 578) | GGCTAGTCCGTTATCAACTTGAAAAAGTGGCACCGAGTCGGT |
| | | GCTTTTTTTCC (SEQ ID NO: 599) |
| 16 | actctaggctgagaacatct | actctaggctgagaacatctgtaTAGAGCTAGAAATAGCAAGTTAAAATAA |
| | (SEQ ID NO: 579) | GGCTAGTCCGTTATCAACTTGAAAAAGTGGCACCGAGTCGGT |
| | | GCTTTTTTTCC (SEQ ID NO: 600) |
| 17 | gtccccagcaggatgcttac | gtccccagcaggatgcttacgtaTAGAGCTAGAAATAGCAAGTTAAAATAA |
| | (SEQ ID NO: 580) | GGCTAGTCCGTTATCAACTTGAAAAAGTGGCACCGAGTCGGT |
| | | GCTTTTTTTCC (SEQ ID NO: 601) |
| 18 | gccctgtaagcatcctgctg | gccctgtaagcatcctgctggtttTAGAGCTAGAAATAGCAAGTTAAAATAA |
| | (SEQ ID NO: 581) | GGCTAGTCCGTTATCAACTTGAAAAAGTGGCACCGAGTCGGT |
| | | GCTTTTTTTCC (SEQ ID NO: 602) |
| 19 | cagggcagatggcaaaaaaa | cagggcagatggcaaaaaaagtaTAGAGCTAGAAATAGCAAGTTAAAATA |
| | (SEQ ID NO: 582) | AGGCTAGTCCGTTATCAACTTGAAAAAGTGGCACCGAGTCGG |
| | | TGCTTTTTTTCC (SEQ ID NO: 603) |
| 20 | gaggtggagttttagtcagg | gaggtggagttttagtcagggtaTAGAGCTAGAAATAGCAAGTTAAAATAA |
| | (SEQ ID NO: 583) | GGCTAGTCCGTTATCAACTTGAAAAAGTGGCACCGAGTCGGT |
| | | GCTTTTTTTCC (SEQ ID NO: 604) |
| 21 | aaacggcatcataaagaaaa | aaacggcatcataaagaaaagtttTAGAGCTAGAAATAGCAAGTTAAAATA |
| | (SEQ ID NO: 584) | AGGCTAGTCCGTTATCAACTTGAAAAAGTGGCACCGAGTCGG |
| | | TGCTTTTTTTCC (SEQ ID NO: 605) |

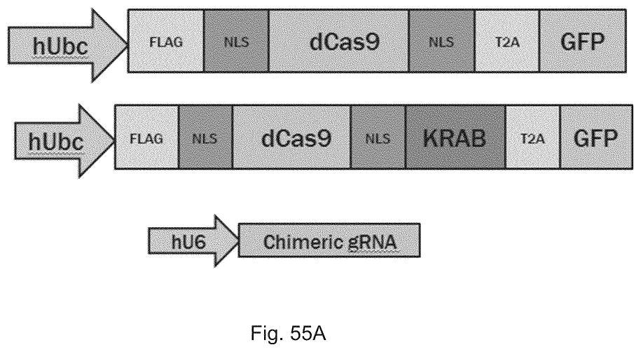

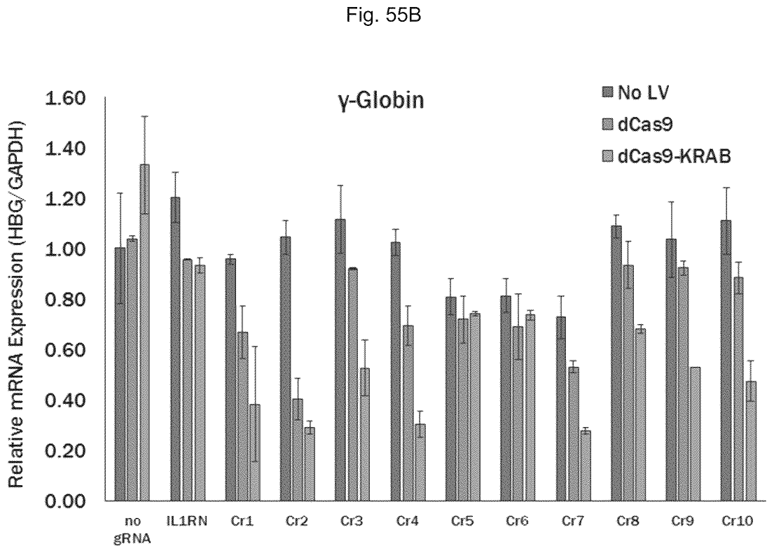

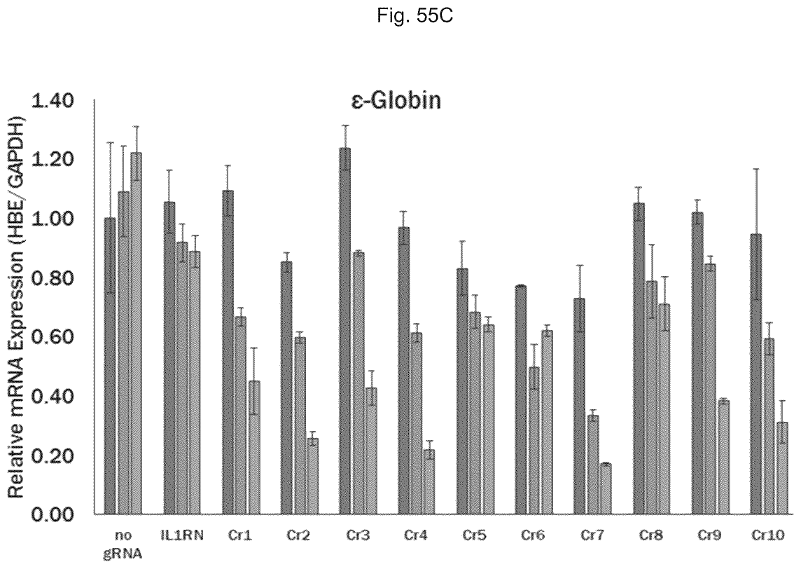

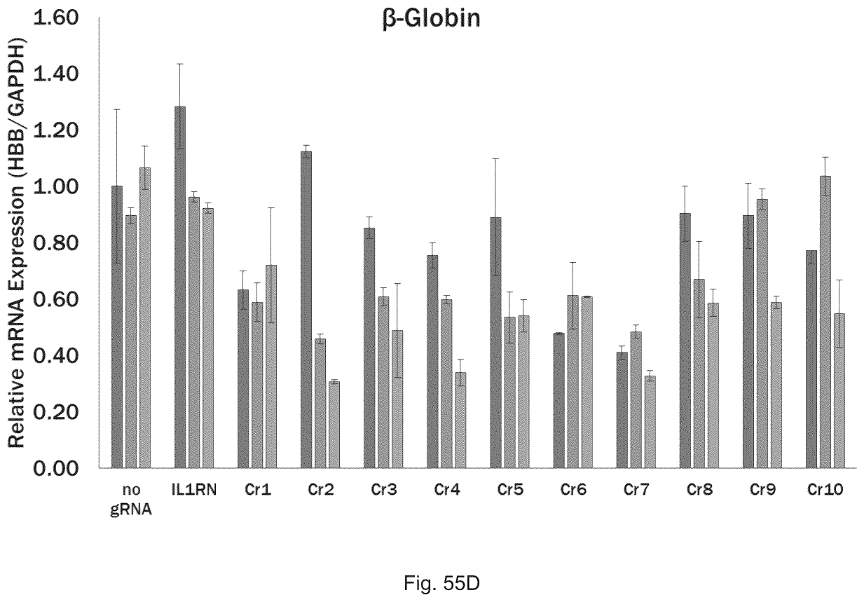

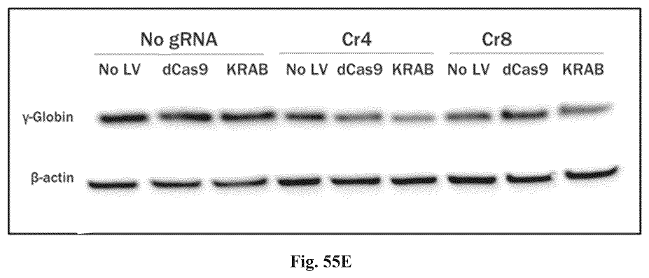

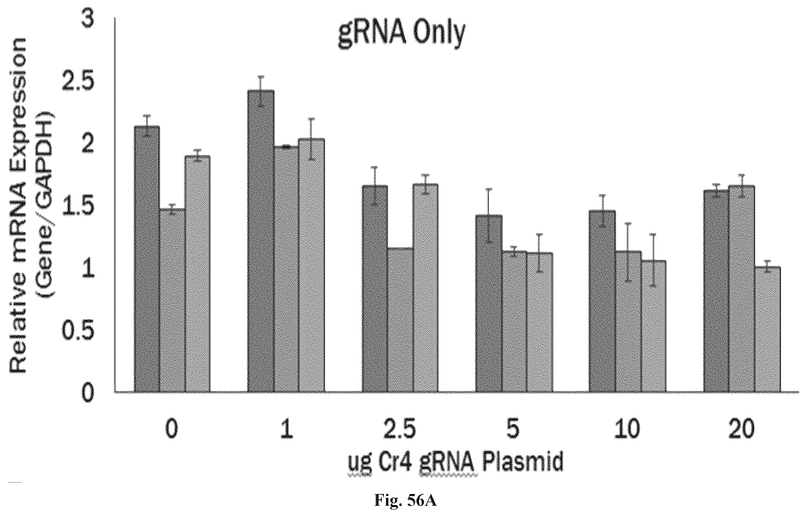

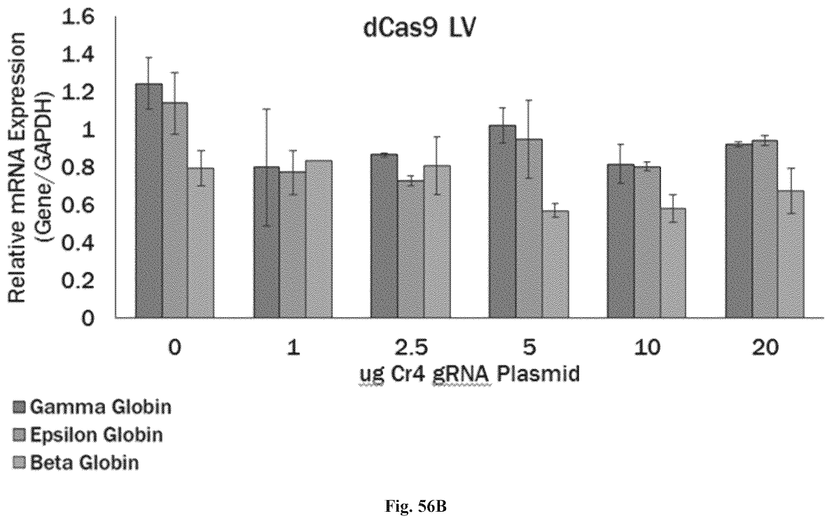

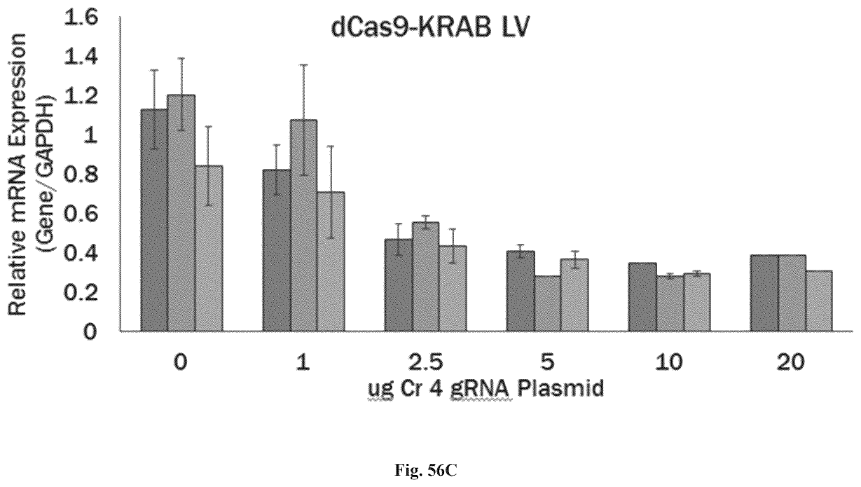

Screening single gRNAs at the globin locus by CRISPR/dCas9. dCas9 and dCas9-KRAB effectors were delivered lentivirally 5-8 days prior to electroporation with 5 μg of a plasmid encoding the U6-sgRNA expression (FIG. 55A). Cells that were not electroporated with gRNA (no gRNA) and cells treated with a gRNA targeting a different locus (IL1RN) were included as controls. Multiple gRNAs effected potent repression of ε-, γ-, and β-globin genes when assayed 3 days post-transfection, with up to 80% knockdown achieved (FIGS. 55B, 55C, 55D). Expressing a gRNA with either dCas9 or dCas9-KRAB inhibited gene expression at the globin locus. Generally, treatment with dCas9-KRAB resulted in stronger repression for a given gRNA compared to dCas9 alone, suggesting an important role for the KRAB domain in recruiting heterochromatin factors that enhance repression. The repression levels achieved are dependent on the amount of gRNA plasmid delivered by transfection only in the dCas9-KRAB treated cells (FIGS. 56A, 56B, 56C). Increasing the dose of Cr4 gRNA plasmid up to 10 μg increases silencing levels of the globin genes in dCas9-KRAB-treated cells.

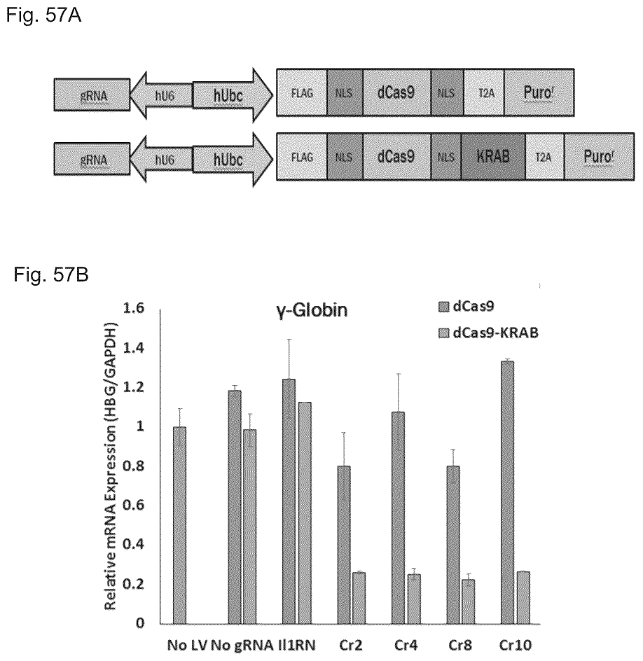

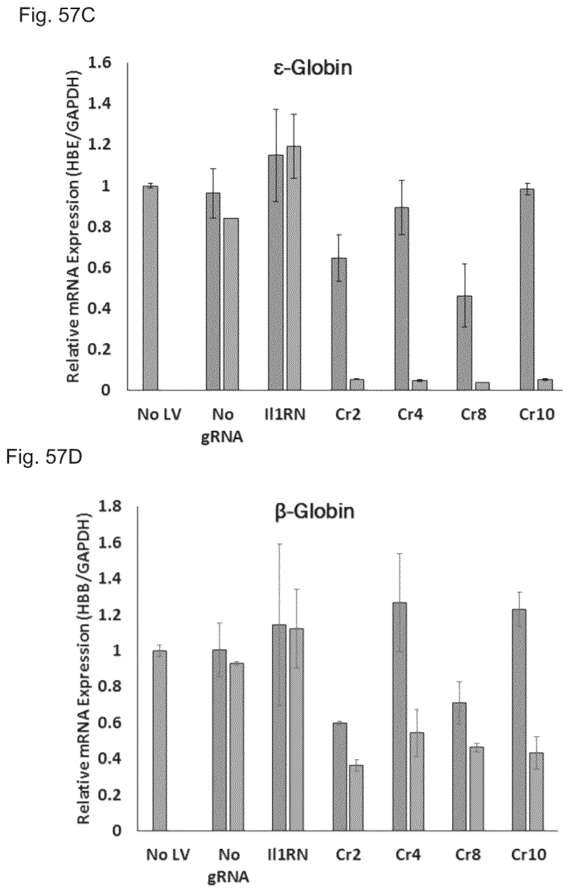

Stable silencing of globin genes by dCas9-KRAB. dCas9/dCas9-KRAB were co-expressed with single gRNAs lentivirally in K562s (FIG. 57A). Cells that were not treated with lentivirus (NT), treated with dCas9/dCas9-KRAB without a gRNA (no gRNA), and with dCas9/dCas9-KRAB and gRNA targeting a different locus (IL1RN) were included as controls. Cells treated with lentivirus were selected from days 4 to 7. Multiple gRNAs effected potent transcriptional repression of ε-, γ-, and β-globin genes when assayed 7 days after transduction, with up to 95% knockdown achieved (FIGS. 57B, 57C, 57D). Expression of ε-globin was silenced the most in response gRNAs targeted to the HS2 enhancer. Treatment with dCas9-KRAB with a gRNA resulted in dramatically more repression than treatment with dCas9 and gRNA.

These findings demonstrate that dCas9-KRAB targeted to the HS2 enhancer by gRNAs effects potent repression of the distal globin genes. This is the first example of targeted epigenetic control of distal regulatory elements in mammalian cells by the CRISPR/Cas9 system. Enhancers regulate development and disease, and this disclosure provides a method to probe and control enhancer function and may be used to determine the effects of dCas9-KRAB on local chromatin accessibility and genome-wide expression.

### Example 23

dCas9-p300

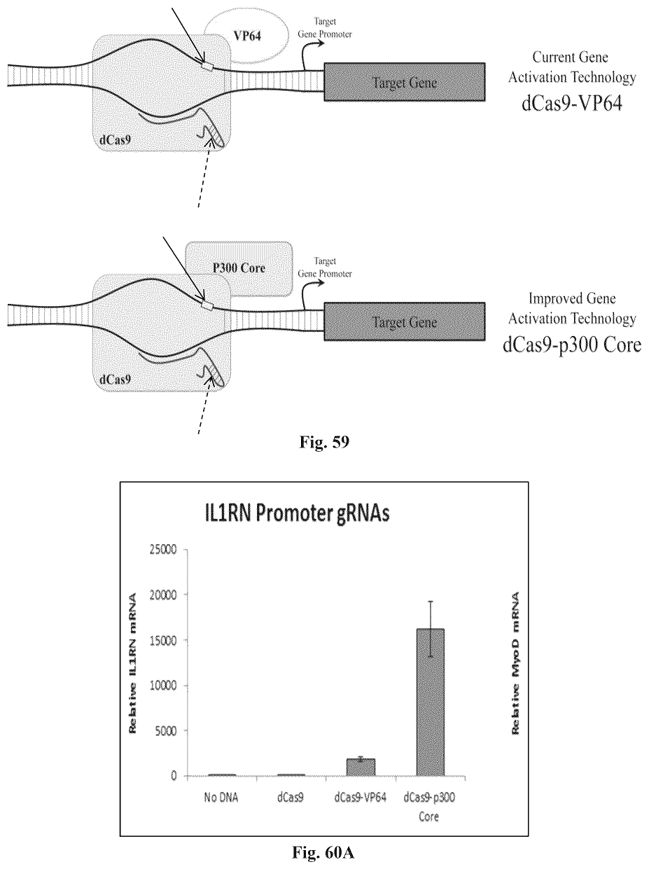

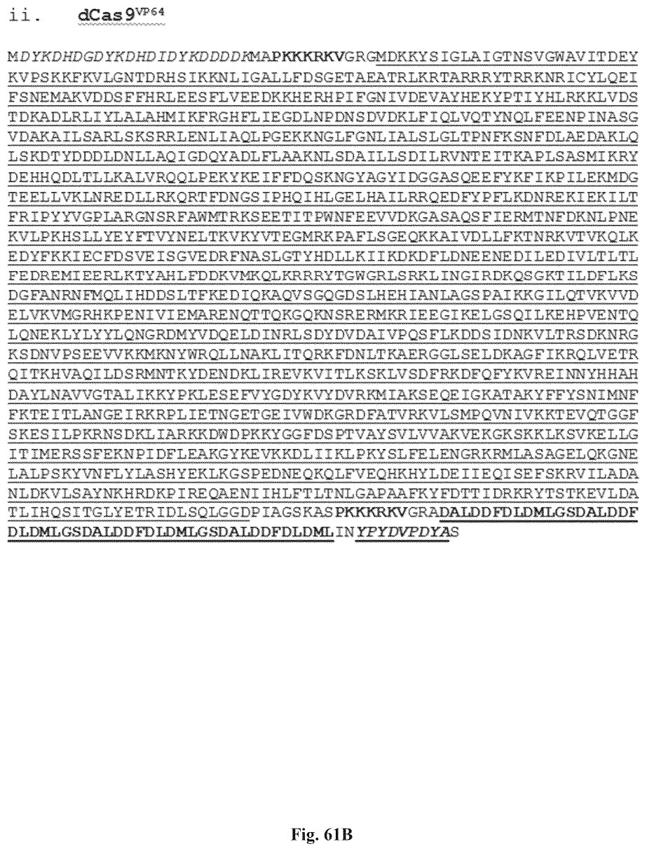

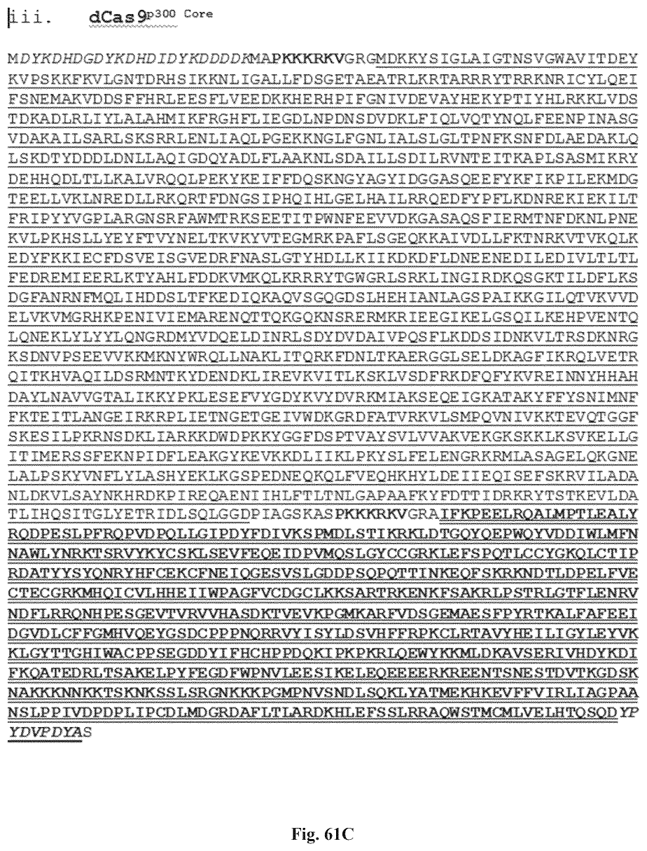

A dCas9-p300 fusion protein was designed and compared to dCas9-VP64 fusion protein (see FIG. 59). The amino acid constructs of dCas9 constructions are shown in FIG. 61A-61C. Cells from the Human Embryonic Kidney tissue culture line HEK293T (ATCC; CRL-11268) were seeded at a density of 1.5e5 cells per well in 24-well tissue culture dishes one day prior to transfection with Lipofectamine 2000 transfection reagent (Life Technologies). 24 hrs later cells were transfected with 1 μL Lipofectamine 2000, 375 ng dCas9 expression construct (dCas9, dCas9VP64, or dCas9p300 respectively), and 125 ng of pooled gRNA expression plasmids (4 each at equimolar ratios). Table 13 shows the gRNA information.

TABLE 13
gRNA information

| | | Genomic | |
|---|---|---|---|
| | | Location | |
| Target | Protospacer Sequence | (GRCh38 | |
| Location | (5′-3′) | Primary Assembly) | Reference |
| IL1RN | TGTACTCTCTGAGGTGCTC | Chr2: | Perez-Pinera et |
| Promoter | (SEQ ID NO: 606) | 113117865- | al., Nat Methods, |
| | | 113117883 | 2013 |
| IL1RN | ACGCAGATAAGAACCAGTT | Chr2: | Perez-Pinera et |
| Promoter | (SEQ ID NO: 607) | 113117714- | al., Nat Methods, |
| | | 113117732 | 2013 |
| IL1RN | CATCAAGTCAGCCATCAGC | Chr2: | Perez-Pinera et |
| Promoter | (SEQ ID NO: 608) | 113117781- | al., Nat Methods, |
| | | 113117799 | 2013 |
| IL1RN | GAGTCACCCTCCTGGAAAC | Chr2: | Perez-Pinera et |
| Promoter | (SEQ ID NO: 609) | 113117749- | al., Nat Methods, |
| | | 113117767 | 2013 |
| MyoD Promoter | CCTGGGCTCCGGGGCGTTT | Chr11: | Perez-Pinera et |
| | (SEQ ID NO: 610) | 17719509- | al., Nat Methods, |
| | | 17719527 | 2013 |
| MyoD Promoter | GGCCCCTGCGGCCACCCCG | Chr11: | Perez-Pinera et |
| | (SEQ ID NO: 611) | 17719422- | al., Nat Methods, |
| | | 17719440 | 2013 |
| MyoD Promoter | CTCCCTCCCTGCCCGGTAG | Chr11: | Perez-Pinera et |
| | (SEQ ID NO: 612) | 17719350- | al., Nat Methods, |
| | | 17719368 | 2013 |
| MyoD Promoter | AGGTTTGGAAAGGGCGTGC | Chr11: | Perez-Pinera et |
| | (SEQ ID NO: 613) | 17719290- | al., Nat Methods, |
| | | 17719308 | 2013 |
| Oct4 Promoter | ACTCCACTGCACTCCAGTCT | Chr6: | Hu et al Nucleic |
| | (SEQ ID NO: 614) | 31170953- | Acids Res, 2014 |
| | | 31170934 | |
| Oct4 Promoter | TCTGTGGGGGACCTGCACTG | Chr6: | Hu et al Nucleic |
| | (SEQ ID NO: 615) | 31170885- | Acids Res, 2014 |
| | | 31170866 | |
| Oct4 Promoter | GGGGCGCCAGTTGTGTCTCC | Chr6: | Hu et al Nucleic |
| | (SEQ ID NO: 616) | 31170855- | Acids Res, 2014 |
| | | 31170836 | |
| Oct4 Promoter | ACACCATTGCCACCACCATT | Chr6: | Hu et al Nucleic |
| | (SEQ ID NO: 617) | 31170816- | Acids Res, 2014 |
| | | 31170797 | |

The 3 days post-transfection cells were harvested and assayed by RT-QPCR for mRNA expression. The RT-QPCR primer sequences are listed in Table 14.

TABLE 14
RT-QPCR Primers

| Primer | Primer Sequence | Reference |
|---|---|---|
| Target | (5′-3′) | |
| GAPDH | CAATGACCCCTTCATTGACC | Perez-Pinera et al., |
| Forward | (SEQ ID NO: 618) | Nat Methods, 2013 |
| GAPDH | TTGATTTTGGAGGGATCTCG | Perez-Pinera et al., |
| Reverse | (SEQ ID NO: 619) | Nat Methods, 2013 |
| IL1RN | GGAATCCATGGAGGGAAGAT | Perez-Pinera et al., |
| Forward | (SEQ ID NO: 620) | Nat Methods, 2013 |
| IL1RN | TGTTCTCGCTCAGGTCAGTG | Perez-Pinera et al., |
| Reverse | (SEQ ID NO: 621) | Nat Methods, 2013 |
| MyoD | CTCTCTGCTCCTTTGCCACA | Perez-Pinera et al., |
| Forward | (SEQ ID NO: 622) | Nat Methods, 2013 |
| MyoD | GTGCTCTTCGGGTTTCAGGA | Perez-Pinera et al., |
| Reverse | (SEQ ID NO: 623) | Nat Methods, 2013 |
| Oct4 | CGAAAGAGAAAGCGAACCAGTATCGAGAAC | Hu et al., Nucleic |
| Forward | (SEQ ID NO: 624) | Acids Res, 2014 |
| Oct4 | CGTTGTGCATAGTCGCTGCTTGATCGC | Hu et al., Nucleic |
| Reverse | (SEQ ID NO: 625) | Acids Res, 2014 |

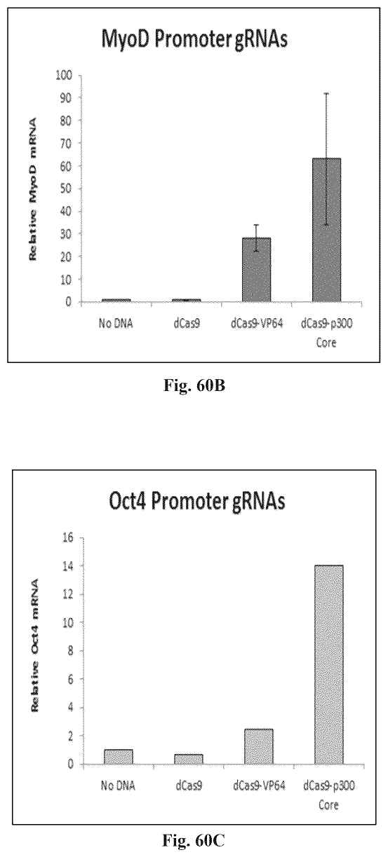

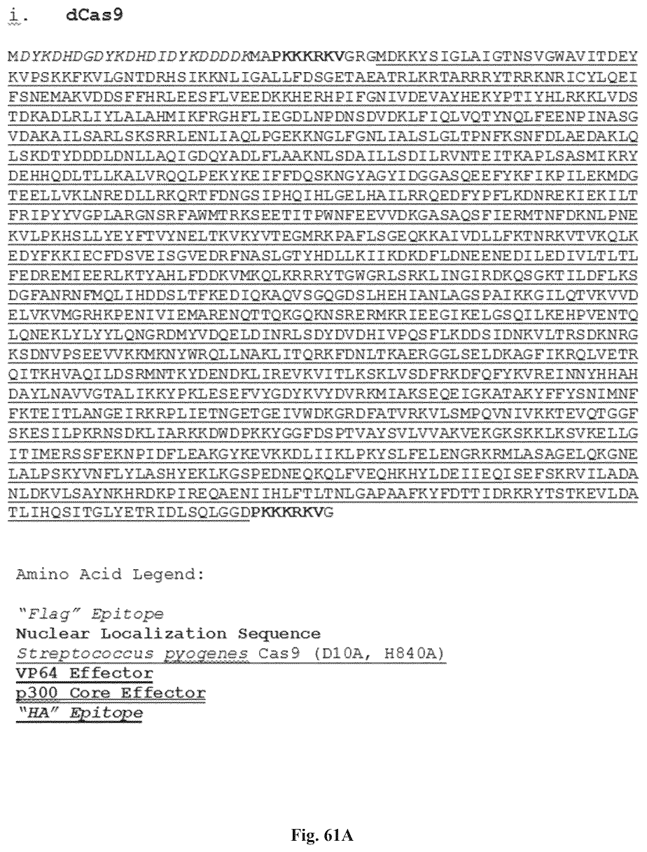

RT-QPCR was normalized to GAPDH expression using the ΔΔCₜ method. Results are expressed as fold-increase expression of the gene of interest relative to cells treated with Lipofectamine only without DNA transfected (“No DNA”) (See FIGS. 60A-60C).

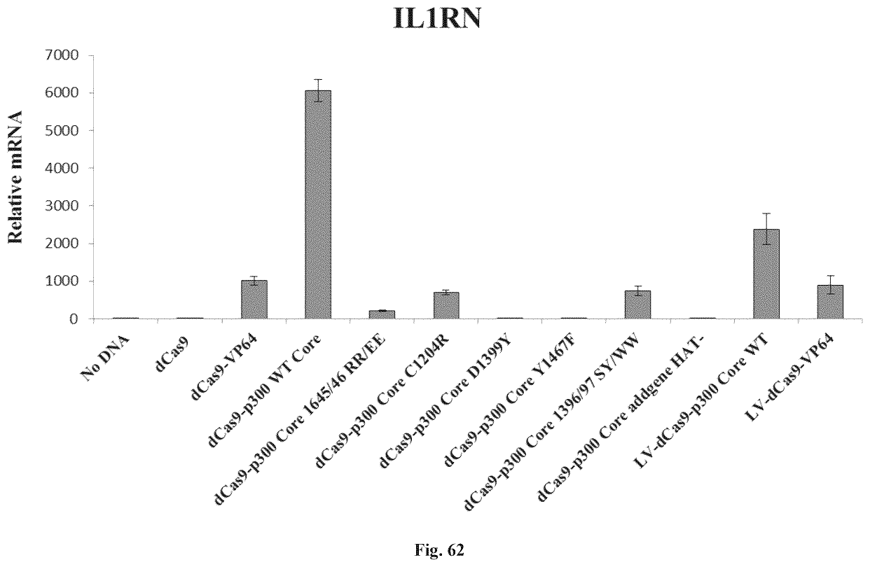

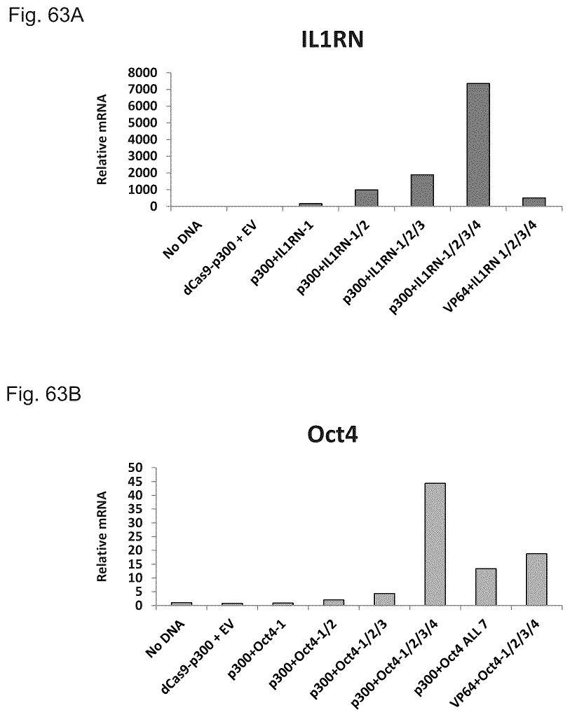

FIG. 62 shows that mutating residues in the p300 HAT domain causes a loss of its ability to activate gene expression. FIGS. 63A-63B show that multiple gRNAs work synergistically with dCas-9-p300, as showed with dCas-9-VP64.

### Example 24

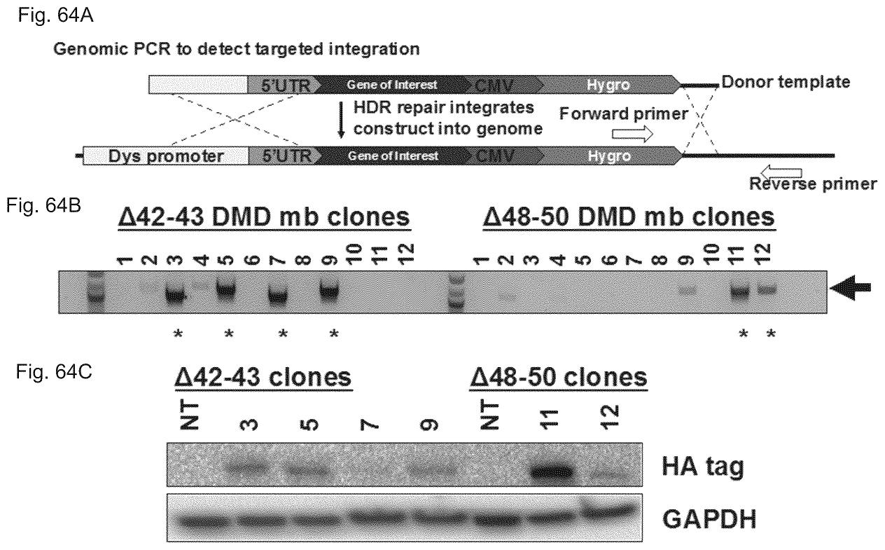

FIGS. 64A-64C show TALEN mediated integration of minidystrophin at the 5′UTR of the Dp427m skeletal muscle isoform of dystrophin in skeletal myoblast cell lines derived from human DMD patients carrying different deletions in the dystrophin gene. DMD patient cells were electroporated with constructs encoding a TALEN pair active at the 5′UTR locus and a donor template carrying the minidystrophin gene. FIG. 64A shows a schematic of how minidystrophin was integrated into the 5′UTR. FIG. 64B shows that hygromycin-resistant clonal cell lines were isolated and screened by PCR for successful site-specific integrations at the 5′UTR using the primers shown in FIG. 64A. Asterisks indicate clones selected for further analysis in FIG. 64C. FIG. 64C shows that clonally isolated DMD myoblasts with detected integration events were differentiated for 6 days and assessed for expression of an HA tag fused to the C terminus of minidystrophin.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

APPENDIX
hCas9-T2A-GFP DNA sequence: SEQ ID NO: 145 (SpCas9 human optimized sequence, HA
tag, T2A peptide, eGFP sequence)
gccaccATGGACAAGAAGTACTCCATTGGGCTCGATATCGGCACAAACAGCGTCGGCTGGGCCGTCATTACGGACGAGTA
CAAGGTGCCGAGCAAAAAATTCAAAGTTCTGGGCAATACCGATCGCCACAGCATAAAGAAGAACCTCATTGGCGCCCTCC
TGTTCGACTCCGGGGAGACGGCCGAAGCCACGCGGCTCAAAAGAACAGCACGGCGCAGATATACCCGCAGAAAGAATCGG
ATCTGCTACCTGCAGGAGATCTTTAGTAATGAGATGGCTAAGGTGGATGACTCTTTCTTCCATAGGCTGGAGGAGTCCTT
TTTGGTGGAGGAGGATAAAAAGCACGAGCGCCACCCAATCTTTGGCAATATCGTGGACGAGGTGGCGTACCATGAAAAGT
ACCCAACCATATATCATCTGAGGAAGAAGCTTGTAGACAGTACTGATAAGGCTGACTTGCGGTTGATCTATCTCGCGCTG
GCGCATATGATCAAATTTCGGGGACACTTCCTCATCGAGGGGGACCTGAACCCAGACAACAGCGATGTCGACAAACTCTT
TATCCAACTGGTTCAGACTTACAATCAGCTTTTCGAAGAGAACCCGATCAACGCATCCGGAGTTGACGCCAAAGCAATCC
TGAGCGCTAGGCTGTCCAAATCCCGGCGGCTCGAAAACCTCATCGCACAGCTCCCTGGGGAGAAGAAGAACGGCCTGTTT
GGTAATCTTATCGCCCTGTCACTCGGGCTGACCCCCAACTTTAAATCTAACTTCGACCTGGCCGAAGATGCCAAGCTTCA
ACTGAGCAAAGACACCTACGATGATGATCTCGACAATCTGCTGGCCCAGATCGGCGACCAGTACGCAGACCTTTTTTTGG
CGGCAAAGAACCTGTCAGACGCCATTCTGCTGAGTGATATTCTGCGAGTGAACACGGAGATCACCAAAGCTCCGCTGAGC
GCTAGTATGATCAAGCGCTATGATGAGCACCACCAAGACTTGACTTTGCTGAAGGCCCTTGTCAGACAGCAACTGCCTGA
GAAGTACAAGGAAATTTTCTTCGATCAGTCTAAAAATGGCTACGCCGGATACATTGACGGCGGAGCAAGCCAGGAGGAAT
TTTACAAATTTATTAAGCCCATCTTGGAAAAAATGGACGGCACCGAGGAGCTGCTGGTAAAGCTTAACAGAGAAGATCTG
TTGCGCAAACAGCGCACTTTCGACAATGGAAGCATCCCCCACCAGATTCACCTGGGCGAACTGCACGCTATCCTCAGGCG
GCAAGAGGATTTCTACCCCTTTTTGAAAGATAACAGGGAAAAGATTGAGAAAATCCTCACATTTCGGATACCCTACTATG
TAGGCCCCCTCGCCCGGGGAAATTCCAGATTCGCGTGGATGACTCGCAAATCAGAAGAGACCATCACTCCCTGGAACTTC
GAGGAAGTCGTGGATAAGGGGGCCTCTGCCCAGTCCTTCATCGAAAGGATGACTAACTTTGATAAAAATCTGCCTAACGA
AAAGGTGCTTCCTAAACACTCTCTGCTGTACGAGTACTTCACAGTTTATAACGAGCTCACCAAGGTCAAATACGTCACAG
AAGGGATGAGAAAGCCAGCATTCCTGTCTGGAGAGCAGAAGAAAGCTATCGTGGACCTCCTCTTCAAGACGAACCGGAAA
GTTACCGTGAAACAGCTCAAAGAAGACTATTTCAAAAAGATTGAATGTTTCGACTCTGTTGAAATCAGCGGAGTGGAGGA
TCGCTTCAACGCATCCCTGGGAACGTATCACGATCTCCTGAAAATCATTAAAGACAAGGACTTCCTGGACAATGAGGAGA
ACGAGGACATTCTTGAGGACATTGTCCTCACCCTTACGTTGTTTGAAGATAGGGAGATGATTGAAGAACGCTTGAAAACT
TACGCTCATCTCTTCGACGACAAAGTCATGAAACAGCTCAAGAGGCGCCGATATACAGGATGGGGGCGGCTGTCAAGAAA
ACTGATCAATGGGATCCGAGACAAGCAGAGTGGAAAGACAATCCTGGATTTTCTTAAGTCCGATGGATTTGCCAACCGGA
ACTTCATGCAGTTGATCCATGATGACTCTCTCACCTTTAAGGAGGACATCCAGAAAGCACAAGTTTCTGGCCAGGGGGAC
AGTCTTCACGAGCACATCGCTAATCTTGCAGGTAGCCCAGCTATCAAAAAGGGAATACTGCAGACCGTTAAGGTCGTGGA
TGAACTCGTCAAAGTAATGGGAAGGCATAAGCCCGAGAATATCGTTATCGAGATGGCCCGAGAGAACCAAACTACCCAGA
AGGGACAGAAGAACAGTAGGGAAAGGATGAAGAGGATTGAAGAGGGTATAAAAGAACTGGGGTCCCAAATCCTTAAGGAA
CACCCAGTTGAAAACACCCAGCTTCAGAATGAGAAGCTCTACCTGTACTACCTGCAGAACGGCAGGGACATGTACGTGGA
TCAGGAACTGGACATCAATCGGCTCTCCGACTACGACGTGGATCATATCGTGCCCCAGTCTTTTCTCAAAGATGATTCTA
TTGATAATAAAGTGTTGACAAGATCCGATAAAAATAGAGGGAAGAGTGATAACGTCCCCTCAGAAGAAGTTGTCAAGAAA
ATGAAAAATTATTGGCGGCAGCTGCTGAACGCCAAACTGATCACACAACGGAAGTTCGATAATCTGACTAAGGCTGAACG
AGGTGGCCTGTCTGAGTTGGATAAAGCCGGCTTCATCAAAAGGCAGCTTGTTGAGACACGCCAGATCACCAAGCACGTGG
CCCAAATTCTCGATTCACGCATGAACACCAAGTACGATGAAAATGACAAACTGATTCGAGAGGTGAAAGTTATTACTCTG
AAGTCTAAGCTGGTCTCAGATTTCAGAAAGGACTTTCAGTTTTATAAGGTGAGAGAGATCAACAATTACCACCATGCGCA
TGATGCCTACCTGAATGCAGTGGTAGGCACTGCACTTATCAAAAAATATCCCAAGCTTGAATCTGAATTTGTTTACGGAG
ACTATAAAGTGTACGATGTTAGGAAAATGATCGCAAAGTCTGAGCAGGAAATAGGCAAGGCCACCGCTAAGTACTTCTTT
TACAGCAATATTATGAATTTTTTCAAGACCGAGATTACACTGGCCAATGGAGAGATTCGGAAGCGACCACTTATCGAAAC
AAACGGAGAAACAGGAGAAATCGTGTGGGACAAGGGTAGGGATTTCGCGACAGTCCGGAAGGTCCTGTCCATGCCGCAGG
TGAACATCGTTAAAAAGACCGAAGTACAGACCGGAGGCTTCTCCAAGGAAAGTATCCTCCCGAAAAGGAACAGCGACAAG
CTGATCGCACGCAAAAAAGATTGGGACCCCAAGAAATACGGCGGATTCGATTCTCCTACAGTCGCTTACAGTGTACTGGT
TGTGGCCAAAGTGGAGAAAGGGAAGTCTAAAAAACTCAAAAGCGTCAAGGAACTGCTGGGCATCACAATCATGGAGCGAT
CAAGCTTCGAAAAAAACCCCATCGACTTTCTCGAGGCGAAAGGATATAAAGAGGTCAAAAAAGACCTCATCATTAAGCTT
CCCAAGTACTCTCTCTTTGAGCTTGAAAACGGCCGGAAACGAATGCTCGCTAGTGCGGGCGAGCTGCAGAAAGGTAACGA
GCTGGCACTGCCCTCTAAATACGTTAATTTCTTGTATCTGGCCAGCCACTATGAAAAGCTCAAAGGGTCTCCCGAAGATA
ATGAGCAGAAGCAGCTGTTCGTGGAACAACACAAACACTACCTTGATGAGATCATCGAGCAAATAAGCGAATTCTCCAAA
AGAGTGATCCTCGCCGACGCTAACCTCGATAAGGTGCTTTCTGCTTACAATAAGCACAGGGATAAGCCCATCAGGGAGCA
GGCAGAAAACATTATCCACTTGTTTACTCTGACCAACTTGGGCGCGCCTGCAGCCTTCAAGTACTTCGACACCACCATAG
ACAGAAAGCGGTACACCTCTACAAAGGAGGTCCTGGACGCCACACTGATTCATCAGTCAATTACGGGGCTCTATGAAACA
AGAATCGACCTCTCTCAGCTCGGTGGAGACAGCAGGGCTGACCCCAAGAAGAAGAGGAAGGTGGCTAGCGAGGGCAGAGG
AAGTCTTCTAACATGCGGTGACGTGGAGGAGAATCCCGGCCCTGgtacCgtgagcaagggcgaggagctgttcaccgggg
tggtgcccatcctggtcgagctggacggcgacgtaaacggccacaagttcagcgtgtccggcgagggcgagggcgatgcc
acctacggcaagctgaccctgaagttcatctgcaccaccggcaagctgcccgtgccctggcccaccctcgtgaccaccct
gacctacggcgtgcagtgcttcagccgctaccccgaccacatgaagcagcacgacttcttcaagtccgccatgcccgaag
gctacgtccaggagcgcaccatcttcttcaaggacgacggcaactacaagacccgcgccgaggtgaagttcgagggcgac
accctggtgaaccgcatcgagctgaagggcatcgacttcaaggaggacggcaacatcctggggcacaagctggagtacaa
ctacaacagccacaacgtctatatcatggccgacaagcagaagaacggcatcaaggtgaacttcaagatccgccacaaca
tcgaggacggcagcgtgcagctcgccgaccactaccagcagaacacccccatcggcgacggccccgtgctgctgcccgac
aaccactacctgagcacccagtccgccctgagcaaagaccccaacgagaagcgcgatcacatggtcctgctggagttcgt
gaccgccgccgggatcactctcggcatggacgagctgtacaagAccggTTAG
AAV/Rosa26 construct (SEQ ID NO: 456)
ggggggggggggggggggttggccactccctctctgcgcgctcgctcgctcactgaggccgggcgaccaaaggtcgcccg
acgcccgggctttgcccgggcggcctcagtgagcgagcgagcgcgcagagagggagtggccaactccatcactaggggtt
cctagatctgaattcggtacccgttacataacttacggtaaatggcccgcctggctgaccgcccaacgacccccgcccat
tgacgtcaataatgacgtatgttcccatagtaacgccaatagggactttccattgacgtcaatgggtggagtatttacgg
taaactgcccacttggcagtacatcaagtgtatcatatgccaagtacgccccctattgacgtcaatgacggtaaatggcc
cgcctggcattatgcccagtacatgaccttatgggactttcctacttggcagtacatctacgtattagtcatcgctatta
ccatggtgatgcggttttggcagtacatcaatgggcgtggatagcggtttgactcacggggatttccaagtctccacccc
attgacgtcaatgggagtttgttttggcaccaaaatcaacgggactttccaaaatgtcgtaacaactccgccccattgac
gcaaatgggcggtaggcgtgtacggtgggaggtctatataagcagagctcgtttagtgaaccgtcagatcgcctggagac
gccatccacgctgttttgacctccatagaagacaccgggaccgatccagcctccggactctagaggatccggtactcgag
gaactgaaaaaccagaaagttaactggtaagtttagtctttttgtcttttatttcaggtcccggatccggtggtggtgca
aatcaaagaactgctcctcagtggatgttgcctttacttctaggcctgtacggaagtgttacttctgctctaaaagctgc
ggaattgtacccgcggcccgggatccaccggTGGCTAGCgtctataggcccacccccTTGGTGGAATTCGCCATGAGGTC
TGACTACAAAGACCATGACGGTGATTATAAAGATCATGACATCGATTACAAGGATGACGATGACAAGATGGCCCCCAAGA
AGAAGaggaaggtgggcctcgaGCCCGGAGAAAAACCGTACAAGTGCCCTGAGTGCGGGAAATCATTCTCCGACCCTGGG
GCGCTCGTCCGGCACCAAAGGACGCATACAGGGGAAAAGCCGTATAAGTGCCCCGAGTGTGGAAAGAGCTTCTCGCAGAG
AGCCCACCTTGAACGACACCAAAGAACACACACTGGTGAGAAACCCTATAAGTGTCCAGAGTGCGGCAAATCGTTTAGCA
GATCCGATGACTTGGTGCGCCACCAGCGGACACACACGGGTGAAAAGCCCTACAAATGCCCGGAGTGTGGGAAGTCGTTT
TCAAGGTCGGATCATCTGACTACCCATCAGCGCACCCATACGGGAGCggccgcccgcgccctGGTGAAGAGCGAGCTGGA
GGAGAAGAAGTCCGAGCTGCGGCACAAGCTGAAGTACGTGCCCCACGAGTACATCGAGCTGATCGAGATCGCCAGGAACC
CCACCCAGGACCGCATCCTGGAGATGAAGGTGATGGAGTTCTTCATGAAGGTGTACGGCTACAGGGGAGAGCACCTGGGC
GGAAGCAGAAAGCCTGACGGCGCCATCTATACAGTGGGCAGCCCCATCGATTACGGCGTGATCGTGGACACAAAGGCCTA
CAGCGGCGGCTACAATCTGCCTATCGGCCAGGCCGACGAGATGCAGAGATACGTGAAGGAGAACCAGACCCGGAATAAGC
ACATCAACCCCAACGAGTGGTGGAAGGTGTACCCTAGCAGCGTGACCGAGTTCAAGTTCCTGTTCGTGAGCGGCCACTTC
AAGGGCAACTACAAGGCCCAGCTGACCAGGCTGAACCGCAAAACCAACTGCAATGGCGCCGTGCTGAGCGTGGAGGAGCT
GCTGATCGGCGGCGAGATGATCAAAGCCGGCACCCTGACACTGGAGGAGGTGCGGCGCAAGTTCAACAACGGCGAGATCA
ACTTCGAGGGCAGAGGAAGTCTTCTAACATGCGGTGACGTGGAGGAGAATCCCGGCCCTAgatcTGACTACAAAGACCAT
GACGGTGATTATAAAGATCATGACATCGATTACAAGGATGACGATGACAAGATGGCCCCCAAGAAGAAGaggaaggtggg
cctcgagccGGGAGAGAAGCCGTACAAGTGTCCCGAATGTGGAAAGAGCTTCTCACAGTCGGGGGACCTTCGGCGCCACC
AGCGCACACATACTGGTGAAAAGCCGTATAAGTGTCCAGAATGTGGCAAATCATTCTCCACATCAGGGAGCCTGGTCAGG
CACCAGCGAACCCACACGGGTGAGAAGCCCTATAAGTGCCCCGAATGCGGGAAGTCCTTTTCGCAGAGAGCCCACTTGGA
GAGGCACCAGAGGACCCATACGGGGGAGAAACCTTACAAGTGCCCTGAATGCGGAAAGTCGTTCTCGACCCATCTGGATC
TCATCAGACATCAGAGAACGCACACTGGAGAGAAACCCTACAAATGTCCCGAGTGTGGGAAGTCGTTTAGCCGAAAGGAC
AATCTCAAAAACCATCAACGGACACACACGGGTGAAAAACCATACAAATGCCCGGAGTGCGGCAAATCGTTTTCCCAACT
TGCGCACTTGCGGGCACACCAACGCACGCATACTGGAGCGGCCGCccgcgccCTGGTGAAGAGCGAGCTGGAGGAGAAGA
AGTCCGAGCTGCGGCACAAGCTGAAGTACGTGCCCCACGAGTACATCGAGCTGATCGAGATCGCCAGGAACCCCACCCAG
GACCGCATCCTGGAGATGAAGGTGATGGAGTTCTTCATGAAGGTGTACGGCTACAGGGGAGAGCACCTGGGCGGAAGCAG
AAAGCCTGACGGCGCCATCTATACAGTGGGCAGCCCCATCGATTACGGCGTGATCGTGGACACAAAGGCCTACAGCGGCG
GCTACAATCTGCCTATCGGCCAGGCCGACGAGATGGAGAGATACGTGGAGGAGAACCAGACACGGGATAAGCACCTCAAC
CCCAACGAGTGGTGGAAGGTGTACCCTAGCAGCGTGACCGAGTTCAAGTTCCTGTTCGTGAGCGGCCACTTCAAGGGCAA
CTACAAGGCCCAGCTGACCAGGCTGAACCACATCACCAACTGCAATGGCGCCGTGCTGAGCGTGGAGGAGCTGCTGATCG
GCGGCGAGATGATCAAAGCCGGCACCCTGACACTGGAGGAGGTGCGGCGCAAGTTCAACAACGGCGAGATCAACTTCTGA
TTAATTAACTAATCTAGAGTcgactagagctcgctgatcagcctcgactgtgccttctagttgccagccatctgttgttt
gcccctcccccgtgccttccttgaccctggaaggtgccactcccactgtcctttcctaataaaatgaggaaattgcatcg
cattgtctgagtaggtgtcattctattctggggggtggggtggggcaggacagcaagggggaggattgggaagacaatag
caggcatgctggggagagatctaggaacccctagtgatggagttggccactccctctctgcgcgctcgctcgctcactga
ggccgcccgggcaaagcccgggcgtcgggcgacctttggtcgcccggcctcagtgagcgagcgagcgcgcagagagggag
tggccaacccccccccccccccccctgcagcccagctgcattaatgaatcggccaacgcgcggggagaggcggtttgcgt
attgggcgctcttccgcttcctcgctcactgactcgctgcgctcggtcgttcggctgcggcgagcggtatcagctcactc
aaaggcggtaatacggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaaaatcgacgctca
agtcagaggtggcgaaacccgacaggactataaagataccaggcgtttccccctggaagctccctcgtgcgctctcctgt
tccgaccctgccgcttaccggatacctgtccgcctttctcccttcgggaagcgtggcgctttctcaatgctcacgctgta
ggtatctcagttcggtgtaggtcgttcgctccaagctgggctgtgtgcacgaaccccccgttcagcccgaccgctgcgcc
ttatccggtaactatcgtcttgagtccaacccggtaagacacgacttatcgccactggcagcagccactggtaacaggat
tagcagagcgaggtatgtaggcggtgctacagagttcttgaagtggtggcctaactacggctacactagaaggacagtat
ttggtatctgcgctctgctgaagccagttaccttcggaaaaagagttggtagctcttgatccggcaaacaaaccaccgct
ggtagcggtggtttttttgtttgcaagcagcagattacgcgcagaaaaaaaggatctcaagaagatcctttgatcttttc
tacggggtctgacgctcagtggaacgaaaactcacgttaagggattttggtcatgagattatcaaaaaggatcttcacct
agatccttttaaattaaaaatgaagttttaaatcaatctaaagtatatatgagtaaacttggtctgacagttaccaatgc
ttaatcagtgaggcacctatctcagcgatctgtctatttcgttcatccatagttgcctgactccccgtcgtgtagataac
tacgatacgggagggcttaccatctggccccagtgctgcaatgataccgcgagacccacgctcaccggctccagatttat
cagcaataaaccagccagccggaagggccgagcgcagaagtggtcctgcaactttatccgcctccatccagtctattaat
tgttgccgggaagctagagtaagtagttcgccagttaatagtttgcgcaacgttgttgccattgctacaggcatcgtggt
gtcacgctcgtcgtttggtatggcttcattcagctccggttcccaacgatcaaggcgagttacatgatcccccatgttgt
gcaaaaaagcggttagctccttcggtcctccgatcgttgtcagaagtaagttggccgcagtgttatcactcatggttatg
gcagcactgcataattctcttactgtcatgccatccgtaagatgcttttctgtgactggtgagtactcaaccaagtcatt
ctgagaatagtgtatgcggcgaccgagttgctcttgcccggcgtcaatacgggataataccgcgccacatagcagaactt
taaaagtgctcatcattggaaaacgttcttcggggcgaaaactctcaaggatcttaccgctgttgagatccagttcgatg
taacccactcgtgcacccaactgatcttcagcatcttttactttcaccagcgtttctgggtgagcaaaaacaggaaggca
aaatgccgcaaaaaagggaataagggcgacacggaaatgttgaatactcatactcttcctttttcaatattattgaagca
tttatcagggttattgtctcatgagcggatacatatttgaatgtatttagaaaaataaacaaataggggttccgcgcaca
tttccccgaaaagtgccacctgacgtctaagaaaccattattatcatgacattaacctataaaaataggcgtatcacgag
gccctttcgtctcgcgcgtttcggtgatgacggtgaaaacctctgacacatgcagctcccggagacggtcacagcttgtc
tgtaagcggatgccgggagcagacaagcccgtcagggcgcgtcagcgggtgttggcgggtgtcggggctggcttaactat
gcggcatcagagcagattgtactgagagtgcaccatatgcggtgtgaaataccgcacagatgcgtaaggagaaaataccg
catcaggaaattgtaaacgttaatattttgttaaaattcgcgttaaatttttgttaaatcagctcattttttaaccaata
ggccgaaatcggcaaaatcccttataaatcaaaagaatagaccgagatagggttgagtgttgttccagtttggaacaaga
gtccactattaaagaacgtggactccaacgtcaaagggcgaaaaaccgtctatcagggcgatggcccactacgtgaacca
tcaccctaatcaagttttttggggtcgaggtgccgtaaagcactaaatcggaaccctaaagggagcccccgatttagagc
ttgacggggaaagccggcgaacgtggcgagaaaggaagggaagaaagcgaaaggagcgggcgctagggcgctggcaagtg
tagcggtcacgctgcgcgtaaccaccacacccgccgcgcttaatgcgccgctacagggcgcgtcgcgccattcgccattc
aggctacgcaactgttgggaagggcgatcggtgcgggcctcttcgctattacgccagctggctgca
HS2 Enhancer Target Sequence (SEQ ID NO: 467)
taagcttcagtttttccttagttcctgttacatttctgtgtgtctccattagtgacctcccatagtccaagcatgagcag
ttctggccaggcccctgtcggggtcagtgccccacccccgccttctggttctgtgtaaccttctaagcaaaccttctggc
tcaagcacagcaatgctgagtcatgatgagtcatgctgaggcttagggtgtgtgcccagatgttctcagcctagagtgat
gactcctatctgggtccccagcaggatgcttacagggcagatggcaaaaaaaaggagaagctgaccacctgactaaaact
ccacctcaaacggcatcataaagaaaatggatgcctgagacagaatgtgacatattctagaatatatt
dSpCas9-KRAB Sequence (SEQ ID NO: 466)
ATGGACTACAAAGACCATGACGGTGATTATAAAGATCATGACATCGATTACAAGGATGACGATGACAAGatggcccccaa
gaagaagaggaaggtgggccgcggaATGGACAAGAAGTACTCCATTGGGCTCGCCATCGGCACAAACAGCGTCGGCTGGG
CCGTCATTACGGACGAGTACAAGGTGCCGAGCAAAAAATTCAAAGTTCTGGGCAATACCGATCGCCACAGCATAAAGAAG
AACCTCATTGGCGCCCTCCTGTTCGACTCCGGGGAAACCGCCGAAGCCACGCGGCTCAAAAGAACAGCACGGCGCAGATA
TACCCGCAGAAAGAATCGGATCTGCTACCtgcaGGAGATCTTTAGTAATGAGATGGCTAAGGTGGATGACTCTTTCTTCC
ATAGGCTGGAGGAGTCCTTTTTGGTGGAGGAGGATAAAAAGCACGAGCGCCACCCAATCTTTGGCAATATCGTGGACGAG
GTGGCGTACCATGAAAAGTACCCAACCATATATCATCTGAGGAAGAAGCTTGTAGACAGTACTGATAAGGCTGACTTGCG
GTTGATCTATCTCGCGCTGGCGCATATGATCAAATTTCGGGGACACTTCCTCATCGAGGGGGACCTGAACCCAGACAACA
GCGATGTCGACAAACTCTTTATCCAACTGGTTCAGACTTACAATCAGCTTTTCGAAGAGAACCCGATCAACGCATCCGGA
GTTGACGCCAAAGCAATCCTGAGCGCTAGGCTGTCCAAATCCCGGCGGCTCGAAAACCTCATCGCACAGCTCCCTGGGGA
GAAGAAGAACGGCCTGTTTGGTAATCTTATCGCCCTGTCACTCGGGCTGACCCCCAACTTTAAATCTAACTTCGACCTGG
CCGAAGATGCCAAGCTTCAACTGAGCAAAGACACCTACGATGATGATCTCGACAATCTGCTGGCCCAGATCGGCGACCAG
TACGCAGACCTTTTTTTGGCGGCAAAGAACCTGTCAGACGCCATTCTGCTGAGTGATATTCTGCGAGTGAACACGGAGAT
CACCAAAGCTCCGCTGAGCGCTAGTATGATCAAGCGCTATGATGAGCACCACCAAGACTTGACTTTGCTGAAGGCCCTTG
TCAGACAGCAACTGCCTGAGAAGTACAAGGAAATTTTCTTCGATCAGTCTAAAAATGGCTACGCCGGATACATTGACGGC
GGAGCAAGCCAGGAGGAATTTTACAAATTTATTAAGCCCATCTTGGAAAAAATGGACGGCACCGAGGAGCTGCTGGTAAA
GCTTAACAGAGAAGATCTGTTGCGCAAACAGCGCACTTTCGACAATGGAAGCATCCCCCACCAGATTCACCTGGGCGAAC
TGCACGCTATCCTCAGGCGGCAAGAGGATTTCTACCCCTTTTTGAAAGATAACAGGGAAAAGATTGAGAAAATCCTCACA
TTTCGGATACCCTACTATGTAGGCCCCCTCGCCCGGGGAAATTCCAGATTCGCGTGGATGACTCGCAAATCAGAAGAGAC
CATCACTCCCTGGAACTTCGAGGAAGTCGTGGATAAGGGGGCCTCTGCCCAGTCCTTCATCGAAAGGATGACTAACTTTG
ATAAAAATCTGCCTAACGAAAAGGTGCTTCCTAAACACTCTCTGCTGTACGAGTACTTCACAGTTTATAACGAGCTCACC
AAGGTCAAATACGTCACAGAAGGGATGAGAAAGCCAGCATTCCTGTCTGGAGAGCAGAAGAAAGCTATCGTGGACCTCCT
CTTCAAGACGAACCGGAAAGTTACCGTGAAACAGCTCAAAGAAGACTATTTCAAAAAGATTGAATGTTTCGACTCTGTTG
AAATCAGCGGAGTGGAGGATCGCTTCAACGCATCCCTGGGAACGTATCACGATCTCCTGAAAATCATTAAAGACAAGGAC
TTCCTGGACAATGAGGAGAACGAGGACATTCTTGAGGACATTGTCCTCACCCTTACGTTGTTTGAAGATAGGGAGATGAT
TGAAGAACGCTTGAAAACTTACGCTCATCTCTTCGACGACAAAGTCATGAAACAGCTCAAGAGGCGCCGATATACAGGAT
GGGGGCGGCTGTCAAGAAAACTGATCAATGGgatcCGAGACAAGCAGAGTGGAAAGACAATCCTGGATTTTCTTAAGTCC
GATGGATTTGCCAACCGGAACTTCATGCAGTTGATCCATGATGACTCTCTCACCTTTAAGGAGGACATCCAGAAAGCACA
AGTTTCTGGCCAGGGGGACAGTCTTCACGAGCACATCGCTAATCTTGCAGGTAGCCCAGCTATCAAAAAGGGAATACTGC
AGACCGTTAAGGTCGTGGATGAACTCGTCAAAGTAATGGGAAGGCATAAGCCCGAGAATATCGTTATCGAGATGGCCCGA
GAGAACCAAACTACCCAGAAGGGACAGAAGAACAGTAGGGAAAGGATGAAGAGGATTGAAGAGGGTATAAAAGAACTGGG
GTCCCAAATCCTTAAGGAACACCCAGTTGAAAACACCCAGCTTCAGAATGAGAAGCTCTACCTGTACTACCTGCAGAACG
GCAGGGACATGTACGTGGATCAGGAACTGGACATCAATCGGCTCTCCGACTACGACGTGGATGCCATCGTGCCCCAGTCT
TTTCTCAAAGATGATTCTATTGATAATAAAGTGTTGACAAGATCCGATAAAAATAGAGGGAAGAGTGATAACGTCCCCTC
AGAAGAAGTTGTCAAGAAAATGAAAAATTATTGGCGGCAGCTGCTGAACGCCAAACTGATCACACAACGGAAGTTCGATA
ATCTGACTAAGGCTGAACGAGGTGGCCTGTCTGAGTTGGATAAAGCCGGCTTCATCAAAAGGCAGCTTGTTGAGACACGC
CAGATCACCAAgcacGTGGCCCAAATTCTCGATTCACGCATGAACACCAAGTACGATGAAAATGACAAACTGATTCGAGA
GGTGAAAGTTATTACTCTGAAGTCTAAGCTGGTCTCAGATTTCAGAAAGGACTTTCAGTTTTATAAGGTGAGAGAGATCA
ACAATTACCACCATGCGCATGATGCCTACCTGAATGCAGTGGTAGGCACTGCACTTATCAAAAAATATCCCAAGCTTGAA
TCTGAATTTGTTTACGGAGACTATAAAGTGTACGATGTTAGGAAAATGATCGCAAAGTCTGAGCAGGAAATAGGCAAGGC
CACCGCTAAGTACTTCTTTTACAGCAATATTATGAATTTTTTCAAGACCGAGATTACACTGGCCAATGGAGAGATTCGGA
AGCGACCACTTATCGAAACAAACGGAGAAACAGGAGAAATCGTGTGGGACAAGGGTAGGGATTTCGCGACAGTCCGGAAG
GTCCTGTCCATGCCGCAGGTGAACATCGTTAAAAAGACCGAAGTACAGACCGGAGGCTTCTCCAAGGAAAGTATCCTCCC
GAAAAGGAACAGCGACAAGCTGATCGCACGCAAAAAAGATTGGGACCCCAAGAAATACGGCGGATTCGATTCTCCTACAG
TCGCTTACAGTGTACTGGTTGTGGCCAAAGTGGAGAAAGGGAAGTCTAAAAAACTCAAAAGCGTCAAGGAACTGCTGGGC
ATCACAATCATGGAGCGATCAAGCTTCGAAAAAAACCCCATCGACTTTCTCGAGGCGAAAGGATATAAAGAGGTCAAAAA
AGACCTCATCATTAAGCTTCCCAAGTACTCTCTCTTTGAGCTTGAAAACGGCCGGAAACGAATGCTCGCTAGTGCGGGCG
AGCTGCAGAAAGGTAACGAGCTGGCACTGCCCTCTAAATACGTTAATTTCTTGTATCTGGCCAGCCACTATGAAAAGCTC
AAAGGGTCTCCCGAAGATAATGAGCAGAAGCAGCTGTTCGTGGAACAACACAAACACTACCTTGATGAGATCATCGAGCA
AATAAGCGAATTCTCCAAAAGAGTGATCCTCGCCGACGCTAACCTCGATAAGGTGCTTTCTGCTTACAATAAGCACAGGG
ATAAGCCCATCAGGGAGCAGGCAGAAAACATTATCCACTTGTTTACTCTGACCAACTTGGGCGCGCCTGCAGCCTTCAAG
TACTTCGACACCACCATAGACAGAAAGCGGTACACCTCTACAAAGGAGGTCCTGGACGCCACACTGATTCATCAGTCAAT
TACGGGGCTCTATGAAACAAGAATCGACCTCTCTCAGCTCGGTGGAGACAGCAGGGCTGACCCCAAGAAGAAGAGGAAGG
TGGctagCGATGCTAAGTCACTGACTGCCTGGTCCCGGACACTGGTGACCTTCAAGGATGTGTTTGTGGACTTCACCAGG
GAGGAGTGGAAGCTGCTGGACACTGCTCAGCAGATCCTGTACAGAAATGTGATGCTGGAGAACTATAAGAACCTGGTTTC
CTTGGGTTATCAGCTTACTAAGCCAGATGTGATCCTCCGGTTGGAGAAGGGAGAAGAGCCCTGGCTGGTGGAGAGAGAAA
TTCACCAAGAGACCCATCCTGATTCAGAGACTGCATTTGAAATCAAATCATCAGTTCCGAAAAAGAAACGCAAAGttGct
agCG

What is claimed is:

1. A fusion protein comprising two heterologous polypeptide domains, wherein the first polypeptide domain comprises a Clustered Regularly Interspaced Short Palindromic Repeats associated (Cas) protein and the second polypeptide domain has an activity selected from the group consisting of transcription activation activity, transcription repression activity, transcription release factor activity, histone modification activity, nuclease activity, nucleic acid association activity, methylase activity, and demethylase activity.

2. The fusion protein of claim 1, wherein the Cas protein comprises Cas9.

3. The fusion protein of claim 2, wherein the Cas9 comprises at least one amino acid mutation which knocks out nuclease activity of Cas9.

4. The fusion protein of claim 3, wherein the at least one amino acid mutation is at least one of D10A and H840A.

5. The fusion protein of claim 3 or 4, wherein the Cas protein comprises iCas9 (amino acids 36-1403 of SEQ ID NO: 1).

6. The fusion protein of any one of the preceding claims, wherein the second polypeptide domain has transcription activation activity.

7. The fusion protein of claim 6, wherein the second polypeptide domain comprises at least one VP16 transcription activation domain repeat.

8. The fusion protein of claim 7, wherein the second polypeptide domain comprises a VP16 tetramer (“VP64”) or a p65 activation domain.

9. The fusion protein of any one of the preceding claims, further comprising a linker connecting the first polypeptide domain to the second polypeptide domain.

10. The fusion protein of any one of the preceding claims, wherein the fusion protein comprises iCas9-VP64.

11. A DNA targeting system comprising the fusion protein of any one of the preceding claims and at least one guide RNA (gRNA).

12. The DNA targeting system of claim 11, wherein the at least one gRNA comprises a 12-22 base pair complementary polynucleotide sequence of the target DNA sequence followed by a protospacer-adjacent motif.

13. The DNA targeting system of claim 11 or 12, wherein the at least one gRNA targets a promoter region of a gene, an enhancer region of a gene, or a transcribed region of a gene.

14. The DNA targeting system of claim 13, wherein the at least one gRNA targets an intron of a gene.

15. The DNA targeting system of claim 13, wherein the at least one gRNA targets an exon of a gene.

16. The DNA targeting system of claim 11 or 12, wherein the at least one gRNA targets a the promoter region of a gene selected from the group consisting of ASCL1, BRN2, MYT1L, NANOG, VEGFA, TERT, IL1B, IL1R2, IL1RN, HBG1, HBG2, and MYOD1.

17. The DNA targeting system of any one of claims 10-16, wherein the at least one gRNA comprises at least one of SEQ ID NOs: 5-40, 65-144, 492-515, 540-563, and 585-625.

18. A DNA targeting system that binds to a dystrophin gene comprising Cas9 and at least one guide RNA (gRNA).

19. The DNA targeting system of claim 18, wherein the at least one gRNA targets an intron of the dystrophin gene.

20. The DNA targeting system of claim 18, wherein the at least one gRNA targets an exon of the dystrophin gene.

21. The DNA targeting system of claim 18, wherein the at least one guide RNA comprises at least one of SEQ ID NOs: 5-40, 65-144, 492-515, 540-563, and 585-625.

22. The DNA targeting system of any one of claims 11-21, wherein the DNA targeting system comprises between one and ten different gRNAs.

23. An isolated polynucleotide encoding the fusion protein of any one of claims 1-10 or the DNA targeting system of any one of claims 11-22.

24. A vector comprising the isolated polynucleotide of claim 23.

25. A cell comprising the isolated polynucleotide of claim 23 or the vector of claim 24.

26. A method of modulating mammalian gene expression in a cell, the method comprising contacting the cell with the fusion protein of any one of claims 1-10, the DNA targeting system of claims 11-22, the isolated polynucleotide of claim 23, or the vector of claim 24.

27. The method of claim 26, wherein the gene expression is induced.

28. A method of transdifferentiating or inducing differentiation of a cell, the method comprising contacting the cell with the fusion protein of any one of claims 1-10, the DNA targeting system of any one of claims 11-17, the isolated polynucleotide of claim 23, or the vector of claim 24.

29. The method of claim 28, wherein the cell is a fibroblast cell or an induced pluripotent stem cells.

30. The method of claim 29, wherein the fibroblast cell is trandifferentiated into a neuronal cell or a myogenic cell.

31. The method of any one of claims 28-30, wherein the DNA targeting system of any one of claims 11-20 is contacted with the cell and at least one gRNA targets a promoter region of at least one gene selected from the group consisting of ASCL1, BRN2, MYOD1, and MYT1L.

32. The method of claim 31, wherein the DNA targeting system comprises at least one gRNA that targets the promoter region of the ASCL1 gene and at least one gRNA that targets the promoter region of the BRN2 gene.

33. The method of claim 31 or 32, wherein the DNA targeting system comprises between one and twenty different gRNAs.

34. The method of claim 33, wherein the DNA targeting system comprises 8 or 16 different gRNAs.

35. The method of any one of claims 28-34, wherein the DNA targeting system comprises dCas9-VP64.

36. The method of any one of claims 28-35, wherein the DNA targeting system is delivered to the cell virally or non-virally.

37. A method of correcting a mutant gene in a cell, the method comprising administering to a cell containing the DNA targeting system of any one of claims 11-22, the isolated polynucleotide of claim 23, or the vector of claim 24.

38. The method of claim 37, wherein the correction of the mutant gene comprises homology-directed repair.

39. The method of claim 38, further comprising administering to the cell a donor DNA.

40. The method of claim 37, wherein the mutant gene comprises a frameshift mutation which causes a premature stop codon and a truncated gene product.

41. The method of claim 40, wherein the correction of the mutant gene comprises nuclease mediated non-homologous end joining.

42. The method of claim 37, wherein the correction of the mutant gene comprises a deletion of a premature stop codon, a disruption of a splice acceptor site, a deletion of one or more exons, or disruption of a splice donor sequence.

43. The method of claim 42, wherein the deletion of one or more exons results in the correction of the reading frame.

44. A method of treating a subject in need thereof having a mutant dystrophin gene, the method comprising administering to the subject the DNA targeting system of any one of claims 11-22, the isolated polynucleotide of claim 23, or the vector of claim 24.

45. The method of claim 44, wherein the subject is suffering from Duchenne muscular dystrophy.

46. A method of correcting a mutant dystrophin gene in a cell, the method comprising administering to a cell containing a mutant dystrophin gene the DNA targeting system of any one of claims 18-22, the isolated polynucleotide of claim 23, the vector of claim 24, or the cell of claim 25.

47. The method of claim 46, wherein the mutant dystrophin gene comprises a premature stop codon, disrupted reading frame via gene deletion, an aberrant splice acceptor site, or an aberrant splice donor site, and wherein the target region is upstream or downstream of the premature stop codon, disrupted reading frame, aberrant splice acceptor site, or the aberrant splice donor site.

48. The method of claim 46, wherein the correction of the mutant dystrophin gene comprises homology-directed repair.

49. The method of claim 48, further comprising administering to the cell a donor DNA.

50. The method of claim 46, wherein the mutant dystrophin gene comprises a frameshift mutation which causes a premature stop codon and a truncated gene product.

51. The method of claim 50, wherein the correction of the mutant dystrophin gene comprises nuclease mediated non-homologous end joining.

52. The method of claim 46, wherein the correction of the mutant dystrophin gene comprises a deletion of a premature stop codon, correction of a disrupted reading frame, or modulation of splicing by disruption of a splice acceptor site or disruption of a splice donor sequence.

53. The method of claim 52, wherein the correction of the mutant dystrophin gene comprises a deletion of exons 45-55 or exon 51.

54. A kit comprising the fusion protein of any one of claims 1-10, the DNA targeting system of claims 11-22, the isolated polynucleotide of claim 23, the vector of claim 24, or the cell of claim 25.

55. A method of modulating mammalian gene expression in a cell, the method comprising contacting the cell with a polynucleotide encoding a DNA targeting system, wherein the DNA targeting system comprises the fusion protein of any one of claims 1-10 and at least one guide RNA (gRNA).

56. The method of claim 55, wherein the DNA targeting system comprises between one and ten different gRNAs.

57. The method of claim 56, wherein the different gRNAs bind to different target regions within the target gene.

58. The method of claim 57, wherein the target regions are separated by at least one nucleotide.

59. The method of claim 57, wherein the target regions are separated by about 15 to about 700 base pairs.

60. The method of claim 56, wherein each of the different gRNAs bind to at least one different target genes.

61. The method of claim 60, wherein the different target genes are located on same chromosome.

62. The method of claim 60, wherein the different target genes are located on different chromosomes.

63. The method of any one of claims 55-62, wherein at least one target region is within a non-open chromatin region, an open chromatin region, a promoter region of the target gene, an enhancer region of the target gene, a transcribed region of the target gene, or a region upstream of a transcription start site of the target gene.

64. The method of any one of claims 55-62, wherein at least one target region is located between about 1 to about 1000 base pairs upstream of a transcription start site of a target gene.

65. The method of any one of claims 55-62, wherein at least one target region is located between about 1 to about 600 base pairs upstream of a transcription start site of a target gene.

66. The method of any one of claims 55-62, wherein the gene expression is induced.

67. The method of any one of claims 55-62, wherein the DNA targeting system comprises two different gRNAs, three different gRNAs, four different gRNAs, five different gRNAs, six different gRNAs, seven different gRNAs, eight different gRNAs, nine different gRNAs, or ten different gRNAs.

68. The method of any one of claims 55-62, wherein at least one guide RNA targets a promoter region of a gene selected from the group consisting of ASCL1, BRN2, MYT1L, NANOG, VEGFA, TERT, IL1B, IL1R2, IL1RN, HBG1, HBG2, and MYOD1.

69. The method of any one of claims 55-68, wherein the at least one guide RNA comprises at least one of SEQ ID NOs: 5-40, 65-144, 492-515, 540-563, and 585-625.

70. The method of any one of claims 55-65, wherein at least one target region is within an intron or an exon of a target gene.

71. A composition for inducing mammalian gene expression in a cell, the composition comprising the fusion protein of any one of claims 1-10 and at least one guide RNA (gRNA).

72. A composition for inducing mammalian gene expression in a cell, the composition comprising an isolated polynucleotide sequence encoding the fusion protein of any one of claims 1-10 and at least one guide RNA (gRNA).

73. The composition of claim 71 or 72, wherein at least one guide RNA targets a promoter region of a gene selected from the group consisting of ASCL1, BRN2, MYT1L, NANOG, VEGFA, TERT, IL1B, IL1R2, IL1RN, HBG1, HBG2, and MYOD1.

74. The composition of claim 71 or 72, wherein the at least one guide RNA comprises at least one of SEQ ID NOs 5-40, 65-144, 492-515, 540-563, and 585-625.

75. A cell comprising the composition of any one of claims 71-74.

76. A kit comprising the composition of any one of claims 71-74 or the cell of claim 74.

77. A kit for inducing mammalian gene expression in a cell, the kit comprising the composition of any one of claims 71-74 or the cell of claim 75.

78. A composition for genome editing in a muscle of a subject comprising a modified adeno-associated virus (AAV) vector and a nucleotide sequence encoding a site-specific nuclease, wherein the muscle is skeletal muscle or cardiac muscle.

79. The composition of claim 78, wherein the modified AAV vector has enhanced cardiac and skeletal muscle tissue tropism.

80. The composition of claim 78 or 79, wherein the site-specific nuclease comprises a zinc finger nuclease, a TAL effector nuclease, or a CRISPR/Cas9 system.

81. The composition of any one of claims 78-80, wherein the site-specific nuclease binds a gene or locus in the cell of the muscle.

82. The composition of claim 81, wherein the gene or locus is dystrophin gene.

83. The composition of anyone of claims 78-82, further comprising a donor DNA or transgene.

84. A method of genome editing in a muscle of a subject, the method comprising administering to the muscle the composition of any one of claims 78-83, wherein the muscle is skeletal muscle or cardiac muscle.

85. The method of claim 84, wherein the genome editing comprises correcting a mutant gene or inserting a transgene.

86. The method of claim 85, wherein correcting a mutant gene comprises deleting, rearranging, or replacing the mutant gene.

87. The method of any one of claim 85 or 86, wherein correcting the mutant gene comprises nuclease-mediated non-homologous end joining or homology-directed repair.

88. A method of treating a subject, the method comprising administering the composition of any one of claims 78-83 to a muscle of the subject, wherein the muscle is skeletal muscle or cardiac muscle.

89. The method of claim 88, wherein the subject is suffering from a skeletal muscle condition or a genetic disease.

90. The method of claim 89, wherein the subject is suffering from Duchenne muscular dystrophy.

91. A method of correcting a mutant gene in a subject, the method comprising administering the composition of any one of claims 78-83 to a muscle of the subject, wherein the muscle is skeletal muscle or cardiac muscle.

92. The method of any one of claims 84-91, wherein the composition is injected into the skeletal muscle of the subject.

93. The method of any one of claims 84-91, wherein the composition is injected systemically to the subject.

94. The method of claim 92, wherein the skeletal muscle is tibialis anterior muscle.

95. A kit comprising the composition of any one of claims 78-83.

96. A modified lentiviral vector for genome editing in a subject comprising a first polynucleotide sequence encoding the fusion protein of any one of claims 1-10 and a second polynucleotide sequence encoding at least one sgRNA.

97. The modified lentiviral vector of claim 96, wherein first polynucleotide sequence is operably linked to a first promoter.

98. The modified lentiviral vector of claim 97, wherein the first promoter is a constitutive promoter, an inducible promoter, a repressible promoter, or a regulatable promoter.

99. The modified lentiviral vector of any one of claims 96-98, wherein the second polynucleotide sequence encodes between one and ten different sgRNAs.

100. The modified lentiviral vector of any one of claims 96-99, wherein the second polynucleotide sequence encodes two different sgRNAs, three different sgRNAs, four different sgRNAs, five different sgRNAs, six different sgRNAs, seven different sgRNAs, eight different sgRNAs, nine different sgRNAs, or ten different sgRNAs.

101. The modified lentiviral vector of claim 100, wherein each of the polynucleotide sequences encoding the different sgRNAs are operably linked to a promoter.

102. The modified lentiviral vector of claim 101, wherein each of the promoters operably linked to the different sgRNAs is the same promoter.

103. The modified lentiviral vector of claim 101, wherein each of the promoters operably linked to the different sgRNAs are different promoters.

104. The modified lentiviral vector of claim 101, wherein the promoter is a constitutive promoter, an inducible promoter, a repressible promoter, or a regulatable promoter.

105. The modified lentiviral vector of any one of claims 95-103, wherein the sgRNA binds to a target gene.

106. The modified lentiviral vector of claim 105, wherein each of the sgRNA binds to a different target region within one target loci.

107. The modified lentiviral vector of claim 105, wherein each of the sgRNA binds to a different target region within different gene loci.

108. The modified lentiviral vector of claim 105, wherein the fusion protein comprises Cas9 protein or iCas9-VP64 protein.

109. The modified lentiviral vector of claim 105, wherein the fusion protein comprises a VP64 domain, a p300 domain, or a KRAB domain.

110. The modified lentiviral vector of claim 108, wherein two or more endogenous genes are transcriptionally activated.

111. The modified lentiviral vector of claim 108, wherein two or more endogenous genes are repressed.

112. A method of activating an endogenous gene in a cell, the method comprising contacting a cell with the modified lentiviral vector of any one of claims 96-111.

113. The method of claim 112, wherein the endogenous gene is transiently activated.

114. The method of claim 112, wherein the endogenous gene is stably activated.

115. The method of claim 112, wherein the endogenous gene is transiently repressed.

116. The method of claim 112, wherein the endogenous gene is stably repressed.

117. The method of claim 112, wherein the fusion protein is expressed at similar levels to the sgRNAs.

118. The method of claim 112, wherein the fusion protein is expressed at different levels to the sgRNAs.

119. The method of claim 112, wherein the cell is a primary human cell.

120. A method of multiplex gene editing in a cell, the method comprising contacting a cell with the modified lentiviral vector of any one of claims 96-107.

121. The method of claim 120, wherein the multiplex gene editing comprises correcting at least one mutant gene or inserting a transgene.

122. The method of claim 121, wherein correcting a mutant gene comprises deleting, rearranging, or replacing the at least one mutant gene.

123. The method of any one of claim 120 or 121, wherein correcting the at least one mutant gene comprises nuclease-mediated non-homologous end joining or homology-directed repair.

124. The method of claim 120, wherein the multiplex gene editing comprises deleting at least one gene, wherein the gene is an endogenous normal gene or a mutant gene.

125. The method of claim 124, wherein the multiplex gene editing comprises deleting at least two genes.

126. The method of claim 124, wherein the multiplex gene editing comprises deleting between two and ten genes.

127. A method of modulating gene expression of at least one target gene in a cell, the method comprising contacting a cell with the modified lentiviral vector of any one of claims 96-107.

128. The method of claim 127, wherein the gene expression of at least two genes are modulated.

129. The method of claim 127, wherein the gene expression of between two genes and ten genes are modulated.

130. The method of claim 127, wherein the gene expression of the at least one target gene is modulated when gene expression levels of the at least one target gene are increased or decreased compared to normal gene expression levels for the at least one target gene.
